(19) United States
(12) Patent Application Publication
(10) Pub. No.: US 2022/0401553 A1
(43) Pub. Date: Dec. 22, 2022

(54) PARAPOXVIRUS FOR CONDITIONING FOR AND TREATMENT OF CORONAVIRUS INFECTIONS

(71) Applicant: AiCuris GmbH & Co. KG, Wuppertal (DE)

(72) Inventors: Daniela Paulsen, Huizen (NL); Alexander Birkmann, Wuppertal (DE); Peter Lischka, Düsseldorf (DE); Tamara Pfaff, Düsseldorf (DE); Holger Zimmermann, Wuppertal (DE)

(21) Appl. No.: 17/765,760

(22) PCT Filed: May 10, 2021
(86) PCT No.: PCT/EP2021/062250

§ 371 Date: Mar. 31, 2022

(30) Foreign Application Priority Data
May 8, 2020 (EP) 20173670.9

(51) Int. Cl. A61K 39/275; A61K 45/06; A61P 31/14

(52) U.S. Cl. CPC A61K 39/275; A61K 45/06; A61P 31/14; A61K 2039/521

(57) ABSTRACT

The present invention relates to the treatment of coronavirus infections and to preparing subjects for such an infection by administering a parapoxvirus. This treatment is to assist the immune system in combatting the virus and to thereby prevent and to ameliorate symptoms of coronavirus disease.

### FIELD OF THE INVENTION

The present invention relates to the treatment of coronavirus infections and to preparing subjects for such an infection by administering a parapoxvirus. This treatment is to assist the immune system in combatting the virus and to thereby prevent and to ameliorate symptoms of coronavirus disease.

### BACKGROUND OF THE INVENTION

Coronaviruses are enveloped, positive-sense single-stranded RNA viruses, which cause diseases in mammals and birds. In humans, they cause respiratory infections, including the common cold, although some viruses such as SARS and MERS coronaviruses can be lethal. Novel coronaviruses emerge periodically in different areas around the world. SARS-CoV (severe acute respiratory syndrome coronavirus) occurred in 2002 and caused 916 deaths during the epidemic, having a mortality rate of about 12%. MERS-CoV (middle east respiratory syndrome coronavirus) emerged in 2012, with 543 dead reported and a mortality rate of about 39%.

Recently, an outbreak of a coronavirus designated SARS-CoV-2 began in Wuhan, China, and has since spread all over the world. The illness caused by SARS-CoV-2, COVID-19 (coronavirus disease 2019) was declared to be a pandemic by the WHO on Mar. 11, 2020. COVID-19 ranges in severity from asymptomatic or mild to severe, and a significant portion of patients with clinically evident infection develop severe disease. The case fatality rate varies by country and is thought to be between 2% and 3%, but the true mortality rate is uncertain, because the total number of cases is unknown. While SARS-CoV-2 apparently has a lower mortality rate than SARS-CoV and MERS-CoV, it appears to be more infectious and has been able spread around the world, with around 3.66 million confirmed cases and 257,000 confirmed deaths by May 6, 2020 and presumably many more unconfirmed ones. The knowledge of COVID-19 is incomplete and evolving.

The recurrent coronavirus epidemics show that these viruses continue to threaten the world as they emerge spontaneously, spread easily and can have severe consequences on human life, including not only health issues but also dramatic social and economic impacts. Therefore, there is a great medical need for prevention and treatment. However, since coronaviruses are known to mutate and recombine often, they present an ongoing challenge for disease control. Neither vaccines nor antiviral drugs for preventing or treating human coronavirus infections are currently available. A vast number of antivirals are being tested around the world for an effect on coronavirus, with promising candidates failing.

Unexpectedly, the inventors found parapoxvirus to be useful for conditioning subjects for and treating coronavirus infections. Parapoxvirus ovis (PPVO) causes acute dermal infections in goat and sheep, and while it leads to no serious disease in humans, it induces a complex innate and adaptive immune response in humans and treats infections with some specific viruses such as HBV (WO 2019/048640 A1). However, the immune response induced by parapoxvirus is complex and not necessarily useful for treating viral infections in general. Therefore, parapoxvirus could not have been expected to have utility in conditioning subjects for and treating coronavirus infections.

### SUMMARY OF THE INVENTION

In a first aspect, the present invention relates to a parapoxvirus agent for use in (i) conditioning a subject for a coronavirus infection or (ii) treating a coronavirus infection in a subject. In a second aspect, the invention relates to a composition or a kit comprising a parapoxvirus agent and a coronavirus agent. In a third aspect, the present invention relates to a pharmaceutical composition or a kit comprising a parapoxvirus agent for the medical use according to the invention, wherein the pharmaceutical composition or the kit comprises a parapoxvirus agent and preferably a further medicament.

### LEGENDS TO THE FIGURES

FIG. 1: Stimulation of inactivated parapoxvirus (iPPVO) induced antiviral activity against SARS-CoV-2 that was enhanced by combinatorial approaches. Whole-blood cultures were stimulated with iPPVO+/−5 μg/ml ConA. Where no ConA was added, PBS was added instead. iPPVO vehicle+/−ConA or PBS+/−ConA, respectively, served as controls. 5 μl/ml ConA was tested as a co-stimulus. Cultures were incubated for 3 days and supernatants were harvested. The supernatant was transferred to Vero-eGFP cells which were 2-4 hours later infected with SARS-CoV-2 to determine the antiviral activity of the respective supernatant samples and incubated for 5 days. eGFP fluorescence as a measure for living cells was analyzed using the ImageJ software. The mean of the cell control to which no virus was added served as positive control (CC mean) and was set to 100% (see upper dotted line). Cells infected with SARS-CoV2 but not treated gave the highest possible destruction of living cells and served as negative control given as the mean virus control (VC mean). The antiviral activity of the samples was calculated relative to the positive control CC mean and given in %.

FIG. 2: iPPVO D1701 and iPPVO NZ2 stimulate antiviral activity against SARS-CoV-2. Whole-blood cultures were stimulated with iPPVO. iPPVO vehicle served as a negative control. iPPVO D1701 served as positive control. Cultures were incubated for 3 days and supernatants were harvested. The supernatant was transferred to Vero-eGFP cells which were 2-4 hours later infected with SARS-CoV-2 to determine the antiviral activity residing in the respective supernatant samples and incubated for 5 days. eGFP fluorescence as a measure for living cells which have been protected against SARS-CoV-2 due to the antiviral activity of cytokines in the respective supernatant was measured using the ImageJ software. The antiviral activity of the positive control was set to 100% (see upper dotted line). The antiviral activity of the samples was calculated relative to the positive control and given in %. The lower dotted line marks the value of the negative control.

FIG. 3: Lung titres of infectious SARS-CoV-2 particles in hamsters treated with iPPVO NZ2. Each dot represents an animal of the respective group. Horizontal lines represent means.

FIG. 4: Survival of mice treated with iPPVO D1701 and infected with SARS-CoV-2. Animals reaching humane endpoints were euthanized. Overlapping lines are partially offset for better readability. Statistical evaluation was performed by Log-rank (Mantel-Cox) test, * indicates significant difference at p≤0.05.

FIG. 5: Body weight of mice treated with iPPVO D1701 and infected with SARS-CoV-2. Animals reaching humane endpoints were euthanized and are marked by a cross (†). Data are presented as means±standard errors. Statistical evaluation was performed by Multiple t-test (Holm-Sidak corrected), ** indicates significant difference at p≤0.01.

FIG. 6: Clinical score of mice treated with iPPVO D1701 and infected with SARS-CoV-2. Animals reaching humane endpoints were euthanized and are marked by a cross (†). Data are presented as means±standard errors. Statistical evaluation was performed by Multiple t-test (Holm-Sidak corrected), * and ** indicate significant difference at p≤0.05 and p≤0.01, respectively.

FIG. 7: Quantification of SARS-CoV-2 viral loads in mice lungs and brains. Data points represent the viral copy number of each animal with geometric mean of each group. Each point represents one mouse. Reduction in viral load of iPPVO treated mice (left) is shown in fold reduction compared to placebo control (right). Statistical evaluation of the data was performed by Mann-Whitney U test in comparison to placebo control (ns: non-significant, **: p≤0.01).

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturers' specifications, instructions etc.), whether supra or infra, is hereby incorporated by reference in its entirety.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments, which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step. In preferred embodiments, “comprise” can mean “consist of”. As used in this specification and the appended claims, the singular forms “a”, “an”, and “the” include plural referents, unless the content clearly dictates otherwise.

In a first aspect, the present invention relates to a parapoxvirus agent for use in (i) conditioning a subject for a coronavirus infection or (ii) treating a coronavirus infection in a subject.

### Parapoxvirus Agent

The parapoxvirus is, in a preferred embodiment, Parapoxvirus ovis (PPVO). This includes any PPVO strain, preferably, any strain of the species PPVO as classified by the International Committee on Taxonomy of Viruses (ICTV). Exemplary strains are NZ2, NZ7, NZ10, D1701, OV/20, OV/7, OV/C2, OV/mi-90, OV-Torino, SAGO, Bo29, orf11, Greek orf strain 155, and Greek orf strain 176. Preferred strains are NZ2 and D1701, especially NZ2.

The parapoxvirus agent is, in a preferred embodiment,

(a) the parapoxvirus itself or a fragment thereof, or
(b) an agent that comprises genetic information encoding for (a).

In a more preferred embodiment, it is

(i) a live parapoxvirus virion,
(ii) an inactivated parapoxvirus virion,
(iii) a fragment of (i) or (ii),
(iv) a nucleic acid encoding for any of (i) to (iii),
(v) a vector comprising the nucleic acid of (iv), or
(vi) a cell comprising the nucleic acid of (iv) or the vector of (v).

The live parapoxvirus virion may or may not be attenuated. Attenuated parapoxvirus virions are known in the art, e.g. virions lacking a virulence gene such as vegf-e and/or gif. The fragment can be recognized by the immune system and preferably stimulates an immune response (i.e. it is an antigen and preferably an immunogen) and can be any fragment, although preferably it is a fragment that is bound by a human pattern recognition receptor (PRR), e.g. TLR9. In one embodiment, the parapoxvirus fragment is a parapoxvirus protein.

Exemplary fragments are (exemplary coding sequence (nucleotide positions of SEQ ID NO: 3 representing strain NZ2) in parenthesis preceding the fragment): (3 to 539) ORF L1, (781 to 449) ORF L2r, (1933 to 1664) ORF L3r, (3269 to 2790) ORF L4r, (2799 to 3851) ORF L5, (2962 to 3753) ORF L6, (3784 to 3122) ORF L7r, (4341 to 4129) ORF L8r, (4904 to 4428) ORF lar, (6517 to 4970) ORF 1r, (8042 to 6684) ORF 2r, (9989 to 8070) ORF 3r, (11195 to 10062) ORF 4r, (11493 to 11227) ORF 5r, (11802 to 12038) ORF 6, (12358 to 12080) ORF 7r, (13980 to 12364) ORF 8r, (14826 to 14053) ORF gar, (15080 to 15394) ORF 10, (16838 to 15423) ORF 11r, (19021 to 16847) ORF 12r, (19704 to 19156) ORF 13r, (20314 to 19736) ORF 14r, (20401 to 22101) ORF 15, (22125 to 22940) ORF 6, (23003 to 23866) ORF 17, (26908 to 23873) ORF 18r, (26926 to 27213) ORF 19, (27626 to 27216) ORF 20r, (29754 to 27616) ORF 21r, (32217 to 29800) ORF 22r, (33380 to 32418) ORF 23r, (33602 to 33393 ORF 24r, (34466 to 33612) ORF 25r, (34735 to 34502) ORF 26r, (35905 to 34739) ORF 27r, (37194 to 35905) ORF 28r, (37200 to 39248) ORF 29; 41037 to 39229) ORF 30r, (41374 to 42066) ORF 31, (42336 to 41731) ORF 32r, (42407 to 41997) ORF 33r, (42410 to 43765) ORF 34, (43770 to 43958) ORF 35, (43980 to 44534) ORF 36, (45727 to 44537) ORF 37r, (45760 to 46557) ORF 38, (46567 to 47568) ORF 39, (47572 to 48303) ORF 40, (48352 to 48621) ORF 41, (49887 to 48634) ORF 42r, (49917 to 50793) ORF 43, (50719 to 51102) ORF 44, (51059 to 51511) ORF 44a, (51584 to 52591) ORF 45, (52509 to 53066) ORF 46, (53523 to 53023) ORF 47r, (53607 to 57473) ORF 48, (58070 to 57528) ORF 49r, (57700 to 58662) ORF 50, (59674 to 58673) ORF 51r, (62089 to 59678) ORF 52r, (62198 to 62881) ORF 53, (62909 to 63862) ORF 55, (63858 to 64271) ORF 56, (64309 to 66831) ORF 57, (67266 to 66799) ORF 58r, (67803 to 67273) ORF 58ar, (67915 to 68607) ORF 59, (68624 to 70984) ORF 60, (70994 to 72898) ORF 61, (72938 to 73507) ORF 62, (73540 to 74211) ORF 63, (76120 to 74207) ORF 64r, (76749 to 76186) ORF 65r, (77698 to 76799) ORF 66r, (79343 to 77709) ORF 67r, (79816 to 79367) ORF 68r, (80529 to 79858) ORF 69r, (80774 to 80529 ORF 70r, (82815 to 80788) ORF 71r, (83835 to 82834) ORF 72r, (83874 to 85583) ORF 73, (85535 to 84402) ORF 74r, (88096 to 85574) ORF 75r, (87759 to 88667) ORF 76, (88920 to 88642) ORF 77r, (91652 to 88938) ORF 78r, (91667 to 92674) ORF 79, (93466 to 92681) ORF 80r, (93761 to 93486) ORF 81r, (94060 to 93788) ORF 82r, (94238 to 94080) ORF 83r, (94508 to 94242) ORF 84r, (95571 to 94498) ORF 85r, (96187 to 95600) ORF 86r, (96202 to 97665) ORF 87, (97915 to 97643) ORF 88r, (98251 to 99537) ORF 89, (99537 to 99974) ORF 90, (100001 to 101140) ORF 91, (101168 to 104650 ORF 92, (106354 to 104795) ORF 93r, (107947 to 106400) ORF 94r, (108256 to 107990) ORF 95r, (108719 to 108300) ORF 96r, (109679 to 108738) ORF 97r, (109861 to 109682) ORF 98r, (110830 to 10033) ORF 99r, (110208 to 110417) ORF 100, (110469 to 110651) ORF 100a, (110915 to 111397) ORF 101, (111419 to 111913 ORF 102, (111949 to 112485) ORF 103, (112593 to 113450) ORF 104, (113323 to 112967) ORF 105r, (113526 to 114152) ORF 106, (114199- to 115236) ORF 107, (115353 to 115787 ORF 108, (115859 o 116551) ORF 109, (116729 to 117523) ORF 110, (117572 to 117114) ORF 111r, (117423 to 118085) ORF 12, (118968 to 118375) ORF 114r, (118508 to 119119) ORF′ 115, (119588 to 120202) ORF 116, (120314 to 21231) ORF 117, (121380 to 123920) ORF 118, (121288 to 122256) ORF 119, (122350 to 123924) ORF 120, (123962 to 125566) ORF 121, (125193 to 124591) ORF 122r, (125689 to 123935 ORF 123r, (123839 to 123297) ORF 123ar, (125652 to 126170) ORF 124, (126121 to 125699) ORF 125r, (126279 to 127769) ORF 126, (127851 to 128408) ORF 127, (128520 to 130076) ORF 128, (130105 to 131700) ORF 129, (131790 to 133283) ORF 130, (133246 to 133920) ORF 131, (133972 to 134370) ORF 132, (134418 to 134693) ORF 133a, (134402 to 134992) ORF R1, (134853 to 134419) ORF R2r, (135628 to 135897) ORF R3, (136780 to 137112) ORF R4, and (137558 to 137022) ORF R5r. The group of fragments includes homologs of the exemplary fragments listed above, i.e. homologous fragments of another parapoxvirus (e.g. species or strains as described above). It also includes functional variants of the exemplary fragments listed above (the function being as defined for the fragments generally above). Thus, the group of fragments includes variants with at least 80%, 85%, 90%, 95%, 96%, 97%, 98% or preferably at least 99% sequence identity to those fragments encoded by the exemplary coding sequences of SEQ ID NO: 3 listed above.

The inactivated parapoxvirus virion can be obtained by any means known in the art suitable for inactivating enveloped viruses, for example by physical inactivation (e.g. exposure to heat, specifically pasteurization, or UV light) or by chemical inactivation (e.g. by exposure to low pH, to a detergent or to an inactivation agent such as ethyleneimine, binary ethyleneimine, formaldehyde, glutaraldehyde, 2,2′-dithiodipyridine, or beta-propiolactone). In a preferred embodiment, the inactivated parapoxvirus virion is an ethyleneimine- or binary ethyleneimine-inactivated virion, preferably a binary ethyleneimine-inactivated virion.

The parapoxvirus agent may comprise genetic information (DNA or RNA) encoding for a heterologous (i.e. not of parapoxvirus) antigen, and/or an immunomodulator, or it may alternatively not comprise genetic information encoding for a heterologous antigen and/or an immunomodulator. In one embodiment, it does not comprise genetic information encoding for a heterologous antigen, but it optionally comprises genetic information encoding for immunomodulator.

In a preferred embodiment, the parapoxvirus agent is a wildtype parapoxvirus. Preferably it is a PPVO virion, more preferably an inactivated PPVO virion (iPPVO).

### Modes of Use

The parapoxvirus agent can be used alone or in combination with a further treatment regimen for (i) conditioning a subject for a coronavirus infection or (ii) treating a coronavirus infection in a subject.

In a preferred embodiment, both of the conditioning and the treating, the further treatment regimen can comprise administering a coronavirus agent.

Specifically for the conditioning, the further treatment regimen can comprise administering a further immune stimulatory agent (e.g. an adjuvant, a TLR agonist, a PRR, e.g. a cytosolic PRR such as RIG-I-like receptors including RIG-I, MDA5 and LGP2, or a mitogen, particularly a T cell mitogen or activator such as phytohemagglutinin (PHA), Concanavalin (Con) A, wheat germ agglutinin (WGA), pokeweed mitogen (PWM), a lectin or an anti-CD3 antibody or antigen-binding fragment thereof), or a B cell mitogen or activator such as PWM, lipopolysaccharide (LPS), an anti-CD40 antibody or antigen-binding fragment thereof, or an anti-(B cell antigen receptor) antibody or antigen-binding fragment thereof. The immune stimulatory agent preferably promotes an antiviral cell state. Specifically for the treating, the further treatment regimen can comprise administering one or more further medicaments and/or one or more further therapies (non-medicament/drug treatments) suitable for the treatment of a subject infected by coronavirus or having coronavirus disease. The one or more further medicaments are preferably selected from the group consisting of a further immune stimulatory agent (as exemplified above), an antiviral, an antibiotic, an adjuvant, a glucocorticoid, an antihypertensive drug (e.g. an ACE inhibitor), a hypoglycaemic drug, an anti-shock drug, and a drug suitable for treating one or more symptoms of coronavirus disease and/or to accompany the one or more further therapies (e.g. a pain reliever, a sedative, an anti-fever drug, an anti-inflammatory drug such as IL-10, cough medication or an immunosuppressant). The one or more further therapies are preferably selected from the group consisting of oxygen therapy and extra-corporeal organ support (ECOS). Oxygen therapy can be passive (i.e. relying on breathing of the subject) or active (i.e. delivering oxygen to the lungs without relying on breathing of the subject). Passive therapies are non-pressurized oxygen therapy and pressurized oxygen therapy. Active therapies are mechanical ventilation and extra-corporeal membrane oxygenation (ECMO, also a specific form of ECOS). Examples for ECOS include ECMO, continuous renal replacement therapy (CRRT), hemofiltration (HF) and hemoperfusion (HP) e.g. to remove inflammatory cytokines, and left ventricular assistance.

Specific examples of further medicaments for conditioning and treating are: Ivermectin, TMPRSS2 inhibitors (e.g. ammonium chloride, serine protease inhibitor camostat mesylate) optionally together with a CatB/L inhibitor (e.g. E-64d), Favipiravir, Pimodivir, Baloxavir optionally together with Marboxil, IL-6 blockers (e.g. sarilumab, tocilizumab), Sarilumab, Zitivekumab, lopinavir optionally together with ritonavir, chloroquine, hydroxychloroquine, Remdesivir, α-Ketoamides (e.g. cyclopentylmethyl, cyclohexylmethyl or α-Ketoamide 13b), Thalidomide, Maraviroc, Tenofovir optionally together with Disoproxil, Dolutegravir, Nelfinavir, Raltegravir, Tipranavir and Type I interferons (IFN-I, specifically IFN-β1). Preferred examples are Remdesivir and Thalidomide.

The parapoxvirus agent can be administered by any route, including but not limited to the intravenous, intramuscular, oral, parenteral, topical, intradermal, or subcutaneous route. In a specific embodiment, it can be administered directly to the respiratory tract, preferably to the lower respiratory tract, more preferably to the lung (pulmonary delivery). As such, it can be administered as an aerosol and/or via inhalation.

The dose of the parapoxvirus agent (in fact of all agents described herein, including a further immune stimulatory agent, a further medicament and a coronavirus agent) is a pharmaceutically effective dose as can be determined by the skilled person. For instance, in case of the agent being a virion (live or inactivated), it can be in the range of from 10⁶ to 10¹⁰ particles. The dose may be administered once or more than once, for example over a period of <12 weeks, <6 weeks, <4 weeks, <2 weeks, or <1 week.

### Coronavirus

The coronavirus is, in a preferred embodiment, an alpha-coronavirus or a beta-coronavirus. More preferably, it is a respiratory coronavirus, i.e. a coronavirus that causes a respiratory disease. Examples of alpha-coronaviruses are HCoV-229E and CoV-NL63, and examples of beta-coronaviruses are SARS-CoV, SARS-CoV-2, MERS-CoV, HCoV-HKU1, and HCoV-0C43. It is preferred that the coronavirus is a MERS or a SARS coronavirus, wherein preferably the SARS coronavirus is a virus of the species “severe acute respiratory syndrome-related coronavirus” as classified by the International Committee on Taxonomy of Viruses (ICTV). In the most preferred embodiment, it is SARS-CoV-2.

The skilled person appreciates that coronaviruses mutate and that new strains of SARS-CoV-2 are isolated daily, with 461 strains having been isolated by Apr. 6, 2020. Thus, in terms of a sequence definition, a preferred virus of the invention has a genome comprising or consisting of a sequence according to SEQ ID NO: 1 (original strain of the 2019 outbreak “Severe acute respiratory syndrome coronavirus 2 isolate Wuhan-Hu-1”, NCBI Reference Sequence NC 045512.2, version of Mar. 30, 2020) or a variant thereof with at least 80%, 85%, 90%, 95%, 96%, 97%, 98% or preferably at least 99% sequence identity. Alternatively or in addition, a preferred virus of the invention has a genome comprising or consisting of a sequence according to SEQ ID NO: 2 (a SARS-CoV-2 consensus sequence of 153 genome assemblies) or a variant thereof with at least 80%, 85%, 90%, 95%, 96%, 97%, 98% or preferably at least 99% sequence identity. It is preferred that the variant is classified as a strain of SARS-CoV-2 by the International Committee on Taxonomy of Viruses (ICTV).

### Coronavirus Agent

A coronavirus agent generally is an agent that is suitable for treating a coronavirus infection. It can be (a) the coronavirus itself or a fragment thereof which is recognized by and preferably stimulates an immune response (e.g. an antigen and preferably immunogen), (b) an agent that comprises genetic information encoding for (a), or (c) an agent which interferes with the infection of a cell by the coronavirus or with the replication of the coronavirus within a cell (in a therapeutic setting: is capable upon administration of an effective amount thereof to a subject infected with coronavirus to interfere with the infection of a cell by the coronavirus or with the replication of the coronavirus within a cell). The coronavirus agent is preferably selected from the group consisting of

(i) an inactivated coronavirus,
(ii) an antigenic, preferably immunogenic fragment of coronavirus (e.g. the spike protein or a fragment thereof),
(iii) a nucleic acid encoding for (i) or (ii),
(iv) a vector comprising the nucleic acid of (iii),
(v) a cell comprising the nucleic acid of (iii) or the vector of (iv),
(vi) an antibody or an antigen-binding fragment thereof binding to coronavirus or a fragment thereof, preferably the envelope,
(vii) a nucleotide/nucleoside analogue, preferably coronavirus-specific,
(viii) a coronavirus-specific RNAi molecule,
(ix) a small molecule, an antibody or an antigen-binding fragment thereof, binding to a site of protein-protein interaction (PPI) between a coronavirus protein and a subject protein, and
(x) blood plasma of a subject that has recovered from a coronavirus infection, preferably from coronavirus disease.

A coronavirus can be inactivated as described for parapoxvirus above.

The PPI of (ix) is preferably an interaction of a SARS-CoV-2 protein or homologue thereof of another coronavirus and a subject protein (Uniprot protein ID of each subject protein is <name recited below>_HUMAN) selected from
SARS-CoV2 E and AP3B1, BRD4, BRD2, CWC27, ZCH18 or CTL2;
SARS-CoV2 M and MPPB, YIF1A, AT1B1, ACADM, ETFA, STOM, VKGC, VATA, PSMD8, REEP5, MPPA, AN06, PREP, ZNT9, FAKD5, ZNT7, GCP3, COQ8B, SAAL1, REEP6, INT4, ODC, GCP2, SY™, RTN4, FA8A1, AASS, AKP8L, AAR2 or BZW2
SARS-CoV2 N and U3IP2, PABP1, CSK22, CSK2B, G3BP1, PABP4, LARP1, FA98A, SNIP1, RENT1, MOV10, G3BP2, DDX21, RBM28 or RL36;
SARS-CoV2 Spike and ACE2, GOGA7 or ZDHC5;
SARS-CoV2 nsp1 and DPOLA, PRI1, PRI2, DPOA2, GT251 or PKP2;
SARS-CoV2 nsp10 and AP2A2, ALR, ERGI1, AP2M1 or GRPE1;
SARS-CoV2 nsp1 l and TBCA;
SARS-CoV2 nsp12 and SBNO1, BCKD, AKAP8, MYCB2, SLUT, RIPK1, UBP2L, TYSD1, PDZ11, PRC2B, UBAP2, ZN318, CRTC3, UBP54, Z3H7A, LAR4B, RBM41, HTF4, PPIL3 or PKHA5;
SARS-CoV2 nsp13 and TBKB1, CTRO, HSBP1, PCNT, FR1OP, KAP2, KAPCA, KAP3, RADI, CENPF, TLE1, TLE3, TLE5, GOGA3, GOGA2, GOGB1, GRAP1, CE350, MYOME, CP135, CEP68, CNTRL, RB612, GCC2, CLIP4, NIN, CE112, MIPO1, UBP13, GCC1, JKIP1, CK5P2, AKAP9, GORS1, FYCO1, CA050, CP250, TBK1, HOOK1 or NINL;
SARS-CoV2 nsp14 and AGAL, IMDH2 or SIR5;
SARS-CoV2 nsp15 NTF2, ARF6 or RNF41;
SARS-CoV2 nsp2 and S27A2, IF4E2, NCPR, GDS1, WASC4, FKB15 or GGYF2;
SARS-CoV2 nsp4 and IDE, TIM10, ALG11, P0210, TIM29, DJC11, T10B or TIM9;
SARS-CoV2 nsp5 and HDAC2;
SARS-CoV2 nsp5_C145A and GPX1 or TRM1;
SARS-CoV2 nsp6 and ATP5L, VAS1, SGMR1 or AT133;
SARS-CoV2 nsp7 and ADAS, CYB5B, ACSL3, NB5R3, RALA, COMT, RAB5C, RAB7A, RAB8A, RAB2A, RAB10, RAB14, RHOA, RAB1A, GBB1, GBG5, LMAN2, MOGS, TOIP1, MARC1, QS0X2, HS2ST, NDUF2, SCPDL, SCRB1, NAT14, DCAKD, F162A, TIM14, SELS, PGES2 or RAB18;
SARS-CoV2 nsp8 and MPP10, SRP72, ATE1, NSD2, SRP19, SRP54, RT25, DDX10, LARP7, MEPCE, NGDN, EXOS8, SYNM, NOL10, CCD86, SPCS, EXOS5, EXOS3, AATF, HECD1, RT02, RT05, EXOS2 or RT27;
SARS-CoV2 nsp9 and T2FB, FBN1, FBN2, NU214, NUP62, DCAF7, IF4H, NUP54, MIB1, NEK9, ZN503, NUP88, NUP58, MAT2B or FBLN5;
SARS-CoV2 orf10 and PPT1, CUL2, MA7D1, THTPA, ZY11B, TIMBB, RBX1, ELOC or ELOB;
SARS-CoV2 orf3a and HMOX1, TRI59, AR6P6, VPS39, CLCC1, VPS11, SUN2 or ALG5;
SARS-CoV2 orf3b and STML2;
SARS-CoV2 orf6 and NUP98, RAE1L or MTCH1;
SARS-CoV2 orf7a and HEAT3 or MDN1;
SARS-CoV2 orf8 and PLOD2, TOR1A, STC2, TPA, ITB1, CISD3, CO6A1, PVR, DNMT1, LYOX, PCSK6, INHBE, NPC2, MFGM, 0S9, NPTX1, PLGT2, PLGT3, ERO1B, PLD3, FXRD2, CHSS2, PUSL1, EMC1, GGH, ERLEC, I17RA, NGLY1, H6ST2, SDF2, NEUR1, GDF15, TM2D3, ERP44, EDEM3, SILL OFUT1, SMOC1, PKHF2, FXL12, UGGG2, CHPF2, ATS1, HYOU1, FKBP7, ADAMS or FKB10;
SARS-CoV2 orf9b and NHRF1, CHM2A, CSDE1, TOM70, MARK3, MARK2, DPH5, DCTP1, MARK1, PTBP2 or BAGS; and
SARS-CoV2 orf9c and UBXN8, GPAA1, WFS1, MRP1, PAR2, SCAP, D19L1, SGMR2, ZNT6, TAPT1, ERMP1, NLRX1, RETR3, PIGO, FACR2, ECSIT, ALG8, TM39B, GHITM, ACAD9, NFIP2, BCS1, CIA30, TMED5, NDUB9 or PIGS.

An antibody or an antigen-binding fragment thereof to a PPI site can be generated by routine methods. For instance, antibodies or antigen-binding fragments thereof to one of the interacting proteins can be generated and then screened for interfering with the interaction.

The small molecule of (ix) is preferably selected from the group consisting of Loratadine, Daunorubicin, Midostaurin, Ponatinib, Silmitasertib, Valproic Acid, Haloperidol, Metformin, Migalastat, S-verapamil, Indomethacin, Ruxolitinib, Mycophenolic acid, Entacapone, Ribavirin, E-52862, Merimepodib, RVX-208, XL413, AC-55541, Apicidin, AZ3451, AZ8838, Bafilomycin A1, CCT 365623, GB110, H-89, JQ1, PB28, PD-144418, RS-PPCC, TMCB, UCPH-101, ZINC1775962367, ZINC4326719, ZINC4511851, ZINC95559591, 4E2RCat, ABBV-744, Camostat, Captopril, CB5083, Chloramphenicol, Chloroquine, PI4K-IIM inhibitor Compound 10 (Rutaganira, F. U. et al., J. Med. Chem. 59, 1830-1839 (2016)), Cyclophilin inhibitor Compound 2 (Mackman, R. L. et al., J. Med. Chem. 61, 9473-9499 (2018)), CPI-0610, Dabrafenib, DBeQ, dBET6, IHVR-19029, Linezolid, Lisinopril, Minoxidil, ML240, MZ1, Nafamostat, Pevonedistat, PS3061, Rapamycin (Sirolimus), Sanglifehrin A, Sapanisertib (INK128/M1N128), FK-506 (Tacrolimus), Ternatin 4 (DA3), Tigecycline, Tomivosertib (eFT-508), Verdinexor, WDB002, Azacitidine, Selinexor, and Zotatifin (eFT226).

### Coronavirus Infection/Disease

A coronavirus infection is defined by the entry of coronavirus into at least one cell of a subject and its replication in the at least one cell. Preferably, the infection is an infection of the respiratory tract, including the upper respiratory tract (nose and nasal passages, paranasal sinuses, the pharynx, and the portion of the larynx above the vocal folds (cords)) and the lower respiratory tract (portion of the larynx below the vocal folds, trachea, bronchi, bronchioles and the lungs including the respiratory bronchioles, alveolar ducts, alveolar sacs and alveoli). More preferably, it is an infection of the lower respiratory tract, most preferably of the lungs (including one or more of respiratory bronchioles, alveolar ducts, alveolar sacs and alveoli). The infection can further be characterized immunologically by the presence of at least one coronavirus-antigen-specific immune factor, preferably selected from the group consisting of B cells, follicular helper T cells (T_{FH} cells), activated CD4⁺ T cells and CD8⁺ T cells (particularly also CD38⁺HLA-DR⁺), IgM antibodies, and IgG antibodies. Alternatively or in addition, it can be characterized immunologically by a coronavirus-specific cytokine profile, preferably in the blood or at the site of infection as specified above (most preferably in the lung). These profiles can be determined by the skilled person without undue burden, e.g. by taking a tissue sample, e.g. from the lung, infecting it with the coronavirus and determining the expression of cytokines. Examples of coronavirus-specific cytokine profiles are (a) for SARS-CoV: upregulation of one or more of, preferably all of IFN-I (e.g. IFN-β), IFN-II (IFN-γ), IFN-III (IFN-λ1, IFN-λ2, and IFN-λ3), IL-1beta, IL-6, IL-12, IL-8, MCP-1, MIP-1alpha, RANTES, CXCL1, CXCL2, CXCL5, and CXCL9, and preferably no upregulation of CXCL10; and (b) for SARS-CoV-2: upregulation of one or more of, preferably all of IL-6, MCP-1, CXCL1, CXCL5, and/or CXLC10, and preferably no upregulation of any IFN and/or of any one or all of IL-1beta, IL-12, IL-8, MIP-1alpha, RANTES, CXCL2, and CXCL9.

The preferred subject is human. While to the inventors' best knowledge coronavirus infects all humans equally, there are risk factors for contracting a coronavirus infection: (i) Recent (within incubation period, i.e. within the past 27, 24, or 19 days, preferably 14 (CDC, NHC) or 10 (WHO) days) presence in or travel from an area with ongoing spread of coronavirus infection. The area is preferably an ‘Affected Area’ as determined by the WHO. “Recent” can mean within the incubation period of the coronavirus, preferably within the past 27, 24, or preferably 19 days, more preferably 14 days or most preferably 10 days.

(ii) Contact with a person infected with coronavirus, in particular a person having one or more symptoms of coronavirus disease. “Contact” can mean exposed to respiratory droplets of the person (e.g. within range of respiratory droplets in air or in contact with surfaces touched by person or by respiratory droplets).
(iii) Being in an increased-risk-profession, which usually necessitates being in contact with the general public (contact being defined as above) regularly (e.g. at least daily or at least weekly), e.g. being a medical professional, in particular in a hospital, being a care worker (e.g. for children or the elderly) or retail worker (for instance in a supermarket or pharmacy).

A coronavirus infection may or may not cause symptoms of coronavirus disease in a subject. The terms “coronavirus infection” and “coronavirus disease” are distinguished herein by the presence of at least one coronavirus disease symptom. As long as the infection is not accompanied by at least one symptom of coronavirus disease, it (or the subject) is asymptomatic (includes presymptomatic). The term “coronavirus disease” as used herein requires the presence of a coronavirus infection and at least one symptom of coronavirus disease (also referred to herein as “symptomatic infection”).

Coronavirus symptoms include dry cough, fever (≥37.8° C.), runny and/or blocked nose, fatigue, breathing difficulty, pneumonia, organ (e.g. heart, lung, liver and/or kidney) failure, itchy throat, headache, joint pain, nausea, diarrhoea, shivering, lymphophenia, loss of smell and/or loss of taste. Preferably, the coronavirus disease is characterized by the presence of two or more, three or more, or four or more symptoms, preferably including one or two or more of dry cough, fever (≥37.8° C.), breathing difficulty, loss of smell and/or loss of taste.

The coronavirus disease is preferably a respiratory disease (e.g. SARS or MERS), more preferably Covid-19. The coronavirus disease can take a mild (non-severe) or a severe course. A mild course is characterized by the presence of one or more only mild symptoms (i.e. no severe symptoms) of coronavirus disease. Mild symptoms are selected from the group consisting of dry cough, mild fever (≥37.8° C. to <40° C.), runny and/or blocked nose, fatigue, itchy throat, headache, joint pain, nausea, diarrhoea, shivering, lymphophenia, loss of smell and loss of taste. Severe symptoms are selected from the group consisting of breathing difficulty in particular acute respiratory distress syndrome, pneumonia, organ (e.g. heart, lung, liver and/or kidney) failure and high fever (≥40° C.). A severe course is characterized clinically by one or more of (i) the presence of one or more severe symptoms of coronavirus disease, (ii) need for hospitalization, in particular need for admission to intensive care unit (ICU) and/or need for oxygen therapy (as defined above), in particular for a respirator (also referred to as ventilator), and/or (iii) death of the subject. A need for hospitalization is characterized by one or more of: clinical or radiological evidence of pneumonia, acute respiratory distress syndrome, and/or the presence of two or more symptoms including at least fever and persistent cough (with or without sputum), in particular persistent cough (e.g. of at least 3 days). A need for ICU admission is characterized by severe pneumonia and/or one or more severe complications. Severe pneumonia is characterized by tachypnea (age younger than 2 months: 60 or more breaths per minute; age 2 to 11 months: 50 or more breaths per minute, age 1 to 5 years: 40 or more breaths per minute, age >5 years: 30 or more breaths per minute), respiratory distress and/or inadequate oxygenation (e.g. SpO₂ of 93% or less for adults, and 90% or less for children). Severe complications are complications that are lethal when untreated and include, for example, septic shock, acute respiratory distress syndrome and organ failure. Alternatively or in addition, a severe course can be characterized immunologically e.g. by the presence or development of a cytokine storm, in particular in the lung. A coronavirus cytokine storm is characterized by hyperinflammation, preferably by an increase (vs. infected but asymptomatic subject, preferably as after the incubation period, or average or mean of a plurality of such subjects) of one or more of IL-2, IL-7, TNF-alpha, MIP-1alpha, MCP-1, G-CSF and/or CXCL10, wherein the increase can be characterized as excessive and/or uncontrolled. In particular, the cytokine storm is a Covid-19 cytokine storm. Furthermore, the cytokine storm is preferably in the respiratory tract as defined for the infection above (including preferred parts thereof).

While the coronavirus disease can take a severe course in any subject regardless of age or pre-existing condition, there are nevertheless risk factors for having a severe course of coronavirus disease, including:

(i) age, e.g. at least 40, 50, 60, 70, or 80 years with risk increasing with age,
(ii) male gender,
(iii) smoking, and
(iii) having one or more comorbidities, preferably selected from the group consisting of cardiovascular disease, diabetes, respiratory disease (in particular chronic), hypertension, AIDS, cancer, liver disease, kidney disease and lung disease. A particular comorbidity as risk factor is a respiratory disease.

### Conditioning a Subject

The conditioning of a subject is meant to prepare a subject for a coronavirus infection, which is afforded by the immunomodulation resulting in the medical use of the invention. It can also be termed “preparatory treatment”, “prophylactic treatment” or, with respect to the disease resulting from the invention, “preventative treatment” or “preventive treatment”. It is not meant to necessarily prevent a coronavirus infection. As such, the subject is not yet infected by coronavirus.

Thus, the conditioning can have one or more immunological effects or (preferably and) it can have one or more clinical effects, both once a subject is infected. These immunological effects are preferably selected from the group consisting of

(I-i) increasing the number of one or more of coronavirus-antigen-specific immune factors (as defined above) in the subject or in a part of the subject,
(I-ii) inhibiting (i.e. reducing or stopping) coronavirus replication in the subject or in a part of the subject,
(I-iii) reducing the coronavirus load in the subject or in a part of the subject, and
(I-iv) preventing or ameliorating a coronavirus-specific cytokine profile or a cytokine storm (as defined above) in the subject or in a part of the subject.
Therein, the part of the subject is preferably the respiratory tract (upper or preferably lower respiratory tract, more preferably the lung). (I-ii) is preferred over (I-i), and (I-iii) over (I-ii). Further (I-ii) may involve (I-i), and (I-iii) may involve (I-i) and/or (I-ii). (I-iv) is generally desired.

The clinical effects are preferably selected from the group consisting of

(C-i) reducing contagiousness,
(C-ii) delaying the onset of coronavirus disease,
(C-iii) reducing the degree of one or more prospective symptoms of coronavirus disease, preferably of one or more prospective severe symptoms of coronavirus disease,
(C-iv) preventing one or more symptoms of coronavirus disease, preferably one or more severe symptoms of coronavirus disease,
(C-v) preventing a severe course of coronavirus disease, and (C-vi) preventing coronavirus disease.
Therein, (C-ii) is preferred over (C-i), (C-iii) over (C-ii), (C-iv) over (C-iii), (C-v) over (C-iv), and (C-vi) over (C-v).

Prospective symptoms are symptoms that are not yet present but can or will arise. Thus, the reduction of the degree of prospective symptoms refers to a prophylactic treatment of symptoms.

Accordingly, the parapoxvirus agent can be for use in one or more of (I-i) to (I-iv) and/or one or more of (C-i) to (C-vi) in a subject not yet infected by coronavirus. In other words, the parapoxvirus agent is capable upon administration of an effective amount thereof to achieve one or more of (I-i) to (I-iv) and/or one or more of (C-i) to (C-vi).

In a preferred embodiment, the subject to be conditioned is characterized by one or more risk factors for contracting a coronavirus infection and/or by one or more risk factors for having a severe course of coronavirus disease.

In another embodiment, the subject to be conditioned is selected by a method according to the corresponding further aspect of the invention described below.

### Treating a Subject

The treating of a coronavirus infection (i.e. of a subject that is infected) is not meant necessarily to treat or prevent coronavirus disease, although it is preferred that it does. As such, the treating can be of an asymptomatic infection (i.e. of an infected subject not having symptoms) or of coronavirus disease (i.e. of an infected subject having one or more symptoms).

The treating can have one or more immunological effects or (preferably and) it can have one or more clinical effects. These immunological effects are preferably as defined for the conditioning above, for either an asymptomatic infection or for the disease. The clinical effects for an asymptomatic infection are preferably selected from the group consisting of (C-i) to (C-vi) as defined for the conditioning above. The clinical effects for a symptomatic infection (i.e. on coronavirus disease) are preferably selected from the group consisting of

(C-i), (C-vi), both as defined for the conditioning above,
(C-vii) ameliorating one or more (or ideally all) symptoms of coronavirus disease,
(C-viii) preventing, one or more (or ideally all) further symptoms of coronavirus disease, and
(C-ix) ameliorating, preferably preventing, a severe course of coronavirus disease.
Therein, “further symptoms” are those not yet characterizing the symptomatic infection.

Accordingly, the parapoxvirus agent can be for use in one or more of (I-i) to (I-iv) and/or one or more of (C-i) to (C-ix) in a subject infected by coronavirus. In other words, the parapoxvirus is capable upon administration of an effective amount thereof to achieve one or more of (I-i) to (I-iv) and/or one or more of (C-i) to (C-ix). In a preferred embodiment, the parapoxvirus agent is for use in preventing or treating coronavirus disease.

As indicated above, the subject to be treated may be asymptomatic. Alternatively, the subject to be treated may have coronavirus disease. The subject having coronavirus disease may not have any severe symptoms, e.g. it has only one or more mild symptoms. In another embodiment, it may have a severe course of coronavirus disease (excluding (iii) death; preferably (i) one or more severe symptoms). In a preferred embodiment, the subject to be treated (e.g. any of the afore-mentioned subjects except those already having a severe course of coronavirus disease) is characterized by one or more risk factors for having a severe course of coronavirus disease.

In a further preferred embodiment, the subject is characterized by at least one of:

(i) no cytokine storm in the lung, and preferably no cytokine storm at all,
(ii) age up to 75, 70 or preferably 65 years, more preferably 18 to 65 years, and/or
(iii) HScore of 169 or less, preferably 150 or less, 130 or less, or 110 or less,
Preferably, the subject is characterized by (ii) and/or (iii), (ii) and HScore of 169 or less, or (iii) and age 18 to 65 years old.

The HScore generates a probability for the presence of secondary haemophagocytic lymphohistoicytosis (sHLH), a hyperinflammatory syndrome with a cytokine storm profile similar to that of coronavirus disease (Pehta et al., The Lancet Vol 395 Mar. 28, 2020). An HScore of greater than 169 is 93% sensitive and 86% specific for sHLH. The HScore is calculated using the following criteria: temperature (<38.4° C.: 0 points, 38.4-39.4° C.: 33 points, >39.4° C.: 49 points), organomegaly (none: 0 points, hepatomegaly or splenomegaly: 23 points, hepatomegaly and splenomegaly: 38 points), number of cytopenias* (one lineage; 0 points, two lineages: 24 points, three linages: 34 points), triglycerides (<1.5 mmol/1: 0 points, 1.5-4.0 mmol/1: 44 points, >4.0 mmol/1: 64 points), fibrinogen (>2.5 g/L: 0 points, ≤2.5 g/L: 30 points), ferritin (<2000 ng/ml: 0 points, 2000-60000 ng/ml: 35 points, >6000 ng/ml: 50 points), serum aspartate aminotransferase (<30 IU/L: 0 points, ≥30 IU/L: 19 points), haemophagocytosis on bone marrow aspirate (no: 0 points, yes: 35 points), and known immunosuppression† (no: 0 points, yes: 18 points). *Defined as either haemoglobin concentration of 9.2 g/dL or less (≤5.71 mmol/L), a white blood cell count of 5000 white blood cells per mm³ or less, or platelet count of 110000 platelets per mm³ or less, or all of these criteria combined. †HIV positive or receiving long-term immunosuppressive therapy (e.g. one or more of glucocorticoids, cyclosporine and/or azathioprine).

In another embodiment, the subject to be treated is selected by a method according to the corresponding further aspect of the invention described below.

### Further Medical Use

In a further medical use aspect, the invention relates to a parapoxvirus agent for use as a medicament promoting an antiviral (preferably anticoronaviral) cell state in a subject (i) having a coronavirus infection or (ii) characterized by one or more risk factors for contracting a coronavirus infection and/or by one or more risk factors for having a severe course of coronavirus disease.

In one embodiment, the one or more risk factors for contracting a coronavirus infection are selected from factors (i) and (ii) as defined above.

In another embodiment, the one or more risk factors for having a severe course of coronavirus disease comprise at least (ii) and (iii), or (ii) and (iv) as defined above, or comprise at least a comorbidity selected from respiratory disease (in particular chronic) and lung disease. In addition, the age of the subject is preferably at least 60, at least 70, or more preferably at least 80 years.

The phrase “promoting an antiviral cell state” refers to the promotion of the transcription of cellular antiviral genes coding for host defence proteins, e.g. as promoted by IFN-α and/or (3. Preferably it inhibits (i.e. prevents or at least reduces) virus replication in the cell and/or induces apoptosis (to prevent further virus replication in the cell), and more preferably it reduces the amount of infectious virions released by an infected cells compared to a cell in which the antiviral cell state was not promoted by the medicament. Thereby, it stops or at least slows down virus propagation. “Promoting” includes an induction (e.g. for cells not yet in an antiviral state) and a reinforcement (i.e. potentiation) (e.g. for cells already in an antiviral state). Although not limited thereto, the former is preferred for a subject not (yet) having a coronavirus infection, e.g. characterized by one or more risk factors as specified above, and the latter is preferred for a subject having a coronavirus infection.

### Methods of Treatment

The invention further relates to the following methods

A method of administering a parapoxvirus agent to a subject (i) having a coronavirus infection or (ii) characterized by one or more risk factors for contracting a coronavirus infection and/or by one or more risk factors for having a severe course of coronavirus disease.

A method of promoting an antiviral (preferably anticoronaviral) cell state in a subject (i) having a coronavirus infection or (ii) characterized by one or more risk factors for contracting a coronavirus infection and/or by one or more risk factors for having a severe course of coronavirus disease, the method comprising administering a parapoxvirus agent to the subject.

A method of (i) conditioning a subject for a coronavirus infection or (ii) treating a coronavirus infection in a subject, the method comprising administering a parapoxvirus agent to the subject.

A method of treating coronoavirus disease in a subject, the method comprising administering a parapoxvirus agent to the subject.

Definitions and embodiments described below, in particular under the header ‘Definitions and further embodiments of the invention’ apply to the first aspect of the invention.

In a second aspect, the invention relates to a composition or a kit comprising a parapoxvirus agent and a coronavirus agent.

In a preferred embodiment, the composition or kit further comprises one or more pharmaceutically acceptable diluents, carriers, and/or preservatives; and/or it is a pharmaceutical composition or kit. Pharmaceutically acceptable diluents, carriers, and/or preservatives are described below and may in addition thereto include an (e.g. aqueous) medium suitable for structurally maintaining a parapoxvirus, in particular an inactivated parapoxvirus, or lyophilisate of this medium. Specifically, the composition or kit may be for any of the medical uses of the invention described herein.

The composition can be formulated for any route of administration as defined above. Also, it can be formulated for simultaneous or separate administration or for simultaneous or separate release of the parapoxvirus agent and the coronavirus agent.

In a preferred embodiment, the kit comprises a composition of the second aspect.

In another embodiment, the kit comprises a leaflet with instructions for use of the parapoxvirus agent and the coronavirus agent, or of the composition of the second aspect, preferably according to a medical use described herein.

Definitions given and embodiments described with respect to the first aspect apply also to the second aspect, in as far as they are applicable. Also, definitions and embodiments described below, in particular under the header ‘Definitions and further embodiments of the invention’ apply to the composition or kit of the second aspect.

In a third aspect, the invention relates to a pharmaceutical composition or kit for any of the medical uses of the invention described herein, wherein the pharmaceutical composition or kit comprises a parapoxvirus agent and preferably a further medicament. The pharmaceutical composition usually comprises one or more pharmaceutically acceptable diluents, carriers, and/or preservatives.

In a preferred embodiment, the kit comprises a composition of the third aspect.

In another embodiment, the kit comprises a leaflet with instructions for use of the parapoxvirus agent and the further medicament, or of the composition of the third aspect, preferably according to a medical use described herein.

Definitions given and embodiments described with respect to the first and second aspect (e.g. formulation) apply also to the third aspect, in as far as they are applicable. Also, definitions and embodiments described below, in particular under the header ‘Definitions and further embodiments of the invention’ apply to the composition or kit of the third aspect.

In a further aspect, the invention relates to a method of selecting a subject for any of the medical uses of the invention described herein, wherein the method comprises the steps of (i) determining the presence or absence of a cytokine storm in the subject, and (ii) selecting the subject if a cytokine storm is absent.

In one embodiment, step (i) comprises determining the HScore of the subject, and step (ii) comprises selecting the subject if the HScore is 169 or less.

In another embodiment, step (i) comprises detecting in a biological sample of the subject one or more signs of hyperinflammation, and step (ii) comprises selecting the subject if no sign of hyperinflammation is detectable. Hyperinflammation therein is an increased level, e.g. excessive and/or uncontrolled, (vs. infected but asymptomatic subject, preferably as after the incubation period, or average or mean of a plurality of such subjects) of one or more of IL-2, IL-7, TNF-alpha, MIP-1 alpha, MCP-1, G-CSF and/or CXCL10; or of one or more of IL-6, ferritin, platelet count and/or erythrocyte sedimentation rate (ESR). The biological sample is preferably selected from the group consisting of blood or a blood-derived sample (e.g. plasma or serum), sputum, saliva, and a sample derived from the lung (e.g. by bronchoscopy including bronchial lavage, bronchial alveolar lavage, bronchial brushing, and bronchial abrasion).

The method of the fourth aspect is preferably an ex vivo method.

Definitions given and embodiments described with respect to the first, second and third aspect apply also to the fourth aspect, in as far as they are applicable. Also, definitions and embodiments described below, in particular under the header ‘Definitions and further embodiments of the invention’ apply to the method of the fourth aspect.

### Disclaimers

In a select embodiment, one or more of the following disclaimers apply to the invention:
the parapoxvirus is not canine distemper virus (CDV),
the coronavirus is not porcine epidemic diarrhea virus (PEDV), and/or
the subject is not a pig (pig including sow and piglet).

### Definitions and Further Embodiments of the Invention

The specification uses a variety of terms and phrases, which have certain meanings as defined below. Preferred meanings are to be construed as preferred embodiments of the aspects of the invention described herein. As such, they and also further embodiments described in the following can be combined with any embodiment of the aspects of the invention and in particular any preferred embodiment of the aspects of the invention described above.

The term “identity” or “identical” in the context of polynucleotide sequences refers to the number of residues in the two sequences that are identical when aligned for maximum correspondence. Specifically, the percent sequence identity of two sequences is the number of exact matches between two aligned sequences divided by the length of the shorter sequence and multiplied by 100. Alignment tools that can be used to align two sequences are well known to the person skilled in the art and can, for example, be obtained on the World Wide Web, e.g. Needle (EMBOSS) (https://www.ebi.ac.uk/Tools/psa/emboss_needle/), MUSCLE (http://www.ebi.ac.uk/Tools/msa/muscle/), MAFFT (http://www.ebi.ac.uk/Tools/msa/mafft/) or WATER (http://www.ebi.ac.uk/Tools/psa/emboss_water/). The alignments between two sequences may be carried out using default parameters settings, e.g. for Needle preferably MATRIX: BLOSUM62, Gap Open: 10.0, Gap Extend: 0.5, for MAFFT preferably: Matrix: Blosum62, Gap Open 1.53, Gap Extend 0.123, for WATER polynucleotides preferably: MATRIX: DNAFULL, Gap Open: 10.0, Gap Extend 0.5 and for WATER polypeptides preferably MATRIX: BLOSUM62, Gap Open: 10.0, Gap Extend: 0.5. Those skilled in the art understand that it may be necessary to introduce gaps in either sequence to produce a satisfactory alignment. The “best sequence alignment” is defined as the alignment that produces the largest number of aligned identical residues while having a minimal number of gaps. Preferably, it is a global alignment, which includes every residue in every sequence in the alignment.

The term “variant” refers generally to a modified version of the polynucleotide, e.g. a mutation, so one or more nucleotides of the polynucleotide may be mutated. Generally, the variant is functional, meaning e.g. with regard to a virus that the virus is capable of infecting a host cell. The functionality is generally that described for the polynucleotide the variant is from. A “mutation” can be a nucleotide substitution, deletion and/or insertion (“and” may apply if there is more than one mutation). Preferably, it is a substitution.

The term “antigen” refers to any substance, e.g. protein or peptide, that is capable of being bound by an antibody, a B cell receptor and/or a T cell receptor. An antigen comprises at least one epitope preferably comprising at least 8 amino acids and more preferably comprising between 8 and 17 amino acids. The epitope can be a T cell and/or a B cell epitope. A T cell epitope is an epitope that can be presented on the surface of an antigen-presenting cell, where it is bound to an MHC molecule. In humans, professional antigen-presenting cells are specialized to present MHC class II peptides, whereas most nucleated somatic cells present MHC class I peptides. T cell epitopes presented by MHC class I molecules are typically peptides between 8 and 11 amino acids in length, whereas MHC class II molecules present longer peptides, e.g. 13-17 amino acids in length. A B cell epitope is an epitope that is recognised as three-dimensional structures on the surface of native antigens by B cells. Epitopes can be predicted with in silico tools, e.g. the online B- or T-cell prediction tools of the IEDB Analysis Resource.

The term “immunogen” refers to an antigen that is capable of inducing an immune response.

“Pattern recognition receptor” or “PRR”, as used herein, refers to a germline-encoded host sensor which detects a pathogen-associated molecular pattern (PAMP), which is specific for a pathogen, and/or a damage-associated molecular pattern (DAMP), which is specific for components of a host cell that are released during cell damage or death. A PRR can be a membrane-bound PRR (cell-surface PRR) or a cytosolic PRR.

An “adjuvant” is a substance that accelerates, prolongs and/or enhances the quality and/or strength of an immune response to an antigen/immunogen, in comparison to the administration of the antigen alone, thus, reducing the quantity of antigen/immunogen necessary, and/or the frequency of injection necessary in order to generate an adequate immune response to the antigen/immunogen of interest. Examples of adjuvants that may be used in the context of the composition according to the present invention are gel-like precipitates of aluminum hydroxide (alum); AlPO₄; alhydrogel; bacterial products from the outer membrane of Gram-negative bacteria, in particular monophosphoryl lipid A (MPLA), lipopolysaccharides (LPS), muramyl dipeptides and derivatives thereof; Freund's incomplete adjuvant; liposomes, in particular neutral liposomes, liposomes containing the composition and optionally cytokines; non-ionic block copolymers; ISCOMATRIX adjuvant (Drane et al., 2007); unmethylated DNA comprising CpG dinucleotides (CpG motif), in particular CpG ODN with a phosphorothioate (PTO) backbone (CpG PTO ODN) or phosphodiester (PO) backbone (CpG PO ODN); synthetic lipopeptide derivatives, in particular Pam₃Cys; lipoarabinomannan; peptidoglycan; zymosan; heat shock proteins (HSP), in particular HSP 70; dsRNA and synthetic derivatives thereof, in particular Poly I:C; polycationic peptides, in particular poly-L-arginine; taxol; fibronectin; flagellin; imidazoquinoline; cytokines with adjuvant activity, in particular GM-CSF, interleukin-(IL-)2, IL-6, IL-7, IL-18, type I and II interferons, in particular IFN-γ, TNF-α; 25-dihydroxyvitamin D3 (calcitriol); and synthetic oligopeptides, in particular MHC-II-presented peptides. Non-ionic block polymers containing polyoxyethylene (POE) and polyoxypropylene (POP), such as POE-POP-POE block copolymers may be used as an adjuvant (Newman et al., 1998). This type of adjuvant is particularly useful for compositions comprising nucleic acids as active ingredient.

The term “recombinant” refers in particular to a virus that is modified to comprise a heterologous polynucleotide sequence in its genome.

The term “immunomodulator” refers to a drug used to regulate or normalize the immune system by inducing, enhancing, suppressing and/or weakening an immune response in a subject (“and” meaning that some parts of the immune system are selectively induced or enhanced and others are selectively suppressed or weakened). Examples are cytokines, non-cytokine agonists or antagonists of cytokine receptors (e.g. antibodies or small compounds), antibodies or small compounds binding (preferably neutralizing) cytokines, and soluble cytokine receptors (e.g. for trapping cytokines). It does not include an antigen.

The term “vector” as used herein includes any vectors known to the skilled person including plasmid vectors, cosmid vectors, phage vectors such as lambda phage, viral vectors such as adenovirus (Ad) vectors), adeno-associated virus (AAV) vectors, alphavirus vectors (e.g., Venezuelan equine encephalitis virus (VEE), sindbis virus (SIN), semliki forest virus (SFV), and VEE-SIN chimeras), herpes virus vectors, measles virus vectors, pox virus vectors (e.g., vaccinia virus, modified vaccinia virus Ankara (MVA), NYVAC (derived from the Copenhagen strain of vaccinia), and avipox vectors: canarypox (ALVAC) and fowlpox (FPV) vectors), and vesicular stomatitis virus vectors, or virus like particles. As used herein, the term “virus-like particle” or “VLP” refers to a non-replicating, empty viral shell. VLPs are generally composed of one or more viral proteins, such as, but not limited to those proteins referred to as capsid, coat, shell, surface and/or envelope proteins. They contain functional viral proteins responsible for cell penetration by the virus, which ensures efficient cell entry. Methods for producing particular VLPs are known in the art.

The term “mitogen” refers to an agent, e.g. a peptide or protein that induces a cell to begin cell division. The term “cell activator” refers to an agent, e.g. a peptide or protein that binds and cross-links cell receptors, e.g. T cell or B cell receptors.

The term “nucleoside analogue” refers to a nucleoside containing a nucleic acid analogue and a sugar. The term “nucleotide analogue” refers to a nucleotide containing a nucleic acid analogue, a sugar and a phosphate group with 1 to 3 phosphates. The term “nucleic acid analogue” refers to a compound that is structurally similar to RNA and DNA in that it has one or more of phosphate backbone, pentose sugar and/or the nucleobase altered such that it has different base pairing and/or base stacking properties. Examples include peptide nucleic acid (PNA), Morpholino and locked nucleic acid (LNA), as well as glycol nucleic acid (GNA) and threose nucleic acid (TNA).

The term “RNAi molecule” refers to a molecule capable of neutralizing mRNA molecules in a target-sequence-specific manner. Examples include dsRNA, miRNA and siRNA.

Particular preferred pharmaceutical forms for the administration of the agents according to the invention are forms suitable for injectable use and include sterile aqueous solutions or dispersions and sterile powders for the extemporaneous preparation of sterile injectable solutions or dispersion. Typically, such a solution or dispersion will include a solvent or dispersion medium, containing, for example, water-buffered aqueous solutions, e.g. biocompatible buffers, ethanol, polyol, such as glycerol, propylene glycol, polyethylene glycol, suitable mixtures thereof, surfactants or vegetable oils. Infusion or injection solutions can be accomplished by any number of art-recognized techniques including but not limited to addition of preservatives like anti-bacterial or anti-fungal agents, e.g. parabene, chlorobutanol, phenol, sorbic acid or thiomersal. Further, isotonic agents, such as sugars or salts, in particular sodium chloride may be incorporated in infusion or injection solutions.

Preferred diluents of the present invention are water, physiological acceptable buffers, physiological acceptable buffer salt solutions or salt solutions. Excipients which can be used with the various pharmaceutical forms of the pharmaceutical according to the invention can be chosen from the following non-limiting list:
a) binders such as lactose, mannitol, crystalline sorbitol, dibasic phosphates, calcium phosphates, sugars, microcrystalline cellulose, carboxymethyl cellulose, hydroxyethyl cellulose, polyvinyl pyrrolidone and the like,
b) lubricants such as magnesium stearate, talc, calcium stearate, zinc stearate, stearic acid, hydrogenated vegetable oil, leucine, glycerids and sodium stearyl fumarates,
c) disintegrants such as starches, croscaramellose, sodium methyl cellulose, agar, bentonite, alginic acid, carboxymethyl cellulose, polyvinyl pyrrolidone and the like.

The term “pharmaceutically acceptable carrier” refers to any substrate which serves to improve the selectivity, effectiveness, and/or safety of drug administration. Such carriers can be used to control the release of a drug into systemic circulation. This can be accomplished either by slow release of the drug over a long period of time (typically diffusion) or by triggered release at the drug's target by some stimulus, such as changes in pH, application of heat, and activation by light. Carriers can also be used to improve the pharmacokinetic properties, specifically the bioavailability, of many drugs with poor water solubility and/or membrane permeability. A wide variety of drug carrier systems have been developed and studied. Examples include liposomes, polymeric micelles, microspheres, nanoparticles, nanofibers, protein-drug conjugates, erythrocytes, virosomes and dendrimers. Different methods of attaching the drug to the carrier can be used, including adsorption, integration into the bulk structure, encapsulation, and covalent bonding.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The invention is described by way of the following examples which are to be construed as merely illustrative and not limitative of the scope of the invention.

### Example 1

### Aim

Testing the antiviral activity of inactivated parapoxvirus orf (iPPVO) virions against coronavirus. PPVO virions stimulate the innate as well as the adaptive arm of the immune system.

### Approach

In whole blood, APCs (antigen-presenting cells) take up the iPPVO virion, which leads to their activation and the release of cytokines. This in turn activates T cells as well as NK cells which also start to proliferate and release cytokines. After 3 days of incubation the cells are spun down and the supernatant containing soluble factors like cytokines are harvested. These supernatants are assayed for their activity on coronavirus.

### Test Substances

iPPVO of strain D1701 (available from Pfizer); 230 IFN units reconstituted in 2 ml water
iPPVO vehicle (PPVO buffer only, i.e. without virus), negative control
PBS, negative control
Concavalin (Con) A, co-stimulus, stock solution 1 mg/ml PBS=10×(used in well at 5 μg/ml)

### Further Materials

Whole blood of a healthy donor: 9 ml whole blood freshly collected in a Sarstedt Monovette, Li-Heparin
Medium for whole blood incubation: RPMI (500 ml) supplemented with 1% GlutaMAX™ and 0.25 ml Heparin-Sodium (5000 U)
Cells of coronavirus assay: VeroE6-EGFP propagated in growth medium prepared by supplementing DMEM with 10% v/v heat-inactivated FCS and 5 mL sodium bicarbonate 7.5%, and cultured in T150 bottle and split ¼ twice a week. Pen-strep added directly to the T150 bottle at a 1/100 dilution.
Coronavirus assay medium: prepared by supplementing DMEM with 2% v/v heat-inactivated FCS and 5 mL sodium bicarbonate 7.5%.

### Preparation of Samples

Per well in a 96 well round-bottom plate: 120 μl cell medium for whole blood incubation +20 μl ConA or PBS+20 μl iPPVO, iPPVO vehicle or PBS+40 μl whole blood.

The plate was incubated at 37° C., 5% CO₂ for 72 hrs and then centrifuged for 5 min at 230 g. The supernatant was aliquoted to new 96 well plates (plate layout maintained). Sample plates were frozen at −80° C. until further use.

### Coronavirus Assay

Dilution of the samples (test substance as prepared above): 100 μL assay medium were added to wells of a Greiner Bio One 655090 plate and 15 μL were removed again from wells (except from wells for controls of this assay: virus control (VC) controlling for virus replication and effect on cells, and cell control (CC) for controlling cell viability without virus). 15 μL from wells of sample plate was added to wells of which 15 μL medium was removed.

Preparation of the cell suspension: A confluent culture (monolayer) of VeroE6-EGFP in T150 bottle was washed with DPBS and 10 mL trypsine 0.25% trypsine/EDTA was added. The bottle was incubated for 1 minute at room temperature, and trypsine/EDTA was removed except for 2 mL. The bottle was then incubated for 15 minutes at 37° C. After incubation, the cells were resuspended in 10 mL assay medium, passed through a Cell Strainer (FALCON CAT NO 352350) and counted using coulter (3 samples of 10 μL in 10 mL were counted). 3000 cells were then resuspended in 50 μL assay medium and 50 μL of the cell suspension were seeded to each well of the Greiner plate.

Assay: Plates with cell suspension as prepared above were incubated overnight (37° C./5% CO₂), after which SARS-CoV-2 (strain BetaCoV/Belgium/GHB-03021/2020, stock titer 4.8×10⁷ TCID50/ml) was added to all wells (except the cell control wells) at a final dilution of 1/200,0000 in 200 μl/well (3000 cells/well, resulting in an MOI of 0.016 TCID₅₀/cell). The plates were incubated at 37° C./5% CO₂ for 5 days, after which GFP signal was measured using whole-well fluorescence, HCl (High Content Imaging) and the ImageJ software. Reduced EGFP expression is a marker for virus-induced cytopathic activity. See also Ivens et al. (Journal of Virological Methods 129, 2005, 56-63).

### Results

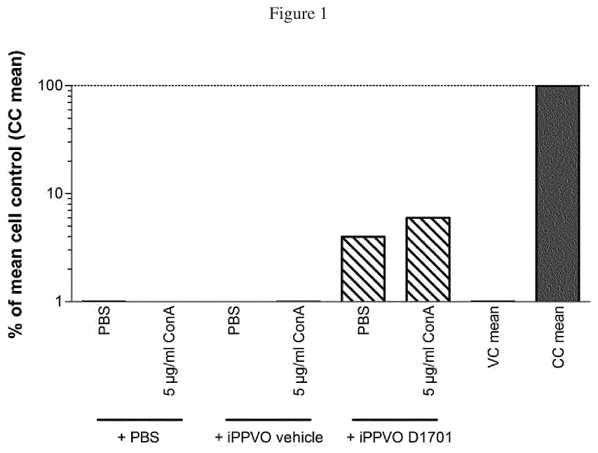

The results are shown in FIG. 1. Cells not infected with coronavirus (CC mean) were viable and expressed eGFP. Cells infected with coronavirus (VC mean) died and no eGFP expression was detectable. Results for PBS and iPPVO vehicle alone were similar to VC mean, i.e. PBS and iPPVO vehicle alone had no effect on cell viability, i.e. against coronavirus, as expected. Also 5 μg/ml ConA alone (in PBS) did not retain eGFP expression, and it also did not affect eGFP expression when given together with iPPVO vehicle as shown by the comparison to ConA alone in PBS. iPPVO D1701 alone (in PBS) also had a clear positive effect on cell viability, and eGFP expression was further increased when iPPVO D1701 was used in combination with 5 μg/ml ConA (which by itself showed no effect on eGFP expression at this concentration). Accordingly, iPPVO and ConA act synergistically.

These results show the efficacy of parapoxvirus in promoting an antiviral cell state which is protective and inhibits coronavirus replication and propagation and thereby the infection of further cells. This effect can be improved by adding an immune stimulatory agent such as ConA, which acts synergistically, i.e. the efficacy of iPPVO is enhanced in a combinatorial approach.

### Example 2

### Aim

Determining whether the proof-of-principle shown in Example 1 applies to parapoxvirus in general by testing the antiviral activity of a further parapoxvirus strain against coronavirus.

### Approach

See Example 1.

Test substances
iPPVO of strain NZ2 (chemically inactivated by binary ethyleneimine, BEI), internal designation AIC649, 1×10⁹ lyophilized virions reconstituted in 1 ml water
iPPVO of strain D1701, see Example 1
iPPVO vehicle, negative control, see Example 1

### Further Materials, Preparation of Samples, Coronavirus Assay

See Example 1. iPPVO and iPPVO vehicle were used alone. iPPVO D1701 shown to increase cell viability in Example 1 was used as a positive control in Example 2.

### Results

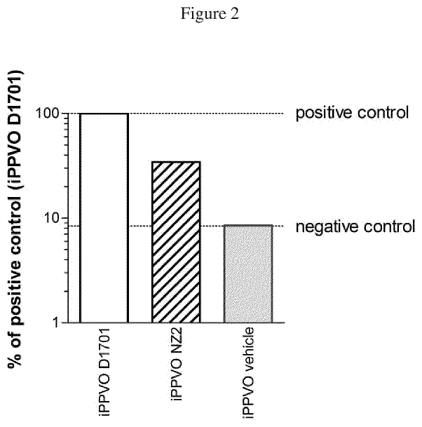

The results are shown in FIG. 2. iPPVO D1701, which in Example 1 was shown to be effective, was used as a positive control and as a 100% benchmark. Both PPVO strains D1701 and NZ2 are active against coronavirus as shown by the comparison to the negative control. Accordingly, the promotion of an antiviral cell state which is effective against coronavirus is a hallmark of parapoxvirus in general and not only of the strain tested in Example 1.

### Example 3

### Aim

Investigating the timing of parapoxvirus addition, specifically determining how the antiviral activity of parapoxvirus shown in Examples 1 and 2 is modulated by adding the supernatants at different time points to the cells of the coronavirus assay.

### Approach

See Examples 1 and 2, but with different time points for adding the supernatants of the sample plate to the cells of the coronavirus assay (before or after the infection of the VeroE6-EGFP cells with SARS-CoV-2).

### Test Substances

iPPVO of strain NZ2, see Example 2
iPPVO of strain D1701, see Example 1
iPPVO vehicle, negative control, see Example 1
ConA, see Example 1

### Further Materials, Preparation of Samples, Coronavirus Assay

See Examples 1 and 2. Whole blood was incubated with iPPVO or iPPVO vehicle (alone or in combination with ConA). Supernatants were generated and added at different timepoints before or after the infection of the VeroE6-EGFP cells with the coronavirus (SARS-CoV-2).

### Example 4

### Aim

Testing further combinatorial approaches, specifically confirming that the strength of the antiviral activity of parapoxvirus shown in Examples 1, 2, and 3 can be enhanced in combinatorial approaches using further combination partners, e.g. an immune stimulatory agent or an antiviral as described herein. This includes investigating the timing of parapoxvirus and combination partner addition (parapoxvirus to blood samples and combination partner to blood sample or directly to cells of coronavirus assay), i.e. relative to each other, and also the timing of adding the coronavirus relative to parapoxvirus and combination partner. It also includes testing the influence of the concentrations of parapoxvirus and combination partner.

### Approach

See Examples 1, 2, and 3, but with different time points for adding the supernatants (of blood treated with parapoxvirus and/or combination partner, at the same or different time points) of the sample plate to the cells of the coronavirus assay (before or after the infection of the VeroE6-EGFP cells with SARS-CoV-2), and with different time points for adding the combination partner (if not already added to the blood sample) to the cells of the coronavirus assay (before or after the supernatant addition and/or the SARS-CoV-2 addition).

### Test Substances

iPPVO of strain NZ2, see Example 2
iPPVO of strain D1701, see Example 1
iPPVO vehicle, negative control, see Example 1
ConA, see Example 1 (combination partner control)
Thalidomide (combination partner)
Remdesivir (combination partner)

### Further Materials, Preparation of Samples, Coronavirus Assay

See Examples 1 and 2. Whole blood was incubated with iPPVO or iPPVO vehicle (alone or in combination with one or more combination partners). Supernatants were added at different timepoints before or after the infection of the VeroE6-EGFP cells with the coronavirus (SARS-CoV-2). One or more combination partners not used for incubation of the blood samples were added at different time points to the VeroE6-EGFP cells (before both the supernatant and the coronavirus, after both or between the two). Different concentrations of the combination partners were used.

### Example 5

### Aim

Investigating the effect of parapoxvirus on the infectious viral load of coronavirus in the lung in a hamster model.

### Approach

The hamster infection model of SARS-CoV-2 described by Boudewijns et al. (STAT2 signaling as double-edged sword restricting viral dissemination but driving severe pneumonia in SARS-CoV-2 infected hamsters. bioRxiv 2020, 2020.04.23.056838) is used. Six hamsters are treated via the intraperitoneal (i.p.) route with 1.6×10⁹ VP of iPPVO one day prior to infection with SARS-CoV-2. A second group of six hamsters is treated with iPPVO vehicle (placebo control) instead. For infection, hamsters are anesthetized with ketamine/xylazine/atropine and inoculated intranasally with SARS-CoV-2 (1.89×10⁶ TCID₅₀ in 50 μL). At day 4 post-infection (pi), hamsters are euthanized by i.p. injection of 500 μL Dolethal (200 mg/mL sodium pentobarbital, Vétoquinol SA). Lungs are collected and infectious virus is quantified by end-point virus titration. Efficacy is determined based on viral load in homogenized lung tissues on day 4 post-infection.

### Animals

Syrian Golden Hamster, females, 6-10 weeks old

### SARS-Cov-2

SARS-Cov-2 strain BetaCov/Belgium/GHB-03021/2020 (EPI ISL 109 40797612020-02-03) was recovered from a nasopharyngeal swab taken from a RT-qPCR confirmed asymptomatic patient who returned from Wuhan, China in the beginning of February 2020. A close relation with the prototypic Wuhan-Hu-1 2019-nCoV (GenBank accession 112 number MN908947.3) strain was confirmed by phylogenetic analysis. Infectious virus was isolated by serial passaging on HuH7 and Vero E6 cells (Boudewijns et al., supra); passage 6 virus was used for the study described here. The titre of the virus stock was determined by end-point dilution on Vero E6 cells by the Reed and Muench method (supra).

### Test Substances

iPPVO strain of NZ2, see Example 2
iPPVO vehicle (PPVO buffer only, i.e. without virus)

### End-Point Virus Titration

Lung tissues were homogenized using bead disruption (Precellys) in 350 μL minimal essential medium and centrifuged (10,000 rpm, 5 min, 4° C.) to pellet the cell debris. To quantify infectious SARS-CoV-2 particles, endpoint titrations were performed on confluent Vero E6 cells in 96 well plates. Viral titres were calculated using the Reed and Muench method (Reed and Muench, The American Journal of Hygiene, 1938. 27(3): p. 493-497) and the Lindenbach calculator and were expressed as 50% tissue culture infectious dose (TCID50) per mg tissue.

### Results

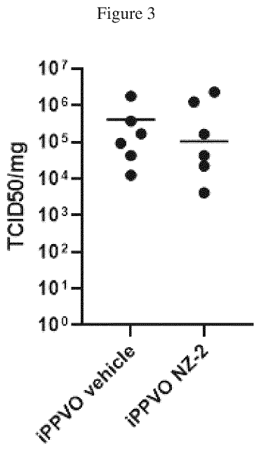

Prophylactic treatment with iPPVO reduced the number of infectious SARS-CoV-2 particles in the lung: The mean value of TCID50/mg lung tissue of placebo treated hamsters was measured to be 6.3×10⁵ compared to iPPVO-treated hamsters, in which the TCID50/mg lung tissue was measured to be 2.1×10⁵. See FIG. 3.

### Example 6

### Aim

Investigating the effects of parapoxvirus on coronavirus infection in a mouse model.

### Approach

10 mice are intravenously inoculated with 100 μl of iPPVO (1 vial dissolved in 500 μl supplied buffer, which equals a dose of 1×10⁹ virus particles (VP)/animal) prior to coronavirus infection. A second group of 10 mice serves as an infection control group (intravenous inoculation with 100 μl iPPVO buffer). One day after treatment, mice of both groups are infected intranasally under isoflurane anesthesia (inhalation of 3% isoflurane) with 900 focus forming units (FFU) of SARS-CoV-2 (German isolate) in 50 μl total volume. Mice are scored daily. Euthanasia is performed on day 10 after infection. Earlier euthanasia is performed for mice reaching humane endpoints as determined by the clinical score. Response to treatment is assessed by a clinical score determination (clinical symptoms of infection) and body weight loss. Also, SARS-CoV-2 load is determined in lungs and brains.

### Animals

Transgenic K18-hACE2 mouse

### Test Substances

iPPVO strain of D1701, see Example 1
iPPVO vehicle (PPVO buffer only, i.e. without virus)

### Determination of Clinical Score

The clinical score was determined by adding the score of each clinical parameter according to Table 1 below. Animals were euthanised upon reaching a clinical score of >20 or scoring 20 for one clinical parameter (humane endpoints).

TABLE 1
Clinical score.

| Clinical | | | |
|---|---|---|---|
| parameter | Score criteria | Score | Welfare measures |
| Body weight | Up to max. 7% reduction or increase/ | 0 | |
| reduction/ | balanced skeletal-muscular ratio. | | |
| body | 8-10% reduction/visible skeletal | 5 | Offer moistened food |
| condition | attachments in the area of the spine | | |
| | (sunken) | | |
| | 11-19% reduction/prominent skeletal | 10 | Offer moistened food, presentation |
| | structure spine and beginning at the | | to veterinarian and lead |
| | pelvis (emaciated) | | experimenter, administration of |
| | | | metamizole in the drinking water |
| | 20% reduction/very prominent | 20 | Euthanasia |
| | skeletal structure of spine and pelvic | | |
| | bones (emaciated) | | |
| General | Open, clear eyes; coat: smooth, shiny | 0 | |
| condition | and close fitting; clean and dry orifices; | | |
| (coat, eyes, | even, hardly visible respiration. | | |
| respiration) | Coat somewhat dull, partly erect | 2 | |
| | (piloerection). | | |
| | Coat altogether slightly shaggy and | 5 | Cleaning of body orifices, eye |
| | possibly erect; body openings unkempt; | | ointment |
| | clear secretion (smear film) on one or | | |
| | both eyes. | | |
| | Very shaggy/straggly coat, body | 10 | 2 x daily adspection, offer |
| | openings slightly stuck together, eye(s) | | moistened food, cleaning of body |
| | no longer fully open. | | orifices, eye ointment, presentation |
| | | | to veterinarian and lead |
| | | | experimenter, administration of |
| | | | metamizole in the drinking water |
| | Coat very shaggy and very puffy | 20 | Euthanasia |
| | general impression with strongly stuck | | |
| | or reddened or swollen eyes or | | |
| | completely closed eyes, visibly | | |
| | increased breathing with possibly | | |
| | relieving posture | | |
| Behaviour | regular food/water intake, active and | 0 | |
| (spontaneous | attentive/curious (exploration | | |
| and | behaviour), social contacts, nest- | | |
| provoked) | building instinct, personal hygiene | | |
| | Slightly reduced activity or slight | 5 | Offer moistened food |
| | hyperkinetics | | |
| | Clearly reduced activity, possibly | 10 | 2 x daily adspection, offer |
| | seclusion from the group with defence | | moistened feed, presentation to |
| | against cage mates, provoked behaviour: | | veterinarian and lead |
| | Aggressiveness when touched/ | | experimenter, administration of |
| | grabbing or reduced reaction to touch. | | metamizole via drinking water |
| | Strongly reduced activity, no exploration | 20 | Euthanasia |
| | behaviour and/or nest-building | | |
| | instinct, strongly reduced social | | |
| | behaviour up to self-isolation, apathy; | | |
| | Provoked behaviour: Sounds of pain when | | |
| | grasping and/or no reaction to touch. | | |
| Posture/ | Normal gait, including raising on hind | 0 | |
| motor | legs; normal spreading of hind limbs | | |
| function | when lifted by tail | | |
| (includes | Gait abnormalities with slowed | 5 | Offer moistened food |
| rare SARS- | movement on cage floor; hind legs not | | |
| CoV-2 | fully spread when lifted on tail | | |
| specific | Reduced movement pattern with stiff | 10 | 2 x daily adspection, offer moistened |
| neurological | gait, coordination problems; when lifted | | feed, presentation to veterinarian and |
| deficits). | by tail, hind legs not spread and | | lead experimenter, administration of |
| | paralysis of one limb | | metamizole via drinking water |
| | Curved posture (tucked up abdomen, | 20 | Euthanasia |
| | painful hump); toe-tapping gait; Tremor/ | | |
| | paralysis (dragging behind one limb); | | |
| | when lifted by the tail hind legs not | | |
| | spread and paralysis of two limbs | | |

### Determination of Organ Viral Load

Organs were homogenized in 2 ml PBS. Viral RNA was isolated from 140 μl of homogenates using QIAamp Viral RNA Mini Kit (Qiagen). RT-qPCR reactions were performed using TaqMan® Fast Virus 1-Step Master Mix (Thermo Fisher) and 5 μl of isolated RNA as a template. Synthetic SARS-CoV2-RNA was used as a quantitative standard to obtain viral copy numbers.

### Results

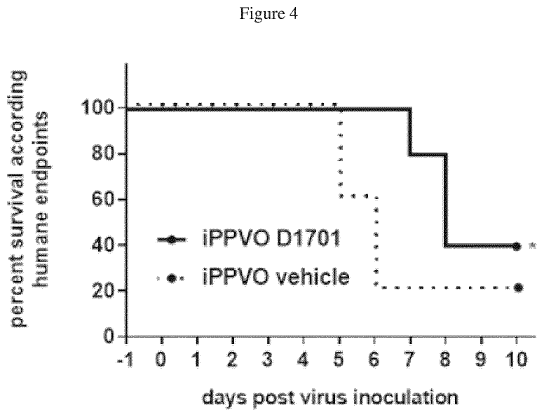

The survival rate of iPPVO treated and SARS-CoV-2 infected mice was significantly higher in comparison to untreated mice after 10 days: 40% in the iPPVO treated group compared to 20% in the control group. Also, mortality was delayed. See FIG. 4.

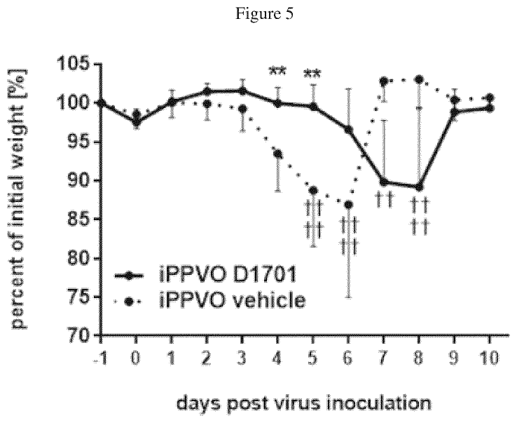

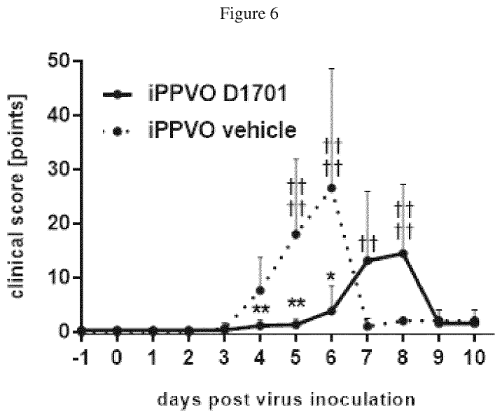

In line with this, the decrease in body weight and onset of clinical signs/symptoms were delayed in the iPPVO treatment group. At days 4 to 6 after SARS-CoV-2 infection, body weight was higher and the clinical score was lower in the iPPVO treated mice compared to the control group. At day 7 this was reversed due to euthanasia of mice in the control group that reached humane endpoints, i.e. of the mice worst affected by SARS-CoV-2 infection. These mice were not included in the data points after being euthanised, so means of the control group include only the healthiest mice from day 7 on. At days 7 and 8, mice of the iPPVO treatment group reached human endpoints and had to be euthanised. Body weight and clinical score were similar in the surviving mice of both treatment and control groups (day 9 and 10). See FIGS. 5 and 6.

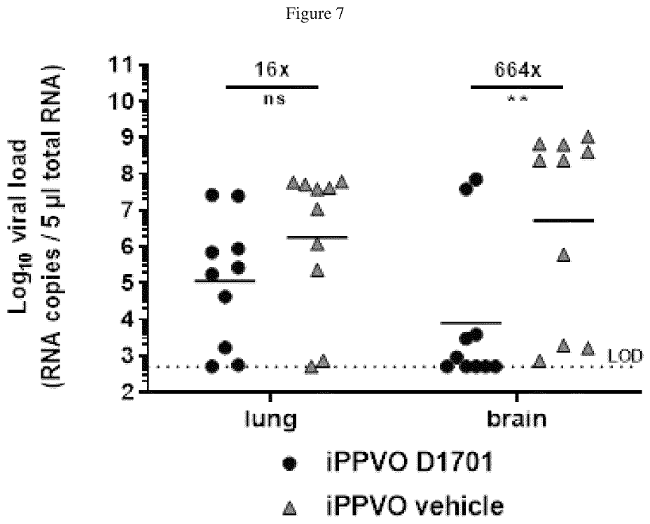

The SARS-CoV-2 viral load was reduced dramatically in both lungs (16× compared to the control group) and brains (664× compared to placebo) of infected mice. See FIG. 7.

Overall, these data clearly demonstrate a beneficial effect of PPVO treatment on the clinical outcome of SARS-CoV-2 infected mice, suggesting an improvement of the clinical situation of SARS-CoV-2 infected patients, including a delay in the onset of symptoms and a milder course of the disease.

1-15. (canceled)

16. A method of (i) preparing a subject for a coronavirus infection or (ii) treating a coronavirus infection in a subject, the method comprising administering an effective amount of a Parapoxvirus ovis (PPVO) agent to the subject.

17. The method according to claim 16, wherein the PPVO agent is:
(a) PPVO itself or a fragment thereof, or
(b) an agent that comprises genetic information encoding for (a).

18. The method according to claim 17, wherein the PPVO agent is an inactivated PPVO virion.

19. The method according to claim 16, wherein the coronavirus is a SARS coronavirus.

20. The method according to claim 19, wherein the coronavirus is SARS-CoV-2 or a variant thereof with at least 80% sequence identity to SEQ ID NO: 1 or 2.

21. The method according to claim 17, wherein the subject to be prepared is characterized by one or more risk factors for contracting a coronavirus infection and/or by one or more risk factors for having a severe course of coronavirus disease.

22. The method according to claim 16, wherein the subject to be treated is asymptomatic.

23. The method according to claim 16, wherein the subject to be treated has coronavirus disease.

24. The method according to claim 23, wherein the coronavirus disease is Covid-19.

25. The method according to claim 23, wherein the subject has no severe symptoms.

26. The method according to claim 23, wherein the subject has a severe course of coronavirus disease.

27. The method according to claim 16, further comprising administering a further treatment regimen for (i) or (ii).

28. The method according to claim 27, wherein the further treatment regimen for (i) comprises administering a coronavirus agent and/or an immune stimulatory agent, and/or the further treatment regimen for (ii) comprises administering a coronavirus agent, one or more further medicaments and/or one or more further non-drug treatments.

29. The method according to claim 28, wherein the coronavirus agent is selected from the group consisting of:
(i) an inactivated coronavirus,
(ii) an antigenic, preferably immunogenic fragment of a coronavirus,
(iii) a nucleic acid encoding for (i) or (ii),
(iv) a vector comprising the nucleic acid of (iii),
(v) a cell comprising the nucleic acid of (iii) or the vector of (iv),
(vi) an antibody or an antigen-binding fragment thereof binding to a coronavirus or a fragment thereof, preferably the envelope,
(vii) a nucleotide/nucleoside analogue, preferably coronavirus-specific,
(viii) a coronavirus-specific RNAi molecule,
(ix) a small molecule, an antibody or an antigen-binding fragment thereof, binding to a site of protein-protein interaction (PPI) between a coronavirus protein and a subject protein, and
(x) blood plasma of a subject that has recovered from a coronavirus infection, preferably from coronavirus disease;
the further medicament is selected from the group consisting of an immune stimulatory agent, an antiviral, an antibiotic, an adjuvant, a glucocorticoid, an antihypertensive drug, a hypoglycaemic drug, an anti-shock drug, and a drug suitable for treating one or more symptoms of coronavirus disease; and/or
the one or more further therapies are preferably selected from the group consisting of oxygen therapy and extra-corporeal organ support (ECOS).

30. The method according to claim 29, wherein the small molecule is selected from the group consisting of Loratadine, Daunorubicin, Midostaurin, Ponatinib, Silmitasertib, Valproic Acid, Haloperidol, Metformin, Migalastat, S-verapamil, Indomethacin, Ruxolitinib, Mycophenolic acid, Entacapone, Ribavirin, E-52862, Merimepodib, RVX-208, XL413, AC-55541, Apicidin, AZ3451, AZ8838, Bafilomycin A1, CCT 365623, GB110, H-89, JQ1, PB28, PD-144418, RS-PPCC, TMCB, UCPH-101, ZINC1775962367, ZINC4326719, ZINC4511851, ZINC95559591, 4E2RCat, ABBV-744, Camostat, Captopril, CB5083, Chloramphenicol, Chloroquine, PI4K-IIIβ inhibitor Compound 10, Cyclophilin inhibitor Compound 2, CPI-0610, Dabrafenib, DBeQ, dBET6, IHVR-19029, Linezolid, Lisinopril, Minoxidil, ML240, MZ1, Nafamostat, Pevonedistat, PS3061, Rapamycin (Sirolimus), Sanglifehrin A, Sapanisertib (INK128/MIN128), FK-506 (Tacrolimus), Ternatin 4 (DA3), Tigecycline, Tomivosertib (eFT-508), Verdinexor, WDB002, Azacitidine, Selinexor, and Zotatifin (eFT226).

31. A composition comprising a Parapoxvirus ovis (PPVO) agent and a coronavirus agent.

32. A kit comprising a Parapoxvirus ovis (PPVO) agent and a coronavirus agent.


SEQUENCE LISTING (3 sequences)

SEQ ID NO: 1 - RNA, 29903 nt, Artificial Sequence
  SARS-CoV-2
auuaaagguuuauaccuucccagguaacaaaccaaccaacuuucgaucucuuguagaucuguucucuaaacgaacuuuaaaaucuguguggcugucacucggcugcaugcuuagugcacucacgcaguauaauuaauaacuaauuacugucguugacaggacacgaguaacucgucuaucuucugcaggcugcuuacgguuucguccguguugcagccgaucaucagcacaucuagguuucguccgggugugaccgaaagguaagauggagagccuugucccugguuucaacgagaaaacacacguccaacucaguuugccuguuuuacagguucgcgacgugcucguacguggcuuuggagacuccguggaggaggucuuaucagaggcacgucaacaucuuaaagauggcacuuguggcuuaguagaaguugaaaaaggcguuuugccucaacuugaacagcccuauguguucaucaaacguucggaugcucgaacugcaccucauggucauguuaugguugagcugguagcagaacucgaaggcauucaguacggucguaguggugagacacuugguguccuugucccucaugugggcgaaauaccaguggcuuaccgcaagguucuucuucguaagaacgguaauaaaggagcugguggccauaguuacggcgccgaucuaaagucauuugacuuaggcgacgagcuuggcacugauccuuaugaagauuuucaagaaaacuggaacacuaaacauagcagugguguuacccgugaacucaugcgugagcuuaacggaggggcauacacucgcuaugucgauaacaacuucuguggcccugauggcuacccucuugagugcauuaaagaccuucuagcacgugcugguaaagcuucaugcacuuuguccgaacaacuggacuuuauugacacuaagagggguguauacugcugccgugaacaugagcaugaaauugcuugguacacggaacguucugaaaagagcuaugaauugcagacaccuuuugaaauuaaauuggcaaagaaauuugacaccuucaauggggaauguccaaauuuuguauuucccuuaaauuccauaaucaagacuauucaaccaaggguugaaaagaaaaagcuugauggcuuuauggguagaauucgaucugucuauccaguugcgucaccaaaugaaugcaaccaaaugugccuuucaacucucaugaagugugaucauuguggugaaacuucauggcagacgggcgauuuuguuaaagccacuugcgaauuuuguggcacugagaauuugacuaaagaaggugccacuacuugugguuacuuaccccaaaaugcuguuguuaaaauuuauuguccagcaugucacaauucagaaguaggaccugagcauagucuugccgaauaccauaaugaaucuggcuugaaaaccauucuucguaaggguggucgcacuauugccuuuggaggcuguguguucucuuauguugguugccauaacaagugugccuauuggguuccacgugcuagcgcuaacauagguuguaaccauacagguguuguuggagaagguuccgaaggucuuaaugacaaccuucuugaaauacuccaaaaagagaaagucaacaucaauauuguuggugacuuuaaacuuaaugaagagaucgccauuauuuuggcaucuuuuucugcuuccacaagugcuuuuguggaaacugugaaagguuuggauuauaaagcauucaaacaaauuguugaauccugugguaauuuuaaaguuacaaaaggaaaagcuaaaaaaggugccuggaauauuggugaacagaaaucaauacugaguccucuuuaugcauuugcaucagaggcugcucguguuguacgaucaauuuucucccgcacucuugaaacugcucaaaauucugugcguguuuuacagaaggccgcuauaacaauacuagauggaauuucacaguauucacugagacucauugaugcuaugauguucacaucugauuuggcuacuaacaaucuaguuguaauggccuacauuacaggugguguuguucaguugacuucgcaguggcuaacuaacaucuuuggcacuguuuaugaaaaacucaaacccguccuugauuggcuugaagagaaguuuaaggaagguguagaguuucuuagagacgguugggaaauuguuaaauuuaucucaaccugugcuugugaaauugucgguggacaaauugucaccugugcaaaggaaauuaaggagaguguucagacauucuuuaagcuuguaaauaaauuuuuggcuuugugugcugacucuaucauuauugguggagcuaaacuuaaagccuugaauuuaggugaaacauuugucacgcacucaaagggauuguacagaaaguguguuaaauccagagaagaaacuggccuacucaugccucuaaaagccccaaaagaaauuaucuucuuagagggagaaacacuucccacagaaguguuaacagaggaaguugucuugaaaacuggugauuuacaaccauuagaacaaccuacuagugaagcuguugaagcuccauugguugguacaccaguuuguauuaacgggcuuauguugcucgaaaucaaagacacagaaaaguacugugcccuugcaccuaauaugaugguaacaaacaauaccuucacacucaaaggcggugcaccaacaaagguuacuuuuggugaugacacugugauagaagugcaagguuacaagagugugaauaucacuuuugaacuugaugaaaggauugauaaaguacuuaaugagaagugcucugccuauacaguugaacucgguacagaaguaaaugaguucgccuguguuguggcagaugcugucauaaaaacuuugcaaccaguaucugaauuacuuacaccacugggcauugauuuagaugaguggaguauggcuacauacuacuuauuugaugagucuggugaguuuaaauuggcuucacauauguauuguucuuucuacccuccagaugaggaugaagaagaaggugauugugaagaagaagaguuugagccaucaacucaauaugaguaugguacugaagaugauuaccaagguaaaccuuuggaauuuggugccacuucugcugcucuucaaccugaagaagagcaagaagaagauugguuagaugaugauagucaacaaacuguuggucaacaagacggcagugaggacaaucagacaacuacuauucaaacaauuguugagguucaaccucaauuagagauggaacuuacaccaguuguucagacuauugaagugaauaguuuuagugguuauuuaaaacuuacugacaauguauacauuaaaaaugcagacauuguggaagaagcuaaaaagguaaaaccaacagugguuguuaaugcagccaauguuuaccuuaaacauggaggagguguugcaggagccuuaaauaaggcuacuaacaaugccaugcaaguugaaucugaugauuacauagcuacuaauggaccacuuaaagugggugguaguuguguuuuaagcggacacaaucuugcuaaacacugucuucauguugucggcccaaauguuaacaaaggugaagacauucaacuucuuaagagugcuuaugaaaauuuuaaucagcacgaaguucuacuugcaccauuauuaucagcugguauuuuuggugcugacccuauacauucuuuaagaguuuguguagauacuguucgcacaaaugucuacuuagcugucuuugauaaaaaucucuaugacaaacuuguuucaagcuuuuuggaaaugaagagugaaaagcaaguugaacaaaagaucgcugagauuccuaaagaggaaguuaagccauuuauaacugaaaguaaaccuucaguugaacagagaaaacaagaugauaagaaaaucaaagcuuguguugaagaaguuacaacaacucuggaagaaacuaaguuccucacagaaaacuuguuacuuuauauugacauuaauggcaaucuucauccagauucugccacucuuguuagugacauugacaucacuuucuuaaagaaagaugcuccauauauagugggugauguuguucaagaggguguuuuaacugcugugguuauaccuacuaaaaaggcugguggcacuacugaaaugcuagcgaaagcuuugagaaaagugccaacagacaauuauauaaccacuuacccgggucaggguuuaaaugguuacacuguagaggaggcaaagacagugcuuaaaaaguguaaaagugccuuuuacauucuaccaucuauuaucucuaaugagaagcaagaaauucuuggaacuguuucuuggaauuugcgagaaaugcuugcacaugcagaagaaacacgcaaauuaaugccugucuguguggaaacuaaagccauaguuucaacuauacagcguaaauauaaggguauuaaaauacaagagggugugguugauuauggugcuagauuuuacuuuuacaccaguaaaacaacuguagcgucacuuaucaacacacuuaacgaucuaaaugaaacucuuguuacaaugccacuuggcuauguaacacauggcuuaaauuuggaagaagcugcucgguauaugagaucucucaaagugccagcuacaguuucuguuucuucaccugaugcuguuacagcguauaaugguuaucuuacuucuucuucuaaaacaccugaagaacauuuuauugaaaccaucucacuugcugguuccuauaaagauugguccuauucuggacaaucuacacaacuagguauagaauuucuuaagagaggugauaaaaguguauauuacacuaguaauccuaccacauuccaccuagauggugaaguuaucaccuuugacaaucuuaagacacuucuuucuuugagagaagugaggacuauuaagguguuuacaacaguagacaacauuaaccuccacacgcaaguuguggacaugucaaugacauauggacaacaguuugguccaacuuauuuggauggagcugauguuacuaaaauaaaaccucauaauucacaugaagguaaaacauuuuauguuuuaccuaaugaugacacucuacguguugaggcuuuugaguacuaccacacaacugauccuaguuuucuggguagguacaugucagcauuaaaucacacuaaaaaguggaaauacccacaaguuaaugguuuaacuucuauuaaaugggcagauaacaacuguuaucuugccacugcauuguuaacacuccaacaaauagaguugaaguuuaauccaccugcucuacaagaugcuuauuacagagcaagggcuggugaagcugcuaacuuuugugcacuuaucuuagccuacuguaauaagacaguaggugaguuaggugauguuagagaaacaaugaguuacuuguuucaacaugccaauuuagauucuugcaaaagagucuugaacgugguguguaaaacuuguggacaacagcagacaacccuuaaggguguagaagcuguuauguacaugggcacacuuucuuaugaacaauuuaagaaagguguucagauaccuuguacgugugguaaacaagcuacaaaauaucuaguacaacaggagucaccuuuuguuaugaugucagcaccaccugcucaguaugaacuuaagcaugguacauuuacuugugcuagugaguacacugguaauuaccaguguggucacuauaaacauauaacuucuaaagaaacuuuguauugcauagacggugcuuuacuuacaaaguccucagaauacaaagguccuauuacggauguuuucuacaaagaaaacaguuacacaacaaccauaaaaccaguuacuuauaaauuggaugguguuguuuguacagaaauugacccuaaguuggacaauuauuauaagaaagacaauucuuauuucacagagcaaccaauugaucuuguaccaaaccaaccauauccaaacgcaagcuucgauaauuuuaaguuuguaugugauaauaucaaauuugcugaugauuuaaaccaguuaacugguuauaagaaaccugcuucaagagagcuuaaaguuacauuuuucccugacuuaaauggugaugugguggcuauugauuauaaacacuacacacccucuuuuaagaaaggagcuaaauuguuacauaaaccuauuguuuggcauguuaacaaugcaacuaauaaagccacguauaaaccaaauaccugguguauacguugucuuuggagcacaaaaccaguugaaacaucaaauucguuugauguacugaagucagaggacgcgcagggaauggauaaucuugccugcgaagaucuaaaaccagucucugaagaaguaguggaaaauccuaccauacagaaagacguucuugaguguaaugugaaaacuaccgaaguuguaggagacauuauacuuaaaccagcaaauaauaguuuaaaaauuacagaagagguuggccacacagaucuaauggcugcuuauguagacaauucuagucuuacuauuaagaaaccuaaugaauuaucuagaguauuagguuugaaaacccuugcuacucaugguuuagcugcuguuaauagugucccuugggauacuauagcuaauuaugcuaagccuuuucuuaacaaaguuguuaguacaacuacuaacauaguuacacgguguuuaaaccguguuuguacuaauuauaugccuuauuucuuuacuuuauugcuacaauuguguacuuuuacuagaaguacaaauucuagaauuaaagcaucuaugccgacuacuauagcaaagaauacuguuaagagugucgguaaauuuugucuagaggcuucauuuaauuauuugaagucaccuaauuuuucuaaacugauaaauauuauaauuugguuuuuacuauuaaguguuugccuagguucuuuaaucuacucaaccgcugcuuuagguguuuuaaugucuaauuuaggcaugccuucuuacuguacugguuacagagaaggcuauuugaacucuacuaaugucacuauugcaaccuacuguacugguucuauaccuuguaguguuugucuuagugguuuagauucuuuagacaccuauccuucuuuagaaacuauacaaauuaccauuucaucuuuuaaaugggauuuaacugcuuuuggcuuaguugcagagugguuuuuggcauauauucuuuucacuagguuuuucuauguacuuggauuggcugcaaucaugcaauuguuuuucagcuauuuugcaguacauuuuauuaguaauucuuggcuuaugugguuaauaauuaaucuuguacaaauggccccgauuucagcuaugguuagaauguacaucuucuuugcaucauuuuauuauguauggaaaaguuaugugcauguuguagacgguuguaauucaucaacuuguaugauguguuacaaacguaauagagcaacaagagucgaauguacaacuauuguuaaugguguuagaagguccuuuuaugucuaugcuaauggagguaaaggcuuuugcaaacuacacaauuggaauuguguuaauugugauacauucugugcugguaguacauuuauuagugaugaaguugcgagagacuugucacuacaguuuaaaagaccaauaaauccuacugaccagucuucuuacaucguugauaguguuacagugaagaaugguuccauccaucuuuacuuugauaaagcuggucaaaagacuuaugaaagacauucucucucucauuuuguuaacuuagacaaccugagagcuaauaacacuaaagguucauugccuauuaauguuauaguuuuugaugguaaaucaaaaugugaagaaucaucugcaaaaucagcgucuguuuacuacagucagcuuaugugucaaccuauacuguuacuagaucaggcauuagugucugauguuggugauagugcggaaguugcaguuaaaauguuugaugcuuacguuaauacguuuucaucaacuuuuaacguaccaauggaaaaacucaaaacacuaguugcaacugcagaagcugaacuugcaaagaauguguccuuagacaaugucuuaucuacuuuuauuucagcagcucggcaaggguuuguugauucagauguagaaacuaaagauguuguugaaugucuuaaauugucacaucaaucugacauagaaguuacuggcgauaguuguaauaacuauaugcucaccuauaacaaaguugaaaacaugacaccccgugaccuuggugcuuguauugacuguagugcgcgucauauuaaugcgcagguagcaaaaagucacaacauugcuuugauauggaacguuaaagauuucaugucauugucugaacaacuacgaaaacaaauacguagugcugcuaaaaagaauaacuuaccuuuuaaguugacaugugcaacuacuagacaaguuguuaauguuguaacaacaaagauagcacuuaagggugguaaaauuguuaauaauugguugaagcaguuaauuaaaguuacacuuguguuccuuuuuguugcugcuauuuucuauuuaauaacaccuguucaugucaugucuaaacauacugacuuuucaagugaaaucauaggauacaaggcuauugaugguggugucacucgugacauagcaucuacagauacuuguuuugcuaacaaacaugcugauuuugacacaugguuuagccagcguggugguaguuauacuaaugacaaagcuugcccauugauugcugcagucauaacaagagaaguggguuuugucgugccugguuugccuggcacgauauuacgcacaacuaauggugacuuuuugcauuucuuaccuagaguuuuuagugcaguugguaacaucuguuacacaccaucaaaacuuauagaguacacugacuuugcaacaucagcuuguguuuuggcugcugaauguacaauuuuuaaagaugcuucugguaagccaguaccauauuguuaugauaccaauguacuagaagguucuguugcuuaugaaaguuuacgcccugacacacguuaugugcucauggauggcucuauuauucaauuuccuaacaccuaccuugaagguucuguuagagugguaacaacuuuugauucugaguacuguaggcacggcacuugugaaagaucagaagcugguguuuguguaucuacuagugguagauggguacuuaacaaugauuauuacagaucuuuaccaggaguuuucugugguguagaugcuguaaauuuacuuacuaauauguuuacaccacuaauucaaccuauuggugcuuuggacauaucagcaucuauaguagcuggugguauuguagcuaucguaguaacaugccuugccuacuauuuuaugagguuuagaagagcuuuuggugaauacagucauguaguugccuuuaauacuuuacuauuccuuaugucauucacuguacucuguuuaacaccaguuuacucauucuuaccugguguuuauucuguuauuuacuuguacuugacauuuuaucuuacuaaugauguuucuuuuuuagcacauauucaguggaugguuauguucacaccuuuaguaccuuucuggauaacaauugcuuauaucauuuguauuuccacaaagcauuucuauugguucuuuaguaauuaccuaaagagacguguagucuuuaaugguguuuccuuuaguacuuuugaagaagcugcgcugugcaccuuuuuguuaaauaaagaaauguaucuaaaguugcguagugaugugcuauuaccucuuacgcaauauaauagauacuuagcucuuuauaauaaguacaaguauuuuaguggagcaauggauacaacuagcuacagagaagcugcuuguugucaucucgcaaaggcucucaaugacuucaguaacucagguucugauguucuuuaccaaccaccacaaaccucuaucaccucagcuguuuugcagagugguuuuagaaaaauggcauucccaucugguaaaguugaggguuguaugguacaaguaacuugugguacaacuacacuuaacggucuuuggcuugaugacguaguuuacuguccaagacaugugaucugcaccucugaagacaugcuuaacccuaauuaugaagauuuacucauucguaagucuaaucauaauuucuugguacaggcugguaauguucaacucaggguuauuggacauucuaugcaaaauuguguacuuaagcuuaagguugauacagccaauccuaagacaccuaaguauaaguuuguucgcauucaaccaggacagacuuuuucaguguuagcuuguuacaaugguucaccaucugguguuuaccaaugugcuaugaggcccaauuucacuauuaaggguucauuccuuaaugguucaugugguaguguugguuuuaacauagauuaugacugugucucuuuuuguuacaugcaccauauggaauuaccaacuggaguucaugcuggcacagacuuagaagguaacuuuuauggaccuuuuguugacaggcaaacagcacaagcagcugguacggacacaacuauuacaguuaauguuuuagcuugguuguacgcugcuguuauaaauggagacaggugguuucucaaucgauuuaccacaacucuuaaugacuuuaaccuuguggcuaugaaguacaauuaugaaccucuaacacaagaccauguugacauacuaggaccucuuucugcucaaacuggaauugccguuuuagauaugugugcuucauuaaaagaauuacugcaaaaugguaugaauggacguaccauauuggguagugcuuuauuagaagaugaauuuacaccuuuugauguuguuagacaaugcucagguguuacuuuccaaagugcagugaaaagaacaaucaaggguacacaccacugguuguuacucacaauuuugacuucacuuuuaguuuuaguccagaguacucaauggucuuuguucuuuuuuuuguaugaaaaugccuuuuuaccuuuugcuauggguauuauugcuaugucugcuuuugcaaugauguuugucaaacauaagcaugcauuucucuguuuguuuuuguuaccuucucuugccacuguagcuuauuuuaauauggucuauaugccugcuaguugggugaugcguauuaugacaugguuggauaugguugauacuaguuugucugguuuuaagcuaaaagacuguguuauguaugcaucagcuguaguguuacuaauccuuaugacagcaagaacuguguaugaugauggugcuaggagaguguggacacuuaugaaugucuugacacucguuuauaaaguuuauuaugguaaugcuuuagaucaagccauuuccaugugggcucuuauaaucucuguuacuucuaacuacucagguguaguuacaacugucauguuuuuggccagagguauuguuuuuauguguguugaguauugcccuauuuucuucauaacugguaauacacuucaguguauaaugcuaguuuauuguuucuuaggcuauuuuuguacuuguuacuuuggccucuuuuguuuacucaaccgcuacuuuagacugacucuugguguuuaugauuacuuaguuucuacacaggaguuuagauauaugaauucacagggacuacucccacccaagaauagcauagaugccuucaaacucaacauuaaauuguuggguguugguggcaaaccuuguaucaaaguagccacuguacagucuaaaaugucagauguaaagugcacaucaguagucuuacucucaguuuugcaacaacucagaguagaaucaucaucuaaauugugggcucaauguguccaguuacacaaugacauucucuuagcuaaagauacuacugaagccuuugaaaaaaugguuucacuacuuucuguuuugcuuuccaugcagggugcuguagacauaaacaagcuuugugaagaaaugcuggacaacagggcaaccuuacaagcuauagccucagaguuuaguucccuuccaucauaugcagcuuuugcuacugcucaagaagcuuaugagcaggcuguugcuaauggugauucugaaguuguucuuaaaaaguugaagaagucuuugaauguggcuaaaucugaauuugaccgugaugcagccaugcaacguaaguuggaaaagauggcugaucaagcuaugacccaaauguauaaacaggcuagaucugaggacaagagggcaaaaguuacuagugcuaugcagacaaugcuuuucacuaugcuuagaaaguuggauaaugaugcacucaacaacauuaucaacaaugcaagagaugguuguguucccuugaacauaauaccucuuacaacagcagccaaacuaaugguugucauaccagacuauaacacauauaaaaauacgugugaugguacaacauuuacuuaugcaucagcauugugggaaauccaacagguuguagaugcagauaguaaaauuguucaacuuagugaaauuaguauggacaauucaccuaauuuagcauggccucuuauuguaacagcuuuaagggccaauucugcugucaaauuacagaauaaugagcuuaguccuguugcacuacgacagaugucuugugcugccgguacuacacaaacugcuugcacugaugacaaugcguuagcuuacuacaacacaacaaagggagguagguuuguacuugcacuguuauccgauuuacaggauuugaaaugggcuagauucccuaagagugauggaacugguacuaucuauacagaacuggaaccaccuuguagguuuguuacagacacaccuaaagguccuaaagugaaguauuuauacuuuauuaaaggauuaaacaaccuaaauagagguaugguacuugguaguuuagcugccacaguacgucuacaagcugguaaugcaacagaagugccugccaauucaacuguauuaucuuucugugcuuuugcuguagaugcugcuaaagcuuacaaagauuaucuagcuagugggggacaaccaaucacuaauuguguuaagauguuguguacacacacugguacuggucaggcaauaacaguuacaccggaagccaauauggaucaagaauccuuugguggugcaucguguugucuguacugccguugccacauagaucauccaaauccuaaaggauuuugugacuuaaaagguaaguauguacaaauaccuacaacuugugcuaaugacccuguggguuuuacacuuaaaaacacagucuguaccgucugcgguauguggaaagguuauggcuguaguugugaucaacuccgcgaacccaugcuucagucagcugaugcacaaucguuuuuaaacggguuugcgguguaagugcagcccgucuuacaccgugcggcacaggcacuaguacugaugucguauacagggcuuuugacaucuacaaugauaaaguagcugguuuugcuaaauuccuaaaaacuaauuguugucgcuuccaagaaaaggacgaagaugacaauuuaauugauucuuacuuuguaguuaagagacacacuuucucuaacuaccaacaugaagaaacaauuuauaauuuacuuaaggauuguccagcuguugcuaaacaugacuucuuuaaguuuagaauagacggugacaugguaccacauauaucacgucaacgucuuacuaaauacacaauggcagaccucgucuaugcuuuaaggcauuuugaugaagguaauugugacacauuaaaagaaauacuugucacauacaauuguugugaugaugauuauuucaauaaaaaggacugguaugauuuuguagaaaacccagauauauuacgcguauacgccaacuuaggugaacguguacgccaagcuuuguuaaaaacaguacaauucugugaugccaugcgaaaugcugguauuguugguguacugacauuagauaaucaagaucucaaugguaacugguaugauuucggugauuucauacaaaccacgccagguaguggaguuccuguuguagauucuuauuauucauuguuaaugccuauauuaaccuugaccagggcuuuaacugcagagucacauguugacacugacuuaacaaagccuuacauuaagugggauuuguuaaaauaugacuucacggaagagagguuaaaacucuuugaccguuauuuuaaauauugggaucagacauaccacccaaauuguguuaacuguuuggaugacagaugcauucugcauugugcaaacuuuaauguuuuauucucuacaguguucccaccuacaaguuuuggaccacuagugagaaaaauauuuguugaugguguuccauuuguaguuucaacuggauaccacuucagagagcuagguguuguacauaaucaggauguaaacuuacauagcucuagacuuaguuuuaaggaauuacuuguguaugcugcugacccugcuaugcacgcugcuucugguaaucuauuacuagauaaacgcacuacgugcuuuucaguagcugcacuuacuaacaauguugcuuuucaaacugucaaacccgguaauuuuaacaaagacuucuaugacuuugcugugucuaaggguuucuuuaaggaaggaaguucuguugaauuaaaacacuucuucuuugcucaggaugguaaugcugcuaucagcgauuaugacuacuaucguuauaaucuaccaacaaugugugauaucagacaacuacuauuuguaguugaaguuguugauaaguacuuugauuguuacgaugguggcuguauuaaugcuaaccaagucaucgucaacaaccuagacaaaucagcugguuuuccauuuaauaaaugggguaaggcuagacuuuauuaugauucaaugaguuaugaggaucaagaugcacuuuucgcauauacaaaacguaaugucaucccuacuauaacucaaaugaaucuuaaguaugccauuagugcaaagaauagagcucgcaccguagcuggugucucuaucuguaguacuaugaccaauagacaguuucaucaaaaauuauugaaaucaauagccgccacuagaggagcuacuguaguaauuggaacaagcaaauucuauggugguuggcacaacauguuaaaaacuguuuauagugauguagaaaacccucaccuuauggguugggauuauccuaaaugugauagagccaugccuaacaugcuuagaauuauggccucacuuguucuugcucgcaaacauacaacguguuguagcuugucacaccguuucuauagauuagcuaaugagugugcucaaguauugagugaaauggucauguguggcgguucacuauauguuaaaccagguggaaccucaucaggagaugccacaacugcuuaugcuaauaguguuuuuaacauuugucaagcugucacggccaauguuaaugcacuuuuaucuacugaugguaacaaaauugccgauaaguauguccgcaauuuacaacacagacuuuaugagugucucuauagaaauagagauguugacacagacuuugugaaugaguuuuacgcauauuugcguaaacauuucucaaugaugauacucucugacgaugcuguuguguguuucaauagcacuuaugcaucucaaggucuaguggcuagcauaaagaacuuuaagucaguucuuuauuaucaaaacaauguuuuuaugucugaagcaaaauguuggacugagacugaccuuacuaaaggaccucaugaauuuugcucucaacauacaaugcuaguuaaacagggugaugauuauguguaccuuccuuacccagauccaucaagaauccuaggggccggcuguuuuguagaugauaucguaaaaacagaugguacacuuaugauugaacgguucgugucuuuagcuauagaugcuuacccacuuacuaaacauccuaaucaggaguaugcugaugucuuucauuuguacuuacaauacauaagaaagcuacaugaugaguuaacaggacacauguuagacauguauucuguuaugcuuacuaaugauaacacuucaagguauugggaaccugaguuuuaugaggcuauguacacaccgcauacagucuuacaggcuguuggggcuuguguucuuugcaauucacagacuucauuaagauguggugcuugcauacguagaccauucuuauguuguaaaugcuguuacgaccaugucauaucaacaucacauaaauuagucuugucuguuaauccguauguuugcaaugcuccagguugugaugucacagaugugacucaacuuuacuuaggagguaugagcuauuauuguaaaucacauaaaccacccauuaguuuuccauugugugcuaauggacaaguuuuugguuuauauaaaaauacauguguugguagcgauaauguuacugacuuuaaugcaauugcaacaugugacuggacaaaugcuggugauuacauuuuagcuaacaccuguacugaaagacucaagcuuuuugcagcagaaacgcucaaagcuacugaggagacauuuaaacugucuuaugguauugcuacuguacgugaagugcugucugacagagaauuacaucuuucaugggaaguugguaaaccuagaccaccacuuaaccgaaauuaugucuuuacugguuaucguguaacuaaaaacaguaaaguacaaauaggagaguacaccuuugaaaaaggugacuauggugaugcuguuguuuaccgagguacaacaacuuacaaauuaaauguuggugauuauuuugugcugacaucacauacaguaaugccauuaagugcaccuacacuagugccacaagagcacuauguuagaauuacuggcuuauacccaacacucaauaucucagaugaguuuucuagcaauguugcaaauuaucaaaagguugguaugcaaaaguauucuacacuccagggaccaccugguacugguaagagucauuuugcuauuggccuagcucucuacuacccuucugcucgcauaguguauacagcuugcucucaugccgcuguugaugcacuaugugagaaggcauuaaaauauuugccuauagauaaauguaguagaauuauaccugcacgugcucguguagaguguuuugauaaauucaaagugaauucaacauuagaacaguaugucuuuuguacuguaaaugcauugccugagacgacagcagauauaguugucuuugaugaaauuucaauggccacaaauuaugauuugaguguugucaaugccagauuacgugcuaagcacuauguguacauuggcgacccugcucaauuaccugcaccacgcacauugcuaacuaagggcacacuagaaccagaauauuucaauucaguguguagacuuaugaaaacuauagguccagacauguuccucggaacuugucggcguuguccugcugaaauuguugacacugugagugcuuugguuuaugauaauaagcuuaaagcacauaaagacaaaucagcucaaugcuuuaaaauguuuuauaaggguguuaucacgcaugauguuucaucugcaauuaacaggccacaaauaggcgugguaagagaauuccuuacacguaacccugcuuggagaaaagcugucuuuauuucaccuuauaauucacagaaugcuguagccucaaagauuuugggacuaccaacucaaacuguugauucaucacagggcucagaauaugacuaugucauauucacucaaaccacugaaacagcucacucuuguaauguaaacagauuuaauguugcuauuaccagagcaaaaguaggcauacuuugcauaaugucugauagagaccuuuaugacaaguugcaauuuacaagucuugaaauuccacguaggaauguggcaacuuuacaagcugaaaauguaacaggacucuuuaaagauuguaguaagguaaucacuggguuacauccuacacaggcaccuacacaccucaguguugacacuaaauucaaaacugaagguuuauguguugacauaccuggcauaccuaaggacaugaccuauagaagacucaucucuaugauggguuuuaaaaugaauuaucaaguuaaugguuacccuaacauguuuaucacccgcgaagaagcuauaagacauguacgugcauggauuggcuucgaugucgaggggugucaugcuacuagagaagcuguugguaccaauuuaccuuuacagcuagguuuuucuacagguguuaaccuaguugcuguaccuacagguuauguugauacaccuaauaauacagauuuuuccagaguuagugcuaaaccaccgccuggagaucaauuuaaacaccucauaccacuuauguacaaaggacuuccuuggaauguagugcguauaaagauuguacaaauguuaagugacacacuuaaaaaucucucugacagagucguauuugucuuaugggcacauggcuuugaguugacaucuaugaaguauuuugugaaaauaggaccugagcgcaccuguugucuaugugauagacgugccacaugcuuuuccacugcuucagacacuuaugccuguuggcaucauucuauuggauuugauuacgucuauaauccguuuaugauugauguucaacaaugggguuuuacagguaaccuacaaagcaaccaugaucuguauugucaaguccaugguaaugcacauguagcuaguugugaugcaaucaugacuaggugucuagcuguccacgagugcuuuguuaagcguguugacuggacuauugaauauccuauaauuggugaugaacugaagauuaaugcggcuuguagaaagguucaacacaugguuguuaaagcugcauuauuagcagacaaauucccaguucuucacgacauugguaacccuaaagcuauuaaguguguaccucaagcugauguagaauggaaguucuaugaugcacagccuuguagugacaaagcuuauaaaauagaagaauuauucuauucuuaugccacacauucugacaaauucacagaugguguaugccuauuuuggaauugcaaugucgauagauauccugcuaauuccauuguuuguagauuugacacuagagugcuaucuaaccuuaacuugccugguugugaugguggcaguuuguauguaaauaaacaugcauuccacacaccagcuuuugauaaaagugcuuuuguuaauuuaaaacaauuaccauuuuucuauuacucugacaguccaugugagucucauggaaaacaaguagugucagauauagauuauguaccacuaaagucugcuacguguauaacacguugcaauuuagguggugcugucuguagacaucaugcuaaugaguacagauuguaucucgaugcuuauaacaugaugaucucagcuggcuuuagcuuguggguuuacaaacaauuugauacuuauaaccucuggaacacuuuuacaagacuucagaguuuagaaaauguggcuuuuaauguuguaaauaagggacacuuugauggacaacagggugaaguaccaguuucuaucauuaauaacacuguuuacacaaaaguugaugguguugauguagaauuguuugaaaauaaaacaacauuaccuguuaauguagcauuugagcuuugggcuaagcgcaacauuaaaccaguaccagaggugaaaauacucaauaauuuggguguggacauugcugcuaauacugugaucugggacuacaaaagagaugcuccagcacauauaucuacuauugguguuuguucuaugacugacauagccaagaaaccaacugaaacgauuugugcaccacucacugucuuuuuugaugguagaguugauggucaaguagacuuauuuagaaaugcccguaaugguguucuuauuacagaagguaguguuaaagguuuacaaccaucuguaggucccaaacaagcuagucuuaauggagucacauuaauuggagaagccguaaaaacacaguucaauuauuauaagaaaguugaugguguuguccaacaauuaccugaaacuuacuuuacucagaguagaaauuuacaagaauuuaaacccaggagucaaauggaaauugauuucuuagaauuagcuauggaugaauucauugaacgguauaaauuagaaggcuaugccuucgaacauaucguuuauggagauuuuagucauagucaguuaggugguuuacaucuacugauuggacuagcuaaacguuuuaaggaaucaccuuuugaauuagaagauuuuauuccuauggacaguacaguuaaaaacuauuucauaacagaugcgcaaacagguucaucuaaguguguguguucuguuauugauuuauuacuugaugauuuuguugaaauaauaaaaucccaagauuuaucuguaguuucuaagguugucaaagugacuauugacuauacagaaauuucauuuaugcuuugguguaaagauggccauguagaaacauuuuacccaaaauuacaaucuagucaagcguggcaaccggguguugcuaugccuaaucuuuacaaaaugcaaagaaugcuauuagaaaagugugaccuucaaaauuauggugauagugcaacauuaccuaaaggcauaaugaugaaugucgcaaaauauacucaacugugucaauauuuaaacacauuaacauuagcuguacccuauaauaugagaguuauacauuuuggugcugguucugauaaaggaguugcaccagguacagcuguuuuaagacagugguugccuacggguacgcugcuugucgauucagaucuuaaugacuuugucucugaugcagauucaacuuugauuggugauugugcaacuguacauacagcuaauaaaugggaucucauuauuagugauauguacgacccuaagacuaaaaauguuacaaaagaaaaugacucuaaagaggguuuuuucacuuacauuuguggguuuauacaacaaaagcuagcucuuggagguuccguggcuauaaagauaacagaacauucuuggaaugcugaucuuuauaagcucaugggacacuucgcaugguggacagccuuuguuacuaaugugaaugcgucaucaucugaagcauuuuuaauuggauguaauuaucuuggcaaaccacgcgaacaaauagaugguuaugucaugcaugcaaauuacauauuuuggaggaauacaaauccaauucaguugucuuccuauucuuuauuugacaugaguaaauuuccccuuaaauuaagggguacugcuguuaugucuuuaaaagaaggucaaaucaaugauaugauuuuaucucuucuuaguaaagguagacuuauaauuagagaaaacaacagaguuguuauuucuagugauguucuuguuaacaacuaaacgaacaauguuuguuuuucuuguuuuauugccacuagucucuagucaguguguuaaucuuacaaccagaacucaauuacccccugcauacacuaauucuuucacacgugguguuuauuacccugacaaaguuuucagauccucaguuuuacauucaacucaggacuuguucuuaccuuucuuuuccaauguuacuugguuccaugcuauacaugucucugggaccaaugguacuaagagguuugauaacccuguccuaccauuuaaugaugguguuuauuuugcuuccacugagaagucuaacauaauaagaggcuggauuuuugguacuacuuuagauucgaagacccagucccuacuuauuguuaauaacgcuacuaauguuguuauuaaagucugugaauuucaauuuuguaaugauccauuuuuggguguuuauuaccacaaaaacaacaaaaguuggauggaaagugaguucagaguuuauucuagugcgaauaauugcacuuuugaauaugucucucagccuuuucuuauggaccuugaaggaaaacaggguaauuucaaaaaucuuagggaauuuguguuuaagaauauugaugguuauuuuaaaauauauucuaagcacacgccuauuaauuuagugcgugaucucccucaggguuuuucggcuuuagaaccauugguagauuugccaauagguauuaacaucacuagguuucaaacuuuacuugcuuuacauagaaguuauuugacuccuggugauucuucuucagguuggacagcuggugcugcagcuuauuauguggguuaucuucaaccuaggacuuuucuauuaaaauauaaugaaaauggaaccauuacagaugcuguagacugugcacuugacccucucucagaaacaaaguguacguugaaauccuucacuguagaaaaaggaaucuaucaaacuucuaacuuuagaguccaaccaacagaaucuauuguuagauuuccuaauauuacaaacuugugcccuuuuggugaaguuuuuaacgccaccagauuugcaucuguuuaugcuuggaacaggaagagaaucagcaacuguguugcugauuauucuguccuauauaauuccgcaucauuuuccacuuuuaaguguuauggagugucuccuacuaaauuaaaugaucucugcuuuacuaaugucuaugcagauucauuuguaauuagaggugaugaagucagacaaaucgcuccagggcaaacuggaaagauugcugauuauaauuauaaauuaccagaugauuuuacaggcugcguuauagcuuggaauucuaacaaucuugauucuaagguuggugguaauuauaauuaccuguauagauuguuuaggaagucuaaucucaaaccuuuugagagagauauuucaacugaaaucuaucaggccgguagcacaccuuguaaugguguugaagguuuuaauuguuacuuuccuuuacaaucauaugguuuccaacccacuaaugguguugguuaccaaccauacagaguaguaguacuuucuuuugaacuucuacaugcaccagcaacuguuuguggaccuaaaaagucuacuaauuugguuaaaaacaaaugugucaauuucaacuucaaugguuuaacaggcacagguguucuuacugagucuaacaaaaaguuucugccuuuccaacaauuuggcagagacauugcugacacuacugaugcuguccgugauccacagacacuugagauucuugacauuacaccauguucuuuugguggugucaguguuauaacaccaggaacaaauacuucuaaccagguugcuguucuuuaucaggauguuaacugcacagaagucccuguugcuauucaugcagaucaacuuacuccuacuuggcguguuuauucuacagguucuaauguuuuucaaacacgugcaggcuguuuaauaggggcugaacaugucaacaacucauaugagugugacauacccauuggugcagguauaugcgcuaguuaucagacucagacuaauucuccucggcgggcacguaguguagcuagucaauccaucauugccuacacuaugucacuuggugcagaaaauucaguugcuuacucuaauaacucuauugccauacccacaaauuuuacuauuaguguuaccacagaaauucuaccagugucuaugaccaagacaucaguagauuguacaauguacauuuguggugauucaacugaaugcagcaaucuuuuguugcaauauggcaguuuuuguacacaauuaaaccgugcuuuaacuggaauagcuguugaacaagacaaaaacacccaagaaguuuuugcacaagucaaacaaauuuacaaaacaccaccaauuaaagauuuuggugguuuuaauuuuucacaaauauuaccagauccaucaaaaccaagcaagaggucauuuauugaagaucuacuuuucaacaaagugacacuugcagaugcuggcuucaucaaacaauauggugauugccuuggugauauugcugcuagagaccucauuugugcacaaaaguuuaacggccuuacuguuuugccaccuuugcucacagaugaaaugauugcucaauacacuucugcacuguuagcggguacaaucacuucugguuggaccuuuggugcaggugcugcauuacaaauaccauuugcuaugcaaauggcuuauagguuuaaugguauuggaguuacacagaauguucucuaugagaaccaaaaauugauugccaaccaauuuaauagugcuauuggcaaaauucaagacucacuuucuuccacagcaagugcacuuggaaaacuucaagauguggucaaccaaaaugcacaagcuuuaaacacgcuuguuaaacaacuuagcuccaauuuuggugcaauuucaaguguuuuaaaugauauccuuucacgucuugacaaaguugaggcugaagugcaaauugauagguugaucacaggcagacuucaaaguuugcagacauaugugacucaacaauuaauuagagcugcagaaaucagagcuucugcuaaucuugcugcuacuaaaaugucagaguguguacuuggacaaucaaaaagaguugauuuuuguggaaagggcuaucaucuuauguccuucccucagucagcaccucaugguguagucuucuugcaugugacuuaugucccugcacaagaaaagaacuucacaacugcuccugccauuugucaugauggaaaagcacacuuuccucgugaaggugucuuuguuucaaauggcacacacugguuuguaacacaaaggaauuuuuaugaaccacaaaucauuacuacagacaacacauuugugucugguaacugugauguuguaauaggaauugucaacaacacaguuuaugauccuuugcaaccugaauuagacucauucaaggaggaguuagauaaauauuuuaagaaucauacaucaccagauguugauuuaggugacaucucuggcauuaaugcuucaguuguaaacauucaaaaagaaauugaccgccucaaugagguugccaagaauuuaaaugaaucucucaucgaucuccaagaacuuggaaaguaugagcaguauauaaaauggccaugguacauuuggcuagguuuuauagcuggcuugauugccauaguaauggugacaauuaugcuuugcuguaugaccaguugcuguaguugucucaagggcuguuguucuuguggauccugcugcaaauuugaugaagacgacucugagccagugcucaaaggagucaaauuacauuacacauaaacgaacuuauggauuuguuuaugagaaucuucacaauuggaacuguaacuuugaagcaaggugaaaucaaggaugcuacuccuucagauuuuguucgcgcuacugcaacgauaccgauacaagccucacucccuuucggauggcuuauuguuggcguugcacuucuugcuguuuuucagagcgcuuccaaaaucauaacccucaaaaagagauggcaacuagcacucuccaaggguguucacuuuguuugcaacuugcuguuguuguuuguaacaguuuacucacaccuuuugcucguugcugcuggccuugaagccccuuuucucuaucuuuaugcuuuagucuacuucuugcagaguauaaacuuuguaagaauaauaaugaggcuuuggcuuugcuggaaaugccguuccaaaaacccauuacuuuaugaugccaacuauuuucuuugcuggcauacuaauuguuacgacuauuguauaccuuacaauaguguaacuucuucaauugucauuacuucaggugauggcacaacaaguccuauuucugaacaugacuaccagauuggugguuauacugaaaaaugggaaucuggaguaaaagacuguguuguauuacacaguuacuucacuucagacuauuaccagcuguacucaacucaauugaguacagacacugguguugaacauguuaccuucuucaucuacaauaaaauuguugaugagccugaagaacauguccaaauucacacaaucgacgguucauccggaguuguuaauccaguaauggaaccaauuuaugaugaaccgacgacgacuacuagcgugccuuuguaagcacaagcugaugaguacgaacuuauguacucauucguuucggaagagacagguacguuaauaguuaauagcguacuucuuuuucuugcuuucgugguauucuugcuaguuacacuagccauccuuacugcgcuucgauugugugcguacugcugcaauauuguuaacgugagucuuguaaaaccuucuuuuuacguuuacucucguguuaaaaaucugaauucuucuagaguuccugaucuucuggucuaaacgaacuaaauauuauauuaguuuuucuguuuggaacuuuaauuuuagccauggcagauuccaacgguacuauuaccguugaagagcuuaaaaagcuccuugaacaauggaaccuaguaauagguuuccuauuccuuacauggauuugucuucuacaauuugccuaugccaacaggaauagguuuuuguauauaauuaaguuaauuuuccucuggcuguuauggccaguaacuuuagcuuguuuugugcuugcugcuguuuacagaauaaauuggaucaccgguggaauugcuaucgcaauggcuugucuuguaggcuugauguggcucagcuacuucauugcuucuuucagacuguuugcgcguacgcguuccauguggucauucaauccagaaacuaacauucuucucaacgugccacuccauggcacuauucugaccagaccgcuucuagaaagugaacucguaaucggagcugugauccuucguggacaucuucguauugcuggacaccaucuaggacgcugugacaucaaggaccugccuaaagaaaucacuguugcuacaucacgaacgcuuucuuauuacaaauugggagcuucgcagcguguagcaggugacucagguuuugcugcauacagucgcuacaggauuggcaacuauaaauuaaacacagaccauuccaguagcagugacaauauugcuuugcuuguacaguaagugacaacagauguuucaucucguugacuuucagguuacuauagcagagauauuacuaauuauuaugaggacuuuuaaaguuuccauuuggaaucuugauuacaucauaaaccucauaauuaaaaauuuaucuaagucacuaacugagaauaaauauucucaauuagaugaagagcaaccaauggagauugauuaaacgaacaugaaaauuauucuuuucuuggcacugauaacacucgcuacuugugagcuuuaucacuaccaagaguguguuagagguacaacaguacuuuuaaaagaaccuugcucuucuggaacauacgagggcaauucaccauuucauccucuagcugauaacaaauuugcacugacuugcuuuagcacucaauuugcuuuugcuuguccugacggcguaaaacacgucuaucaguuacgugccagaucaguuucaccuaaacuguucaucagacaagaggaaguucaagaacuuuacucuccaauuuuucuuauuguugcggcaauaguguuuauaacacuuugcuucacacucaaaagaaagacagaaugauugaacuuucauuaauugacuucuauuugugcuuuuuagccuuucugcuauuccuuguuuuaauuaugcuuauuaucuuuugguucucacuugaacugcaagaucauaaugaaacuugucacgccuaaacgaacaugaaauuucuuguuuucuuaggaaucaucacaacuguagcugcauuucaccaagaauguaguuuacagucauguacucaacaucaaccauauguaguugaugacccguguccuauucacuucuauucuaaaugguauauuagaguaggagcuagaaaaucagcaccuuuaauugaauugugcguggaugaggcugguucuaaaucacccauucaguacaucgauaucgguaauuauacaguuuccuguuuaccuuuuacaauuaauugccaggaaccuaaauuggguagucuuguagugcguuguucguucuaugaagacuuuuuagaguaucaugacguucguguuguuuuagauuucaucuaaacgaacaaacuaaaaugucugauaauggaccccaaaaucagcgaaaugcaccccgcauuacguuugguggacccucagauucaacuggcaguaaccagaauggagaacgcaguggggcgcgaucaaaacaacgucggccccaagguuuacccaauaauacugcgucuugguucaccgcucucacucaacauggcaaggaagaccuuaaauucccucgaggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugagggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccuaaacucaugcagaccacacaaggcagaugggcuauauaaacguuuucgcuuuuccguuuacgauauauagucuacucuugugcagaaugaauucucguaacuacauagcacaaguagauguaguuaacuuuaaucucacauagcaaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugugauuuuaauagcuucuuaggagaaugacaaaaaaaaaaaaaaaaaaaaaaaaaaaaaaaaa

SEQ ID NO: 2 - RNA, 30005 nt, Artificial Sequence
  SARS-CoV-2 strain consensus, n can be any nucleotide
  misc_feature (1)..(132): n is a, c, g, or u
  misc_feature (161)..(161): n is a, c, g, or u
  misc_feature (168)..(169): n is a, c, g, or u
  misc_feature (176)..(177): n is a, c, g, or u
  misc_feature (181)..(181): n is a, c, g, or u
  misc_feature (197)..(197): n is a, c, g, or u
  misc_feature (243)..(243): n is a, c, g, or u
  misc_feature (254)..(255): n is a, c, g, or u
  misc_feature (257)..(259): n is a, c, g, or u
  misc_feature (261)..(263): n is a, c, g, or u
  misc_feature (265)..(265): n is a, c, g, or u
  misc_feature (267)..(267): n is a, c, g, or u
  misc_feature (278)..(278): n is a, c, g, or u
  misc_feature (298)..(298): n is a, c, g, or u
  misc_feature (311)..(311): n is a, c, g, or u
  misc_feature (389)..(389): n is a, c, g, or u
  misc_feature (411)..(411): n is a, c, g, or u
  misc_feature (547)..(547): n is a, c, g, or u
  misc_feature (565)..(579): n is a, c, g, or u
  misc_feature (671)..(671): n is a, c, g, or u
  misc_feature (711)..(711): n is a, c, g, or u
  misc_feature (743)..(751): n is a, c, g, or u
  misc_feature (890)..(890): n is a, c, g, or u
  misc_feature (941)..(941): n is a, c, g, or u
  misc_feature (1116)..(1116): n is a, c, g, or u
  misc_feature (1120)..(1120): n is a, c, g, or u
  misc_feature (1159)..(1159): n is a, c, g, or u
  misc_feature (1405)..(1405): n is a, c, g, or u
  misc_feature (1442)..(1442): n is a, c, g, or u
  misc_feature (1454)..(1454): n is a, c, g, or u
  misc_feature (1487)..(1487): n is a, c, g, or u
  misc_feature (1526)..(1526): n is a, c, g, or u
  misc_feature (1605)..(1605): n is a, c, g, or u
  misc_feature (1748)..(1748): n is a, c, g, or u
  misc_feature (1791)..(1791): n is a, c, g, or u
  misc_feature (1968)..(1968): n is a, c, g, or u
  misc_feature (2148)..(2148): n is a, c, g, or u
  misc_feature (2238)..(2239): n is a, c, g, or u
  misc_feature (2326)..(2326): n is a, c, g, or u
  misc_feature (2334)..(2334): n is a, c, g, or u
  misc_feature (2503)..(2503): n is a, c, g, or u
  misc_feature (2689)..(2689): n is a, c, g, or u
  misc_feature (2774)..(2774): n is a, c, g, or u
  misc_feature (3028)..(3028): n is a, c, g, or u
  misc_feature (3094)..(3094): n is a, c, g, or u
  misc_feature (3156)..(3156): n is a, c, g, or u
  misc_feature (3234)..(3234): n is a, c, g, or u
  misc_feature (3316)..(3316): n is a, c, g, or u
  misc_feature (3468)..(3468): n is a, c, g, or u
  misc_feature (3575)..(3575): n is a, c, g, or u
  misc_feature (3795)..(3795): n is a, c, g, or u
  misc_feature (3835)..(3835): n is a, c, g, or u
  misc_feature (4049)..(4049): n is a, c, g, or u
  misc_feature (4345)..(4345): n is a, c, g, or u
  misc_feature (4364)..(4364): n is a, c, g, or u
  misc_feature (4459)..(4459): n is a, c, g, or u
  misc_feature (4936)..(4938): n is a, c, g, or u
  misc_feature (5109)..(5109): n is a, c, g, or u
  misc_feature (5119)..(5119): n is a, c, g, or u
  misc_feature (5141)..(5141): n is a, c, g, or u
  misc_feature (5537)..(5537): n is a, c, g, or u
  misc_feature (5629)..(5629): n is a, c, g, or u
  misc_feature (5773)..(5773): n is a, c, g, or u
  misc_feature (5841)..(5841): n is a, c, g, or u
  misc_feature (5902)..(5902): n is a, c, g, or u
  misc_feature (6083)..(6083): n is a, c, g, or u
  misc_feature (6088)..(6088): n is a, c, g, or u
  misc_feature (6092)..(6092): n is a, c, g, or u
  misc_feature (6367)..(6367): n is a, c, g, or u
  misc_feature (6558)..(6558): n is a, c, g, or u
  misc_feature (6693)..(6693): n is a, c, g, or u
  misc_feature (6752)..(6752): n is a, c, g, or u
  misc_feature (6876)..(6876): n is a, c, g, or u
  misc_feature (6953)..(6953): n is a, c, g, or u
  misc_feature (7016)..(7016): n is a, c, g, or u
  misc_feature (7053)..(7053): n is a, c, g, or u
  misc_feature (7073)..(7073): n is a, c, g, or u
  misc_feature (7536)..(7536): n is a, c, g, or u
  misc_feature (7923)..(7923): n is a, c, g, or u
  misc_feature (8058)..(8058): n is a, c, g, or u
  misc_feature (8369)..(8369): n is a, c, g, or u
  misc_feature (8445)..(8445): n is a, c, g, or u
  misc_feature (8825)..(8825): n is a, c, g, or u
  misc_feature (8839)..(8839): n is a, c, g, or u
  misc_feature (9044)..(9044): n is a, c, g, or u
  misc_feature (9091)..(9091): n is a, c, g, or u
  misc_feature (9214)..(9214): n is a, c, g, or u
  misc_feature (9216)..(9216): n is a, c, g, or u
  misc_feature (9237)..(9237): n is a, c, g, or u
  misc_feature (9331)..(9331): n is a, c, g, or u
  misc_feature (9531)..(9531): n is a, c, g, or u
  misc_feature (9534)..(9534): n is a, c, g, or u
  misc_feature (9548)..(9548): n is a, c, g, or u
  misc_feature (9568)..(9568): n is a, c, g, or u
  misc_feature (9591)..(9591): n is a, c, g, or u
  misc_feature (9618)..(9618): n is a, c, g, or u
  misc_feature (9981)..(9981): n is a, c, g, or u
  misc_feature (10093)..(10093): n is a, c, g, or u
  misc_feature (10140)..(10140): n is a, c, g, or u
  misc_feature (10289)..(10289): n is a, c, g, or u
  misc_feature (10564)..(10564): n is a, c, g, or u
  misc_feature (11140)..(11140): n is a, c, g, or u
  misc_feature (11264)..(11264): n is a, c, g, or u
  misc_feature (11290)..(11290): n is a, c, g, or u
  misc_feature (11467)..(11467): n is a, c, g, or u
  misc_feature (11807)..(11807): n is a, c, g, or u
  misc_feature (12013)..(12013): n is a, c, g, or u
  misc_feature (12098)..(12098): n is a, c, g, or u
  misc_feature (12172)..(12172): n is a, c, g, or u
  misc_feature (12217)..(12217): n is a, c, g, or u
  misc_feature (12259)..(12259): n is a, c, g, or u
  misc_feature (12265)..(12265): n is a, c, g, or u
  misc_feature (12270)..(12270): n is a, c, g, or u
  misc_feature (12412)..(12412): n is a, c, g, or u
  misc_feature (12435)..(12435): n is a, c, g, or u
  misc_feature (12521)..(12521): n is a, c, g, or u
  misc_feature (12524)..(12524): n is a, c, g, or u
  misc_feature (12530)..(12530): n is a, c, g, or u
  misc_feature (12548)..(12548): n is a, c, g, or u
  misc_feature (12570)..(12571): n is a, c, g, or u
  misc_feature (12591)..(12591): n is a, c, g, or u
  misc_feature (12629)..(12629): n is a, c, g, or u
  misc_feature (12635)..(12635): n is a, c, g, or u
  misc_feature (12639)..(12639): n is a, c, g, or u
  misc_feature (12657)..(12657): n is a, c, g, or u
  misc_feature (12717)..(12717): n is a, c, g, or u
  misc_feature (12742)..(12742): n is a, c, g, or u
  misc_feature (12830)..(12830): n is a, c, g, or u
  misc_feature (12850)..(12850): n is a, c, g, or u
  misc_feature (13129)..(13129): n is a, c, g, or u
  misc_feature (13282)..(13283): n is a, c, g, or u
  misc_feature (14286)..(14286): n is a, c, g, or u
  misc_feature (14465)..(14465): n is a, c, g, or u
  misc_feature (14714)..(14714): n is a, c, g, or u
  misc_feature (14862)..(14862): n is a, c, g, or u
  misc_feature (15250)..(15250): n is a, c, g, or u
  misc_feature (15381)..(15381): n is a, c, g, or u
  misc_feature (15643)..(15643): n is a, c, g, or u
  misc_feature (15654)..(15654): n is a, c, g, or u
  misc_feature (15664)..(15664): n is a, c, g, or u
  misc_feature (15867)..(15867): n is a, c, g, or u
  misc_feature (16304)..(16304): n is a, c, g, or u
  misc_feature (16524)..(16524): n is a, c, g, or u
  misc_feature (16592)..(16592): n is a, c, g, or u
  misc_feature (16934)..(16934): n is a, c, g, or u
  misc_feature (16944)..(16944): n is a, c, g, or u
  misc_feature (16969)..(16969): n is a, c, g, or u
  misc_feature (17057)..(17057): n is a, c, g, or u
  misc_feature (17304)..(17304): n is a, c, g, or u
  misc_feature (17430)..(17430): n is a, c, g, or u
  misc_feature (17433)..(17433): n is a, c, g, or u
  misc_feature (17467)..(17467): n is a, c, g, or u
  misc_feature (17480)..(17480): n is a, c, g, or u
  misc_feature (17531)..(17531): n is a, c, g, or u
  misc_feature (17804)..(17804): n is a, c, g, or u
  misc_feature (17915)..(17915): n is a, c, g, or u
  misc_feature (18117)..(18117): n is a, c, g, or u
  misc_feature (18625)..(18625): n is a, c, g, or u
  misc_feature (18660)..(18660): n is a, c, g, or u
  misc_feature (18871)..(18871): n is a, c, g, or u
  misc_feature (18934)..(18934): n is a, c, g, or u
  misc_feature (19032)..(19032): n is a, c, g, or u
  misc_feature (19122)..(19122): n is a, c, g, or u
  misc_feature (19232)..(19232): n is a, c, g, or u
  misc_feature (19667)..(19667): n is a, c, g, or u
  misc_feature (20338)..(20338): n is a, c, g, or u
  misc_feature (20355)..(20357): n is a, c, g, or u
  misc_feature (20880)..(20880): n is a, c, g, or u
  misc_feature (20993)..(20993): n is a, c, g, or u
  misc_feature (21037)..(21037): n is a, c, g, or u
  misc_feature (21045)..(21045): n is a, c, g, or u
  misc_feature (21194)..(21194): n is a, c, g, or u
  misc_feature (21204)..(21204): n is a, c, g, or u
  misc_feature (21373)..(21373): n is a, c, g, or u
  misc_feature (21442)..(21444): n is a, c, g, or u
  misc_feature (21446)..(21446): n is a, c, g, or u
  misc_feature (21635)..(21635): n is a, c, g, or u
  misc_feature (21704)..(21704): n is a, c, g, or u
  misc_feature (21767)..(21767): n is a, c, g, or u
  misc_feature (21844)..(21844): n is a, c, g, or u
  misc_feature (22051)..(22053): n is a, c, g, or u
  misc_feature (22093)..(22093): n is a, c, g, or u
  misc_feature (22164)..(22164): n is a, c, g, or u
  misc_feature (22284)..(22284): n is a, c, g, or u
  misc_feature (22363)..(22363): n is a, c, g, or u
  misc_feature (22492)..(22492): n is a, c, g, or u
  misc_feature (22845)..(22845): n is a, c, g, or u
  misc_feature (23044)..(23044): n is a, c, g, or u
  misc_feature (23048)..(23048): n is a, c, g, or u
  misc_feature (23245)..(23245): n is a, c, g, or u
  misc_feature (23463)..(23463): n is a, c, g, or u
  misc_feature (24012)..(24012): n is a, c, g, or u
  misc_feature (24094)..(24094): n is a, c, g, or u
  misc_feature (24385)..(24385): n is a, c, g, or u
  misc_feature (24411)..(24411): n is a, c, g, or u
  misc_feature (25623)..(25623): n is a, c, g, or u
  misc_feature (25647)..(25647): n is a, c, g, or u
  misc_feature (25764)..(25764): n is a, c, g, or u
  misc_feature (25835)..(25835): n is a, c, g, or u
  misc_feature (25858)..(25858): n is a, c, g, or u
  misc_feature (26039)..(26039): n is a, c, g, or u
  misc_feature (26204)..(26204): n is a, c, g, or u
  misc_feature (26386)..(26386): n is a, c, g, or u
  misc_feature (26414)..(26414): n is a, c, g, or u
  misc_feature (26518)..(26518): n is a, c, g, or u
  misc_feature (26789)..(26789): n is a, c, g, or u
  misc_feature (27286)..(27286): n is a, c, g, or u
  misc_feature (27336)..(27336): n is a, c, g, or u
  misc_feature (27444)..(27444): n is a, c, g, or u
  misc_feature (27585)..(27585): n is a, c, g, or u
  misc_feature (27866)..(27866): n is a, c, g, or u
  misc_feature (27985)..(27985): n is a, c, g, or u
  misc_feature (28024)..(28024): n is a, c, g, or u
  misc_feature (28137)..(28137): n is a, c, g, or u
  misc_feature (28204)..(28204): n is a, c, g, or u
  misc_feature (28313)..(28313): n is a, c, g, or u
  misc_feature (28427)..(28427): n is a, c, g, or u
  misc_feature (28438)..(28438): n is a, c, g, or u
  misc_feature (28469)..(28469): n is a, c, g, or u
  misc_feature (28717)..(28717): n is a, c, g, or u
  misc_feature (28852)..(28852): n is a, c, g, or u
  misc_feature (28914)..(28914): n is a, c, g, or u
  misc_feature (28923)..(28923): n is a, c, g, or u
  misc_feature (28938)..(28938): n is a, c, g, or u
  misc_feature (28941)..(28943): n is a, c, g, or u
  misc_feature (28976)..(28976): n is a, c, g, or u
  misc_feature (29155)..(29155): n is a, c, g, or u
  misc_feature (29290)..(29290): n is a, c, g, or u
  misc_feature (29314)..(29314): n is a, c, g, or u
  misc_feature (29361)..(29361): n is a, c, g, or u
  misc_feature (29363)..(29363): n is a, c, g, or u
  misc_feature (29587)..(29587): n is a, c, g, or u
  misc_feature (29613)..(29613): n is a, c, g, or u
  misc_feature (29656)..(29656): n is a, c, g, or u
  misc_feature (29695)..(29695): n is a, c, g, or u
  misc_feature (29796)..(29796): n is a, c, g, or u
  misc_feature (29802)..(29802): n is a, c, g, or u
  misc_feature (29810)..(29819): n is a, c, g, or u
  misc_feature (29834)..(29834): n is a, c, g, or u
  misc_feature (29887)..(30005): n is a, c, g, or u
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnuuuaaaaucuguguggcugucacucggcngcaugcnnagugcanncacncaguauaauuaauaanuaauuacugucguugacaggacacgaguaacucgucuaucuucugnaggcugcuuanngnnncnnncnunuugcagccgancaucagcacaucuagguuunguccgggugugancgaaagguaagauggagagccuugucccugguuucaacgagaaaacacacguccaacucaguuugccuguuuuacagnuucgcgacgugcucguacgugncuuuggagacuccguggaggaggucuuaucagaggcacgucaacaucuuaaagauggcacuuguggcuuaguagaaguugaaaaaggcguuuugccucaacuugaacagcccuauguguucaucaaacguucggangcucgaacugcaccucannnnnnnnnnnnnnnugagcugguagcagaacucgaaggcauucaguacggucguaguggugagacacuugguguccuugucccucaugugggcgaaauaccagugncuuaccgcaagguucuucuucguaagaacgguaauaaagnagcugguggccauaguuacggcgccgaucuannnnnnnnngacuuaggcgacgagcuuggcacugauccuuaugaagauuuucaagaaaacuggaacacuaaacauagcagugguguuacccgugaacucaugcgugagcuuaacggaggggcauacacucgcuaugucgauaacaacnucuguggcccugauggcuacccucuugagugcauuaaagaccuucuagcangugcugguaaagcuucaugcacuuuguccgaacaacuggacuuuauugacacuaagagggguguauacugcugccgugaacaugagcaugaaauugcuugguacacggaacguucugaaaagagcuaugaauugcagacaccuuuugaaauuaaauuggcaaagaaauuugacancuunaauggggaauguccaaauuuuguauuucccuuaaauucnauaaucaagacuauucaaccaaggguugaaaagaaaaagcuugauggcuuuauggguagaauucgaucugucuauccaguugcgucaccaaaugaaugcaaccaaaugugccuuucaacucucaugaagugugaucauuguggugaaacuucauggcagacgggcgauuuuguuaaagccacuugcgaauuuuguggcacugagaauuugacuaaagaaggugccacuacuugugguuacuuaccncaaaaugcuguuguuaaaauuuauuguccagcaugunacaauucagaanuaggaccugagcauagucuugccgaauaccaunaugaaucuggcuugaaaaccauucuucguaaggguggungcacuauugccuuuggaggcuguguguucucuuauguugguugccauaacaagugugccuauuggguuccacgugcuancgcuaacauagguuguaaccauacagguguuguuggagaagguuccgaaggucuuaaugacaaccuucuugaaauacuccaaaaagagaaagucaacaucaauauuguuggugacuuuaaacuuaaugaagagaucgccauunuuuuggcaucuuuuucugcuuccacaagugcuuuuguggaaanugugaaagguuuggauuauaaagcauucaaacaaauuguugaauccugugguaauuuuaaaguuacaaaaggaaaagcuaaaaaaggugccuggaauauuggugaacagaaaucaauacugaguccucuuuaugcauuugcaucagaggcugcucguguuguacgaucaauuuucunccgcacucuugaaacugcucaaaauucugugcguguuuuacagaaggccgcuauaacaauacuagauggaauuucacaguauucacugagacucauugaugcuaugauguucacaucugauuuggcuacuaacaaucuaguuguaauggccuacauuacaggugguguuguucaguuganuucgcaguggcuaacuaacaucuuuggcacuguuuaugaaaaacucaaacccguccuugauuggcuugaagagaaguuuaaggaaggugnngaguuucuuagagacgguugggaaauuguuaaauuuaucucaaccugugcuugugaaauugucgguggacaaauugucaccugugcnaaggaaanuaaggagaguguucagacauucuuuaagcuuguaaauaaauuuuuggcuuugugugcugacucuaucauuauugguggagcuaaacuuaaagccuugaauuuaggugaaacauuugucacgcacucaaagggauuguacagaaaguguguuaaauccagagaagaaacnggccuacucaugccucuaaaagccccaaaagaaauuaucuucuuagagggagaaacacuucccacagaaguguuaacagaggaaguugucuugaaaacuggugauuuacaaccauuagaacaaccuacuagugaagcuguugaagcuccauugguugguacaccaguuuguauuaacgggcuuaunuugcucgaaaucaaagacacagaaaaguacugugcccuugcaccuaauaugaugguaacaaacaauaccuucacacucaaaggcngugcaccaacaaagguuacuuuuggugaugacacugugauagaagugcaagguuacaagagugugaauaucacuuuugaacuugaugaaaggauugauaaaguacuuaaugagaagugcucugccuauacaguugaacucgguacagaaguaaaugaguucgccuguguuguggcagaugcugucauaaaaacuuugcaaccaguaucugaauuacuuacaccacugggcauugauuuagaugaguggaguaungcuacauacuacuuauuugaugagucuggugaguuuaaauuggcuucacauauguauuguucuuunuacccuccagaugaggaugaagaagaaggugauugugaagaagaagaguuugagccaucaanucaauaugaguaugguacugaagaugauuaccaagguaaaccuuuggaauuuggugccacuucugcugcucuucaacnugaagaagagcaagaagaagauugguuagaugaugauagucaacaaacuguuggucaacaagacggcagugaggacaaucanacaacuacuauucaaacaauuguugagguucaaccucaauuagagauggaacuuacaccaguuguucagacuauugaagugaauaguuuuagugguuauuuaaaacuuacugacaauguauacauuaaaaaugcagacauuguggaagaagnuaaaaagguaaaaccaacagugguuguuaaugcagccaauguuuaccuuaaacauggaggagguguugcaggagccuuaaauaaggcuacuaacaaugccaugcaanuugaaucugaugauuacauagcuacuaauggaccacuuaaagugggugguaguuguguuuuaagcggacacaaucuugcuaaacacugucuucauguugucggcccaaauguuaacaaaggugaagacauucaacuucuuaagagugcuuaugaaaauuuuaaucagcacgaaguucuacuugcaccauuauuaucagcugguauuuuuggugcugaccnuauacauucuuuaagaguuuguguagauacuguucgcacnaaugucuacuuagcugucuuugauaaaaaucucuaugacaaacuuguuucaagcuuuuuggaaaugaagagugaaaagcaaguugaacaaaagaucgcugagauuccuaaagaggaaguuaagccauuuauaacugaaaguaaaccuucaguugaacagagaaaacaagaugauaagaaaaucaaagcuuguguugaagaaguuacaacaacunuggaagaaacuaaguuccucacagaaaacuuguuacuuuauauugacauuaauggcaaucuucauccagauucugccacucuuguuagugacauugacaucacuuucuuaaagaaagaugcuccauauauagugggugauguuguucaagaggguguuuuaacugcugugguuauaccuacuaaaaaggcugguggcacuacugaaaugcuagcgaaagcuuugagaaaagugccaacagacaauuauauaaccacuuacccgggucaggguuuaaaugguuacacuguagaggangcaaagacagugcuuaaanaguguaaaagugccuuuuacauucuaccaucuauuaucucuaaugagaagcaagaaauucuuggaacuguuucuuggaauuugcgagaaaugcungcacaugcagaagaaacacgcaaauuaaugccugucuguguggaaacuaaagccauaguuucaacuauacagcguaaauauaaggguauuaaaauacaagagggugugguugauuauggugcuagauuuuacuuuuacaccaguaaaacaacuguagcgucacuuaucaacacacuuaacgaucuaaaugaaacucuuguuacaaugccacuuggcuauguaacacauggcuuaaauuuggaagaagcugcucgguauaugagaucucucaaagugccagcuacaguuucuguuucuucaccugaugcuguuacagcguauaaugguuaucuuacuucuucuucuaaaacaccugaagaacauuuuauugaaaccaucucacuugcugguuccuauaaagauugguccuauucuggacaaucuacacaacuagguauagaauuucuuaagagaggugauaaaaguguauauuacacnnnuaauccuaccacauuccaccuagauggugaaguuaucaccuuugacaaucuuaagacacuucuuucuuugagagaagugaggacuauuaagguguuuacaacaguagacaacauuaaccuccacacgcaaguuguggacaugucaaugacauauggacaacaguuuggucnaacuuauuungauggagcugauguuacuaaanuaaaaccucauaauucacaugaagguaaaacauuuuauguuuuaccuaaugaugacacucuacguguugaggcuuuugaguacuaccacacaacugauccuaguuuucuggguagguacaugucagcauuaaaucacacuaaaaaguggaaauacccacaaguuaaugguuuaacuucuauuaaaugggcagauaacaacuguuaucuugccacugcauuguuaacacuccaacaaauagaguugaaguuuaauccaccugcucuacaagaugcuuauuacagagcaagggcuggugaagcugcuaacuuuugugcacuuaucuuagccuacuguaauaagacaguaggugaguuaggugauguuagagaaacaaugaguuacuuguuucaacaunccaauuuagauucuugcaaaagagucuugaacgugguguguaaaacuuguggacaacagcagacaacccuuaaggguguagaagcuguuaunuacaugggcacacuuucuuaugaacaauuuaagaaagguguucagauaccuuguacgugugguaaacaagcuacaaaauaucuaguacaacaggagucaccuuuuguuaugaugucagcaccaccugcucaguaugaacuuaancaugguacauuuacuugugcuagugaguacacugguaauuaccaguguggucacuauaaacauauaanuucuaaagaaacuuuguauugcauagacggugcuuuacuuacaaaguccucagaauacaangguccuauuacggauguuuucuacaaagaaaacaguuacacaacaaccauaaaaccaguuacuuauaaauuggaugguguuguuuguacagaaauugacccuaaguuggacaauuauuauaagaaagacaauucuuauuucacagagcaaccaauugaucuuguaccaaaccaaccauauncaaangcangcuucgauaauuuuaaguuuguaugugauaauaucaaauuugcugaugauuuaaaccaguuaacugguuauaagaaaccugcuucaagagagcuuaaaguuacauuuuucccugacuuaaauggugaugugguggcuauugauuauaaacacuacacacccucuuuuaagaaaggagcuaaauuguuacauaaaccuauuguuuggcauguuaacaaugcaacuaauaaagccacguauaaaccaaauaccugguguauacguugucuuuggagnacaaaaccaguugaaacaucaaauucguuugauguacugaagucagaggacgcgcagggaauggauaaucuugccugcgaagaucuaaaaccagucucugaagaaguaguggaaaauccuaccauacagaaagacguucuugaguguaaugugaaaacuaccgaaguuguaggagacauuauacuuaaacnagcaaauaauaguuuaaaaauuacagaagagguuggccacacagaucuaauggcugcuuauguagacaauucuagucuuacuauuaagaaaccuaaugaauuaucuagaguauuagguuugaaaacccuugcuanucaugguuuagcugcuguuaauagugucccuugggauacuauagcuaauuaugcuaagncuuuucuuaacaaaguuguuaguacaacuacuaacauaguuacacgguguuuaaaccguguuuguacuaauuauaugccuuauuucuuuacuuuauugcuacaauuguguacuuuuacuagaanuacaaauucuagaauuaaagcaucuaugccgacuacuauagcaaagaauacuguuaagagugucgguaaauuuugunuagaggcuucauuuaauuauuugaagucaccuaauuuuucuaaacugauaaauauuauaauungguuuuuacuauuaaguguuugccuagguucuuuaancuacucaaccgcugcuuuanguguuuuaaugucuaauuuaggcaugccuucuuacuguacugguuacagagaaggcuauuugaacucuacuaaugucacuauugcaaccuacuguacugguucuauaccuuguaguguuugucuuagugguuuagauucuuuagacaccuauccuucuuuagaaacuauacaaauuaccauuucaucuuuuaaaugggauuuaacugcuuuuggcuuaguugcagagugguuuuuggcauauauucuuuucacuagguuuuucuauguacuuggauuggcugcaaucaugcaauuguuuuucagcuauuuugcaguacauuuuauuaguaauucuuggcuuaugugguuaauaauuaaucuuguacaaauggccccgauuucagcuaugguuagaauguacaucuucuuugcaucauuuuauuauguauggaaaaguuaugugcauguuguagacgguuguanuucaucaacuuguaugauguguuacaaacguaauagagcaacaagagucgaauguacaacuauuguuaaugguguuagaagguccuuuuaugucuaugcuaauggagguaaaggcuuuugcaaacuacacaauuggaauuguguuaauugugauacauucugugcugguaguacauuuauuagugaugaaguugcgagagacuugucacuacaguuuaaaagaccaauaaauccuacugaccagucuucuuacaucguugauaguguuacagugaagaaugguuccauccaucuuuacuuugauaaagcuggucaaaagacuuaugaaagacauucucucucucauuuuguuaacuuagacaaccugagagcuaauaacacuaaagnuucauugccuauuaauguuauaguuuuugaugguaaaucaaaaugugaagaaucaucugcaaaaucagcgucuguuuacuacagucagcuuaugugucaaccuauacuguuacuagaucaggcauuagugucugnuguuggugauagugcggaaguugcaguuaaaauguuugaugcuuacguuaauacguuuucaucaacuuuuaacguaccaauggaaaaacucaaaacacuaguugcaacugcagaagcugaacuugcaaagaauguguccuuagacaaugucuuaucuacuuuuauuucagcagcucggcaaggguuuguugauucagauguagaaacuaaagauguuguugaaugucuuaaauugucacaucaaucugacauagaaguuacuggcgauaguuguaauaacuauaugcucaccuauaacaaaguugaaaacnugacaccccgugaccuuggugcuuguauugacuguagugcgcgucauauuaaugcgcagguagcaaaaagucacancauugcuuugauauggaacguuaaagauuucaugucauugucugaacaacuacgaaaacaaauacguagugcugcuaaaaagaauaacuuaccuuuuaaguugacaugugcaacuacuagacaaguuguuaauguuguaacaacaaagauagcacuuaagggugguaaaauuguuaauaauugguugaagcaguuaauuaaaguuacacuuguguuccuuuuuguugcugcuauuuucuauuuaauaacaccuguucaugucaugucuaaacauacugacuuuucaagugaaaucauaggauacaaggcuauugaugguggugucacucgugacauagcaucuacagauacuuguuuugcuaacaaacaugcugauuuunacacaugguuuagncagcguggugguaguuauacuaaugacaaagcuugcccauugauugcugcagucauaacaagagaaguggguuuugucgugccugguuugccuggcacgauauuacgcacaacuaauggugacuuuuugcauuucuuaccuagaguuuuuagugcaguugguaacaucuguuacacaccaucaaaacuuauagaguacacugacnuugcaacaucagcuuguguuuuggcugcugaauguacaauuuuuaangaugcuucugguaagccaguaccauauuguuaugauaccaauguacuagaagguucuguugcuuaugaaaguuuacgcccugacacacguuaugugcucauggauggcucuauuauucaauuncnuaacaccuaccuugaagguunuguuagagugguaacaacuuuugauucugaguacuguaggcacggcacuugugaaagaucagaagcugguguuuguguaucuacuagugguagnuggguacuuaacaaugauuauuacagaucuuuaccaggaguuuucugugguguagaugcuguaaauuuacuuacuaauauguuuacaccacuaauucaaccuauuggugcuuuggacauaucagcaucuauaguagcuggugguauuguagcuaucguaguaacaugccuugccuacuauuuuaugagguuuagaagagnuunuggugaauacagunauguaguugccuuuaauacnuuacuauuccuuaugucauucanuguacucuguuuaacaccaguuuacunauucuuaccugguguuuauucuguuauuuacuuguacuugacauuuuaucuuacuaaugauguuucuuuuuuagcacauauucaguggaugguuauguucacaccuuuaguaccuuucuggauaacaauugcuuauaucauuuguauuuccacaaagcauuucuauugguucuuuaguaauuaccuaaagagacguguagucuuuaaugguguuuccuuuaguacuuuugaagaagcugcgcugugcaccuuuuuguuaaauaaagaaauguaucuaaaguugcguagugaugugcuauuaccucuuacgcaauauaauagauacuuagcucuuuauaauaaguacaaguauuuuaguggagnaauggauacaacuagcuacagagaagcugcuuguugucaucucgcaaaggcucucaaugacuucaguaacucagguucugauguucuuuaccaaccaccacaaaccucuaunaccucagcuguuuugcagagugguuuuagaaaaauggcauucccaunugguaaaguugaggguuguaugguacaaguaacuugugguacaacuacacuuaacggucuuuggcuugaugacguaguuuacuguccaagacaugugaucugcaccucugaagacaugcuuaacccuaauuaugaagauuuacucauunguaagucuaaucauaauuucuugguacaggcugguaauguucaacucaggguuauuggacauucuaugcaaaauuguguacuuaagcuuaagguugauacagccaauccuaagacaccuaaguauaaguuuguucgcauucaaccaggacagacuuuuucaguguuagcuuguuacaaugguucaccaucugguguuuaccaaugugcuaugaggcccaauuucacuauuaaggguucauuccuuaaugguucaugugguaguguugguuuuaanauagauuaugacugugucucuuuuuguuacaugcaccauauggaauuaccaacuggaguucaugcuggcacagacuuagaagguaacuuuuauggaccuuuuguugacaggcaaacagcacaagcagcugguacggacacaacuauuacaguuaauguuuuagcuugguuguacgcugcuguuauaaauggagacaggugguuucucaaucgauuuaccacaacucuuaaugacuuuaaccuuguggcuaugaaguacaauuaugaaccucuaacacaagaccauguugacauacuaggaccucuuucugcucaaacuggaauugccguuuuagauaugugugcuucauuaaaagaauuacugcaaaaugguaugaauggacguaccauauuggguagugcuuuauuagaagaugaauuuacaccuuuugauguuguuagacaaugcucagguguuacuuuccaaagugcagugaaaagaacaaucaaggguacacaccacugguuguuacucacaauuuugacuucacuuuuaguuuuaguccagaguacucaauggucuuuguucuuuuuuuunuaugaaaaugccuuuuuaccuuuugcuauggguauuauugcuaugucugcuuuugcaaugauguuugucaaacauaagcaugcauuucucuguuuguuuuuguuaccuucucuugccacuguancuuauuuuaauauggucuauaugccngcuaguugggugaugcguauuaugacaugguuggauaugguugauacuaguuugucugguuuuaagcuaaaagacuguguuauguaugcaucagcuguaguguuacuaauccuuaugacagcaagaacuguguaugaugauggugcuaggagaguguggacacuuaugaaugucuunacacucguuuauaaaguuuauuaugguaaugcuuuagaucaagccauuuccaugugggcucuuauaaucucuguuacuucuaacuacucagguguaguuacaacugucauguuuuuggccagagguauuguuuuuauguguguugaguauugcccuauuuucuucauaacugguaauacacuucaguguauaaugcuaguuuauuguuucuuaggcuauuuuuguacuuguuacuuuggccucuuuuguuuacucaaccgcuacuuuagacugacucuugguguuuaugauuacuuaguuucuacacaggaguuuagauauaugaauucacagggacuanucccacccaagaauagcauagaugccuucaaacucaacauuaaauuguuggguguugguggcaaaccuuguaucaaaguagccacuguacagucuaaaaugucagauguaaagugcacaucaguagucuuacucucaguuuugcaacaacucagaguagaaucaucaucuaaauugugggcucaauguguccaguuacacaauganauucucuuagcuaaagauacuacugaagccuuugaaaaaaugguuucacuacuuucuguuuugcuuuccaugcagggugcuguanacauaaacaagcuuugugaagaaaugcuggacaacagggcaaccuuacaagcuauagccucagaguuuaguucncuuccaucauaugcagcuuuugcuacugcucaagaagcuuaugancaggcuguugcuaauggugauucugaaguuguucuuaaaaanuugaanaagunuuugaauguggcuaaaucugaauuugaccgugaugcagccaugcaacguaaguuggaaaagauggcugaucaagcuaugacccaaauguauaaacaggcuagaucugaggacaagagggcaaaaguuacuagugcuaugcanacaaugcuuuucacuaugcuuanaaaguuggauaaugaugcacucaacaacauuaucaacaaugcaagagaugguuguguucccuugaacauaauaccucuuacaacancanccaaanuaaugguugucauaccanacuauaacacauauaaaaauannugugaugguacaacauuuanuuaugcaucagcauugugggaaauccaacagguuguanaugcanauanuaaaauuguucaacuuanugaaauuaguauggacaauucaccuaauuuagcauggccucuuauuguaacagcuuuaanggccaauucugcugucaaauuacanaauaaugagcuuaguccuguugcacuacgacagaugucuugugcugccgguacuacacaaacugcuugcacugaugacaaugcguuancuuacuacaacacaacaaanggagguagguuuguacuugcacuguuauccgauuuacaggauuugaaaugggcuagauucccuaagagugauggaacugguacuaucuauacagaacuggaaccaccuuguagguuuguuacagacacaccuaaagguccuaaagugaaguauuuauacuuuauuaaaggauuaaacaaccuaaauagagguaugguacuugguaguuuagcugccacaguacgucuacaagcugguaaugcaacagaagugccugccaauucaacuguauuaucuuunugugcuuuugcuguagaugcugcuaaagcuuacaaagauuaucuagcuagugggggacaaccaaucacuaauuguguuaagauguuguguacacacacugguacuggucaggcaauaacaguuacaccggaagccaauauggaucaagaaucnnuugguggugcaucguguugucuguacugccguugccacauagaucauccaaauccuaaaggauuuugugacuuaaaagguaaguauguacaaauaccuacaacuugugcuaaugacccuguggguuuuacacuuaaaaacacagucuguaccgucugcgguauguggaaagguuauggcuguaguugugaucaacuccgcgaacccaugcuucagucagcugaugcacaaucguuuuuaaacggguuugcgguguaagugcagcccgucuuacaccgugcggcacaggcacuaguacugaugucguauacagggcuuuugacaucuacaaugauaaaguagcugguuuugcuaaauuccuaaaaacuaauuguugucgcuuccaagaaaaggacgaagaugacaauuuaauugauucuuacuuuguaguuaagagacacacuuucucuaacuaccaacaugaagaaacaauuuauaauuuacuuaaggauuguccagcuguugcuaaacaugacuucuuuaaguuuagaauagacggugacaugguaccacauauaucacgucaacgucuuacuaaauacacaauggcagaccucgucuaugcuuuaaggcauuuugaugaagguaauugugacacauuaaaagaaauacuugucacauacaauuguugugaugaugauuauuucaauaaaaaggacugguaugauuuuguagaaaacccagauauauuacgcguauacgccaacuuaggugaacguguacgccaagcuuuguuaaaaacaguacaauucugugaugccaugcgaaaugcugguauuguugguguacugacauuagauaaucaagaucucaaugguaacugguaugauuucggugauuucauacaaaccacgccagguaguggaguuccuguuguagauucuuauuauucauuguuaaugccuauauuaaccuugaccagggcuuuaacugcagagucacauguugacacugacuuaacaaanccuuacauuaagugggauuuguuaaaauaugacuucacggaagagagguuaaaacucuuugaccguuauuuuaaauauugggaucagacauaccacccaaauuguguuaacuguuuggaugacagaugcauucugcauugugcaaacuuuaauguuuuauucucuacaguguucccacnuacaaguuuuggaccacuagugagaaaaauauuuguugaugguguuccauuuguaguuucaacuggauaccacuucagagagcuagguguuguacauaaucaggauguaaacuuacauagcucuagacuuaguuuuaaggaauuacuuguguaugcugcugacccugcuaugcacgcugcuucugguaaucuauuacuagauaaacgcacuacgugcuuuucaguagcugcacuuacuaacaauguugnuuuucaaacugucaaacccgguaauuuuaacaaagacuucuaugacuuugcugugucuaaggguuucuuuaaggaaggaaguucuguugaauuaaaacacuucuucuuugcucaggaugguaaugcugcuaucagcgauuaugacuanuaucguuauaaucuaccaacaaugugugauaucagacaacuacuauuuguaguugaaguuguugauaaguacuuugauuguuacgaugguggcuguauuaaugcuaaccaagucaucgucaacaaccuagacaaaucagcugguuuuccauuuaauaaaugggguaaggcuagacuuuauuaugauucaaugaguuaugaggaucaagaugcacuuuucgcauauacaaaacguaaugucaucccuacuauaacucaaaugaaucuuaaguaugccauuagugcaaagaauagagcucgcaccguagcuggugucucuaucuguaguacuaugaccaauagacaguuucaucaaaaauuauugaaaucaauagccgccacuagaggancuacuguaguaauuggaacaagcaaauucuauggugguuggcacaacauguuaaaaacuguuuauagugauguagaaaacccucaccuuauggguugggauuauccuaaaugugauagagccaugccuaanaugcuuagaauuauggccucacuuguucuugcucgcaaacauacaacguguuguagcuugucacaccguuucuauagauuagcuaaugagugugcucaaguauugagugaaauggucauguguggcgguucacuauauguuaaaccagguggaaccucaucaggagaugccacaacugcuuaugcuaauaguguuuuuaacauuugucaagcugucacggccaauguuaaugcacuuuuaucuacugaugguaacaaaauunccgauaaguanguccgcaaunuacaacacagacuuuaugagugucucuauagaaauagagauguugacacagacuuugugaaugaguuuuacgcauauuugcguaaacauuucucaaugaugauacucucugacgaugcuguuguguguuucaauagcacuuaugcaucucaaggucuaguggcuagcauaaagaacuuuaagucaguucuuuauuaucaaaanaauguuuuuaugucugaagcaaaauguuggacugagacugaccuuacuaaaggaccucaugaauuuugcucucaacauacaaugcuaguuaaacagggugaugauuauguguaccuuccuuacccagauccaucaagaauccuaggggccggcuguuuuguagaugauaucguaaaaacagaugguacacuuaugauugaacgguucgugucuuuagcuauagaugcuuacccacuuacuaaacauccuaaucaggaguaugcugaugucuuucauuuguacuuacaauacauaagaaagcuacaugaugaguuaacaggacacauguuagacauguauucuguuaugcuuacuaaugauaacacuucaagguauugggaaccugaguuuuaugaggcuauguacacaccgcauacagucuuacaggcuguuggggnuuguguucuuugcaauucacagacuucauuaagauguggugcuugcauacguagaccauucuuauguuguaaaugcuguuacgaccaugucauaucaacaucacauaaauuagucuugucuguuaauccguauguuugcaaugcuccagguugugaugucacagaugugacucaacuuuacuuaggagguaugagcuauuauuguaaaucacauaaaccncccauuaguuuuccauugugugcuaauggacaaguuuuugguuuauauaaaaauacauguguugguancgauaauguuacugacuuuaaugcaauugcaacaugugacuggacaaaugcuggugauuacauuuuagcuaacaccuguacugaaagacucaagcuuuuugcagcagaaacgcucaaagcuacugaggagacauuuaaacugucuuaugguauugcuacuguacgugaagugcugucugacagagaauuacaucuuucaugggaaguugguaaaccuagaccaccacuuaaccgaaauuaugucuuuacugguuaucguguaacuaaaaacaguaaaguacaaauaggagaguacaccuuugaaaaaggugacuauggugaugcuguuguuuaccgagguanaacaacuuanaaauuaaauguuggugauuauuuunugcugacaucacauacaguaaugccauuaagugcaccuacacuagugccacaagagcacuauguuagaauuacuggcuuauacccaanacucaauaucucagaugaguuuucuagcaauguugcaaauuaucaaaagguugguaugcaaaaguauucuacacuccagggaccaccugguacugguaagagucauuuugcuauuggccuagcucucuacuacccuucugcucgcauaguguauacagcuugcucucaugccgcuguugaugcacuaugugagaaggcauuaaaauauuugccuauagauaaauguaguagaauuauaccugcacgngcucguguagaguguuuugauaaauucaaagugaauucaacauuagaacaguaugucuuuuguacuguaaaugcauugccugagacgacagcagauauaguugucuuugaugaaauuucaauggcnacnaauuaugauuugaguguugucaaugccagauuangugcuaagcacunuguguacauuggcgacccugcucaauuaccugcaccacgcacauugcuaanuaagggcacacuagaaccagaauauuucaauucaguguguagacuuaugaaaacuauagguccagacauguuccucggaacuugucggcguuguccugcugaaauuguugacacugugagugcuuugguuuaugauaauaagcuuaaagcacauaaagacaaaucagcucaaugcuuuaaaauguuuuauaaggguguuaucacgcaugauguuucaucugcaauuaacaggccacaaauaggcgugguaagagaauuccuuacacguaaccnugcuuggagaaaagcugucuuuauuucaccuuauaauucacagaaugcuguagccucaaagauuuugggacuaccaacucaaacuguugauucaucacagggcucagaaunugacuaugucauauucacucaaaccacugaaacagcucacucuuguaauguaaacagauuuaauguugcuauuaccagagcaaaaguaggcauacuuugcauaaugucugauagagaccuuuaugacaaguugcaauuuacaagucuugaaauuccacguaggaauguggcaacuuuacaagcugaaaauguaacaggacunuuuaaagauuguaguaagguaaucacuggguuacauccuacacaggcaccuacacaccucaguguugacacuaaauucaaaacugaagguuuauguguugacauaccuggcauaccuaaggacaugaccuauagaagacucaucucuaugauggguuuuaaaaugaauuaucaaguuaaugguuacccuaacauguuuaucacccgcgaagaagcuauaagacauguacgugcauggauuggcuucgaugucgaggggugucaugcuacuagagaagcuguugguaccaauuuaccuuuacagcuagguuuuucuacagguguuaaccuaguugcuguaccuacagguuauguugauacaccuaauaauacagauuuuuccagaguuagugcuaaaccaccgccuggagaucaauuuaaacaccucauaccacuuauguacaaaggacuuccuuggaauguagugcguauaaagauuguacaaauguuaagugacacacuuaaaaaunucucugacagagucguauuugucuuaugggcacanggcuuugaguugacaucuaugaaguauuuugugaaaauaggaccugagcgcaccuguugucuaugugauagacgugccacaugcuuuuccacugcuucagacacuuaugccuguuggcaucauucuauuggauuugauuacgucuauaauccguuuaugauugauguucaacaaugggguuuuacagguaaccuacaaagcaaccaugaunuguauugucaaguccaugguaaugcacauguagcuaguugugaugcaaucaugacuaggugunuagcuguccacgagugcuuuguuaagcguguugacuggacuauugaauauccuauaauuggugaugaacugaagauuaaugcggcuuguagaaagguncaacacaugguuguuaaagcugcauuauuagcagacaaauucccaguucuucacgacauugguaacccuaaagcuauuaaguguguaccncaagcugauguagaauggaaguucuaugaugcacagccuuguagugacaaagcuuauaaaauagaagaauuauucuauucuuaugccacacauucugacaaauucacagnugguguaugccuauuuuggaauugcaaugucgauagauauccugcuaauuccauuguuuguagauuugacacuagagugcuaucuaaccuuaacuugccugguugugaugguggcaguuuguauguaaauaaacaugcauuccacacaccagcuuuugauaaaagugcuuuuguuaauuuaaaacaauuaccauuuuucuauuacucugacaguccaugugagucucauggaaaacaaguagugucagauauagauuauguaccacuaaagucugcuacguguauaacacguugcaauuuagguggugcugucuguagacaucaugcuaaugaguacagauuguaucucgaugcuuauaacaugaugaucucagcuggcuuuagcuuguggguuuacaaacaauuugauacuuauaaccucuggaacacuuuuanaagacuucagaguuuagaaaauguggcuuuuaauguuguaaauaagggacacuuugauggacaacagggugaaguaccaguuucuaucauuaauaacacuguuuacacaaaaguugaugguguugauguagaauuguuugaaaauaaaacaacauuaccuguuaauguagcauuugagcuuugggcuaagcgcaacauuaaaccaguaccagaggugaaaauacucaauaauuuggguguggacauugcugcuaauacugugaucugggacuacaaaagagaugcuccagcacauauaucuacuauugguguuuguucuaugacugacauagccaagaaaccaacugaaacgauuugugcaccacucacugucuuuuuugaugguagaguugauggucaaguagacuuauuuagaaaugcccguaaugguguucuuauuacagaagguaguguuaaagguuuacaaccaucuguaggucccaaacaagcuagucuuaauggagucacauuaauuggagaagccguaaaaacacaguucaauuauuauaagaaaguugaugguguuguccaacaauuaccugaaacuuacuuuacucagaguagaaauuuacaagaauuuaaacccaggagucaaauggaaauugauuucuuagaauuagcuauggaugaanucauugaacgguauaannnagaaggcuaugccuucgaacauaucguuuauggagauuuuagucauagucaguuaggugguuuacaucuacugauuggacuagcuaaacguuuuaaggaaucaccuuuugaauuagaagauuuuauuccuauggacaguacaguuaaaaacuauuucauaacagaugcgcaaacagguucaucuaaguguguguguucuguuauugauuuauuacuugaugauuuuguugaaauaauaaaaucccaagauuuaucuguaguuucuaagguugucaaagugacuauugacuauacagaaauuucauuuaugcuuugguguaaagauggccauguagaaacauuuuacccaaaauuacaaucuagucaagcguggcaaccggguguugcuaugccuaaucuuuacaaaaugcaaagaaugcuauuagaaaagugugaccuucaaaauuauggugauagugcaacauuaccuaaaggcauaaugaugaaugucgcaaaauauacucaacugugucaauauuuaaanacauuaacauuagcuguacccuauaauaugagaguuauacauuuuggugcugguucugauaaaggaguugcaccagguacagcuguuuuaagacagugguugccuacggguangcugcuugucgauucagaucuuaaugacuuugucucugaugcanauucaacnuugauuggugauugugcaacuguacauacagcuaauaaaugggaucucauuauuagugauauguacgacccuaagacuaaaaauguuacaaaagaaaaugacucuaaagaggguuuuuucacuuacauuuguggguuuauacaacaaangcuagcucunggagguuccguggcuauaaagauaacagaacauucuuggaaugcugaucuuuauaagcucaugggacacuucgcaugguggacagccuuuguuacuaaugugaaugcgucaucaucugaagcauuuuuaauuggauguaauuaucuuggcaaaccacgcgaacaaauanaugguuaugucaugcaugcaaauuacauauuuuggaggaauacaaauccaauucaguugucuuccuaunnnunuuuauuugacaugaguaaauuuccccuuaaauuaagggguacugcuguuaugucuuuaaaagaaggucaaaucaaugauaugauuuuaucucuucuuaguaaagguagacuuauaauuagagaaaacaacagaguuguuauuucuagugauguucuuguuaacaacuaaacgaacaauguuuguuuuunuuguuuuauugccacuagucucuagucaguguguuaaucuuacaaccagaacucaauuacccccugcanacacuaauucuuucacacgugguguuuauuacccugacaaaguuuucagauccucaguuuuanauucaacucaggacuuguucuuaccuuucuuuuccaauguuacuugguuccaugcuauacaugucucugggaccaangguacuaagagguuugauaacccuguccuaccauuuaaugaugguguuuauuuugcuuccacugagaagucuaacauaauaagaggcuggauuuuugguacuacuuuagauucgaagacccagucccuacuuauuguuaauaacgcuacuaauguuguuauuaaagucugugaauuucaauuuuguaaugauccauuuuugggugunnnuuaccacaaaaacaacaaaaguuggauggaaagugaguunagaguuuauucuagugcgaauaauugcacuuuugaauaugucucucagccuuuucuuauggaccuugaagnaaaacaggguaauuucaaaaaucuuagggaauuuguguuuaagaauauugaugguuauuuuaaaauauauucuaagcacacgccuauuaauuuagugcgugaucucccucaggguuuuunggcuuuagaaccauugguagauuugccaauagguauuaacaucacuagguuucaaacuuuacuugcuuuacauagaagnuauuugacuccuggugauucuucuucagguuggacagcuggugcugcagcuuauuauguggguuaucuucaaccuaggacuuuucuauuaaaauauaaugaaaauggaaccauuacagaugcuguaganugugcacuugacccucucucagaaacaaaguguacguugaaauccuucacuguagaaaaaggaaucuaucaaacuucuaacuuuagaguccaaccaacagaaucuauuguuagauuuccuaauauuacaaacuugugcccuuuuggugaaguuuuuaacgccaccagauuugcaucuguuuaugcuuggaacaggaagagaaucagcaacuguguugcugauuauucuguccuauauaauuccgcaucauuuuccacuuuuaaguguuauggagugucuccuacuaaauuaaaugaucucugcuuuacuaaugucuaugcagauucauuuguaauuagaggugaugaagucanacaaaucgcuccagggcaaacuggaaagauugcugauuauaauuauaaauuaccagaugauuuuacaggcugcguuauagcuuggaauucuaacaaucuugauucuaagguuggugguaauuauaauuaccuguauagauuguuuaggaagucuaaucucaaaccuuuugagagagauauuucaacugaaaucuaucangccnguagcacaccuuguaaugguguugaagguuuuaauuguuacuuuccuuuacaaucauaugguuuccaacccacuaaugguguugguuaccaaccauacagaguaguaguacuuucuuuugaacuucuacaugcaccagcaacuguuuguggaccuaaaaagucuacuaauuugguuaaaaacaaaugugucaauuunaacuucaaugguuuaacaggcacagguguucuuacugagucuaacaaaaaguuucugccuuuccaacaauuuggcagagacauugcugacacuacugaugcuguccgugauccacagacacuugagauucuugacauuacaccauguucuuuugguggugucaguguuauaacaccaggaacaaauacuucuaaccagguugcuguucuuuaucaggnuguuaacugcacagaagucccuguugcuauucaugcagaucaacuuacuccuacuuggcguguuuauucuacagguucuaauguuuuucaaacacgugcaggcuguuuaauaggggcugaacaugucaacaacucauaugagugugacauacccauuggugcagguauaugcgcuaguuaucagacucagacuaauucuccucggcgggcacguaguguagcuagucaauccaucauugccuacacuaugucacuuggugcagaaaauucaguugcuuacucuaauaacucuauugccauacccacaaauuuuacuauuaguguuaccacagaaauucuaccagugucuaugaccaagacaucaguagauuguacaauguacauuuguggugauucaacugaaugcagcaaucuuuuguugcaauauggcaguuuuuguacacaauuaaaccgugcuuuaacuggaauagcuguugaacaagacaaaaacacccaagaaguuuuugcacaagucaaacaaauuuacaaaacaccaccaauuaaagauunuggugguuuuaauuuuucacaaauauuaccagauccaucaaaaccaagcaagaggucauuuauugaagaucuacuuuucaanaaagugacacuugcagaugcuggcuucaucaaacaauauggugauugccuuggugauauugcugcuagagaccucauuugugcacaaaaguuuaacggccuuacuguuuugccaccuuugcucacagaugaaaugauugcucaauacacuucugcacuguuagcggguacaaucacuucugguuggaccuuuggugcaggugcugcauuacaaauaccauuugcuaugcaaauggcuuauagguuuaaugguauuggaguuacacagaauguucucuaugagaaccaaaanuugauugccaaccaauuuaauagugnuauuggcaaaauucaagacucacuuucuuccacagcaagugcacuuggaaaacuucaagauguggucaaccaaaaugcacaagcuuuaaacacgcuuguuaaacaacuuagcuccaauuuuggugcaauuucaaguguuuuaaaugauauccuuucacgucuugacaaaguugaggcugaagugcaaauugauagguugaucacaggcagacuucaaaguuugcagacauaugugacucaacaauuaauuagagcugcagaaaucagagcuucugcuaaucuugcugcuacuaaaaugucagaguguguacuuggacaaucaaaaagaguugauuuuuguggaaagggcuaucaucuuauguccuucccucagucagcaccucaugguguagucuucuugcaugugacuuaugucccugcacaagaaaagaacuucacaacugcuccugccauuugucaugauggaaaagcacacuuuccucgugaaggugucuuuguuucaaauggcacacacugguuuguaacacaaaggaauuuuuaugaaccacaaaucauuacuacagacaacacauuugugucugguaacugugauguuguaauaggaauugucaacaacacaguuuaugauccuuugcaaccugaauuagacucauucaaggaggaguuagauaaauauuuuaagaaucauacaucaccagauguugauuuaggugacaucucuggcauuaaugcuucaguuguaaacauucaaaaagaaauugaccgccucaaugagguugccaagaauuuaaaugaaucucucaucgaucuccaagaacuuggaaaguaugagcaguauauaaaauggccaugguacauuuggcuagguuuuauagcuggcuugauugccauaguaauggugacaauuaugcuuugcuguaugaccaguugcuguaguugucucaagggcuguuguucuuguggauccugcugcaaauuugaugaagacgacucugagccagugcucaaaggagucaaauuacauuacacauaaacgaacuuauggauuuguuuaugagaaucuucacaauuggaacuguaacuuugaagcaaggugaaaucaaggaugcuacuccuucagauuuuguucgcgcuacugcaacgauaccgauacaagccucacucccuuucggauggcuuauuguuggcguugcacuucuugcuguuuuucanagcgcuuccaaaaucauaacccunaaaaagagauggcaacuagcacucuccaaggguguucacuuuguuugcaacuugcuguuguuguuuguaacaguuuacucacaccuuuugcucguugcugcuggccuugaagccccnuuucucuaucuuuaugcuuuagucuacuucuugcagaguauaaacuuuguaagaauaauaaugaggcuuungcuuugcuggaaaugccguuccnaaaacccauuacuuuaugaugccaacuauuuucuuugcuggcauacuaauuguuacgacuauuguauaccuuacaauaguguaacuucuucaauugucauuacuucaggugauggcacaacaaguccuauuucugaacaugacuaccagauuggugguuauacugaaaaaugggaaucugnaguaaaagacuguguuguauuacacaguuacuucacuucagacuauuaccagcuguacucaacucaauugaguacagacacugguguugaacauguuaccuucuucaucuacaauaaaauuguugaugagccugaagaacauguccaaauucacacaaucgacgnuucauccggaguuguuaauccaguaauggaaccaauuuaugaugaaccgacgacgacuacuagcgugccuuuguaagcacaagcugaugaguacgaacuuauguacucauucguuucggaagagacagguacguuaauaguuaauagcguacuucuuuuucuugcuuucgugguauucuugnuaguuacacuagccauccuuacugcgcnucgauugugugcguacugcugcaauauuguuaacgugagucuuguaaaaccuucuuuuuacguuuacucucguguuaaaaaucugaauucuucuagaguuccunaucuucuggucuaaacgaacuaaauauuauauuaguuuuucuguuuggaacuuuaauuuuagccauggcagauuccaacgguacuauuaccguugaagagcuuaaaaagcuccuugaacaauggaaccuaguaauagguuuccuauuccuuacauggauuugucuucuacaauuugccuaugccaacaggaauagguuuuuguauauaauuaaguuaauuuuccucuggcuguuauggccaguaacuuuagcuuguuuugugcuugcugcnguuuacagaauaaauuggaucaccgguggaauugcuaucgcaauggcuugucuuguaggcuugauguggcucagcuacuucauugcuucuuucagacuguuugcgcguacgcguuccauguggucauucaauccagaaacuaacauucuucucaacgugccacuccauggcacuauucugaccagaccgcuucuagaaagugaacucguaaucggagcugugauccuucguggacaucuucguauugcuggacaccaucuaggacgcugugacaucaaggaccugccuaaagaaaucacuguugcuacaucacgaacgcuuucuuauuacaaauugggagcuucgcagcguguagcaggugacucagguuuugcugcauacagucgcuacaggauuggcaacuauaaauuaaacacagaccauuccaguagcagugacaauauugcuuugcuuguacaguaagugacaacagauguuucaucucguugacuuucagnuuacuauagcagagauauuacuaauuauuaugaggacuuuuaaaguuucnauuuggaaucuugauuacaucauaaaccucauaauuaaaaauuuaucuaagucacuaacugagaauaaauauucucaauuagaugaagagcaaccaauggagauuganuaaacgaacaugaaaauuauucuuuucuuggcacugauaacacucgcuacuugugagcuuuaucacuaccaagaguguguuagagguacaacaguacuuuuaaaagaaccuugcucuucuggaacauacgagggcaauucnccauuucauccucuagcugauaacaaauuugcacugacuugcuuuagcacucaauuugcuuuugcuuguccugacggcguaaaacacgucuaucaguuacgugccagaucaguuucaccuaaacuguucaucagacaagaggaaguucaagaacuuuacucuccaauuuuucuuauuguugcggcaauaguguuuauaacacuuugcuucacacucaaaagaaagacagaaugauugaacuuucauuaauugacuucuauuugugcuuuuuagccuuucuncuauuccuuguuuuaauuaugcuuauuaucuuuugguucucacuugaacugcaagaucauaaugaaacuugucacgccuaaacgaacaugaaauuucuuguuuucuuaggaaucaucanaacuguagcugcauuucaccaagaauguaguuuacagunauguacucaacaucaaccauauguaguugaugacccguguccuauucacuucuauucuaaaugguauauuagaguaggagcuagaaaaucagcaccuuuaauugaauugugcnuggaugaggcugguucuaaaucacccauucaguacaucgauaucgguaauuauacaguuuccuguunaccuuuuacaauuaauugccaggaaccuaaauuggguagucuuguagugcguuguucguucuaugaagacuuuuuagaguaucaugacguucguguuguuuuagauuunaucuaaacgaacaaacuaaaaugucugauaauggaccccaaaaucagcgaaaugcaccccgcauuacguuugguggacccucagauucaacuggcaguaaccagaauggagaangcaguggggcncgaucaaaacaacgucggccccaagguuuanccaauaauacugcgucuugguucaccgcucucacucaacauggcaaggaagaccuuaaauucccucgaggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaaganggcaucauauggguugcaacugagggagccuugaauacaccaaaagaucacauuggcacccgcaauccugcuaacaaugcugcaaucgugcuacaacuuccucaaggaacaacauugccaaaaggcuucuacgcngaagggagcagaggcggcagucaagccucuucucguuccucaucacguagucgcaacaguunaagaaauunaacuccaggcagcanuannngaacuucuccugcuagaauggcuggcaauggcngugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuunggcagacgugguccagaacaaacccaaggaaauuuuggggaccaggaacuaaucagacaaggaacugauuacaaacauuggccgcaaauugcacaauuugcccccagcgcuucagcguucuucggaaugucgcgnauuggcauggaagucacaccuucnggaacgugguugaccuacacaggugccaucaaauuggaugacaaagnuncaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucangccuaaacucaugcagaccacacaangcagaugggcuauauaaacguuuucgcuuuuccguuuacgaunuauagucuacucuugugcagaaugaauucucguaacuanauagcacaaguagauguaguuaacuuuaaucucacauagcaaucuuuaaucaguguguaacauuagggaggacuugaaagagccaccacauuuucaccgangccacncggaguannnnnnnnnnuacagugaacaaugnuagggagagcugccuauauggaagagcccuaauguguaaaauuaauuuuagunnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 3 - DNA, 137560 bp, Orf virus
ggatccgcggcttcgcgggcggcggccggctcccgcggcggctgagccgcggtgccgcgacgaacgcggaccaggagttcctgcgggaggagctacggcagaggctggaactgctgaatgctttcgaggacgggcgtccgcgggaacgcgactccgcggaggcggcaccccgcagccgcgagacctcgctctggagtcagggagtcagggagtcgggagtcagggagtcgggagtcagggagtcgggagtcagggagtcgggagtcagggagtcgggagtcagggagtcgggagtcagggagtcgggagtcagggagtcgggagtcagggagtcgggagtcagggagtcgggagtcagggagtcgggagtcagggagtcgggagtcagggagtcgggagtcagggagtcgggagtcagggagtcgggaggtcagggagtcgggagtcagggaaacagaagtccaagtagtacgggtgagacaggagtcaggtggtcgggtgaccgcgccgtccgagtcccgcaaaaagtttttagactctgagagaggccgacctgcctccaacggttccgcggaaaagtttttataaaaagttttcgggagaggccgactgccttccaacggttcctcctctcgcgtgccgcgggcggccgctctcccgcgacggtcccgccaacgcgtttacaaagttttaaaagttttcgagagaggccgacctgtcttccaacggttgcgcgaaaaggttctgcggaggtttggaagagccgcccgccctccgacatcctccgaaaagttttcgcaaaaagtttttaaaggtttcgcgaggagttttcgagggaggcgacctgcctccgaggttccgcgtaaacgtttttacaaagcgtcggaggtgggggcgacctgcccttcctctcctccgaaaagctcttgagagtgtgggagaggcggccggccttcgcggtcgcgcgaaaaggttctgcgggagttcgcgggagctgacccgccctccgcgcccctccgaaaagtttttgcacgaagtcttttggcgttctcgagaggacgcctaccccgacggtaacgcggaacgtctcgggaggtcggcctctccgctcccgcggtcgcggcggcggcccgtctccggaggttccgcgaaaagttttataaaaagttttgagggaggtaccgacctcctaaagttttacagagagttctcgcaaaagttttgggaggtcgacctgacctcctaaagttatcgaagagttctcgcgaagagttctcgcaaagagtttgagaggccgacctgcctccaacggttccgcggaaaagttttataagagttttgagagaggtcgacctaccttccaacggttccgcggaaaagttttataagagttttgagagaggtcgacctaccttccaacggttccgcggaaaagttttataagagttttgagagaggtcgacctactctccgaggttccgcggaaagttttataaaaagtttttacaaagtttttgagggaggtcgacacctcggatcctccgcctcgcgtgccgcgggcggcctccaagagttttacggaacgttctggagaggagaggccgacctcccaaagattttgcggaacgttttggagaggaggactggcctcgaaaagattttacaaagagtttggagggaggactggcctccaacggttccgcgaaaaagttttgcaagagtttggagagaggtcgaccaccctccgaggttccgcgaaagtgtttgcggagagtccgagagaggcagacactgcctgcgaaaagttttcgcggacggttgagcgggtcaccgacctccgacagtttacaaacgttttacggagagttagagaggcagcagaccgacctccgaacagtttagttttacaaagttttggagggtgaactgtcatcgggaaagttttacgaagagttttgggagaggccgacggttagccgagcgagcgcgcgagcgagtttacgttctctcgctcgtgtgcgcgttaactcgcactggttccctctctaactcggagtgggcgagcgagtggttgactcgtcctccgctctcactctgagtggtgataactgttaaccattaactcgtcctccactctctcactctctcactctgagtgaggattgacttgttaactcgtcctccactctctcgctctctcgctctgagtgagtgaggattaactgttcaactcgtcaactcgtcctctgcctctcatccaagactgagtgggtagttgactgctcttgttctcttactccgaggggtgattgagttagcaacagctactcgttctcttgttctctcacaccgagtgagcgagtgagcaacgttacttgttctctcacaccgagtgagcgagtgagcaacggttaactcgttctctcgttctctcgttctattattccgaggagtggcgagtgaggtaacggttactgttacttgttctctgttcccttcctcggtgagcggtgcgtcaacttgttctcgtgagtgagtacggtcacttgttctctcgttctctacctcgaggagtgagtcaacttgttctcgtgagtgagttgaccgtaaccgttcccttacctcaagtgagtggcggtcgcttgttctcgtgagtgggttaaccgcgttcccttaccggagtgagcgggcgggcgataaaaataatcaattgactgattcgctcgtgagcgagcgaagggcggcggacaagggcgcgggatgctggtctaatctactaaggccgattacaaaaacggatgggagaccgggagggagagggtcacagctccgagcggtgcatccgcgccagctggcggcgccactcggccgcgggccgcggccgcccaggccgcgttgtagccgcccgccaccgcgacgcaggtcagctcgaactccggcccgagcgcgcgcacgtcgtagatgtgcacggtcgcgacgttcagcagcagcgcgcccttgcgcagcgaggcgaaggtcgcgtgcatgccggcggcgagcgggtacaccggcgagagcgtcccgccgccgtgcgcgaccaccgccatgtgccgcccgtcgccgccgacggtcaggcaggtcaccgaggcggagccgttcgcgcggacggggcccgcctccacgagggcggccggcagcggcggcgccgcggggcggaagagcccgccgaggaggaagcccagcgccaccagcgcgagcgcgccgagcagcctgcgcggcgaggggtgcatgcttgttggctgcggtgttggcgtctggcgggtggaaggcgggtgtggggtgtccgcggctgcggcgggagggtctcgactgctaggcggtcctttttcactttgctccgtggcgcctggtccggggcaagggctgcggcgggcgcccgggcgggcgggccgctaccccgccgcgcggcccgcggccgccagcgccgcggccagccgccgccaccgcgggcccgccgcgcgcagcagccccgccgccgagcgccccgcgccgccgcgggcgcgcgccgaggccgcggccccgcgccgcgccagcagcagcggcagccgcgcgtccagcgggccgccgcggcgcagcgccgcgcgcagcagcgccgccagcggcagccgccccgccgcgtccgggcccgccgcgcgcgcgcccgccgccagcagcgcgcgcaccagcgccggcgagggcgccgcgcgcggaccaggtgctccacgagcagggtggtgagcaaggattctcgagaagtaggagtcatgtgtgacgacaaggagagacgttatattaggcgcgtcctacttcactttgaagatggtgtaaagtgttaaaacttgaacaccgttcactccaccactgccgttaccgtgtcctgccccaaaagcaaccacagtgctttttccaccacctgttccaaatccgttccaaaagctcccatccattgttgttagaactttcagatgtttctctaggttgtttagttccactgcaagtttcgaccattatcgttactggacatgctgttggtaatgagtttaataaccaatcataaaaatagttataatttgttataaagctaataaagtagcaaacactttaatgttatattttgcctaaccctccgttaacaccaccattaacaccaccacttaagcttttactaccaccactaccacctccaacacacattcttttctctaaaggtccccaaattccacctcctgaacttggacgttttacagcacctccgggtgtacttgcgtaccctttagaagttccactgtgactgtagatatgatactgtccttctccaggcatgattaaagtgtgttgtaattagtgttatctacgcaactgtgcgagactctcgaataaaaagaagctacattttacaattttgattagctgatgtaccacgctgtatcgcggccaccacaagcacccgatccagtagaaccaaatccagagtcgccgcggtcagtgttgtccaagcagttaacctcttgaactgctgggcacgatatgcgttcgcatattagctgagctatcctgtctcccttcttaacctcaaagtcactgtttccaaagttaaacagcaccactccgacgttgcctcggtagtcttcgtcgatcacgccagcgcccacgtcgataaagtgtttgactgcaaggccagaacgtggtgctatgcgtccgtagcaaccagaagggggctttatcagaaggtcagtaaatactacgcgactgcaatgcgaagggatgacacagtcgtatgcactacataggtctaatcctgcggcaccaggagatcctctggctggtatagtggcgttttggctgaggcgaacaacctgaagagtttccgtgtggcagaactccatggctagggtggcgagcggccgatcgactacggggtgtacaatttacactttctccagaaaaatcaggggcgggtcagcatggcgcggcgcaagtccagcagcgagtcgtacgacaggaagcacaggatggaggtcacgatctccggcggcagggcgcacgggcacatgaggccggcgatctgctcggccagcgagacgcgcagccgcatcatgcagatcttgccgaagagcgccgtcccgtagatggggaactcggccgcgcgctccaaaaaggcgttctgcacgaagagcgccttcgcgtcgtccgacgcgcgcagcacgtccaacagcgtcgcgtccgtgtggcagcgcaccgcgcgcatgcttgcgatctcctgctcgcacgcgcggatcacggtcgcgtagtccgccagcgcgcgctccgccagcatgcgcgcgccctcgccgcgcagcgccagctcctgcacgcacagcagcgcggcctccgagcgtcggaacacgtggccccattgctcggatgtgatcagcgcgcgcgcgagcagctccgtcggcggccggcgcgcgagcacggccgccgtcgcgcgcacgttgttgcggcgcagcatctccgaaaccgcgcataggcccgaggccgacatgtgctcgagctccgcgcccatgcgcaccagccggcagcaggcgccgtggctgaacaccgccgcgcggtgcagcgcggtctgcaggttgttgttgcgcaggttcaggtccagcccgcgctcgagcacgaagtccacgacgccgcgctcgcagctcccgtaggtcgccatgtagtgcagcatggtgttcccacacgcgtctacggcggccgggtccacgcctaggcccgtgagcgtgcgcaccatgccctcggagatcttggccgtgcgcgcgaggtggtgcagcgttgtgcgcccgtacgcgtccacgacgcacgcgtccgcgcccgcgcgcagcatcatgtccacgagcgcggcggagacgccgccggagcacagcagcgccgccagcggcgtcaagccgttgcagtcgcaggcgtttgggttcgcgccgcgctcgagcagcagccgcagcacgtcctcgcggatccactggttcttggcgtacacgtgcagcggcgttacgccgtaggtgttgccctcgttcacgcgcgcgcccgcgtccagcagcagccgcgcgacctcgagctcggcgccgtcggggccgcagaaagccaggaaggaggagagcacgctgtcgcagacaacgacgctggcgtcgcagaccacgtccgcgcccgcctccagcatgagcgcgaccacctccggccgcacgccgtcgtactgcacgtaggcgtgcagcggcgtgcggccgcaggagtccttggctttcacgtccgcaccggcctccagcagcacgcgcacgatctccgcgcactgctcgtgccgcgcgaagtgcacgcagaggtgcagcggcgtgcgcccgtgctcgccgcggaagttcacgtctgcgtcggtggctacgagcgcgcggaccgtttcgaggtccacctgcccggactccaggtagcggaagagcaggtccgcgtgcgggaccacgacggactcccgcgagagcatggcggcgtttacaaatattgaaatcttttttcactcatctttatggcgctgaacgcgcaataagggtgagagtaaaaaacttctacaaaaagcgtacaaaaggtacaaaaggtaaaaaaggcggggcggggacgggctggggtgctgcgagctgaattggcctctacacagggacgccctcgccggagccggtgagccggtagccggcgccggcgatcatggtcaagcgctgcacgagctcgttgcgcttgacgccggcctctgaaacgcacaccatgtggtggatgtaccgctcgatgcactcgcagcgcgggagagtggagtcaagatcggatgcgagttgcagaatgtcatcccagagctcggagaacttgctgtacagttctcggaggtctctctccattcgagccgtaagagagtcaggatgcggtgttccttcgggagtctgagcgaacaccgcgaacaggctggttatgccgtgttctagaatagagtggttccgcgttaatgccgcagacaagggtcgtcgtccgcgcaacgactggcggcagagcgctgtttgtgccgcaccgcccattcctctggcgatcgcatccaccgacgcagtgatcatctgcgcgccgacgtcattgtagcgcgcgttaaactcagtaatcatgattacgagattgcagatttcatagtagcacttttccaagtcgacgcgcagtttcacgatctggttgacaatcttgcacgcctttcgccgcgtctccgccacgttggcgactcggacttgcgcttcctggtcgatggacggcggaaacacttcaaacccaaggtcgcacagttcagcggtggggactagcgtcacgatgatgtactccgcatcgccacccacttgcggcaggaagaacaccgaccgcgcggcgggaacgaccagaacgtcgccttcctgcatgttggtttttagaaacttagtgttgttcacggagatgccggccatgccctcgtttttgacacatattatggtgacgtacgcggcgaccgtgggggccatgtggtggcgcatgtaccactcgtcgtgcttgagtttcagaccgtgagattcgccaacctcgaagtgcatgttggcgtctctgacgtagcgcgagaactcgctgcgacagattcgcgcgggcgcccggtggaacgtcgactcgaagagactgatggctgtccattcgcccacatgagtgaccaccgaagaagtgttttcgatccgagtctcgaacaccgagtccacgagcaccggacagttggttccgggcaccgtcagcaccaagggccgcgcctccacgggggcgacggacgaagccacggagtcggtgtccccgtacccgtagtcgtcgtcggagtcgccgcctccgtcggccccgtcgcgcggcctccgcagcggcatgcagccggcggtgggaacgcactggtttcggccacggccgaagcggccaaacagtctcgccagggctgacatccttggacggccacaccaaaaccaaaaaaacatattttatcagttatttgtcgattttcaccggctcaccgagggcaggacctcctggatcccggacacccccgccaggcagcgggccgcgcgctcgcgcacccaaaagcggtcgtagccgtgccggagcacgaaggccgccgtggcgtggcagtccacgcgctcgatgaagccgtggacggcgcggcgcgcgtagctcgccgcgaaggcgcggaccaccgccgagcagcgccccgagggcgagtcgtccgtctccagcgccagcggcatgctcgcgatgcgcgacatcaggttggaggtctgcgggatgttgagctcgcgcgtggcggtcatctgcgcctcgagcccggccttgagcacctcgtcgcagcggccccactccagcgcgcagaccacgcggatctcgtaccccttgagccgcagcgcggtctcgatgtccacggaggtgagcaccgcgctgaagcgcaggcgctcttcgtccgcggggtcgaagagcacggggatcttaacctccgcgctgcgcgtgacctcgcagagcgcgatcgcgagcagcccgcgcgtgagcttgctcaccatgcgcggcttgcccacggggtacagctggctcgcgacctcgcgcagcgggtacgccagtcggaagcagcgcgcgtccgcgggcaccgggctcgcgcccgtctcctcgaggaagagcgcggcctcaaccatgttcagcgcggagaagtgcaccgggcaggcggcgcagccgcgcgcggcgttcgcgagcaccatctcgcgcagcccgcggaaggccgccatgtcgcaggaggggaagatgcgcgcgagcgcggcctggtgcgcgagcgccgcgtccgagagcgcctgcgcgccgcggcggcgctcctcggcggcggccgcgctctcgtccgcggagaccacgtcttcgggcacgtccacgcagacgccgccccagaactcgcagtactcggagaagagcgtcgcgggcgcaaagcgcgcgaggtccacgaaggcgacgcggttgccgagcctggagagcagcgtgttctccgagatgcgcgtccagcccttgccggcgagctccatgacctgccgcgtgtcgaagagggagctgtagaagccgtacacggtgatgttttccttgcacgtcgtcagccacatgaggaagtcgcgcaccaccagcttcgcgcagtctccggagaacacggggccggcgttcgtcgcgatggagttcaggcgcacggtgccgtcactgccgaagcggtacacgaaccaggcggccacgctgttgccagagggcgtgtgaacgtgtggctgcgcccaggagtcggcgctcgcggcggtgcgcacgtcgtgcgagagcacctcggtgtcggggcgcgagtaggtgctggggtctttgatccagatggcgtagctgcccacgcagcacacgttcatgaggtcgagcagcgtctgccggcgcagcggcgtgccgagccggcgcacggcgtcgtgcgagaccatgcgcaggtcgtagaggcccacgtccgagagccactggttgagctcgtccatggacagggcgtcgcgggggggcgggctgtcttcgaaggcggcgcggagctcgggctccgtctccgcgcgctgccgcaggatgtccaggaaggggctggaggagtcggggatgtagcagtcggggtcgtgcctggacactatagcgaaccgctgcgtcgcgggcggcggggctagcgcgtcggcgcgtgcgtcgatgaaggtgcacgatatacgcacggacttgagcgaggggaggacgactgcggcggcgcgcgcgccctccgcgtcgaagatcatcgtctttccgtccctcgcctttgcgagcgcgtattctccaggcacgaggtccgtcggcggcggctcgtcccaggcctgccggtcagggacgccgccgcacacctttccccagaaccccagcatcctccaaaatacctataaggacggccaatagcggggcttgcgggcgttcggaccttccgcgctttaattttaatttattggcttgcagaactccgagcgccagtcccgctcgaagaccgcggacaggtccttgacgatgtcgcccttctcggcgttcacgctcacgaaggcgtggtagcggtagtgcgtgccgtcgaggttggcgaccgtgaggtgcgcgaaggtgtcgtccacgatgagcagcttagtgttgttcgcggcgtcgtcccggccgggtaccacgaacttgcgcacggacatgtccacgctgccgacgccaaagtcgtcgaggctgcgcgcggccgagaccgaaagcgggtccgcgttcttccactcggtaatgatcacgcgcacgcgcacgccgcggttgatggccgcgcgcagcagcgcgtcaatgatctgcggccagtactccacggcgctggcgtgcttgatcaccggcaccatcgagagcagcgagaggtcgatgctgttcttggcgttctcgatgcggtgcagcacgaggtcctcgtcgagcgtgcggtagaagcctaggaagcgctccggcgagtccgagaagaatacgccgcccccggagtggtcgaggtggaagttcgtggccgtgggcgtgacgatggcgcagcagagccgcgtgaacggcaccttcggctccacgatcatggagtagaaggtgttgtagcggttcatgaggtcccaggccaggtgcttgttggtggagtagagcccgaggttcttgatggtggacacggacccgcccgtgagcgaggcgcttcccacgtaccagtgcccggcgtccgagagccagaagctgccgagaaggttgccgacgccctccttggtggacaccttgaccttgtagtagttgacgcccgcctcgcgcagctcgtccgcgtccttgtccttgctctgcacgtccacgagcagcgtgacgtctacgccctccttggcgagcgtgcagagcttgtccttgacgtcgacgccctccttggtggagctcaggttgcagcagaagctgcagatgtacaagaacttcttcgcggactcggcgatagcggtgaagcagtcgagggtgctcatgttgccctgcgccaaagacgccacctctgcgggcagcgtctccacgacgcggcagtcggcgcccagggggatggaggagaacggccacatttatttatctcacaaaaataatagggcttcagggaaagtcttttagcaggcgggcgagttcttcgagttcgcttaggagttcttccatttcttcggaagtcagcaactggagctcggacttgatttgaatatcttcgaggaaaccgtctagcatgttcgccatgtcttccggggagcactgcgccacatcttcggggacaggatcgggtgtgggcattaggtctccgcttacttgaacgtcgtccatcatcctgtcgatgaggtcttcgacttctagacggggtccgtagatcagcatatttggtgatggaggtagtttaaggtgcgagagttagtgttatacgaccgccaacgtgtgtttatcgcgcgtacattttcaataattaacaaactccccttcctgcgcctgctcgagaagcagctcgtccagctcctcctgtcggcgcgcggccacgcgtctttccgcgaagagtaccatcagctccagccccacgccgcacagacccaggacgccgaacaccaccgccgccgagatcgacagacccagcagcaccgacatcctcacgcgggcatccggctatttaatcgttctggaaacgtattaatatgggcgtcgtcatgtgcgggtgtctgtttgtgtgggcgggctggatcgcgcgccgcgtgcgcggcctctgcgcggcgctgcgccagagggtgtcgcgcgacaagggctacgtggccgtcatccagacctgcgacgacgactacttcacagaggaggagttcgacgacggcaagcaggtggtcgcgctcctgcgcgacgtctcgcgcgtggttgccgcgcccgcgggcgtgacggaataagttaggataaggagtcgaggggagaaaaacagcggtcacactataaactcgcgcgaggccgattttgacgtgctcatgtctggaagctccgctttctgcagcgcggagcggcacacgaagcacacttccgtgttggtgggagttatgcagtggacgtggtagccgtgcccgcacaccatgacttggaacggacacgcgccgggacaggccgcgtttatgcatccttccggcgagcgcttgttgcagatgtagcacacgtctgagcacgccagcgtgcaggacacggccagcttccactgcttaaccttaacgggcatggctagttgaacacgaccatgggcgagtcgcgagcctcgagtcgggggttcagggcaaaccgtttcacgccgtcaacggttcttctctttgcaattttctctctgcacaggctcgtcagcgtcatctcggccaggcgcgcgtcgttgcctaggtgccgcgcggcgtcctcgaccgtcacgcccgtcttgccggcctcgtccatgagcacaatgcataccaggtgcgcgctagagcatatgacctcctgctcgcgcccgccggcagcggggatggttagctccgcgcgcccgaaggccgccagcggcgccacgtcgtaggcagtgtctgctcgggcgagcgccgactccacggcaccgcggagcgactccggcggcgtcagcgcggccagcggcaccggcgtgggcacggtgtacacgttcacgggcatgagcaccatctccgggtcgtggtggccgctctcttcgccgtcgtgctccatgggctgcggcggcggcagcagcgggagcagcagccgtccggacatgagccggcgcacaaggtcgttgagcgcggacgaggccatcggcgggtacagctccatggccagcttcagcgataggtgcttctcgaggttgacgccggtgtagacgctcttcacgatgcgcgcgaaggccacgcgcgcgaaggccgccagctcctcgcgcggcaggcgctcgatgtaggagagcagcatgtcggtgtcgcacggcggcgccgcgaccaccgcgccgtagagcgccttgcccgagagcttttccagcgcccttgcgtgcaggccgtgggtcttgagcacgtccacgtagttcacgtacaggcagagcgcgcgatcgaggttgctctccgcgacgtgcgtctcgatgcactccacgatgagcgggcccatgcggtccttgatgaggtctatgagcccgccgtaggcgactcgcgcgctcatgaggcacgtgcggcagtacgccatcaggccctcgaggtccgcggctatcacgtcctcgaccacgttcgccacgacgcgctgccagagccgcaccttgctcacgttctggtgccgcaccatgtccacgagctcgtcgtacgagccgccgggctcgtgcgcgcgatcgacgatgcacctcgccatggtgcggctctggcgcatgagctcgttcgtgaagcgcacgcacgcgtcctcggagaagagcgcgctcaggcaggagtagcagcggtccgcgacgaggtgcgggaagcggcactccacgacgccgcggccgatccgcagcacgcactcgccgtacatctcgtccatggcctcgcgcagacagtcgtccagcacgtccgcgttgtgcgcccactggatcacgcagaggtagggctcgatgttctcgcgcgcgttttccacctcctgcaccatgtactcgagcacggtcatgtcctcgtggatgtcggtgcccagcatgcgcccgggcggcagccagctcttgcgcgcgatcgcctctcgcaggcacgccaccgccgtgaaggtgttgacgcggagcttggtcagcagccgccgcagtcgggagatgtgtgccacggagaggtccatctccatggcctgggcgatgaggcgcgtgagttcctcctccatggcggcggctccgcgggcagatatacgcgaacaacggtaagccgtgctatttcatttttggacaaaaagctagtcgtcgacgcgcatgttgtcgaggttccggcacagcgagagcacgtcgtcgcgcgcgcgcctccggcgcagttgattgttcgcgcgccgcgcgtccgcgagcgcctgtctgtacatcgcggagtccgcgtacccgtgcagcggcgagcgccgagcgccgggcctcgggctcgcgcggcgcgggagcggcgttggcgcgcgcctcgagcgccgcgcgaagtgcgcctgcatggccagcaggcaaccgaacggcaccatgtatcggtccatgaggcactggctggccgcggacggctcgcgcgggtgcatcacgccgccgcccacgtcctccatgacgtcgcgcagcacgcagcgcagcatggtctccatgctgcgctgcgtgaaccgcaccggggaggcgtcgacgtagaagccgtcggccacgaagtagagcgcgtccagcccgccgagtttctcgccgagaccgacgaagagctcgtccacgtgccagtccaccaccgaggccttgaagagcaccacgtgccggatgtcgtgcgagcgcgcgagctcccaggtgtcctcgccgatgttgctggcgtcgatgcggcctgtcatgcgcacgctcacgcacggcgtcatcccgttcttgtagcagaactggcgcgcgagctcctcctggcgtacgatgtcgaccatgctctccatgaaggaggtggagagcagcatcgcgccgcgcgcggcgcgggtcgcgttttcgtccacctccacttccatcccgccgccgatcctaatcatctatcgtatttaaattttcggcggagcagacacgcggctgctcgctgcgcgatcgcttcagccgcggcggcgtcacgcacgcgttgcggcggccggcacgcacggacgaccgccggggctgttcgctgagcgagcgccgcggccgcgtgacgcgacagtcgcaggtgggttgccgggagtcgctcgcgcgccttcttcgcatttcgccggaacgccgcgtttatgtaggggattatattttcaacgtaactaaatggacgggggcgtgcacaaacggcctttcatcgtgaacgtggatggcatgggcaaggtgctcgtgctccggtacttgcggatgtgcgaggtgcccgaggccaagtgcgaggggtcgcgcgcgtcctgcgtgctcaagatggaccctccccgctcacccagctgcgagcgcaggccgtctctcccgccgtcccccccatgccccatgcgcacgcctcccgggtcgccgctccaggctcccttgatgcgcacgcagatgctccaggggctgttcgacgctgccaaaaacaacggcgagcagatgtgccgccgccagtaacctaggctgcgcagtacgaaagttagtgcgtgatcacgttttttgcaatgtcgatcacgccgtgcgtgcccgtcttgcgctcgcgctccaccacgctagtcacgggccgcgcgtccgagactagcgaccccagcatcgagcgcacggcgccctccgcggcggggtggcgcgtcagcagcaggaacatcacgatgtgcgcggagacgccgcgccggctcagatcgtgcacggcgtcgccgtccatgagcacggtgttcgagaagtacgtgaacagagtgttgtctcgcaccaggaaggccgagttcgagacgctctcgaagtccacgatctcgtcgtcctgcacgcccatgtccaacagcgtctgcacgagcgcgggctcgtccaggaacaccactgcgcgcgcgaacccgcagtccagcgcgcgcgcgtccgcctccaacacgcgcgaggcgccgccctccggcggcaggaaggcgcagggcagcggcgtgcgtccgtccgccggcgcctccccgagctcctcgagcgcgaaggccagcagcgtctccatgcgcgcgcgcgccttgtcgaagttgtccgcgaggtcgcggatgcggtctgtctgcgagaacatcttcagcatcgccatgagctgcacgaaggggtgcagcacgtatatgttgtccacgagcagcgtgggcagcgcgcgcagcgtggcctgccgcacgttgaagctgtccaggatgtgcccgccctcctcgtcctgcagcaccacgtagttcttcaggtagggcacgcgcagcagcacggtctgccgccccgtgacgaagtatatcaggaaggcgaggttgatcaggaacgggcgcgcgttcgtctgcaccatgtcgatgtcgccgtactctatctcggggttcagcaggtgcagggcgtacgagccgtagcacacgcaccgcttgttgtgtcggcgcaggtgctccttcacgagccgcttgaccacctccaccaggtccgagtgcttgtgccgcgccatcggcgcggcctcctcgggcggcggcagcactgcgtacgagttgagcgcgcggctggcgagcgcgcgcgcgcggggcgcgtccacgcgccgcaccgccgcgttgattgcaggcgtcggcgtcgtgagagagcccagcgtgcgcgtgaactcgctcacgatcacgctctgcagctccagcaccgtcaggatctggcccagcttctccaggcgccgctgcctcgagaagtactcctcgatgcgcgcggcgatctccttctcggagccgcctagcttcttgaagaagcgacgacgactctttacaacaagagagagaaaaagcttcctatcgaagttgaggacgcgggtcatgttgcggcgctgcgcgcgcaggagcacgcagcgctccatggaggggcgcgagccgaggtactcttcgatcacgggtggagccatgacagctattttctgaacccgcgattattgtacagcgcaagccgcgcgcagacctgctggcacagcagcgtcgtgtttcgcatgcacacgcgcgaggactcgatcgtgcgcgcgtccggtgcccaggcgcgcagctccatcagttcctgctcgacgaagtccacgggctccacgaagcgctctgcgcagagtccgtccgtgaacgcgttgacgatctgccgcacgagcactaccacgtccacctgctccacgaggcgcacgcccatggcgacgtgcacgaagaggcagcggaagagcgcgtccatggccatctggtggtccgagcagggcccgaccgcggtcttgcagcccagcgcgaagcgcccgatgccgcggtactgcaccatctccgagggcgagaaggagagccgctccatcttgagcacgggcggcgggcccgccggcagtccgcgcgcgaggtccagcaccggcgtccacccgggcgtgaacatgtccgggatcaggaagagcccgtagctggccatgcgcgcgatgtcgaaggcgtggtccacgaccttgttcacggcgctgtccgcgcggtttacgcgcagcgcctgcaggatcacgtttccggaagcgtgccgcgtgatcgcgaggtccgcggtcgcgtacccgcgcaggcccggcaccgcgtacgcggtcagacacacggccaggcgcgcgctgtgctccgaggagaagatctcctgctcgtagccctcctcgggctcctcgcactcgcgagggcgccgcacgtcctcgaccgagcgcagccgcatctctccctccgacaccagacagccaagcgactccctcaccgccggcgcgagcacctccgtggcgcagagcgcgtcgtgcacgcgcttgagtgtgttcggcttcagcgcgtagccgaagagcagccgcgtcatccgcgagcccgagaacgcgaagcggcgcacgtactcctccgcgagctcggggcggtcgttgatccacgaggtagagaagacgtggtcggaggcgaaggggtctgcaccaaccgcgagcagtgtggacagaggtacggtgtcgaggaagtccacgacgtcggggaagttctggcgcacgcaggcctcggcgacgcgtctggtgtgcacgcacatgtcggtgacgggcacccggtggccggactccacgacggacacgcagacgtcctcggtgacggcgtccacgggcatggtccgcagcagcttgccgagcacgtcgccgaacccgccctcgagcgccttgcgccacacgaaccccgcgtcgaacttcccggggaagtccgcgatcaccgaaagctccgcgtgcgagaggttgtccacgttgaggtaggtggcggcgtccacgaagatgggcccgaaggcgccggtgtcggagacgcggtctctgaggtagtccctggcgtagtggaggtactcccgcacctggccggcgcggatgcgctcgagcgcgaaggccttcatggtctcggagcagagcacggagtgccgcagcccgtccagtgtgcgccgcacgtcgtagacgccgcgcatctgcgcgagcatctcgacggcgtccgccggcgtcgccgccgcgagccccgggttcactggaggtatcctgtgttctgcgagcatgcgcttgaggaaacagaggtccagcggccgcgtggtgtacagcgcggaggccatctcggggcgcgtctcgacgatgtcctcgatcatctcgtccgtgaatgccgcgttgatgttgtgcacgctgcgcgcgttcacgtgcaggaggatgtcgcccacgttgtcggggaatcgctcctcgatgagccggacgtcgtcctccgtgatgttcatgtagggaatacagcggcagagcagcgcgtagtccgcgaactgcgcgatgtagggcgtgtggaactcgatgtgtctggcgaagagcgcgccgcagcgccgccgcgagagctcttcgagcaggtcctcgggcgtgacgtgctgcgggcggaagaggtgcaggtgcgtggggtgctcggcggccacgcgcgcgtacagccgccgcgggaggtgtctggggtgcacgccggcgagcacgagatccatggcctctgaggtagacagtgcggcgaacgcgcgctcggtgccgcccgcggcgacagcggcgccgacaaatctcttgagcagctgcagcatcgcgtgtttgggctttcgcggaaggcgcttattttaatgttattggcggtggccggtgcgagataaaaattagaactgatgccgcagttgttgatgatatgatgattgcgctggccggcgcgagataaaaattagaagctgatgccgcagttgttgatgaggatggtgagtgcgctggagcaagcggtgtggcgcgctagcttcttgctggccccgtcggccacggcaacgacctttccggatatcgtgatggtgcaggtgaagcgcggacagtgatcctctccgccagcacgcgtctcgcagaactccagaggtctgtgtgtcatcatgcagaactcgttgaccgcgctgaccgggttaagacttttgaggcgtatcacggcagactgagtcatgatgtcgatgtcgccgccgaaaagcgtatcgcacccagcctcggtctccatgggctcggtgtcggagttttcgtcctcctcggtgggcgcggagggcgcgcactctacgaaccagcggggcgggtttccgtcctcacagcaaacctcgtccgagtccagcaggcggtacagctggcggtttgcctcgtgtttggatatgccgagctccttcgcgatttgcttggccggcagcttgtcgtcggattttctgagaagctcgaggatcagagacgcgcactcgcaggccattgtggcgatttacggggcgtgcgtttttttaggattttggcttgcctttcttttcgcagaacttgggaggattgaaactcttttggcaatttttgcaggcgtacttgatcaagggcggctcgtccgccgagcgcgtctggatcatcatcggcatggtgttcttgctctggcacgaggggcagggcaggttgaacttctcgtcgagcacgttgaagtacccgctgtagtcgtggtccggcacctcctcgatgtcgtagggcacgcgcgcggccacgcacttgaccgcgaagagcaggtaccgcagcgcgtcgtgctccgcgccgctggtcgcgcggatctgcgcgcacaggtccgcgtagtcctcgttcgcgtccacctccaggctgcgcttgttcttgtacgagagccggttcttggcgtccttcgagtactcgatgccgatgttgtgcgcggggtcaaagttggtctcgtcggtgttcgaggtcttggtgttcacgatgttcttcagcgcgaagcgctgcgcgcagtccagcgcccatcgcgcgatgcgcgcggcctccgccgcgtcggtgtgcttcgccgcgaggtcgcgcagccggtcttcgtccatcgcccgattttaggttgggtatattatctcaattccgctcttccgcgggccgcgggcgcgcgcccgcggcaaattaggcgttacaaatggacttcgtgcggcggaagtacatgatacacgccatcgaccgcaacctcgacttcatgaaggccgaggtccagcagaaggtctccatcttctccctcgggcacgtgctcgcgctccactacctggtcaccgcctttccgcaggcggtcatcaccaaggacgtgctcgcgagcacaaacttcttcgtgttcgtgcacatgtcgcagcggcacgaggtcttcgacgccgtgctcaaggcggccttcgacgcgcctcagctctttgtgcgggcgctctcgcggcacttcgaggccttcgttgccgccatccgggcctaccgcgcgacctgcgcggagctgctggccgacgcgcgcttcatggaggtggctgcgcgcgcggccgagctcgcggaggtcattggcgtgaaccacgacatcgccgcgaacccgctcttcgcggacggcgagcccgtgcgcgacgcggagctcattttcgcaaagaccttccgcaagaccgagttccgcgccgtcaagcgcctcgccgtgctgcggctgctggtctgggccttcctcgtgaagaaggaccttggcggcgagtacgcggacaacgaccgccaggacctgtttacgctgctgcagaaggccgcggggcccgtgcgccacagcgcgctcacagagagcatccgcgagtacctcttccccggagacaggcccagccactgggtctggctgaacgcgcgcgtggccgacgacgcagaggtgtaccgcgaccggcccgcgcgcacgctctacgagcgcgtgctcagctacgcgtactcagaggtcaagcaggggcgcgtgaacgccaacacgctcaagctcgtgtaccggctcgaggacgaccccgacatcaagggtctgctgctgcagctcatctacgacgtgcccgcggacatcgtcggcgtcgtggactccgcgaacgaggagtggcggagctacttcgtgagtctgtaccgcgagaacttcgtcgacggacgcaccttcacctcggacgcgcgcttccgcgacgacctcttccgcgtggtcgccgccgtcgatcccgacttcttcgagcccgagcgcatccgcgaggccttcagcgcagacgcgcggctgcgagagcgcttcacggacatggacctcaacaacgccttcatgtcgcacctcatctacgactccgtggaccccgacgtcgccgccgccgagcgcgggctcgcactgcgcgtgcacaacgaggactccgactacttcatccgggagtacaacacctacctcttcctcagcgagaaggacccgctggtgctggaccgcggggcgctcacgcggctctcggacgtccccgccgagcgcttccgcgacctcttcagcgacagtgtgctgcggtacttcctggacgcgaagctgggcacgctcgggctggtgctcgaggactaccgcgaggacgtggtcgccgccatgcttcggcacctgcgccgcgtcgaggacgtgtcttccttcgtgacgtacgccgcgcgcaagaaccccgcctgcgttcccggcgtcgtgcgcgcggtcgtgagcaacttcaaccccgcggtggtcgcggccatgcgccccttcctgcgcgagcacatgacgcgcgtggacgcgctgctggacggaatgccgcacctctcggaggccgaccgtcggtacatccgccgcgtggtgctgcagggccgcgcctgattcgccgtcaataaatcgcgatggtggacagcggcacgcacgacgtggactccgccgcgcaggagcgcacgcccaaccagcagaccttcttcaccaaggggctcagtccgctgatgcgccacacctacatctacaacaactacgcctacggctggattcccgagaccgcgctctggagcagccgtctgggcgactaccgcgtcacggacttctacccgatctcgctgggcatgctcaagaagttcgagttcatgttctcgctgctggcggaccccggcggcgcctgccccgcgtacgagcccaagctcaacaccgagttcctgaaccgcggctccttctcgggacggtacgtgaaccccttccaccgcttcgcggcgctgcccgagcgcgagtacatatccttcctgctgctgagctcggtgcccatcttcaacatcctcttctggtttaagggcgagaccttcgacactgccaagcacagcctgctcggcgccgtgtacaccacgcccgagcggcacatcgagctcgcgcggtacctgcggcgcacgggcgactacaagccgctgttcagccgcctgggcaacgacgacacctactcgaagcccttctctgggttcacgcgcatcagcaaccccacgcccatcgggcggctgccgccctcggacttcgagacgctggccaacctgagcaccattctctactacacgcgctacgacccggtgctctgtttcctggtcttctacgtgccggggctctccgcgaccacgaagatcacgcccggcgtggagttcctcatggagaagctctcgctcgcgcccgagaacgtggtgctgctgtagcctcaaacataaaatataggcgcctttgatcgcactgcttcagttcagacagagctaagatggcttcctacatcagcggcgctagcgccagcgcgaacaccgcccagggcggcgattctcagtacccacagtactattatcacacacgcacctcccaaggcgacatccgcgacgaaagcgaaggttgcttccacaccacggacgacgagcacttggatctgtccgacgactacctcggcgatggcgcaccacactgcggacacagccacaaccacagtcgcagagatggagatcggcaccgccagcgcgcaccgcggctctatgaggacccggtgcccgcgaacatcatggtgcccacgctcagtctagagcagctgctggaggaaacctcggtcgcggggggccttctcggcggcaggacagagagggacgtggaacagctcctggaggagttctccgcactctgtcccggggaccagatcaccgcgctgcgctgcatggcggcctccttttaccgcgacgcgctgttcgcgccgtacgcctgcatgcacctcatcgccagtcggatgcgcgtgcactacgcgcgcgaggtcgtgcacgtggccgaggacctcgcggacgcgatgtctgcgaacagcggcgtctgcttccggcggtaccgaaagcgcgtgctagaggacatgctcgcggaggagatgggcgtgtacaattacctcgcgcgcgccaacgcggacatctgcgaggacaacctgctatcggccgtggagacgctgctgcggcgcttccgtcggatgggctgctaccgctctctgtgcatgctcaagatcctcgcgctgcagcacgaggacctggccggcttcatccgccgcagcataagaaaaacctgcaacttggcacacgcgcgcacgcacacggtttacgtgtagttaccctgtaaagacgggcttgctcccgaacaagcgctcgaagaagagcgtgcacatagccttattgtccagcaagttgactatctctgtacacagcctcttgaagtacacctcgtacatgatccgctcgtttttatccagtctgaaggtcttgtcgaccacgcgctcgtaggacttcacgttcgcgatccggcgccgccaggggccctcctcgcacacgtacgcgaagaagtagcgctcgccgatctcgatggcctccgcgttcgccgcgttgtaccgcgtcactagcgccacgttggggttgtcgggggacttgaagttcttgtggtgcgttcggctcagcaggaaccagtccagcggcatgctgcgcgcctcgaactcgaaggtgagctcgtcctccagcgagcgcaggatctccacgcccacgttcccggagccctcctccgccagcgcgcggcagagcatgtccttgtacttgcggatcatgagcttgtggaagggcgccacgtcgcggcgcgtctcgctggtgcccttgctcacgcgctcgctgccgccgccgtcgctcaccgcaaacttgatcgtggtgtacttcttcttggactgcatgatcaggttgcagtacaccgcttcgaactccaccttgaagttcgcgaagagcacgtgctcgttgatcacgcgctccagacagcgccccacgcgccgcgagaacgcgatgtcggaggcgcccacctccaggaacacggagtcggtgtcgccgtacacgctgcggaagcccacgcgctccgtgcgctcgccggccaccgccgcgtcgatctctagctccgcggcgcggcccgcgaaggcctcgtcgcgcagcagcgggttgtccggcgccgccgccagcgacagccgcgtgccgcacaccgacgcgccgtctagcgtgcgctccaggtacgcgatcatggtgcgcccgatggccgtgcagctcttggccgaggcgtacgagaagagcgcgctgttgcggaagcccatgagcccgtacacggagttggccgtgatcttgtacgtgtactgcatcgagttgtagatctcgcggtccaccgcggtctccgcggccttcatcagcttcttgtacttggcgcgcgcgtccaggaaggagcgcagcagcatcgggatgatgcccttggcctcgcggtcgaagatggccacctcggcgacgagctccggcgagcgcggctcgcagggcaccgcgatgtaccgcggcgccgggaacatccgccggacgtccacggccgcgacctccgcgtcgagccggttgtccgagacgaccacgccgacaagcgtctccggcgacaggttcgcgtagatgcacacgttcgggtacaggctgttgtagtcgaagatgagcacgtgcttgttgtgcatcttctgcttgggcgccatcacgcggccgccctcgtagaagaacttggacttcgtgtccgcgcgcaccatcaccgtgcggttctccagcagcagcttcatcagcgggcccttgatgcaggtgctcgcgcggtactcgaagaccacgctctgcggcagcaggtacgtggacgcggcggccgcgatcttggtctccacgccgtagtgcgaccagaggtagaggcagaggcaggcgtcgtgcaggcagtaccgcgccatgtccagacacacgtccagcgagtagttcgcgtacatgtccgcgaggctgacgtcgtccttgccgaaggccagcgtgacgcggtcgccgggcgcgcgcgccgcggggtccgcgaggtccacggtgaagccgtcctcgtcgacgcgtttgtgcagcacgcggcacacgcgctcgtccacggtcacgtagttgccggtgctgagcacgcgagcgaacacggccgcgttcccgtcggcgtcggtgctgcggtcgccgcgaaagcggaccgcgtccggccgcgcgtcctccacgaccgcggtgcagtggaaggcgttcttggatatggcgtccagcttgtaggagtccagcttctcggtgcgctggatgaaggcgtacaggtcgaagtagatggtcccgttgttgttgttgatgtggaaggtggtgctcgagacgccgccgacgcccttgtggctggacttcgtgcgctcgtacacgcagaagttgactgtctcggtcccgtccggcagccggaagcggatgtgctcgcccgtgagcagcgacagccgcgagtccaggtaccgcaggtcgaagttgtggccgttgaaggtgaccacgaagtccagcggcatctcgagcaggcgcttggccacgcgcagcagcgtcacctcgggacacagcgtgacctccgcgtcgaacttcacgtccgccgggtccaggcagaccgggatctcgcgccgtgccgcctcctcgaggtccgcgtcggagagcatatcggagttcgtaagcgtgaatcgccgctccgcgccgtccttgtccaccacgcagaagctgatgtgcgagacggcgttcttgaagacggaaggaaactttttctcgaagtggcactctatgtcgaggaagagccccgagcgcgtcacgttgaagcgcgggatcttctccgcgaagcacgcgccggggtcgtcgcagtggaagcagttgctccccaggtcgcgcagcagcgcggggtccacgcggtagcacccgtcagggtcgatgtcgtgcgccacgaagaaccaggacacgttcagaaagtcggacatgaagacctctggcggcgcgagcttgcgctcgctggccaccagacacagctctatctccgagcgctggcgctccggaatcttcgccgagcgcgctacgatctcgtcgatgctgaccacggacatgggcccgagcgcgcgcgtccacgccagcggctgggcgatgtcggccaccgcgtccgcgcgcaccacgtagtaaaagtgctgcacgaagcgcaggtacacgacggcgttgtcggcgcggcgcgccttgaggaagaggaaccggctgtcattgccgcggttctcgaaccagttcaaacatttcagctccatttcaaagagcataataacatttcatttaaatggagcctcgcttctggggccgcgccatgtgggcggtgatcttcatcgtgctgcggcgcttcgaggagcaccgcgacctcgagcgctgcaagcggcagctgtacgtgatctgctccacgctgccctgcatcgcgtgccgacgacacgccaccgccgccatcgagaaaaacaacgtcctctccagcgaggaccccaactacgtgctcttcttcttcatcaagctcttcaacaacctcgccttcgacgacagatacaagatcgaccccgcgaaggtgcgcccgctcgtctagagcatgccctcgtacgcgcgcgagttgtccgagtacacggtcaccgcgatgccctcgcgcggcgtcacgtgcacgtgcatggagtccgtgatcacgaagcccacgcccgtgaccggctcctcgcccgcgtcgtccgtgaagagcagcccgtggttgaagaggtagaactcgttctctgcgagcgaaagccgcccgcggtcgcagaggtagtagaagatgtcgtagtcgcgcacggccagcgtgaacgtggactcgctcaccacgatgtacttgtccagctcctccagcacggacgcggccgcgcggcgcgcggtggcgcggcactgcgtcgggcactcgcacgtgtctgcagagtacaggatgcgcgttcgccccgaggcggtctcgagaaaaacgttaatcgcctccatcgcccagaagcgactcgaggatcgcgagcaccgtgcgcagcacgagcccgatggtgcagaagggaacctctcccgactccgagcactcgcggatctccgtctccacgcggtcgtgcacttttatggagggagccgtcgttccagtggcctccatcgcgacggacaccaccttggccacgaactcgcggatcttgctcatgcgccggagcacggtcacgcggaagaagacggccgcgagcaggtactcggccacgcagctcacggcgatgagcgcctgcgcgtgcctggtgttgagcacgtgcgcgtcgggcacgaagtccgagatcttcaggtgcgagagccgcacgagcgcgttggtgtccttgacgccgtcgcaggaaatgttcgcgaagatgagcttctcgtagtcgagcgcctcgaccacgcgctgcgcgtccatgtgccgctcgcccgagagcgcgcggctcaaagggccccagcacaccgagcccgcgaagcaggggtccaccacgccgtgcatcgccagcagcgtcacgtcggccaccgccgcgaggatggccgcgtcgtcgagctcgttcgcgggcgtcgcgcccgtcagggacgcgctgcgcagcgcggacccgcccggcgccgcgtgccgcgcgcagaactcgcacccgcagcccggcggcagcggcggcttcgagagcagacttatgagcccgcccacgtgcccgtgctcggtgatcagaagcgccaggatgctgtcggtgctgccctctatggcggctgtggccgcgctagaaggctctcccgtgacctgccatccgcagacaaccttgagcatcttgcgcttgagctcgttgggcgcctccgaggccagcatcgtgcgcgggaacgtcgcgttgaggcggaagtcctgcagcagcttttcgagcgtcgcggtcttggcgtgccgccctttgcgccactcctcccccaggtgccacatgagctcctcggccgtgtccagcgtcggcgagacgcgggccttgggcacgcgcgccgcgctgcgcatcagcggctccgaggcgcggaagctgccgcgcgcgtgcaggcgcatggacgcagacgaggaccgcatcgagggtctctggtggaagctcgtcatcgtgaagcgccgcgtgagcggacctacctcgtcgcgcgactcgtcgaagtccgggtcagggtccgtcgtgtcggtctcggcgctgtgcgtgctgggcgcgctgctgcgggcgctgcgcgtgctgcgcgtgctgcgggtgctctgcgtgctgaagctgcgcgagcaggagtccgccgtgtccgcttcctcgtagtggaagtccacgtgctcctccggcagccggcgcgagcgcgacttggagatgccgaccatcttgtccgcgccgaccgggcgcacgcagagcgccatcgcgccctcgcgcaccgcctgcgcgaactcgcggctggcaaccatgctggggttgaggaacttgatgatgttgaagtatggccactcgcagacgagccgcgcgcaggtcgcgacgtcgtcgacgcttctgagggccctgttcagcggggggatgttgctgccgtcggccagtgcgacgaggtcctcgggggagatctcgagcttgggaatcatctcgttcacgagcgcggggtcgatggcgtggcagacggtctctacctcggtgcgcgagagcttgagctgcgcgcaggccgcccacggcgcgcacgtgagcgcgaacaccgcggagactcggcccttgccgaccatcgccacaacctcgggctcgtagaccaggccggccttgagcagagcctcgagcacctctatgtcgtcggcggcgagcagcgcgcgcctgtggaagcacaccgcgggcatcatcttcttgcagatgccgcgggtgctcttgagggccgtgataaggtcggggagcctgacgtgctgcggctggaagaacatgacgttggcggggctcgcggccaccgcctcgtcgtacaccgacatcggcaggtcgccgtggagcccgcacggcagcaggctcagcagctgcgcgcgcgagagcttctcggcgcagaagttcttcttggccagggcggccgccgcgtcgcgggccttcttggggtataacaacatggcgggctttaaacacgaaacaaaaatccgggttgtaacatttcaattttgcatgttctgggcctcctcgcagagtttctccaggccgccggccacgatggcgtcgacgaagaggtcggcctcggtgaagcggtggttgccgcgcaccgccaccagcgcgttctcggtgaccaccaccgacagctgctgcgcagccgtgcgcaggtcgaagtgtcggcacgcgagcccgtggcccagagtctggtccagcgccgcgagcacctcctccaggctctcctcgcggtggttgtagaaccacatcagcacgaagtaggccacgtaggtgtagaggtagtgcgcgaccgcgcgggcgcgcaccagcggctggttgcagcgcgcgaaggccattccgctggcgatgaggtcgtcgtccagcgtggcgtagtcgccccagttgagcgcgctgaactgcacgctgtagaccgcgcgcacgaacccggactcgaggatgtcgatctgcgacggcgcgccccaggtcaccagccggtacacgatgccgcgctgcatcagccgcatgaggtcgccgccgacctcgcgcaggcggtgcgtcagcgaggcgaaggccagcttccgctgcgtgtactgcaggcccacggccacgcacccgtccgactccaccagcacgagcttgtggtccgtgcagccgtcgctgcgcaggccgccctcgcgcgccatcatgtcggtgacgaacatgtacttgcgcgcgcgcggcccgaccaggatgcgcttcttgccttccatgcgcagcaccacgtcctctagcagccgcgtgctgtccacgcgctccacctgcgggtggatggtgggctggtagttgtacagcagccgcggggaccagttgtaggcgaacgcgaagaggtgcgtgtccaggagagagatggggaagacgccgccttcggtggcgcgccggaggatggcgagcttggtctccgcgggcagcaggctcccggtcaccgcgccgaagaacatggggtggttgcggaacttgagcgcgtacgcgctcagcacctcctctcgcgagaatatcgagtccgcggggttggagctcgcgggcaggagcacgtcctccacgctcagcacctcgtcgatgaagggcagcaccatgtccacgttggaggcggtgaaccactccatgtccacggcgttgcgctccacgatcacccggatcgtctcctcggatatgttctcggcgaaggcgctctgcagctcgatcaggttctcggtggcgtcgatgctgagcccgaggcgcatgccctgttgcagcacgtcgttgatggggttcacgtacatggccaccgccatgcacccggccggctgcacgcgccagaggttggagtacgcggacacgtccgtgatgcgcagcgtctccagcacgttgtacatcgccgcgcgcatttccagcgtgtccacgtacacctccgcgtgctcgaagatgatgtcgagctcgaacgcggacagcggcagactcacgtagatctggcaggcgtcaaagagttcctgcgcgtactcagagaagcacgcgtacgcgatcacgcccgcgcggttgacgatgtagtccgccttgaacatcttcgtgaggtaggcgtacagcgaccggcaggccacgtgcggccttagctcgtcgagcacggcgtccagcacctcgcggtgctgcagcacggaggggtgcaggaactgcgtgaagtccaccgcggtctcatccatgaagtcggggatggtctcgaccaccaagcggtggttccgcaccgcgcggaagccggtgttcatcggcgcgatgctgtgctggtcgtggacctccagcaggatctcgtagccgatctctcggcagctcagcgccgcgcgcgcctccgtcacgctcgcgttccggaagaactgccgcaggtgctcgtccgtgcggcagagcgtgacggattgggggatgcgggaaaggtcgcaaagagggctgtggacggagatgcgccgcagcgcgtggtctacgaactcaaaaccgcgcctcgacatcgtgaagtcgcggagggcgtacatgttgtaggaacagaggcggaagaggcagtctatgtctagcatgttggaaacgcagtacgcgtgtctgtggaggtgcgggagcatggcgggcacgacctcggtcgtgttctggagcatggtgcatacgaggtcgggcgctgtgaggtcgggggtgacgatgaggatgcaggagctggagaacttgctgagctcggaggccagccggtgaaggttgtagtcgtggcgctggctgaagttgctgtttatcgtgcccgtgaagcccatgagcgccgccagcgtcaggtcctcgcgctcgatggcccagatgtctaggccggaggctatcatgcagcgcaccagcgcggcggcgcggtcgctcgtggggtagctcatggtgctgtcggtcgtgcgaatcagcatgggataatgcttcatttttacggtcgggggttgcggactgtggggcgcacagggcctgcgggcggctcgtgccggtccgcggcgttcgccgaacgcaggaacgggcccatgcgcgcccaggccatccacagccccgccgtcagcgccagcagccagacgaataccacgatcatcttttatgtagctggaactcgcgctcactccccgccgcacggcgacggggagagcccagagccgagctccatgcgcgtgctctgcacggtgagcgactccacgagcttggacacgttcatgcgcgtgttgtcgggcaccaggtgcgcgaggcgcgcgtacacgtcatcgtgcatgcgcttgctgcagcggtccaggctcgcggagagcgcggagactagcgcggtgtgcttcgcgtacacgaagtcgccgagacacacggtctcgagcccgagcacgtcggcgctctccgcgtccttgagcgccacgaatatcttctgctcctcgcgcgtcatcgagcgcatgaggtagtcgtgcagccgcgagcgcgagatgagcccctgagagatctgcgggctgcgcatgaagcgccggcgcatcgcgcacagcagctcctcgtcgacgacgtacatgctgtccttgatggagctcttctcgtcgatcacgagcagaccgtcgttggcgaccacgttgatgaaatcgtccacgtgccgcgcgtctatgtcgtagcgcgtgccgccgcactcgatgtgcgagggcggcgacttgagccggttcgcgagcgccttcacgtcggagacgtcgatgtacagcgaggactcgcgcgggacgcagccgaggatgcgcgtctcgagcggcgtgaggatgagcacgcgctcggcgccgtcgacgagcttgccgtcgtcgggggaaaagaagttgttctccacgatgctcgagacgaggctggcgagcacgccgtcgcggtaggcgccgagcgcaaactcctgcacaaagggcgcgtgcagcaagtccacgggaatgcgcatctccacgcggcgcgcgaccgacttcttcttctgcaggtgccgccggtccaccatctcgtccacgatgtccgatatgcgggagcagaggtacgccttgaggacgttggcgtttaccttgttgaagatgagccgttcgtcctccatttaagctgctcagacgagctttaaatagtggaaacacagcagcacgccgatcgccgccgctatcaggccgattagaaaaacggtggtccaggggacgcccttgggcctatcgcacgccggcttttcggtcattacggtgcgcacgatgtttaggaactcctcgaagtcctcgtccgagttggagaggaaggagccgaagacgccggtgtacagtttgtccatttactactagatattaaacggcgcttccaactcctcgtcctcgaagcccgcgccaggctcgacgacgcccaggccgcgcacgtcctgctcctcgttgaacgtggtctgagtctcgctcatgcgcacacacgtctgctcgccctcgagaccgagcacggtcagcgagcactcgcgcggcatggtgatcttcttaaccgcgaaggtgactttgccctcgccgcccgagcggtagaacaccaccggcgccaggatgagcgtcgccatctgcgcgtcgcgggttgcgaggttttctatctctcgcgtcagcggacatatctgcggctgggtgtcgtcgtcgctggtgaactccaggagagcgccggtcaggcggttgagatacagacatccggacttaaaggtgttgtcgatggccgtttcggtgttgaagttgcgcagcgacggcggaacgcgagcgccggttttgatgttgtcgtatatgttctccagcagctggtagagcagcggactggcgctcacgggcttgaggcgaccgaagtacccgctgtcgttctgccgctgcatgtcggtcttcttgctcgggtagatcttaaactcgcccttcacgacgatgagcggcgagacgagcttggatgctagactttcgacgagacacacgttgatgttggagcagctcgggtactgcgacggagttagagtcacggcctcgatgaccttggtttggctcgacgagagcgacttagcgaagttgatcgcgtcggtgcacgacatcgcctggttctcgccgaaccgcctggcggacgctgcatcctcctgctgaggagcgcggttagacgcgacggtggttttggatacagcgcgtttcattattgcggcgattttaaagtacgtgtatactttcagttttgtcgccgagcgttcagcgcctgcatgcagaggaagtacaggatgatggtgcacgggatcgtggtcagcagcgatacgaagtccatcactgtgaggacgcgcagcgccccgcgcgagcggatgcccagcgagggcgcgccgcggcgcgcgatggtggccccgttcgtcaccactaccagcagcattaggatggtcgcgcccacggcgacgcccaggtcccgcgactccatttatagtacagtatagagcgaccgcgtcacgaactctcggctggccaacacgcgtccgtcgggcgggtgtccgccggccttcccgcggaactccgggacctcgaagctggacttcgtcacgcggtacgtgtacttgccgcgccagaccaggttttccttctggaagacgccgtccatggtcacgcccgccatgaaggcgtccttgacgatgaccagcaccgcgtctagcttgcgcccgttgatgtgcgtgacgaagtccgtgccgctgcggctcgcgcagcggatgtccacgcccgagggcaggtccaccacgaacacgaagcgcttcggcgcgtagagcaccaggtccgaggacggcgacgccgaaggcgccgaggggaactgccggtggtcaaaagggtgcaccacgcccacgatggacgtgacgcggtcgtccgggaactgcgtcgcggcgccgccgccgcggtgccgcgtgaccgtgcttctgcccacgtcgtcgcagaccacgtgcagctccgacacgatcggcagcagcgtggccagcatgcggtcggtctctgtgcgcgtcgcgcagcggtacgcgatcccgcagtgcgcgtcctgcgtgcgcccgaagaagagcaccagcacgctcgcgtcctggtcgaagggacacacggccatcacgcccaccggcggcggcccgtggcctgcgtacgcggaggagaactcctgcacctcgaccacggcgtcctcgcgcgcctcgccgggcaccatcgccgccgccggccgcagcgcccgcacggtctgcttaaccgcacgcgcggcggaggccgcgctcggcgcgactacgcgcacggccgcgtgcgcgcccggcggcggcgcggttccggccatccagcccaccggcgagaagaacacgtcgcagacgtgcacgcccgcggcctgcagcgcgcgcgcgagcgcgcgcacggcctcccactcctcgcgaaaggcgctcgcgaccgcgagcgccttcagcaccgtgtccacggagttgacgggcttctggaagaggttctcgttgttgtagatgaactcggggagctccacgggcactgtgaacagcctaatctcgtgcgcgccgctgggcgtgagccgcgtcgcgggcttgcgcacgccggcgccgatctgcttgaagaagtggttcatggcgccgccggcttctcgggctccggcgggagcagactatttattcgggaggttatcctttccgaaagcacctgcacggacttccgcgtccagcgctccatcttcatgtactccttcatgccgtcgctgagcacctcgacggcctccagcttgggcgctgtcgggtcgaagaggatgctcttgagcagcgtcatcttcttgtccgcgaggaagcggaagtaagtgtagatgcagcgcagcgcgcggaagttctccgggtgcttgatggtgcacaggatcatgaagatgcaggtgaacatgccgcactcggactccatgagctggttgacctcgaggttgatgcagccgcgccgcgccttgaagttgtccacgaagaagcgcatgagcacgtccacgtcgcagttgcggttgtccaggtccgcggtctcggcgttcacgttgaagccgtccgagaaggagtagaagtagaagtacttgcaggggtggaactccgaggggctgttgccgccggagtcgtagaaggacacgagccgcgagacggtgtcgaagatgcagcacttccagtggaacatgtagcagaagccgaacatcacgtagcgccgcccggcgcgctcgatcttgtccttgagcgtgaggctgaccatgttgcagcggaagcggtccgccttttcgtggatggccgcgccgttgaggaagttcaggttgaactggcccaggtacgcgacctcggtgccgaacgcgaagggcgccaccagactctggatgctcacgttgctcatccaggcgctgcggtcgggctttatggcgatgggcactaccttggtgttcacgcccgtgctgacgcccgcgcgcgcgaggtcgtccacgttcagcggcatctgcgagaagtccacggcctcggacaccttctcgcgcaacgagggcttgaagaagaagcccagcgggaccttccactccagcgcgatcgcctcgcggaagccgtagcgacccttgaggctggccagcaacgcggtcttctgcgcgacctcgtccttctcggtgtccggcggcgcggcgtcgatgagcccgcgcttcgcgaagtccagcagcgccgccagcgggatgcacgagacgcgaccggccgtcgcggattcgtcgaagcgccgcaccacgtacccgttgcagttggtcttgaagttggacacgtccaggtgcgcgctgagccccaccaccgagtagatgtggcacagaaggttggtgaaccccagctccgggattttgctcaccactaaatccgtgtacttgtccatttatcatggagaatcatctgccggacatgctgatgtttcccaactgcgtttctgtgtttccctttgagtactcgctggaggacgtgttccgcctccccgaggagcgacggcgcgcgttcgccatggccgtgttcccgctctccaagcaccgctggaggggcgcgcggctccagcgcgacgagcgaagcgtgtggctcagcgtcgaggaggaccgcgggcgcgcgctggacgagcggaactgctcctggctctcggacgtggccgcgcgcatggtcgacgacgagggccgcgcggtcacgcccgaggcgtacgccttcatgcgcgccgcgcccggcgcgcgcgtcgccgagctcgccgcggacgcgggcgtgctagcgggccttgtcgccggcggcaacgcgctgcgcgtcttctcctcggagtccacgcaggcgcgcgagggctggaaggcgcgcagcgtgggcgtgctcggcaacgcggcgccgctggcgcccgtgccgctggcatcgctgcgtccggaagtgcagcgcgagctcttcgccgcctggatcggccgccgccccgtggtgctcacgggcggcacgggcgtggggaagacctcgcaggttcccaagctgctgatgtggttcaactacctcttcggcggcttcgagcgcctggacgccgtccgcgagttcgcggagcgcccgctcgtgctctcgctgccgcgcgtcacgctggtgcgcgcgcacaccgcgacctacctcgcctcgctgggcttcggctcggccgacggctccccggtctcgccgcggtacggcgccatcccggacgccgagcggaacacggccccgcgcgcctacgggctcgtggtggccactcaccggctcacactgactgccatccgccgctacgacacggtcgtagtggacgagatccacgagcacgaccagatgggcgacatcgtggtcgcggtcgcgcggaaactgggctcgaacatgcgatcgctggtgcttatgacggccacgctcgaggacgaccgcgcgcgcctggaggagttcctggaccggcccgcctttgtgcacatagagggcgacacgctcttccccatccgcgaggtctacgtgaagaacacgcaacagccgccgctctcgcgcaagtacgcggaggcggagctgcagaacgtggcgcaggcgctgggcaccttcgtccccgagcagggaaagtgcggcatcctcttcgtagccacggtggcgcagtgcgcgctcttcgcggagaccatcgaggccaagcaccccgggctgctggtgcgcgtggtgcacggaaaggtgccctccgtggccgcggtgctcgaggaggtatacgccgcggaccggcccgcggtgctggtttccacgccgtacctggagtccagcgtgaccgtgcgcaccgccacgcacgtctacgacactgggcgcgtgtacgtgcccgagcccttcggcggccgcgagaccttcgtctccaagtccatgtacacgcagcgcaagggccgcgtgggccgcgtggcgcccggcacctacgtgcgcttcttcgacacgcggctcgcgctgccgctgaagcgcatcgactccgagttcctgcacccgtacgtgctttacgcgcgcatcttcgggctaacgctgcccgacgacctgctcgtgcagcccagcgacctcgcgctgctgcgccgcaccgaggagtacgtcgacggcttcggcatcagcctctcgcgctggacgcagctgctggaccggcactacatgcacatggtcgagtacgcgaaggtgtacgtgcgcggcgggcgcctcgccgccgcgctggacgccttcgagcgcaccggcgtgatgacgcacgaggccaccgaggccatccgcgccgtggacatgctcgcggccgtcctaaacgtgcgcaagtccaaggaccgctaccgcgcggagtgcaaggtgctcttcgggcccttcgcgggcaaaaagttcgtggtcgccgggcggcgtccgcccggctcgcacgtgctcatggtcacagaccgcgtcttcatcgaggccgagcccccattctgaggaccaccttcttggagacgcccgagaagtcgtcggcgacgccgcggcgcgccaccacaaggcagtacgaggtcacgtgcgggcagcgcgcgatgcagcggaaggcttcctcctgcgacagcgagaaggcgaacacgtagaaggtgtgcggggacttcagcggcgtgtggtccatcgagtagatgacaccgagcttcttcatgcgccacataagcgcgttgatgtggtcggcgcgcagcgcgcggcccttgagcacgccgcagacgaagctcgagcaggccacgacgtcgtagcgcgtgttcctgccgaagaccaggtgcggcgcgccggcggcgcgccgcgcggccgcgcgattctccacgatgtcctctatggagcgctcgctcgcaaagaagtccaggaacatgtactggtaggccacggccgggcgcgacttgctgaacttcatgaaggcgtccgagtccatgatggcgtccatgtcctcagcggcgagccggtgctgcagccggatgccctcgaaggtgtggaagagccgcgcgtccgcgtgcatggacagcgcgagagtgacgaagttgagaaggtccgcgtcgccaaagcgcacgagcacgttaccgggcgtgcgcgtcttgcgcatgagccgcgcgggcgcgccgtcgttgtggctgcggcggcgcattttgtcgccgggggactcgggcggcaggtcgatcatgaccagccggtgccgctgcgcgtcctcggcgttgaagatcgaggacgtgaagcccgggtacagcaccacgcagtcgcgctccgagatggcgtgcagcacgtcgcgcttgagcccggccaccagccgctccgcgttctcgacgaagtagttctcgtagtccaggatgtcgtgcgccatccaggggaagttcaggtacgcgttcatggcgtagtcctcggcgtcgaagcagatgcgcgtgtctggcgtcgccgcgatcggaaggtccttgatgccgcggagcagcccgtcgtagtcggactcgtccacgaaggagagcaccacaaagaggtcctcgcccacggtttcgtagtcgaagaggtggtaaagctctcttagcgccagcacggcgagcgcgttgtccagcgaggcgtgcacgcgcgccaggatgctgtagaagggcgtggccatcatcacggccttgccgccctcgcaggcaacggcgcgcgggaaaatgacctccggcgtgcgcggcagccgcccgaacgtcgcgttcagcagcgcgaccgtggccgcgtcgctctggcgcaggaacactaccaccgaggggcccgagatgctgagcatgcgctcgcgcatgcgcgctggcaggtccggcgtggtcacgaggtccgcgaagcggccgccgttgtagaggtcgccgccgccgaggaaggtgagcacgtcgaagcagtgcagcacctcgttgcggaagtagtactcgttctcgagctccttggcgtacgcgcgtatgtccacgttctcgaagtttgttcgcagaccgccgccgtcgaagaaccaggacgcgagctcgcggacggcgtccgcgggcctgttgcggcggctcttgcaccagaagctcatgtagttgcgcgaggtggaggcgttcgccaggaagaagcggtggtcgaaggagatgagcacatgctcgagcaggtgcgcgagccccaggaccgcgcccacgtcgcgcccaaaaccgaagtttgatatccccaggtagacgtcccgtttcatagacggcctcaggaacaccctgacgccgttttccaacactatcattctccggtatttacttacccaaaagtagtatggggagaagtgtttgaacgtcccctcgcctttttaaatcaaaagtagacttctcgcgcccgtgcgccaccgtcacgcgcgcgcggcgcgagtccataccggcgatcaccgcgctgctctgcggtgcgtccggccgcgggaagagcacggtctcggagatcccgtccagctgcgcgtcggtgcgctgccgccacgcgtgcgcgtccgcgagctcgcgcacggccagctgcatcttgttcgtcggcaggaacgtgaacacgtacgccgccgccaggaaggctgcgaagagcacgaactcaaccgcccatgacatttagggagctgattttgttccacgcggcgacgcacgtcgtgacgggcgaccccgaggcgccgcggcgcgcggcctcgctgtgccgcggcttcggcgtggacttccgcgcgattcacgcggagttcgcgcggcggtacccgcgcaccgcggccgccgtggagcgcgcgcagccgctgcccgaagtcgatgccgcctttccgccggacgcgcgccggcaagtcgtgcggctgcgcctcgaggctgcggcgctggtcgtcaaggagtcgcgtgcgctatcggcctccatgcgcggcgtggcggtggtcgacggctgctgcgtgcgcgtgtgccgcgccaacgacgagctgctagagttcctcgcgcggcgctacgaccccgcggtctaccgctacgcggaggtgccctcgccgagcgtgcgcccgggctcgaaagtcttcgcgtgcgcgggccgcagcgtcacctttgcggccgcgcaccggagccgcatcacggccaaccgcccgctgcgcgtggtcgtgaccgaggcctgtgtggacggcgtgctcgcgcgcggcgccgcggaggtcttcgaccgcggctccggcgtgctgccccgcgcgctgcgcgagatcttctaccgcctcgacgaggacggctgtcccacgggccagacgccaggcttcgcggacagtatggcgtcgcgcagctgatctatgtccacctttttctcgtcgatctgcgccacgaccacgaaactgcgaatgtccacagcggccatggtcttggccaccgggtcgtacttgaggagcagcacgtactcgttgccgaagtgctcggtgacctcggtgatgagccggtacacgcccatgccgagcacgttcaccgcaccgtccttggcgaagagcgagaggatgttcacgcacttcagctccatctcgccctcgaggcgcgcgagcatgcgccgggtgacctcgcatactgaacaaagaggcttacctagtaagataagcgttagcttagccgcggtcggtgacgcgtcggaggccatttatggggatcaaaaacttaaaggcgttgctgctcagccacggcgcgctgaccccgcacgagccgggcggcgacgagcgcttccctgccgtgttcgtggacggcttcagcgtcatgatgaccatggcgtactcgtgcgcggacgaagacgagttccgcgcggccgtcgaggagcgcgtgcagcactggatgagcgtgtccgagagcgggcggatcgtggtcttcctcgaccgcggcgagattccgatcaagcagccgctgcgcgaccagcgccgcaaagccacgcgcgaccgcgccgcgcgccaccgcgagttcatcgccgccgcggaggcagacgcggcggcagaggccgttggcgcccgcgaggacaaacaggaggacgagcacgcggagttcgccgaggagatccgcgctgagaagcagctaaagctgcagcgcatccgcttccagctcagcatcgccaaccacgaggtcgttaagtcgctgatagagtccacgctcgcgcgcgctggcgatgccgtggagatcgtcttctgcgacggcgtcgacgcggagatggtcatgtgcgcgcgcggacgcgccgaggccgagcgtcgcgggcgctggccgctgctcgtgaccacggaccaggacgcgcttttgttcacgtccaccgatcgcgacgagaagatagtgagcaccgtctccgcctgctacgcgttcaggcccaccgagacgaccgagtacctgtgcaaacttgcggcgctggccaacggctgcgacttcttccccgggctcggcggcatatgcgtgagtgtggagtcgctgcgccgcgccacgcttttcccggaattctccgtgcgcaacgccgccgtgagtctgtgcacgcggcccatgcggctgtccacgcaggacgcgctggagccagaggccgccgccgaggtcgtggaattcatcaggcggtacgccgccggcgacgagcgcatctaccgcgaggtgccgcccggcgcgtgctgcggacgcgcgtttgtgcgcggagcgctcgcggccgagtgggccgacgcgctgccggcggccacgggtctgagcgtggtcgcggacatgatcgcgtgtctgcccgcgcggcgggaccccgcgcccgaggaggtagagcggctgctggcgctggaggcgcgcgcgcgaggcgcgcgcgtcacggatgcgatgctcgcgcagactgcgcagctgctgggttacggcgcgagtgcgggcgccgacggcgcctccgccttcgcggtctcgggcgccaagggcctgatgtgtcgcctgcgcggcacggccatgttcttcaacgcggagtacgtggaaattgaaagcgaacccagactgttaaagctgcggtagcatggtgttcccgatcgtgtgctcaacgtgcggccgcgacctgtcgcacgagcggtttctgctcatcgtgcgacagcggccgctaaaggttgttttgcggacggtgcgcaacgtctgctgccgtataaagttgtctacacaaatagagccgcaccggaacctgacggtgctgcccatgctcgacataagctgatttttcttttccgctcgtatgcgcgagttcggactcgcggcgcgcatggcccgcgccatcgaggacgtgtgtccgcgcggcgcggtgatattcgtatccagcgccgcgtccatgaccgactgcctgaacccgtcggtgttcaagcacgcggcgatatacgcggggcgcgtggaccgcgcgccgctgccgccgccctcgccggtcccggcggaggccgtgacggagccctgtgcgatagacgccatagcgccttacggcgcgcgcgtggtcctgctctcggagctgctgcggagctgcgtggccgttcaggcctaccgcctggcagtccccggcgccctcgcgctcatgaacctcgcggccgacgcggccttcgagctcgtgggcacgccctacggctttaacagcgaccgaacgtactgcttcaagctcgttgccgactgctttgctagcgtgggcgtgacaacgaagaccaggcgcatcatgggtcgcgacgtcgtgctcagccaggacttcctggagagcggcatgtggaccaaggtgctggactccgccgcggagccgccgtggctggtctagaacagcggcggcgcgcgggtcccgagcacgggccgcgccacctgcagccgctgctgcagcgcgcggcactgcgcctcggcgtcggccgtctcggcggggtcgacgggcgtcggagttgcggaggtggtcctgaacggctgcgtgttcaccgagacgcggatgcgctccttgcaggagcgctgctcgatgcagttggccagcatcttcatcacgtgcaggtactccagcaacacgaacttttcgagggtgatgccgtcgaagggcgacgaccccaccacgcccagcgggctggacaccgcgccgtcgagcacctcgccgcgggactccttgcgcgcgcgctcgagcaggtcctctgtccgagccaccacactgccgaagtcggcggccgcgggggcgggaacaggcgcagcagcgctgtccgcgtccgccggcatctcctcgatcttgagaccggccgcgaactccgaggccgcgtgcacgggcgaggcgccgcgccgcaccatgaagtcgcacagacgcgatagcgcggaggagcgcaccggcatgtcgagcaggcgctcggcctccatctcggcgaccgagtcggcgcacgcgtccggcgcgcccgcccgcacgagctcgtcgcagcaccccgcctccttcatgagcgcgggcatgagcttgtactgcgccatgttcaccagcccgtacttgagctcgagcaggtccgcgagctcggaggccatgggtcggtttttggtgtagatgacgcgctccacggcctccgccatgtccacggcctgcatgagctcgccgacgagcacgctggccacgagcgtggccagcgtgacgcgcacggtgggcacgcagaccgcgaagaaggaggtggagtgggtgaagcgcatgagcgcgccgtgcagacgcgcgaggtccgcgctgttgcccgcgtgcacgaagcgccggcgcagccgcgccagcgcctccacaaggtcctctcgcgtggtcacgcgcacgttcgcgatgcacaggtcgtggatcgcgttggcgatctgcgcgcggcgctgcggcgagctgccgggcagcagccgcgccttggcctcgacgtcgacggtgctcgagagacagccgcaggcggcgccgcggacgacgaacttcaacaacgactcgaacacgcgcgcgcccgcgcggggcgcttgcttggacgactccatttactttaaataatttacgagatcaaaataaaatgactctgcgcatcaaactcgagaagctcaagcagatcgtaacttacttctcggagttcagcgaggaggtctcggtgaacgtggacgtcggcgatggcctcatgtacatattcgcggcgctgggcgggtccgtgaacatctggaccatcgtgccgctcagcgcgagcgtggtatacgacggcgatgtcagccgcgtgttcaacctgcccgtgctcaaggtgaaggcctgtctgtgcagcttccaccccgactcggtggtgagcctggagcccgacctcgaggacaacgtggtgcggctctcgagccaccacgtggtcagcgtggactgcgacaacgagcccgtggcgcaccgcacgaacaccgccatctgcctgggcattaaccagcgcaagtcctacgtgttcaacttccggcgctacgaggagaagtgctgcggccgcaccatcgtcaacctggacctgctgctggggttcatcaagtgcatccaccagtaccagtacatcacggtctgcttccgcgacaagaagatggtgctgcacacgcccgggaaggtggacaacttcttccgcgagtactccatgaccgagtgggcgcccgacctcgagcgcttctcgttcaagatccccatctcctccgtgaacaaactccgcggcttcaagaagcgcgtggtcatgttcgagtcgcgcgtggtcatggacgccgacgacaacatcatcggcatgctcttcaccgaccgcgtgggcatgtaccgcgtgaacgtgttcatgtcctttcaggaccggtctctttcatgcgactaaatactcatgggcgggtcggtgagcctgccctcgcgggacctgccgccgccggtgcgcacgccggagatgaacatcgtgcccgagcgcgacctcgcggacacgatggcgcgcctctccaccgcagacccgccgcagccgctgggcgtcggcgacgacgcgcgcatggccgtgctgaagacgaccttccccgagttcgcgatatcgcggcccgcgacgggcatgctcgccgcgcagcgaatcaggtacgacggcgacccgcgcgtctgctgcggcgggttcgggatctcgcattactgggagaagggggcgcgccgatcgaacgtcgcgttcgagggcgcggcgctgcgcacctgcgaccccacgcgcttcgacgcgggcgcgtgcgacgcgctgctcttccgcgagtgcgccgccggcggcgtcgacgcggacttctgcgcgcactggatcaacgcggccgtgacgcggcgcacggaccgacagtcgcgcgcgcggctgaacgacatgttcgtgcgcgattgccaaaacgacgccgcccggcctcactgcgtggcctggatccgcgcgatgcgaagcgcgcgcgcgacggcggacgacggtctaatagacgccgtgctctcggtgcagagtcccgagttcaagggcaagcacatgcgctgcagctacccctcgccggccactctcgccatggccgcgaacgtggacgagccgcgcgagtgctgggaccccgagtgcgtggccgggaacgtggacttcatgctaagcgataactacacgaacctgggcttgtgtcggctctcgcgctgctccatcggcgtcacacacctgcggattgacgcgcgttcgcggctgcgcatgcggtgcgccggcgcgcttgccgggctcacgaaggcgcccgtgaaccagactgtcgtcgtcggcgacaacctcgcgcgcgccttcgagccgcgcgtggaaacgctcagcgtgttggcgctgtgcgtggtgtatctgctaattgtctggctctaaatgggggccgccgccagcattcagaccaccgtgaccaccgtcagcgagcgcatccgcaacgagctcgagcagagcgcgagcgctagcgcgaccgccgactgcgacgtcaccatcgggagtctgattatccgcaagaacctaggatgcagcgtttccgtccggaacatgtgctcggccaacgccggcgcgcagctggacgccgtcatgaaggccgtgagcagcaccttcaacgacctctcgtcggaccagaaggcctacgtgcccgggctgctcacggccgcgctcaacatccagaccacggtgaacaccgccgtcaaggacttcgagacgtacatgaagcagacctgcacggcggacgcggtcgttcacaacaaaatcaagatccaaaacatcgtcatggaagagtgcgcctctctgccagggagtccggccacgcacctggatttcgtgaacaccggcacggccgtgggcaactgcggcgtgaaggccgtgatggacgtgctcgcgaaggccagcaccaccgtgcgcaacgaccaggaggccggcaagggctaccagaccatcatcatcgcgatcgtggtcgccatcctggcggccatcttcgcctggtacgcgcggcacatgctattcatgtccacctccgacaaaatcaagctcgagctcgccaagaagcccgtggtgcactggaccacctacctggacaccttctttacggaatttccgccgtccgtctagatacgcgcaacattgaaacattatatccacctctcaaacggcggtatggtccgacgcgtcctcctcgagcgcgtggacggcatcgtcgagcactcgcgcgcagaccgacgctacttggaggccattcagcgacacctcgaggggtctacgcccgggctgcggcagatgtggcgcttcctctacgacctgctgctgacggtgttcgtcgtcatgtacatcgtcttccgcctaatcgtgcgcaaccccggcatctgcgccatcctcgcgctcgcggccgcggtgtactacctgtttttgtgtctctttagcatggactgatggcgatcacagacagaccatcgcccgcgcgcgcgtgaccagctccggcgccgcgaagacgtcctgcaccgggaagtcgtcgatctcgaacacggagccgtccgcggaccagatcacgcgcacgttgtcgctcaccgagacctcggtcagcgtcacgcccagcacaaccgcgtcgttggtgctcaccagcaccagcgcgccgggctccgcgcgccggtgcagcggcggccccgagactgagcgccgctgcacgcggaacatgtccgcgaactgcttcgagagcaagtccaggtggttgcggatgatccactcgaagaagtacgcgcaaccgccgccgccgcacaggaagcgcgaacccgcgggcatcagcagccgcacaacgtccatgtagcaggcctgcggcaggctcgcgcggtacagccgcgtcttcggcgagagcaccaccaggctggaggtgctcatctggaagaccagctggctaacggagacggtgagcgtgcacgcgggcacggaaaccacgtccaggcagatgtcgtccagaaagatgctccgctggtagaggtggtacaggatggccacgatctgaaaggccgtggcgtcgctgatggcgcaggggcggtcggcgcagcgcatctgcgcgcaggaccagcccccgaaggactcgaagcagacggtgagcatgcccgtgctcggacagtgtggcgagcgccgacacaccggaaagcccacggccttgcggcagcgcaccatggtcgagagctctatccagcagcctgcctcctcctcgcccatgcccatggctaccggcgtgaaggccgtgacgtcgtcgcagatgcgccgctccagaaaccccacgcccgaggaggggtgcgcggccggcggcgaggtgatgcgcgccgggacgcggctcggagcgggctcgggaggcgagctgcgctcgacccgggcagtcgccgccggccgcgatgccctgcgcgcgggcgcgcgctcgcgcaacttgtttgacttgctggcctcgtcgctagcgtcatcgaagcggtcgttcctgtcgccgcggacgtccgcctcgtcgcccgtcggctgcgcggcgggcgacgtgccgtcccgcgtacggcccgcgttcggcgcgaatgtcacgcgccggtgcacgtacggctccgtagagcccgtgggggcgccgcgcccgcgcccgcggcggaaggcctgccgggacgcgccgaagcgggcgaactcccccttcgcccggccccttttttcttccatgatatttatcacaaaaaaaacttctctaaatgaccaatctgctttcgttggtcgacccggaggacctggccttctgcgccgggttcccgtccttcgacgagaccatgctcgtgatcgcgggggcgcgagtgcgcttcccacgctcgctgctctcgctcttcaacgtggtgccgcgcaccatgacgcgctacgaaaccgagctcgtgggcaccgagatggtggtgggcgccgtgttcaccaccgcgtacaacgtccgccgcaacctaggcctcggcgaggagcccgtgaccatgcgcgacatcgagaagtacttcctggactccgagaacgaggtgctcacgctcatcgtgcacaacaccgacttttccgccatgagcggcgtgcgccggcgcggcggccggcgcatcgccaaccccgtcatcttccgcagcgggtccacgccgctgctcatcgtgatggagtcgcgcaagaagaccaacatctaccgcgagcgcaccgcggagcaggccaacgcctcctacagggaggtcggctcctcgctcgcgctggtcactcggtacgcgggtctgcagctggttgacgtgcacacgcccagctccgtgctaacggtctccgccgtctacggcttcaccgaggacaaggggctcaagaagctgggctccgacaaggagctcgcggactaccagtccacgccgctcaccgaccccatccggctcagcgacttctccaatatattcgacggcgtcaagaagagcatccagctcacgaacgtgcccgtgccctccaccggcgccgaggccgcgccgtaggctttcatgcgcgataaatcggatggcggcgccgacgacgcccgcggtgcacctcacgccggtgttcgtggagcctacgatcgcgcactcgctgctgcgcgcagagtcctacctcgcgatcgcggtccttgagctcgtgctcgcgctcgcgctcgcgctcgtcttcttccgcgacgagctaggctcgctattccgctgcgcgccgcgagcgccttcgccgctggacgcgtacctgcaggcgagcctcgtctgcgacggcgacgcgctgctgatcgagctgcccgagggccgggtgccggcgctcgcgctggacgggcggcccgtcgcgttcccggggtgcgagagccttttgtaccgcataaatggaccacgaaaagtacgtcttgtcgatgttcttggaggaagataactccttcttctcgttcgtcgccgcgctgtccgatgacgaggcgctcggcgccgtgcagtccgctgccgccctcctggacttcctgctctccgtggtggtccgcggcaaggagaagctcgccgccgcggggcaccactacgactccatcgcggacggacgcgcgcgcgccgcgttcgagttccgagacctgcgcgagctggcgcagctcttcgaccggcggccctgcggcgtccaggaccgcgtgcgtgtgcgcgacgggcccgcgcgcgccttcgtggacgcggcactggggctcatgcgcgagcgaggcttcgacggcacgcaggccgcggagcgcgcgcgctacatcgcgccgaacgatctgcccgcgctgggggcaatatcggccacgctctcgccgggtctataacgtaaaaaatattagtaaaattctgaaggtccgtgtgtttcgcgggcggccaacaaaccagtcgcttaaatggagggggtggaaatggacaagccgctcctctacttcgacgagatcgcgggcgcgcgcgactacgacgcggccttcgcggagaagcacgagccgcccaagatccccggccgcggacagatgaagctgctggtctgcgagctcgtgtttctcaaccggctgcacctgcacggcatgctcgacggcagcgtcatcgtgtacgtgggctccgcgcccggacggcacatctgctgcctgcactcgcacttccaggagctcggcgtctcgcttaagtgggtgctcattgacgggcgcaagcacgacccctgtctctcggggctgcggaacgtgaccacggtgacgcgattcgcggacgaggcctacctccgcgagctgcgcggcgagctgcggcgcgccaagatcgtgctcatttcggacatccgctccaaccgcgtggacacagagcccaccaccgcggacctgctgcgcgactacgcgctccagaacaccatggtgagcgtgctcaagcccgtggcctccagcctgaagtggcgctgccccttcccggactcctgggagaaggacttctacgtgccctgcggcaaggagatgctgcagccgttcgcgccgccgttctccgcggagatgcggctgctcaccgtgtactcggagacgcgcccgaagctgcgtctgatcacgctcagcgacgcggtcaactatgaaaagaggatgttctacctcaatagcgtggtccgccagcgcgtaattctgaactttgactatcccaaccaggagtacgacttctttcacatgttctgtctgctctcgtcggtggtgtgctcgtgcgaatttaaatcgcccaaagagaaggtgctgagcctgcagaaccgcttcttccgcttcctgcgcatcccgccctccatcacgctcgggctgcgccggcacgatgaaccgccacaacacgcggtacctggccaagatcctctgcctaaaggccgcggtaagaagcgaccccttcgcggtggtaagtagggacaccgtgcgcatgtacgacatcgaggtcgagtacggcgacctcgtgacggtggtcaccgtcacgcacaaactcgagaccagccgcaccgtcttccaggtcttcaacgagacctctgtcgcgtactcgccgctgccggacgactacggcgagcccatcgtgctcaccacgtacatgcagcgcgagcacaccaagttcccgctctccatgctctacatcgacgtggtcgcctcggacatgttccccacgtttaagcgccccaccgaggaggaggccgcggtggtcgcggccatgcagcgcgtgggcgggcgccgcgagcccgtgctcaagctcccgcgcatgctggacaccgagctcgtgtgcaagatactgcacctgcccgagcacccgctgcgcgtggtgcgcttcctgcgccgaaacatgttcacgggcgtggaggtcgccgaccgctcggtgtccgtggtcctcgactgacgaagggcagcacggtcagcgaggccgccgccaccaagcacagcggcagccacgcgcgcgggtccgccacgggcacgaagacgtgctggttcaggtacttcgcctggaagcgctccgcggtggagtccaccttggacccgcaggcgttggtgaggcgcacgaccgcgtccgcgacgcgcacgtccccgagcgatatcacgcagtcagagacgttgcacccggcgatgtttttcttcagcgcgcgcggcagcagcgcgtccgcgcgcttgcagggcgcgtaccagcagtagtagggcaggcgcgtgtcgcggccggtgtcgaccacggcctggctgggcttgaggcacgcgcagcgctcgtcgtccgggtgcgcgtcgcagaaggcgtaaatctcctcgtcgggcgcgtccggcccgggcgcggtcggcggcgccgcgcgacggcggaagaacatctctgaaaaaatacttcgaccagaaaacgaccaccgatcttatttcaaagataaaaatactattaatacgcactcggagaatcatgtcggtggtggcgcgcgtgtcgtacagcctgtactcgcagagcgagataagcgccacggacgtggtcatcagccagttgaagaacgacgaggacctgggcacggtgaaggacccgcgcctgggcgcctcggacgggtccatatgccgcacctgcgggctcacggagatggagtgtttcgggcactggggcaaggtgcgcatctacgagtcctacatcgtgcgccccgagtacatccccgaggtggtgcggctgctcaaccacctctgcgtgcgctgcgggctgctgcgctcgcgcgacccgtacacgacggacttggccgcgctcagcgtgcacgagatgcgcaagatgaaggaccggatgatgtccaagaagaaggcctgctggaacagcaagtgtctgcagccgtaccagaagatcgtcttctccaagaagaagatctgcttcgtgaacaaggtggacgagatacccgtccccaacgcgctcatctaccagaagctgacctccatccaccgcaagttctggccgctgctggaggtgttccaggaccccgcgaacctgttctacaaggagtacatgcccgtcccgccgctgctcatccggccggcgatcagcttctggatagacaacatccccaaggagaccaacgagctcacctacctgctgggcatgatcgtgaagtactgctccatgaacgccgaggagcaggtcatccagcgcgccgtgatcgagtacgacaacatcaagatcatctcctcgaactcgagcagcatcaacctctcctacatcatcgcgggcaagagcaacatgctgcgcagcttcgtggtcgcgcggcgcaaggaccagaccgcgcgctcggtcatcgggcccgactccgcgctctcggtgtgcgaggtcggcatccccgactacatccggaacacgctcacgcagaaggtgttcgtgaactacctcaccagcaagcgcgtgcgcgcgctgttcgaggaccgcgcggtcaagttctacttcaacaagcggctgcgccagctcacgcgcatcaaggagggcaagttcatcaaggacaagatccacctgctgcccggcgactgggtggagatccccatgtccgagggcacgaacgtgatattcggccgccagccctcgctgcaccgacacaacgtcatatcctcgaccgcgcgcgcctcgcccggctacaccatcaagatcccgcccgggatcgcgaactcgcagaacgcggacttcgacggcgacgaggagtgggccgtgctcgagcagaaccccaagtccgtgatcgagcagagcgtgctcatgtacccggtgactatcttcaagcacgacgcgcacggcgcgccggtgtacgggtccatccaggacgagatcgtggccgcgttctcgctgttccggcaccagaacctctcgctggacgaggtgctgaacctgctcgggcgctacgggcgagacttcgcgccggagcctggccagaagaccttctcgggcgccgacgtcttccgattcatgataggcgcggacataaacttcaagggcgtgctcgagaacgggcgcgtggtggcgccgaacgtcgacagcgacctcgtggtggccatgcgcgcaacctcgctagcggggctgatcgcggactacgccacgaacgtggagggcgtgcgcttcgtggacatggcctcctacgtgtacaagcggtacctggccatctacggcttcggcgtgaccttccgcgacctgcgcccggacccgagtttggttcgccggctgcacgcgctgaacaccgagaagatagagcagatcaaggacgcgtactcgcggtacctgcaggacgtcgcggacgggaagctggtgccgatggcgcccgcggacgaggccgacgcgctggactcgctgctggccaacctgaccaacctcaacgtgcgcgagatcaacgagtacatgcgcgagacgctggagcgcaaccccgataacagcctgctaaagatggcgcgcgccgggtacaaggtcaaccccacagagctcatgtacctgctgggcacctacgggcagcagcgcgtgaacggcgccgtcgccgagaccaagatatacgggcgcgtgctcccgtacgcgttccccgactccgcggacccggaggcgcgcggctacatcatcaactcgctcatgaacggtctctccggctcgcagttctacttcgcgatgctggtggcgcgctcgcagtccacggacatcgtctgcgagacctcgcgcacgggcacgctcgcgcgcaaggtcatcaagaagatggaggacacggtcgtggacgggtacggacagatcgtgagcggctcggtactgctcaagtacgcggccaactacgcgaagatcccggggtccaccaccaagcccgtggagctgctcttcccgcacgagagcatgacctggttcctggagataagcgcgctctggacgaagatccggcacgggttcgtgcgcatgcaccggcagcgcctggccaccaagatcctggcgccgttcaacttcctggtcttcgtgaaaccggcgccctcggaggcggaggcgctctccgcgcgggacctgtaccacatgatccagcgcgtgatgaacgacgtgcgcgagaagtacttcttctcgctggcgaacgtggacttcatggagtacgtcttcctcacgcacctgaacccctcgcgcgtgcgcatcacgcgcgcgaccgccgagctcatcttccgcaagctgtaccagaagctgaacgcgctgctcggcggcggcacgcccgtgggcatcatgtccgcgcaggtgctctgcgagaagttcacgcagcaggcgctctcgagcttccacaccaccgagaagagcggcgccgcgaaggtgaagctgggcttcaacgagttcagcaacctcatcagcatgagccgcaaccacaccgagatagtggcgctgaccgcgccgagcgcggacaagctgatgccgctgaaggtaaacttcgagttcgtgtgtctgggcgagctcgtgcccgagatcgagacccggccctcgggacggccctccgtgcaccgcgtggacatcacggtgcaccgcctgcgcatcaagcgcgcgcacctgaccgaggtcctggtggacaccatcatcgagcgcttcgtgtccttcaacgtgctcgtgaaggagtggggcagcgacatgaccgtggagggcgaccgcgtcacgtacacgctgctgctgcgcttcgtggagccggagcagctcaacttccacaagttcatgctggtgctgcccggcgccgcgaacaagggcaaggtgagcaggttcaagatcccgatcaccgagaccacggtctacgacgacttcgacgccgcgcgcaaggcctaccgcatgaacatcgagctcatgagtctgaaggagctggggatattcgacctcgaggacgtgaacgtggtccccggcatgtggaacaccttcgacatattcggcatcgaggccgcgcgcgggcacctctgcgagagcatgctggacacctacggcacgggcttcgactacctgtttccctcctgcgacctgctcgcgagcctgctctgctccgggtacgagcccgagtccgtaaacaagttcaagttctggaacgcgagcgcgctgaagaaggccaccttcggcgacggccgcgcgctgctgaacgcggcgctgcacaaccgcaccgacgcggtcgcggacaacagcagctgccacttcttcagcaagacgccctgcgtgggcacgggctactacaagtacttcgtgaacgtggagatgttcatgcgcatggagcgcgagatccaggcgcgcgtggcggcgcgcaagatggaggagatcgaggaggccgccgaggaggagttctaggcgcgacggcgccttactttgcgaccgtgtcacgacgacacgacacggttaggacggcgagtcgcagacgaacatttttatgagctggtagcggaagttggcgttttccaggaaggcgccgcggaggtcccggatctcgtagtaggttttgaggaagtacacgaagcgcgcgggctgcgtcatagtcgggttctccgcaagccgcttgtgcatcacgtaccccatggcggcggcgccgctgcggttgacgccggccacgcagtgcacgagcgtgggcttctgctcggcctcgaggcgcgccagcagcttcacgagcgcgggcatgatggaagcgatgttcgtcgtgtcgtcgtctctcagcggaatgtggtacgccgttatccccgcgggcgtcgagtacttggacatggtcatgttaaccagacacttgaagtcgacgccggagtccccccgcagcacggcgcgcgcgtcctcggcgctgcccaagtacacgtggtccgtgagccgcgtcatgcccgagggcagggccagcggcggccccgcgcgcgtgcaccgcagcaggagcctggcgtaccactcgctcttatcgcccatatttatttatatgatacaaatggcagacgtcacaacactgacggccaacggtctgaccctggagttcgcgcgcgagcgcgctctgcgcagtctgcgcgccgcgcgcacctccacgctggtgttcttcacgctcacgctcgcggcctcgctgttcgtgctctggctgcagctaaccgagtttcccgtcttcgaggagctcggcaagtacgcgcgcatcaagagcgcggtgcggtcctggcgcccgctggtggaggctaagacagagatcgagtccgacctcggccggcagaagaccgccgaccggcccgagctcttcgagttcaggtgcgtggacttcggcaagttctacctgccggtgaggtacagccccacgaccttcctgccgcaagccgtgcgccgcggcgcgggcgatggctggatggtgcacaaggcggcggccgtggacctcgccgcgcagcagttctgcgagtccgtgctgcggcaccgcgccaacaacgtcatcacatgcgggtcagagatgatgcggctggtgggctacagcggctacttcgaggacgaccactggtgcgccgcgacgtccggcgtgctgacgtgaacgatcacacgatggccgtgaccagcagcccggcgatgaaccacagcagccgcgagttcggcagcagcagcacgagcaccagcaggtacgccaggatgaagatgtcgaccacgtccacgtcgaagagccccatgaaggagaagagcggcgtggtgaggaagtagatggcgccgggccagaagcgcgccagccacgtggcgagcagcgaccacagggagggcgcgccgctgagccgcgtcttcacctgtatgtagtactcggggtagaccacctgctcggcgccggagagcaccacgcgcgccagagagagccgcttctccagcgtgaacacctcggtgagcaggccgctgcgcagccctccctccttgatgatcgcgtcgtagagcttcttcatgccgccgacgctgatgatgtaggcgtctagcgagacgtcgtacccgccggggtagaccatgagctcggggtcgccggtgccggggacgttggtggccagcgcgccggtcatgtaggtctccttgagctgcgtcatgtaccagccgttcgccttcatcgcctcgatgagcggcttcaccatctcgggcttgcggaaggtcatgtcgttgtcgaccaccaggatgaagtcatcgtcggagtacttggtggggacagtgccggccgatatgctctcccagaggttgaggtggtgcgctgcgcggcgctgcatctccttcggacacgtggacttgcacatgtccgtgaagaagtgcgggtagtctttggagtccacgtctttccattccaccgccttgagcacgtggtcgcccttggggtgcggcgcgggaggagatggcttgggtgccggcgcgggggccggtgcgggggctggggcaggagagggagcaggcgcgggttgaggcttgggcgggtcgtcggcgaggcccaccaggtacggcagcgtggggaacacctccttggtcccgcggccttcggcaaccccgattatgtaggccgtgatttcgggtggatccatttagttattaaaattaatcatatacaactcttttatggcggctatggattcggctatccagtccttgaccgagcccacgatgcccgccaggaacaggaagaaggcgaactccaggtccacgcggttcagagagtcgctgaagtacacgaagacgtcgctgtccgggaagaagctgcgccggaacatgttgtacccgttgaccttgtgcgcgacgtgctccgcgctcagcagcgtctcgtcgaaggggtacgggtcgctgaagcggaacacgtacatggccgggtttgcgtagtagtacttcatggtgtttgtgacgaagaggctcgccagcgagatgatgatttttttcttctcgatctcgatcttgatgtggtcctcgaagcgcttcatgttgtaggcgttggtgtcgtgcacgcggatgagcacgcgcgagtccgacatgatgtcctggaactccgcgcgcgcgtcggggctctcggcgggcgtctccgcgggccgcgccacctccgcgcacaccgtcggcctagcgcgcggcggcgtgcgcatgggccgcgcccccacgcgctgcgaagcgaaaaactccacggcgcgagcctcgcccgcgtccgcgtacgactccaccaggtagttgcggctgcgcgtggtgcggccgatggtgttcagccggtgcagctccgcgaccagccggcggtagtgcgcctccagctcctcgggcatgatggaggtgtacacctcggtgagcagcatcacggtgtcgaagtcctccttgccgcagacgcgcgtcttcacgaggaagtggtgcacagccgtcgcgatagagagccgcagcgtggactcggtgacctcgacgctggcgtccttggtcttcttcgcgctccgcgaggccatgaacgagacgaggaagtccgcgctgctgttgagcacgatgaccagcgcgacgatgaagttgaggttcagcgtcttcgcggactggaacagctcggtggccgacgcgtgcacgtcgagcaggttcgcggagagccgcaggaagaacacgccgcgcttgatctcggccgcgaagcgacgttcgtactcctgccggcgcgcgttgatcgcgatgaggaagttcaggatgagccggttgatgttgtacttcacggcccaggtctgcgtcttcatgatggtgtcgaaggacatcacgatgttgaagatgaagcgctggctgtgcgagaagtagctgtagggctcgctgaggaagatggacttgttggtcgcgggcaccaccacgcccgcgcgcgcgccggacgcgtcggtgttcaggtccgggatgttcatgccgcagatgcggcagtaggccatgccgtcctcaaagtacacgaactcctccacgaactcgttgatcttggcgaagtagtccacgtccacgcgcatcgcgaccgcgagccggatctggtgctcgcagggcggcgactcgaagcgcaccccctcgccccagcccggcggctcgcgcacgaccagcgcggtgcgcgaggccgggcggaacttggcgtcgcgcgcgttgagcagcgccgggaagaggtcgcagaggtgccggctcgagaggaacacgtacttgtacagcagccggcgcgcgtccgcggccatggcgtccacgaaggcgcggccccactccgcgaccgcgggctgctcctccgcaaagttgttcgggtagaccttgtccgtggccgcgaggaacaccttcttcacgtcgaggaagtcgcggatcacgatggggacgcgcgcgccgtcgagctcgtacatgaacacgtagcgcaggttgagcttgcgccgcgagaccgggatgccgatgtgccgacacaggtacgcgaactcgaggtacttcttcgagaagcggatgcggtccaggttcttggagacgtactgcagcatgttgcgcatgttgaaggggatctcgcgcacggcgggctccgcggcgtcgtcgaaggcggtgcgcagatcgctggtgcgctgtacgaccacggcttcgccggtggcgtcgtcgtgcaccagcacgttaacgcgccgctgccggatgaccatgtcgaaggtgttgaagaacatctcgtacatgctgtgccgagtgtcgtccgcgatgcgctcgcccaccgagaggctcgcggtggcgtcgtcacgcacctgcttctcgaacttgtacccgatgtaggagaatatcgagatcagcgtggcgtcgtcggcgtcggggttctgctccatggtcgcgaagagcaggcggatgtcgtcctccgtgatcgcgtccacgttgtacaggttgaccacgaagatggacttgttctcggcgatgaagtccgtgtaggacttggtggccgtgttcgggtcgcgcatgtacgcgcggatcttcggcacgatgctcgcgaggatggactccctggaatccatttaaggacggcaagggcgcgcgagaccgtctcaaaactgaaatcgtataaactcttaaaaaatcggtattgaaagtacgcaccaccaaataaagcgtcgaggtcgggcatgtcttcgtggcgactcaaaatgagcaagtgttcaggttccagcagcgtccagactctcgaggatctgcgtaatcgtcttcgctccgaggccttgggcaacgattgccaagagccccgcgacgacctcttccccagcggcgaggagtgtctggacatcgacgggccctgcccttgcgatgaggcggagcaggagatcgaccaggagcagttgcccgtgcccgaaaccgtgcccgaaccgccggccaagactcctaagcgccgaccagtgaagaaggataaggcagataaggcagataaggacaagtcgaccagaggcgcaaagaaaccgtgcccttcggacgacaaggatgacgagctcaagagcaacgacgtcgacaacaacgaagagtccggcgacacagacggcggcgcgagcgcccgaagccccagcgacatcgacaacgtggacgaaatggacgactccgacctcatggtggcgttctccaccatcctcgcagacttcaaggaccttacccaacgagtgaaagctctttcgtccgtgctcacggacgtgcaggcggccggcatacgcaggagcttctcgacgctcggcaaggctctgacggaggcggcccacatcgccaacaccggatctaagccagtcactgcgcctcgcaagaagaaggccgccgcctgcaagaagtaggcgcactaaatagcgaggctcggtatgcgggcgctgcacctgtcagacggcaaacttttttttgacaaggagctgacgcagccggtccccgacgacaaccccgcgtacgctgtccttgcgaagatccggatcccaccgcacctctcggatgtggtcgtgtacgagcaggacctcgagtctgcgcagcagggcctcatcttcgtcgggcgcgacgccaagggccgaaagcagtacttctacgggcgcggacacgtggagcggcgcacggccgtccgcaacgccgtgttcgtgcgcgtgcaccgcgtcatgaacaagataaacgccttcatcgacgaccacctcgcctccggcagcgaggccgaggcgcagatggccgccttcctgctcatggagacgagcttcttcatccgcgtcggcaagacgcgctacgagcgcgagagcggcaccgtgggcatgctcacgctgcgcaacaagcacctcgccgaggccgagggcggtgaggagatccgcgtcgccttcgtgggcaaggaccgagtcgcgcacgagtttgccgtgcgcgaggggcagcggctcttcgcggcgctgcgtcggctctgggacccgggcgcgcccgacaggctgctgttcgaccggctgagcgagcgccgcgtgtacaccttcatgcgacgcttcggcatccgcgtcaaggacctgcgcacctacggcgtgaactacaccttcctgtacaacttctggtccaacgtgcgctcgctggagccgcgtccctccgtgaagtcgctcatctgcacctccgtgcggcagaccgccgagacggtggggcacacgccctcgatctcgcgcagcgcctacatggccaccgcggtgctcgagctcgtcagggacggcgcgttcctggacagagtcgccgccaccgacacgctcgacgacttcgtggacatcgtcgtggactatgtaaataactctgagcaggtaaatggatgaggcgctgcgcgtggcggcgcgcgtcgtggacgggctccggccgctggacgtggccgtgtgtctcgcgcagctgcgcggagccgcgcccgagcgccgcttcccggcgctcgacgagtgctccggcgaggccttcctggactttgagttcgccggcggggacgtggcgtcgcggtacctctccgcgcacacgcgcgagctccgtgcggcggagcggcgcgagcacatggccgcgatcgcgcgctgcgtcaccgaggccgacctggcgctcgcagaccgcccccggggcaaggcgcgcgcggcgctgcgcgtgtgccgcaaccgcgagaaagtcgcgcgcttggcgaggctgctgcgcgacgccgagagcagcggcgcggacttcgccttcatacgcgcggccgtggcgtagcaaaacgtaaaaacaacacattccctaaatcgccatggacgcgccaagtctcgactgcatgctcgccgcactcgcggcgaaggcggcctcggtggaccgaggcgctcccgaggacgaggtgcaccacgaagtggagctcgtgctcgtggacccgccgctgtccaccctggccgccacgctgcgcctggcctcggagacggagtccttcatcctcttcacggtgaccgcgctcgccaaggaggagggcaagctgcgcgcgcgcgtgcccatgtcgcgcgtcgtcggcctggacgtgaagaacgtgcagctggtcaacgccatcgacagcatcgtctgggagcgcaaggcgctcgtggaggagaccgcgctgcaggaaggctgtctgctgcgccactccaccgagcggcggcacctcttcgtggactacaagaagtacctctcggccatccgcgtggagctggtaaaccgcgtgcgcgtgcgctccaaagaagtcgtcgcggacttcaagttcaagtactttctggggtccggcgcgcaggccaagagctcgctgctgcacgcactcaaccaccccaaggtgcggccctcgcccacgctggagttcgaggtcgtccccgcgggcgaggccgtggacgaggccgccgtgctcgcggagctgcgcgccgtggcgaaggcgctcttcatggcgcccaccgacgccgtcttcctggcgccgccggccgagatgccggtgcgcacgctcatgctgcagaagcaggagatccccgcgctagacctcgacggccttttcgcggtctccaagacggacggcgtctccgcgagcgtgtgcgtggacgaggacggcgtcttctgcgcgttctcgcacctcgcgtacaccatccggtacccgctcgcgcgcgaagtgcagggccggtaccggctctggtgcgaggccgtgcggcccgtgggcgagcgcgtgtggtccatgttcgtgctggtcgtggaggagcctgcgggcgatgaccgcgtcgcggccgtggccggcgccgtggaggcgctgcgcggcgtgtgtgcacgcgtcgagttcaaacctaagcgcgtggacgggcccttctcggcgacctccgagctggtggagcacatcaagagcgcgctgcagacggagccagagggcgtggtgctcttctacgcgcgcggagagaagtccaagcgcgacctcaaggtcaagcgcgacaacacggtggaccagaccacgaacgtgatgttccggtacatgtccagcgagcccatcgtcttcggcgagggctccaccttcctggagttcaagcggtacagcaacgaccgcgggttccccaaggagtacggcgcggggcgcatcttcctgcgcgaggacgtggtctaccacaacaacatctactgcatcgagttcacgaagacgcacctggaggtgggcctccgcagcgtggtcgtgcccgtgaagttcatcggcgagttctcgcaggaggggtacctgctgcggccgcgcctggccaaaacggagtgctacttccgcaacccctcattctacgggaaccagcactcggtggtgctcgagcacactcgcgaccagctgctctcggtgggggacgtgttcgacgagagccgcatggccgccgtcgggcagacgctggccaacgacgccttccgcctgaacccggacacgccctacttcaccaaccgacgcacgcgcgggccgctgggcgtgctctccaactacgtgaagacgctcatgatatcgctgtactgctcgaagaccttcctgaacaacgccgagcgacgcaaggtgctggccgtggacttcggcaacggcgcggacctggagaagtacttcttcggcgagatcgcgtccatggtggccacggacccggacgcgcgcgcgatcgagcgcgccatggagcgctacaaccgcctcaacgcggggctgaagtcgcgctactacaagtttaactacatccaggagaccatccgatccgagacctacgtggagagcatccgccaggtcatgtacttcgggcgcttcaacatcgtggactggcagatggccatccactactccttccacccgcggcacttcgccacggtgatgcgcaacctgcgcgagctcaccgcgcccggctgcaaggtgctcatcaccaccatggacggggacttcctgtcgacgctctccgagaagaccagcttcgtgatcaaccgcaacctgcaggagagcgaaaacttcatgtcgatcgagcgcgtggccgatgaccaggtcatggtctacgcgccctcgaccatggcgcagcccatgacggagtacatcgtgcgccgcgcggacatcgtcaagctcttcgcggacaacggcttcgacctcgtggaccacgcgaacttcgagaccgtgatccggcgcagccgccgcttcgtcgagggcgtctcgcggctggagacgcggccctccaccaagaacttcttcgagctcaaccgcaacgcgctcacggagatggacagcaccgacgtggccgcgctgctaaagatctacgtgctgtacgtcttcagcaagcggtaggcagaaccagggcgtcgattccgcgcccgcgccggcgcggaaggcgttgaacagctccgccagccaggctgcggtctcgcgcgcgtcgatcgggccgccgtcgtccggcggcggctcgcgcgccgcgcgcaacaccagcgtctccgcgggcggcagaggctccagagcctcgaagaccgcgcggctcgggaacagcgcgcgcatcatgcgcgcgcggtggccgaacaccgccttgaccgcgcgcagtgccgagcggttgtccagccgcagcgctcggtcaaaacgatgcacgcgcgcgggcgcgccgcggtggtcgcgctccacgagcacgtgccgccacgccagcgccgcgccgacgcggtccaggctgggcgcgagcgccaccaggcttttcagcgcatgtaaatctccgcgcatggccgacggctccatttactactgcggaggaacgcacgtggtcgcggccgcgccgggcgccgcgcttgtggtgctggacgcgcccggtgcggcggcggcggccgcgcccgcggggcagcgcgtcttcttcgccgagtacggcctcgagaagcgggccggcggcccgatcacggcgcggctgcgccgctccgggttccgcggcgccgcgaacgcctgggcctccgtggcggacttcgaggccggcggccgtccctccgcgtggacgctgcgcgcggaggaggcttcgcgcgtgccgctgccgacggacgcggcgctggtcctggcctggggcgcgcgcgaggagccgctgcgggcgtgcgtgctggcgcgcgcggcagacgcggaggcgccggtgggcgccgcgctcaaagaagccgccttcgacgcgcgggcgccggcggccgcgctgttcgcggcgctgggcgcgcccgcgctcgcgcccccgctgcgggcgcggctagtggcgccgccgggcgcgccgccgcggacgcggctctgcgagaacccggccatgctgcgcgcgttcgcggtgggctggttcggcgcgcagctgggcgaggcctccgaaaatgaaaaggtatttgccgcctttgataaggcgaggtcgtgtttggacgaccgctgatggcgacgcccgcgaacgcgcccgcgctgctcgtcgcggcgctgcgacaccgcccgtaccgcgtggagtaccacccggactgggagccggtcatcgagacgctggtggacgagtacgacgcggtcgcgccctggctgctgcgcgacgcgacgagccccgagcccgagcgcttcttcgcgcagctggcgaagccgctggcggacaagcgagtgtgcgtgtgcggcatcgacccgtacccgcgcggcggcaccggcgtgcccttccagtccccggacttcagcaagaagaccatccgcgcgatcgcgagctcggtcgcgcgcacgaccggcacgcagggctacgcgaactacgacctggacgcggttccgggcgtgctgccctggaactactacctctcctgccgcgagggcgagaccaagagccacgcgatgtactgggagcgcatctcgcggctgctgctgcagcacgtggccaagcacgtgagcgtgctctactgcatggggcgcacggacttccagaacgtgcgcgcgcgcctggacgtgccggtgacgctggtggtgggcttccaccccgcggcgcgcgacgggcagttcgcgcgcgagcgggccttcgaggtcatcaacgccttattggagctcaacgggaagtctcaagtggactgggcgcgaggattttctttttatagtgaaaattaatccgtggtcctaaatggcggcgcccatatgcgataactctcacgtgttcctcctcaagcgcctgggcgtgccgtcttcctgccggcgctcggaggacccgcgcttcgtggagatcctgactcccttcgagctctcaaactacatcgagcggcacccgggatgctgcctcttcgagacgctgcgcgacgaggaggactgctccgtcgtgcgcgtcttcgcggacgtggacatggacagcgtgctcgaggaggaggacttcgtcgcggcgctggaggacctcatcgtggagctcgcggccttcttcgaccgcttcgcgagcggctcctgcggcaccgtgcccggcgaggtcaagcgcgccatgctcgcgaacttctcggtcacgcgatccacggccgagcacaagaccagcttccacctgatcttcacggagacgtacaccacgctggacacgctggtggcggcgaagcgcccgctgctggacctgtgccggcgctcggacaacgtgctgctgcgcgcgctggacacggccgtgtaccgccgcggcgcgacgctgcgcgtggtgggcacgcgcaagacgccggagtcgagcgcggtccaccgcatgcagtcgcccgacgacgacatcaaggactacctgttcacgttcgtggagctctcggacgcgagcgtgtacttcgagctcgcggagcgcgagcagcacacgctgagcaccgtctgctgggagacctcctacatccccttcggcgacgcgatgcggcgcgtgtgccaggcggtggtcaacgacatcgtgaacctccgcgacatcaccgaggacaacttcctcgacacgccgctggtcatcgactacgcgacgcgctgcgcgctgtgcaagaagcccaagcacaagcacgcgcaccacatcaccatgggcaacggctgcctgcgcctggtcaagggcgggaacgcgcacagctgcaaggtcaagatcatccagctcgagggcaaccggctcttcacggccgcgcagatcatcatcgcgtccgaggtcgtgaagctcaccgagcgcaacgactacatcgtgtggctgaacaactcctggcgcttcagcgcggaggagtcgctcatcaccaagctcatcctggacgtgcggcactcgctgcccgcggactacgccaacgacatgctgtgtccgcgcaagcgcaaggtcgtggagaccaacatccgcgacatgctcgtggacatctccgagacggacacgcagtacgacaagctgcccttcacgaacggcgtgctggacctggccacgggcgagttcctcaccggcgaccgcgcgaaggcctgcgtgtgcacggtctccaccgggtacgccttctcgcgcgaggagttcgcggccgcggcggactcggaggccatgcgccggctggtcggcgtcatcgacgacatccagccggacacgcccgagaacgccgataaccgcgcgctgtacgagcgcgccatgtccagcgcgctctgcggcgccacgaagacggtcatcgtcttcttctacggcgacaccatgaccggcaagtccacgagcaagcgtctgctcatgtccgcgctcggcggactcttcatcgagaccgggcagaccgtgctcacggacgtgctcgacaagggcccgaaccccttcgtggccaacatgcacctgcggcgcgcggtcttctgcagcgagctcccggacttcgcctgcaacaacgcgcgcaagctgcgctccgacaacttcaagaagctgaccgagccctgcatcgtgggccggccctgcttctccaacaagatccacaaccgcaaccacgccaccttcatcatcgacaccaactaccgcccggtcttcgaccgcgtggacaacgcgctcatgcgccgcgtggcgctggtgcgcttccgcacgcacttctcctcgtcggccactcgcgcggccgccgcgcacaacgtcgagtacagcgcggtcaaggagatggacgagagcctggacaccaagatccagcgcaactacttccgctacgccttcctgcgcctgctcgtgcagtggttcggcaagtaccacgtcccgcaggtctcgctggcgcccacgcccgacgcggtccccgacttcgccttccaccgccgcgtggccgagctggtggtggccagcaacgacgcgcaccgccgcgcgatggagtcgctgtccaagctggggtacgtgctcgtgggcggcaacgtggccatgcccgcggacgccttccggcagcggctggccgcgcacttcaacgcgcgcgtgcacggcggcgacatagacgccttcatgttcaagcacaagaaggtcgtcaacgtaacggaggagtacgtggagtacgtattcatcgaagatgtcgagaataaatagacgggtatgaactcggacgtgataaagctgttcgtcgggcacgacgagtccgtgcccggcatcctgccgcaccagctcgcgaccgtggacttcctgatacgccgcgttctagacgacaacgtcagcgtgcttctcttccacatcatgggctctgggaagaccgtcatcgcgctgctgttcgcgatggtggcctcgcgcaccaagaaggtgtacatcctggtgcccaacgtgaacgtcatgaacatattcaactacagcatggtcatggtcgctaacctgttcaacgcgcccttcgtggccgagaacatattcgtgtactcgacgactagtttttattcgctaaactgcaacgacggcgtcataaactacaacggcctcggcaagtacgagaactcggtcttcgtggtcgacgaggcgcacaacatcttcgggaacaacaccggcgagctcatgatggtgatcaagaacaagacgcgcgtgcccttcctgctgctctcggcctcgccgatcacgaacacgccgctcacgctcagcagcatcatcagcctcatgtccgagaaggacgtggacgtcggcgacatcgtggtgcagggcaagaaggtgttccagatcctgctgaacgagcacggcgtgcgcgtgatccgcgaggtgctcaaggggcgcatctcctactacgagatgccggacacggacatgcccgaggtgctctaccacgggcgccgcttcctggacacgcgcgtggtctactgccgcatgtcgcgccggcaggaggacgactacctcaccgtgcgccggctctgcaacaacgagatgttcgagaagaacatgaacaacgtgtccatggcggtgctgggcccgctgaacctggtgaacaacctggacgtgctcttccaggcgcaggacaaggacctgtacccgaacctgcgcatcagcaacggcgtgctctacgggaacgagctcaccaagctggacatcagctgcaagttcaagttcttcatctcgaaggtgggcgccatgcgcgggaagcacttcatctacttctccaactcgacctacggcagcctggtcatccgcaacgtgatgctcagcaacgggtactcggagttcggcggctcgcagagcaacaatccgcacaccacgcccgacgggcgcgccaagaccttcgcgatcgtgaccagcaagatgaaggcctcgctggaggagctgctcgaggtgtacaactccgcggagaacaacgacggtggcaagctcatgttcctcttctcctcgaacatcatgtccgagtcctacacgctcaaggaggtgcggcacatctggttcatgaccatccccgacaccttctcgcagttcaaccagatcctgggccgcgccgtgcgcaagttctcctacgcggacgtggccgcgcccgtgaacgtgtacctcatggcggcggtgtactcggacttcgacgaggacatcgtctcgctggaggactacagcgtggaggacatcaacgcgctgcccttcgacgtgaagaagctcttctacctcaagttcaaggccaaggaaaccaaccgcgtgtacgccatcctgcaggagctctcggacgcgtactccgcgcgcccgcacccgcagctcgtggacgtggtgctgggggagatcgtgcgccagttcttcgcacggcactgccgcgtgcccgccgaggacgccgcgctcgtggccgccgtcgaggccgttctcggcacgcgcgaggcagcggccgagtacatccgcgcgatagtggacggacacttcttcgtgaccaacaagaccttcgggaagtgcctgctcttccggcacgagcgcgacatcgtgaccgtgcccttcgagctcgagcacgaccccttcgcgtgggcgatcaacttccgcaaggaggtcagtgtggtgaatatataacggcaaacataaatagaaagactgtccttttgcgcgatgtcgaccttccggcagacggtgtacctggcggtgacgctgcagccgcacgagctcacgctcgacttccgcggcaacgtcgcggaggcggtcatgcgcgagtacctctacaaggagaagggcgggctcatggccaccgacatcgaggtctgcctcggaaacgagatgccgctggggcgcatcgtgaacaacgcggttgtggtctcggtgccctgcaacgtgaccttcaagtactaccgcgtcggcgacaccgtgagcggcacgctcaacgtcgaggacgagaccaacgtcttcgtggactgcggagacctcatctgccagctcggcaagagctcgggcggcgtgaccttcaacgagtccaagtactgcctcgtgcgcaacggagtcgtctacgagcacggcagccgggtctcggctgtgctgcgcgaggcgcgctccggacgcgagtccgcgttcgtgttctccgcagtgctgctggacggcgtccccgccgaggagaaggacgagaagaaggacgagggcgagaaggccgcggaggaggagacgcccgcgagccccgccgccaaaaactagcattattgggccgcgcgaaccttcgataaatgcgcacgtacacgtcgctgctctcgaagctgctcaagagcaaccggcggctcgggagcacgcgcgtcttccgcgacccgctgcagcacatcagcgcgaccgcctttgtgcaccggcgcatcgaccggcaccggcgcgtctccatctgcgccgtgctcaccaccaccgacgggctcgtggtcgcgtgccggcgccggtactcctttttgtcctccgagctcgcggagacgcgctcgcccgcgcggcgcgtgctgctcgcaaccaagcacgcggacgctctcgcgcgcctcggcgccgcgcgcccgcgcgacgacgtcatgtttccgggcggcgccccgctgtccggggagtcgccgctggcgtgcgtgctgcgcgaggtcgaggaggagaccgggctgcgcggcgaccaggtcagcgtggacgagcggctgttcgtgcacgccttcatcgacgacctggtctcgggccgcgacttcgacgcgatcatcttcacgggcgcagtcgcgctttcgagcgcggaggtggcgaagcagttccggcccaacgacgaggtcaaggggctggtcttcctgcaccccgaggacgcggagggcgtgggcgtgatggcgcggctggcggcgttcgcgcgctgcgcggcgcgcctgcgctgctggggcgcggccgtcacgcgatagaggcggggtccaccacgtacacgaggcgcccgccgctcacgcgcacggtgggcgggtcgcccagcgcggtcaggaagttcccgtcgtcgtcgaagaggcgcccgccgcgctcgaggaagcccttgcgcaccgtgaccagcgccgtggaggtggagtaccacacgctctgcccgtccgcgagccgcgccgcgcgcgcgggcccgcgcgcgtccgccgggcgcgccaccagcgcggaccagccggagtcgtcctccagcggcgcgaagtccgtgaaggcctcgcgcacccactccagcgagcagcgcttgagcacgcggaagagctgcgtgaactgccgggacttgtcgcggatgagggccagcaggtcctcgtccacggtggcggcgccggagtcctggcgcgcgaccacgaagtgcacgttcacgtagcggcggtcgggcggcgtcatctcgtggctgttcaggcgcaccgcgcggcccacgatctggcgcagcgaggcctcgttccaggtcatgtccaggatgaagatgtcgttgatggagaggaagctgaggccctcggagccgctcagcgagaacacacagaccttgatcttctcgccgtcggtgttgtcgcaggcgttgaaggcgtccacgagcttggcgcgcgtgtcgcgcgtgcgcgaggagaactccacgctggagacgccgaaggcgcggaagtagagcagcagcatctcgatgccggtcacgttgacgaagggctcgaagaccagacacttgcccggcgaggccaggatgcgcaggcagacctcggtgtacttgcagctgcgctcgcgcagctccgcgagcagcgagacgtccgcggaggtcatgcggtcgccgctgacgggcgcgccgctgcggaagagccgcatggccgcctccgagaagacgcggtccttgacggcgcgcgcgaagtccaggaagagcgcggccacggcctcgtcgtactcctgcttggagagcacggacttgtcgggcgcgtcctcgaaggcgaaggtggccgcgatgcgccggtacacgcggaagaccgcggcgccggacttgcgctccatggcggccgcgcggcggtaggcctcggtctgcttcgcggtcatgtccacgtacatcatgcgcacgcgcttgcgcgcgaaggcggcggagccgtcgacgtcgtcgaagatggaggcctcgttggtgactaagtacgagcacaggccgccgagcttgtccacgaggtcctcggggttcgcgagcgcgccgccgttgaagagcggcgtctgcccgaccacgccggggcgcagcaggttcacggccatggagaactccttgacgctgttcaccaccggcgtggccgtgaggcagagcagcttcccgcggcccatggggatgttcttcgcgaggtagttgtacaccgtgcgcgcgggccgctggcgcccgtcctccttggtcagcgacatcgagatgaagttgtggaactcgtcgatgaccacgcagacgcggctgctcgacgaggcggtcttcatcagcgtgaagaagcggtggtggaagcgcgggtcgtcgtagttgatgaaggtgcacccgggcacggcctcgggcgcgaagcgcatcatcgtcgaggtccagggctgctccacgagcgccttcttcacgagcacgaccaccgtccagtccgtgaagacgtcgcgcaggtgcttgagcacgtacaccgcggtcacggtcttgcccacgcccgtctcgtggaagagcagcagcgagtgcatgctgtccaggcccaggaacacgcgcgccacgaagagctggtagtccttgaggcgcacggactcctccacgccctgcatctcggagggcatgtgcgcggtgcgccgcagcgcgtagtcgatgtaggccgcgtgcgcgctggtcatggcgacggtcggcgctccttttacggggtctgtcgtctatctattgtcggcgcgggtctgatttaggggcagtagttacaaaaacgtttccgctgctcggcgcggcgtttggaggagcggttgcggccgcggcggcgcaggcgcgcgcggcgcgtcttcgtggtgcggtggccgaaccagcgccggtgcatgaccgggtgcgagaccgcggccgcgcgatccgcgctcatgcaggttgcgtaggtgcggcacatgctgcgcagcacgcgccgcgtgcgccgctccacggcgtcgagccgcctcgcgacgatgggaaagagccggcgccagccgcgcacggcgaagagcgggcgctcgcagaccgggcgcgcgagcgcgtggtaggcgcccagcagccgcgggtccagcgagcgcacgtaggtctccacgaagccgttgccgaagacgatggcctgtgcgcatagcgggttcgtcatctccctcttggaggcgatggcgtcgcccacgaaggcgcgcacgccgcagtgccgcagcaccaggcgccgccgcgggaagtgcaggtgcgggccgagcgccgcgcgggcggcggggatgtgcagccgcggagaaaaacgcgcgcgtccctccatggcatctaagcgctccgtctgttttcagttatatcgccgcgggcggctactgcagcagcagcttgagcttgcgctggctctcgttctcgatgctcttggactcggaggtcatgctctcgtagagcagcgagtgcgtgacgtagagcgcctcgtacacgcggctggcgaaggccacgaagcggtccacgaactcgctctctacggggtccttgagcacgcggaagggcacggccagcgcgtcgcgccaggcggcggccttggtgcgctcgcgcacgtgcgtcacgaaggcggcgatggccgcgcgccgcggctcgctggcgaccatgacggcgctgtccttgagccagctgtcgctcacgcacttgaagaggcgcacggtgccgaagaggctgcagtacacgcgcagcgcgtgcaccacgtcggtgccgaagagcgtgggcagcttgagcaccacgaagcgctcctgcgtgatctcgagcagcgggcgcatcaccgcgaaggtgatcgcgtggtagtcagccacgtagaggttgttctcggtgaggtggttgttggagcggatggcgccgcgctccttgcggtagagccggttgcgcgcgctgaggtcgagcacgaccgcgtcggccctgccgcgcgctctggagcgcacgctggtgatgccgtgcgcctcgagcaccttctcgacgtcgcgctcgtcgatcatgaggtcgtgcgtgtacagactcagcatctccgtgggcatgcggttgatgtcgttcacgcgcgagcactgcaggaagtagttggtcccgtaggccaggctgggcaggtgcccgacgccgagctgcaggtccagcgcgggcgtcgagtcgaaggtgggcagcgtcacgctgagcccctcgcggatgctgcggcgcacggcctccaccgcgtccatggccgatttattggacgcacagtctgttttcatttcgcggctactgcgcagtcaccttctcggccacgatccccgcgtcgtagctgagccggtacacctcgttgcacaccacgaccatctgccgcggcacgtacatgagcgggttgtgcgcctccatgtgcgcggtggtcacgcgcaccgccagcttgtccttgcccctggagacgttggagttcagcgcggtgggcgagaagaaggtgctgggcgtaaagttgaactgcagcgtgcgcacgccgggcgtcttgccgaggatctcgccgaagacgcgcgagactgcgctgttctccgagtacagcacctcgttgccgaagcgcacgtccatgcgcgcgatgacgtcgatcttgttcttgaagtccacgcccttgaggaaggggtcggccacgaagaggtccttggcgcgcgcctccggcgagcggttgtcgccgttgtacacgttgcgctggcaggtccacacgcccacgggcacggaggcgtcgccgatgttcacggagtggatggcggtcgtgaagcggatgcgggaggtcgcgcggctgtaggcccccgtgatggcggagaacttcttggacatgttgtacacgacggagttcttcctggtggcgaacactaggatgttcgtgtgcaggaacacgcgcatgcccacgggcacgtcgtcgatgcgcacgaagacgtcggtgtcctggatggacacgacgcccgacgggggcacctcgactatctccgcggtctcggggaagccctcggggtagcagttcgagacgatcaccatgtcctccagcaggcgctccacgaaggccatcacgaagtcgccctcggactgctggaagccggggtacgatatgaagcggttgttggcgtcgctgagcacgggcttcatgtacacggacagggaggtgcacgcgtgcacgtccgtgatcaccgcggtggtgtggttgatctgctccacgcgccgccgcggcatctcgatgaaggccgggcgcgggcacaggttcttgaccatgtagccgatgaagctcagctccatggagtaggggaactccttggcgagcttggcggcgtcgaaggtctcgtcgtagaccatgacgcaggcgacggggttcagcgtgaccgtgaccgtgaccttgctgtcgctgagcttgagcgtgctgaaggtcttgtccgcgtcaaagggcgtcttgatgtaggcgtgcacgcaggcggcctccttgatgacgtcgttgggcgagctcccggtggagaggtcgttgagctcgcgcgagaagcccgagagctccatcacgcgctcgttgtccaggcaggagtcgaacagctcctcgccggaggtctcccagatggtgtccgcggcggagttcacggccacgtggcggatgagcttgtacgcgatgtagggcacgtagcacatcttgcccacgccctttatctcgggcaggtccacgctcagcacgaagttgttcatggccgagatgtacttgtcgcggatctcgaaggtcacggtgaccgcgtcgctggtggtgtccaccacgccctgcgtggtgatgtactgcggcatgtacaccgtgggcgcgcggtggtccgtggcgaacacgctggcgcgccgcacggcgtcgtcgccgcccaccaggctcaccacggagttattcatttattccctgggaaaaccagttaaataaggctcttcagagccatgcgcaccgtccggccgtcgggctccaggtagcagcgcccgtagacgccctccgtggcgcgcgtctcgttgatgagcgcgcgcacgcggtcggggtccgcgtacatctccagcggcagcagctcgatcttgggctcctcgcgcagcgcgacgaggtgccggatggagcccgcgaaggagtcgcggcacaaccgcgagcagaactcgcccacggcgccgccgtcgagcgtctccacggccagcgcggccgtgcccacgcgctgacggcagaaccagcacgtgccgtccgcggcgcgcagcgccagccgctccgcggacaccgtgttgaagtacttcggcagcacgtactcgatgcggcacgccgccggcggcgcgcaggccgacgcccgcggcgcggatatgtccacccgcgagagcgcgatgcgcttcatgggcggcggtggctgctatttatgtcgcccgcggcttttcaaaggtcgagcgagcacgccgcgaagcgcgcgggcgagaacacgtactcgtggccgaactccgggatctgcgcggcgcgcttgcgcgcgcgcatgtgcgcgaggaagttctcccaggtgagctggttgctgttgttcttcgcgtagttcttcacggtctgcggacgcaggttgcgcgtcacgcccgtgacctcgaagatcttgtccaggaagaaggagtagttgatggttttggtgggcgtgatctcctggcagaagaagaccagctgcttgaatatctcgatgacctcgttgatcttctcggtgctgaggtccagcttctcgtttttgacctggttgatgatctcgaagaccagcttgtagtccttcttgttgatcatttcgctgtccttgaggaagctggagacgtagttggcgtccacgtcctcgggccggatctggtgccggtccatcatcgcgcgcaggtcgcggatgacctcctccgagcactgcttggagagcagccgccggagcacgttccgcaggtggatgagcttgttcgacacgtggaagttggacctcttctgcacgcggatgcccatgggaaacacggtctcgcagaacaggcagaactcgtagtccgcgtcggacacgagcccgttgcggcggcagccgccgcacatgcgcaggttcatgcttgctccagccccagcacgcgcaggatctcgcggtccagcactttagtgtccagcgtgcgggttctacagaactggaggaagcccgcgagcgcgcgcgcgcgccctggctgcgagagcagcagcatgcgcgcgttctcggggtcctcgttgatgacgcgcgtgaggttcagcgagcaccgcgtgcagcgccgcggcgggtcgagctcgaccgagtacgccgcgaaccagacgttgtcgcccatgtattatttattaacacagaacgtcgcacatgttgcgcgaggacatgtacgggtcgtactcctgcccgtagatgaggatggtgcagtaccgcgagatcatgagcatggcctcctccatggtgagcaggtcgtcctcgaacatggccttgtgctgcatgtgctggctctgcttggcggccatcgcggccgcgccgtcgccgcgcagccactcgttcagacactggccgtcgccgccggcgccgctctcctgcgcgaaggtgttgcgcatggcgcgcatgagcctggcctccctggccgagcggctgagcacggtcatggggtcgtagagccaggggcctgcgtccgtgaacaggatggtgcagtacccgttggcgaaggcgtccgcgccgctgcagttgtcgatgccgtcgccgaccctgtagcacaccgcggagaccagccggtacatgatgccgttgagcatcatgtcctgcgacacctcgatgggaatgtcgctgatcacgggccgcatgttcgtgaagcagtcgcccgtgctggccatgcccccgcgccggttcaccaggaacaccagcacgccgttcgtgatcacgggcgcgcggtcgcgctcgtagaggtagccgctcgccgcgcacacggcggacgccacgtccgtgcgcgagaccgcgcccatgttctgggcgggcatgtacagcactcgcccggcctccgcggtgcacgagaagggctgctccccgcccacgtggatgggcgccgtcgatgtcgtgatcatcttgctggagtccaccaccaggtagggcaccgtgtgcatggccatgtcgcccatgcccccgatccccgttccgaacgacggccgcgacacgctcacgagcgtcggcttgaacgagactatggagaagatggaggccaagatctgctcctcgtcggtcatgatggaggcgcacgaggggtggatgatcttcattagggcgttgtcgatggactcgtcgctctcgcagtagaagacgcccatgcggaggttcaggatgcaccgccgcaggttggtgtgcagcaccgcgcgctggatctccatggacacggagtcgccgacgccgggcatcacgatgggcgtttcctcggtgagcttgttcaccagcagctggtagttgttgggtcgcacgcggctgttgtggtggagctgcgccaggagcgagaggctgtccccgttgacgaacgcggactcgatggccggcagctttacgccgaacagcgccatggcgatggggtgcacgaagcccacggagtcggacgacttgaacgagaagagcaggtcgcccgaggagctcatgtctttgaagtgcacggactggaactgcgtggcggagagcaagttctggtagctggacatgctctgcaggtcgtcgatctccttcatctgcttgtttacgcgggtcgagttcctcccgtagatgatcaccagcgggtgcgtctggctcacggatagccccgagtccgtcatggcggcgcgcacgctgttcagcagatcgaacagctccgaccggtctctgttctggatcccgacctttgccatcacagacatgagctcctggatggtcatgttcttgtggtctcggcgcgtggaccgcaggtagtcggcgatcatctcgccctccttacggatcttcatctgccagtcgtgcatggaggtcatgcggtccacaggcatgagcacgctgtcggaggacgactgcgcggcgctgccggactggcgcgagccagggcgcgcggacgacgggcgcgcggagctgccgcggctggaggaggacctggacctccgcgaggatcttcgctgcgaagagctgcgcacgggccgctgggcgcgcgccccggcggaaaccatgtcctcgcggtttatgctgaggagcgagctgcagaccgcgcacgacagcgactgctttggaatgtggatgtggtcgcactccagggacatgcccgcattgtcgtagcccgggaccaagtcgaactttgcattaaaaaaatctgatgcgcacgcgggcgattccatttataccggaagtttttatgaggtgccggtattatccacgcgatctcgcagtgtgctgggagactctagcgtagccacggaccccgtgagcagacgacgcaagtcgttgatggccgactgcgtgacggacttcgccgtctcgatgtcgcgcgtaaggctgagcgactcggcgttgaggtcgcgcacgctgtccgcgatgtcggccagctcctttttgatgaaatccttatcattatcggcgttgatgactttgtccggcactctagactctagaaccggtgacgcggcgggcgccttgatcgtcgggcagctggacgccgggtactggggcggcggcaatgattgctgtaggaagatgggcttagcggcaggcgccggttttgtcggcaagggcggcgccggcgggcactgtcgtgtagacggcgggcacgccggcgcggggcacgccgccggtggcggacatgtcggcgcagttgcgggtggacacgcgggcgcgggcgccggcgcgggacacgtcgcggcaggagccgggcacgcgggagccggagctggagccgggcacggcgcggcaggagccgggcacgtcgcggcaggcgcagggcacgcgggagccggagccgggcacgccggcgcaggaggacatgccggcgcagcggcaggacagaccaccgctgtcgcggagcacgcggcaaccggcgtggagcacgcggcgggcattacgttcagaggcgtcgactggaccttggcaccgcggggcagtagcgatggcgctaggggcgactgtccggtggttggacgctgcatgcaggcagtcgcagatttcagacgggactggtaatacctgcccgcttccttcaccgtgtacttgtcgacggagtctacctcctcatctggaggacatggctgctccggtgcgggaacgactgtttgaggacacttggtgaacagactggagctggtctccgatagcaccagtttagacttggccaagtcggaggcaaaccttcttctgagatccatttaagccttcaaaattgaacgtgtacgccgaccgctaaatggaagaatcggtggccgtcgagtacgcggacgaggacgaagatgagattgaggagtacgaggaggaggacgatgacgaggaggaagagtctgccgagggcgccgccgcctcctcggtcagcgacgtagcgctctctgccgccgagaagctggtggcctcggaggtcccggacgacgcggctgccgcggacaccaacgtgcgtcaacgcgtcaccgcgcgcgtggaggagcttaaggcgcgctacacacggcggatgagtctatttgagctcaccggaattgtagcagagagtttcaatcttctgtgtcgcgggcggctgccgctcgtggcggacgccgcagacccggcgctcgacaacgagctaaaagtggtggttcgggagctcgaggagggcgtctgccccatcgtcatcgagaaaaacggcgagttcctctcgccgggcgacttcgaccccgagtgcctgcgctaccacctgacgtacatgaccgacctctggaagtcccaggggcgcatgtagccgcggctacgccgactcggcggcctccgcgattttttcttttatcatgtccagcagctcgcgcaccacgatggggcggccgcagtacgtgatgccgttttcggatatcacgctctgtgcgatgtccaccagcgagccctcgcgctcccagtactcgcgcgcgagcacctccttgtacaacgcgcggtggttggccacgtaccgcaccagcgtctggatgttcttcacgcccacgctcttgaggtcctgcggcgagaacttctcgcgcagcgacacgaagacgtcccggacgagcttgccgatctccacgttggtcttgaactcgttgtacagcaccacgtagagcttgcacacgaccgtagcgaacttcgcgggcttgagatccttgttctggaagaccagcatgctgctcatcaccttcttcatgaaggccaggtacttcgcgcggtcgccctcgacgctcacgctcccgacctcgaggtcggacacgcagcggattccgtgctccgcgctctccgcggagacgcgcagcagctcctggtactccttgagcttctgcttgtccgtcatcagcgagttgtcgaataccgccaccagcttgagcacgtagttctcgtccgagaagaccttgttcagacacttcaccaggaagctgtagtggctctgcaggatcttcatgaccgcgttggcgccgctggctccgcggacgtgcgatatcatctccatgatcttcttagagtcgtcgatgatctcctcggtgtcgttgcgcatgttgcggtacattgcgttcagcgagaccagcgtctgcgcggccaggagcacgtcgcggaacacgcgcgcgaactcgcgcttctcctccgcgtcggtgatgctgttgtacacggacttcgccaccgcgttcgacttcaggaaccagaaggagagcgcctggtagttgaagtgcttcatcagcgccagcacgtccgcctcgctcatttccggcgcaatggggcacaccgagctctcgagcacgggcaccatgctgacgagcgtgtccacgtccgtgtcgaagtccaggcagtccacgcagagcccggtgccgcggctcaggtgatcgcggctgatgtcgtagaagcgctcgtagcaggtgcggaggcggtccatgtcggctgcgttttagagagacacacactcttgaattatggctgcgggtagaactcctgcagcagcgccggcgcacgcgcggagtccggctccactcccagcttcagcgcgcagttcacggaccaggtcttcatgaagcggtcgggcgcgtccgtgaccacgtgccggaagagcttcgcgaagtggcggctcacggcgttgggcacggtcgcgttgcgcacgaaggccgtgaagcgcgaggtcagcttcggcgcgaagcgcttgccgtccacgaagaagcccgaggtggtgagcgagagcccgttctcctcgcgcaccacgcgtcgcgcggccttgtgcggaaacatgctcgcgaggcgcccgctcgcgtcctggtctaggtggatggcgtccgtggccgcgtccttgcggatgcgcaccacgtcgtgcacgatctcctggatgaggatgcgcgtggccgcggtctccgccagccgcatggggaagtagaccatgtccccggagatgagcacgttcccgctagcgtttacgtagctcactatctcggacacggtgcgcagacgcacgatcgcgccttcgcagcagtgcaccacgtagtacccagcggtggcgcgcaggcgcttgttgtccgcctcgaagtccgcctccaacccctcgttgaagtacttgtcgaatatgatgggcaggaaggatagttttgactcggtgaccacctttccgaagttgaggatgtacgggttcagcgcgctgcggtcgacctcttcgtcgtacacgcaggacttgaaggtgtcggtgtgcgcctggctgcgcaggaagcagcacggaatgcagatgcgctgcaggcggtggaagatggagaggaagcccacgctgttgtagcgcccgtcggggtccatgcacgaaaacatgacgccgtttccgtttacgaagacctcgcgcgtctcggacttgaagaagttgttgctgaccttggccatgttcgcgtccagcgactgcacgatcacgggcttgcggttcttggtcttggtgttctggcagatgcgcgaccagtacacggtctccaccttggtgaagtccgaggactgcttcacgttgttgaacatcacgctgatggccacgatcaagaacgtgaagtacttctcgatgttcgggatgtagttctttaccttcacggacacgtgcgacttcgcgaggatgatcgagatgcgcttgtccgtggagagcaggatgttgttggtcgccgtctccacgaagatgaagctcgtttccatgtccagcttcatcttagacgtgatggtcgtgttcagcgacaccttgtaggtgatgtcgcccttgacgcggtccatctttacgtccatgctctcgatgagcttcgtgaacagactcacgtcgttcaccgtgagcgtcttcccgtcgctcgagatgaccaggtcgccttccgggccccacaccgagaggttcagcggctcgtccaccagcaggaagcgagtccccgtcatcgacacgaagaagtcgtccgtcttcgagagcaggatgtcgaagtcgcccacctccgctcgcttgtccggcgactgctgcgcgatcgcgcggagcccggactcgcgcaggttcgtgcggaagatgttgttgaacttggtctccacgttcatgtttaggtcgaggttcgcgaactcgcggatgagccgctcctcgaacttgaggatggagtcgttgggctcctcgaaggagccgaactccggcgcggaggtgtccgccgcgcgcgccacccagaccaccaggaagttgcacgcgtccgcgtacgcgttgtagaggatgccgtccgtgcggatgagcgttttcttttgcgtgggcgagaacgggttgaagatggtgttgtccacgtagctgtactccaggttgttcttgtgcgagtacacgatgatctcgtcctgcaggcccagcaggctccccaagtaccccttgagctgccgcacgcgcatggtcagcaagatgtgtctgcgcacgtgctcggggtccttctggatgtactgcttcgcgaagaagtagatcggcgaggcctcgtccacggagtcgtacagcgataggtacagcacgcgctcgatctcctggtggcgccccaccagcaccaccagctgcggcgcgacggtgtagagcatgttcgcgcgggcgtatttatagccggcgttaaactgaaataaaatacgcgggtcgcgaggcagcgccatgttccagccggtgcccgacatggccgccgaggccgacatcgacctcggcgacgtcagcgtggacgcgacgcgcgcgggcgcgcgcgagaagaccgtcttcttcgcgcgcaacaagcgcatgtacccgcaccgcagcaaggacgaggagcgcaagctgtcgctgggcttcttcttgcagcggctggacttcctcacgtcgcgcgaggtcaacctgcagttccggtcgctggacgcgctgcgcaccgagaacgtcatgaagaagaacaacgtgctcgtggcgccgtacatcctcatcgcgacgctcgcggggcgcggtttccgcatgacggagaccatggtcgagctctacttccccgagctgtaccgcgagaccagcaagcgcttccgcttctgcgcgcagataaaggtcatccaggacttcctggggttcgcccacgacagctaccacacttacgacttcgagacgtacttcgcgttcgtggcgctggtgctgcgcggcgcggactctgcggccgaggccttcgacgtccgcgccgagagcgggcttgtgcgcagcctcaccgagatcacgtaccggctctacgtgatgcagctgcgctccgacgccgcgcagtggagcgtgagcaccggcgccgtagtctcgcaggcggtgaacaccgtgctgtcggtcgtcggcgacctcgctgcgcgcgcggaggccgagcggctcacgcccgtgtgcgacctcgcgcgcgagaacccgctctcgctcgaggacctgcgcaagtacggcccgcggctgcgctcgctgctcacgaccatggcgcgcgcgcgatccttcaagacgaaccggcgggacaaggacgcgctgtcccggttctgccgactgacggcgggccctagcccgtctgcgtgccgcgcgtcgccataggcgtcggcgcgcgctcgccgccggaacactcggggtcgctgaacatgtagatgagcgcgacgcctagcagcaggtacatgatcatgctgatcacggttttgaacacgacggcggcgaacgtgttggaccgcagtcggtgctcgcagaagtgcatgaacaggtgccgcatgaggtcgatggccccgttggccacctggaaaagggcgaggccgccgatggacttgatcaccgtcacgtagcacggccgcattccgacgacgctatttactcactgtcaaaagaaacggcgccatccgaccggaggttgaggttgcgcttcatgttgttccagtacatctcaccgatgctcgagtagtacgccgtcagccgcgatattttttctcgcaccagctcgtaggccttctgcatctccgcaacgccgatctccgcgtcgcccacgtaccggccgctgcggcgcacgatcagcagcagcgccttcaggttctccagcgcgatcatgtccatgtacagcgacttcgagagctgcacgaagaggttgtaccgctccaggatgctgttcttcacctcgtccgcgatcgggactccgaagatgcgctccgtggtgtacacggactgcgtgagctgcttgaagagcgcggagatgcagcaggtcgcgcgcttgacggcgtcgagctgcttctcggagcgcgcgctcgcgatgctcagcgcgctgttcacgacgttgctcgtgtcgcgcacgtagcgcgtcttcagcgcggcgttgatggcatccgcgatctcgttgctgctcacgctcgagtcgtccgagctgcccgagacctcgtccagcagccccgagatcgtgatgtcgggcgagccgccgacggtcaccagacggtcgagcaggttgcagggcatggacatgaggatgccctcgctcgagaggcagccctcgtcgatcatgctctgcaggttgcgcttgaaggccgtgttttcgggcatgaacccgtccacgctcatgagctcgtcgaccgtgctcgcggaaaagatgcccctcacgttgatgcggtccagcatgcccatgtcctgcgagcacagcaccaccgactggtcggccgtctcggcggcgtccttggcgccgccgtagatgatgcgcggaaaccgccagctcgccggaaaagagaaggagggaaaccggcactgcgcgctcgggcctcggtagccctgcgcgtcgcgcacgttggtggccgtgaccatgaactgcagcaggtcgtgcgcggacgccatgatcttctccacctcctccttgctgcagcagaccttgcccaggctgcgcgcgatgttcgttttgctcactgagggcgagaccgtgacggcggtgtgtcggcggctgccgagcgtgtacgcgctcacgctaacgcggtaccccatggcgccgaagagcagcttcacgaagtccaggtagctctccttattgatgtagtgcggcgcgcccttgtcttccatcctcagcccggcgtaggccatgagcacttccttcatcgccgtctcggggtccgagttgcacaccagccgcagcatctggaagaactgcatgaaggcgcgctgcgagagcccgatgtggtggttgggctgcgtcgaccggcgcgggaactccctgggcgtcatggcgttgatgcccgagagcgtctccatcacgagcgcgcccacggtcttctggcccatgacgcgcgggtaaaagcacacgcggaggggctccttgccggccgcgagcgcgtccgagagcagcgagcagtacgtgacgttgtcgtggtcgaagagcgcgaaggtgtagcagacggagctcatgaagagcgagtcggcggtgctcatggacttgaactccgtgtacgcgattccgtcccagaacaggctctttccgggcgcgatcagcggggacgcgcggtcggcgcgcatcagcatggagagcagcgtcacgtagtaacggatgttggcggaaatgtctacgaactgcatgccgggcgaggccacgcgcagggtcgcgcccgaggtagtgagcacctccaggctgtccatgagcgtcacgctggggtgcagctgcgcaaggcgcgccagctggctctggtagaagatggacacggcgaggctggccacgctgccgcgtgccatgcgcaggttttgcccgttgaaggtgagctggcgcagcgagaacacggagtcgaagtactggaagaaggtgagcaggtacttgagcggcatggtcgtcagctcggtatccacctgcggcgtctgtgcgagcacgattccgttcttggccgcggcgtcggggatgtcgtacatggcgtccattctggcgcgggaggcgtcggtgagcagcgcgcgcacgttgagcagcatgagcaggtcccgcgctagcatggtcccgtcgaccagccgggcgcgaaagccgatctcggcggggccggcgatgttggggtagatcaggttcagcaggtacgtgttgtcgaagctcagcgaggggaaggagatgggagacttcgccgggaggccggtggggtagcgcacgtagccgccgcagatgcgcgcgtgcgcctcaaagctggtcacgcgagtcttcagcaggttgcgggtgaagggcggcacgtccttgaaggactgcgtgcagatcacggggttggcggtgtcggtcagcttgaggttggtgggcttaagctccgcgaagttggggcccagcagcacggggacgaggtgcgagttggcggcgctgtcgagcaggaagttgatgccgaactgcttcacggcgacctcggtttcctcgtcgctggcgagcttctccgcgtcctcgaggaagagcgcgtccagcgggtgcacgtacgtgcggttgacgtcgtagctgggcttgaagtccgaacacagcgtggggagcacggtggagacgagctggaacatgtattccgcgccctccacatggtgcaaggccatgtgcacgtttggggccgtcatttatttagtattaaatgacggccgtaccggtaaccgatattcctggagactacgggccgacgtccttttcggaggacaactacccgctgaacaagcactacgagctcaccaaaggccagctctcgatcctgcgcacggtcaacgacaagctgctcgcgcgcaccgtgcagcactcggacggggagagcgatgagagcgaaagcgaggaggacgacatctccagtccgctgccgccggacgaggaggagccggactcgtgcgtggcccgggtcatgccgcgggacgcggacctggcggcgccaaaaaaggccgacggctacatcattgccgccgagcagcagcgccagcagcgcataaacattctggtatccgatcgagaggccgtcgtggagcgggagccggttcagacgtcgttcgcgcgcgtctcggctatcccgatccacggggacggcgcgcgccgcaccaccgcctccttctccgcgaccacgccgtcgctgggcgccgtgttcgacgacgccaagcgcgtgcggctgctggaggaggaggtcaaggagctccgcagaaagtgcgcgacctctcaggataacggaaacctggagaacttcaccaaggtgctgttcggcaaggcgccgcgcgcgagcgagctgaacaagcgcgtggtcatcgtgaactacgccacgctgaacaacgtgacgctgtccatggaggacctcgagaagtgctccgacgaggaagtggaccgcatgtactcggtcatccggcgctacaacgagacgcggaagaagaagatcctggtcacgaacgtggtcatcatcgggatcaccgtgctcgagcacgtgctggtgaagcttggcttctcggaggtgcgcgggctcagcgccgacctctcgtcggagctcatcgacgtggagatcggcgaggactgcgagcacatcgcggagcgcctggggttcgggaacagcccggtgctaaacgtggcgctcttcgtggtaaagctgttcgtgcggaagctgaacctgatctgatcaacacatgccgccgtcgaggtccatggcgttcatgaggttggaggcgcggcggcgcgcgccggtggaagcggtggaggcgctcgaggtcgtggagcagggagtgttgctggaggaggcgcggcggcgggagctagaagcggaactcgaggttccgctggtggtgctgcggcgactcgtgccgctcgtgccgctcctgctagtgccagtgccagtgccgctgcggcgtgaagtaccggtgccggacctgccgctggagcttttcttgcggccgccgttaacgctgtcgatgccgagcaggtcctcgcacacctcgccgacggttccctgcacgtccaacttgccgttcttgacaaccccgtacacgatcttgccgcagttggacacagcctggatggtggtctcgtcgctgtcaaaggcgttcattccgccgcacccgccgtcgttgtttcttcgagaaggcgcgccgctgcggcgactcctggtgctgccgctggaccgagttccggaggacctggagcccgtggaccggctgccggtcgacctggtgccggtagtgcgctttctggacgaagaggaggaggcgcttccgcggcgggtggacgaactagcctccagcgcaccggcgccgcccacacaatccacgtcggcggcggcgcctccgcgaatgacctgctcgttgttgagctgcgtcaggagagatcgcagctgcggcgcgatcttctgcaaggtgctcacgtagtcgtcgtagctgctctgcgggcgctgcgccattttttcggacgccatttattacgcggaatatctacgacgacgcagcactgaatcggtttctcgcgacgggagattccgcggtcggcgccggtgcggggttgtcaccgggcgacgaggtaaccagcgcgtggaaggcgcgcacctggtcgtccgtcatcttgtcctcgaacgaggacgcgcccggggggagcaggtccttgttgcgcggaacggcgggcgccgagacgcacgaccggcggtacatcatgatgacgatgtagcacacgatcgagatgacgatcacggtcagcagcgcgtcgaggagccccatttattacctgtatatgcccgcgtttaccgggcggtgagctcaatgtcggtgttgtttagccgggcgtacgggacgctgccggagcacttcctgtacatgctgaacacgaacagcccgagcagcagcacggcgcccactatgaagcaggttacgcacagcgcgcgccacacgtagtcggtgacgttgttggtgttcttgctgaaatccacgaaggcgaagacgcaggcggccgtcagcagcagcacgccgcatatcagcactccggagtagtaagagctcaaggtctcgaatatgtccatttatctgaggagaaatttaaattactgaatggacgaagtggaatagaaaccacgagaacacgacggactgcagcacgaagatggtgctcagcttcgtcttcatgggcatgcagaagttcgcggccagcgccatacagaagatgaacacgagcaccgccgggtcgtagtcggacaccatttacactacgctaaaaggcatatctcggcgcgcgacgtccacgagcaccagcacgcggacgcccgcgggcgcgccggcggcgaccgcggcgagctgcccggccgtggggttcaccagcagcagtgcgcgcgcggttcgcgggacggggtcttcgtaggccatggtcggcgtggacccgggacgcagcggccgcccctgtctgtcgaagaggccctcgggaaacgaggtgcccggaacggccacgacgacggtgtcgctatctagaaacatttatggtcttggtttccacggatcgcctcgagtagaccgccacgaagtagaagatgacgcccgccgcgagcgccgccaccaggaagggcggcacggcgggcaggttcgcggacgcgttgtcgcgcacgccggggtccgggtctgcgtagcccgcgcccacggccttgccgcagtcggcgatcatgtgcgcgcgcgagttctgcatgaccaggctgtccacgtcgatgcggcagcccacgtagcggcaccgcgagcgctgctcgtcctggctgaagaagagccacttgcggtcgcgcgactggtccgtgcactcgtgcgcgcgacagacgcgcgggcccaggtacttcccgagcgtggtgcccgcgacacacgcgcactccggcgcggcgcggtgcgcgtcgcagtagcgccgcagcgcggagtcgccgaaggcgaaggaggcgggccgcgccacgcgcacgaactccgagcagaagcgcgcgtccatgtgcttggcgcagagcgccgcgtaggtgtccagcgccgcgtagcggcccgtgcgcagccaggccatgcactcgggcgcgtcaggctccaccgcgcagcggctggccataacgccgtcgcagtgcgcggtcttgtacccgttcgcgaacacggacgggcacccgggctccggatttgtgcagcagcgcgccatggcggcgtccgtgggcggcgccgaggcgccgatctcgaacgcgcacatggtgccctggcgcaggtacggcttcgcgatctcgggaacgtagtctgcgcgcagcagcgagcccgggcggaagaagagcgagtcgcagggcgggcctcgcacgagccgcgcgcggctcgccagctctggcgagaggaagcgcccgcactgcccggggtccatggtcggcagcagacagaaccgcggccgtacggtcttcagcttcgggtcggagaaggtttctgattcttccgcgaaggcgaaggtgtccgtggcgctcgtgtgtgtgacgcgcagcgcgtactcgcctggcgtcggcgtgtcgagcacctccaccttggatacggtgtcccccatttgaagacgctatttacgccgctgcctactcggcgaagaataggtcctccgacttggcgcccgcgtacaccgggcaggcgggcgcggcggagcgagtgcgcacgataccgcggccagtgaggcggaaggcgtagatggcgaacagcaggccgagcacgatgtacatgaaagtggtggcgcccacagacccggtcacgtgcgtcacgatgatggtgacgatggacatgatcgtgcacacgatggccatgccggtgttgttggccgcgtaggggtgcatgatctgcatggccgcacagtatccgatgaccaggcacggcagcgggaggataagtgaggcaatacctatcattactagagcgagcacgggggtggacgtcaaggccaatacaaaaatcacaatacctgttagtatgcggatatcctcgtactggaggacgctgtaaggcgcgatattccctccgggcactggcctggggttagccgggactaggggggagtcggcagtgccggggtccttggggagaaaggcattctgctcctccgggctgaagagctcggcgtcctgaacgccgccggcggtgaactcgtcgttatagtaactaaagtagctttccatttatatgttgaaaaatgtttggaggcgtacaggtggacgacaaactctacgcgtacctaaaaaaactcgccggacgcgggcggccgctgtgtctgttccgcgacaacggcgagttcgtcgaagtcttcgcggggtccgcgttccgctttgtgctgcccgtgggcctcttcgcggacctgcgcgtgcgcacgcgcggcgtggccttcccgaaactgcgcgactccgcgcgcatgcgcggcgtgcgggtggacgcgcacacgctgccctcgctgtaccccaaccagcgcatcgtggtggacgaggtgctcgcggcccgcgaccagttgctggccgcgggccgcgccgtgtacgtgacgctgcatctggcttgcggcttcgggaagacgctgaccgcgtgccacctcatcgccacgcacggccgccgcgcggtggtgtgcgtgcccaaccgcatgctggtgccgcagtggcgcgcggccgtggcggagctgcgggtgcccttcgcggtctcctgtgacggcgcggcctcgctgctgcgctcgggcgagctcgaccgcgccatggtggccatcgtggtcagccggcacttcgccaacgacgacttctgccgcgcggtgagccggcagtttgacgtgctcgtgctcgacgagtcgcacacatacaacctcatgaacaacaccgcggtctcgcgcttcttgaccaagtacccgccgcccatgtgcttcttcctgaccgcgacgccgcgcacggccaaccgcatctactgcaaccgcgtggtgaacgtgtccgtggtcagccgcctcaccaaggtagtgcgcgtggtggacgccttcttcgagccgtacaccacgcccaagatccgcacgctcgagcgcagcctcgagggaccccagaacaagtaccacgtcttcaccgagaagatcctcggcgaggacgtgcaccgcaacaagctcatcgtggacaccgtggtcgcggccatggccgcgggcgaggcgcggcgcgtgctcgtgctcaccaagctgcgcgaacacatggtcgggctgcacgccgcgctctgcgagcgcctcggtgcggagacggtctttctcggcgacgccaagaacaggaagacgcccgaggtcacgcgcgcactgcgcgacaaggaccgcttcgtgctcgtgtccacggtcttcttctcgggcacgggcctggacctgcccaacctggacgcgctcgcggtggccgcggccgtgctcaaccgcatggtcatggagcagatgatcggacgcgtgtgtcgcgagtcgcacgccaacacgcgcacgctgttcgtgttcccggactcctccgtgcgcgcgatccgcgacaccgtgtctgcgtttgcgcagcggctcgtggcgctggcggtggacgggctgggcttcgtccgcgagcgcgccgccgccggcgcgaagaacgagccggcgctgtacagcgccatcagcgggcgagatctcgcagcggtgtaagcgcggacccgcacgccgcgcacgagagcgtgctggagcaggcgagtcccagcgacagtgtggacagcctgtccacgtccttgatgctcaccagccgcgagttgcacgacgagcacacggggtcgctactatcatcgaccaccgtggtgacgcggcggcgtctgcgctttttgtttccagcggcgacatcgaccacgcctcccttagagccccccttcgcccccgccttagctttcaccgcgctcatcttttatttatcataaaaacacgtctgcgtacgcgttcgcgcacacgtcccgcaaatccgcgcgcgcgccgcagcgcgtgaagcgcgcggcgtccgcctccgcgatccgcgcgcacggcagcggcgcgcccttctcgtccgccatcacgcgcgcagagatcccggtggcccccagcgcgtacgacaccaccacgtcgccgacgcagcggtacacgttgccggagccggcgaggcggtcgaacgcggcgccctcctggcgcagcttgtcgaatatgcgaggaacgaggatgttaaaaatgagaacgaaatagcagatcagcaaaaacagcgagatcatgacctccgagagcgatttatataccttgaaagagctaatacgacttcgggactcgctgcacctcgccaccggcgccgccgtcgagcgctacaacgcgctcgtggagtgggccgcgcgcacgtactggacggtcgcggtgctgccctccgcgccgtgcgcctccatcgagaagtactactgcgtgtgcaaacccgactgcgcgctcgagcccggcgagtactccgtgagccggctgcacttcggactcacgcacgcctgggtgcgcggcgccgccttcaactcggccagcggcgccgaggtcgagccgccagaggaggtgcgtagggcctgcgaggcgctcgacgccgccttcgcggacctcaccttcgtgcgcttctcggtcttcggccgcgagtggacggtcgacgacgccgtcacagaccactcctcgcgcgacgaggtgctcgccgcgtgcgccgcctccggcgtgcgcgtcgcgcgcacgctgcgtgtgcgcgtgcgggcgggagagtccttcgcgcgcgcagacttcgacgcggtgcacgccgcgctgcgcgcggagggcgacgtcgctcgcggcaccgcggtctgtctcgcgctgcgcgggtcatcgcgccgctggatagcggaccgcgcgcctcgatgcttcatgcgcgtgcgccgcgtggagctcgagcccgtggacgcgcggcaccactgcccggtgctgatctccgcgcgcggcgaccgggtgctctgccgcggcgtggggcacctcgcggacgcgcgcgcgcgcgagggcgtcttcgtggccgtgcgcaggtacccggagtgtctggtgctctgcgacgaggcggccgccggcgcggcggagtgctcgcgcgaggaggcgctgcggctgctggtgcgccgcttcgggcgcgacttcgccgtcagcgaggagggctacgtcttccgcgtgcaggacatggacttgcgcggcgtgtccgcgcgactggggctcgcgccctgcgcgagcctggaggagctgcgccgagcggtggagcgcgaccgcgcgctgatgaggcggctgcgcgcggagggcgccgtgcgcctcgcgtgcgagtgcgtgggatacccgcgccagaacgcggtggagctcataaataatatgcgctttcaaataacggaagaaggcgcggtggcgaactttgagctggcgaacgcgagctgtctcggcaacccgaccgcggagtccatcttcgcgagcttcgcgcagttcgtgccggtcttcaacgtgctctcggcgatcgcgcgcgcgcagccatgatcgtggcggccttcgaccttggcacgcgcaaccccgcgcgcaccgtgctggaggtgctcgacggcacggtgcgcgtggtggacgtggccaagctggactggagccgcgactgggagaagcgcgtgcaccgcgacgtgaccgccttccccgcgaacgtggtgctcgtggagcgccagtgcaagatgtcgcctttttctaagttcatatacttcatacgcgggctgctctacgacgggcggcgccgcacgcgcgtgctcgcggtgccgccggccatgaccggcagcacctaccggcagcgcaagcgccgctcggtgcgcaccttcctcgcgctcgcggagagcttcggcatcctggacgccgtgcccgcgcggaagaagctcgacgacgtcgcggacagcttcaacatggccatcaattacgtgctccgaacaaactgaaatacgactggaacgaataagtcatgctggcgctgttcgagttcctgcggtccgtggaggactgctaccggcgcaccatcttcaacttccacatcgcgcacagcgccgaggcgggcgatgtctacggcgtgctgcgcgaccgcattttggcggccacgcgcttcgaggaggtagcgccgccagggctcgcggacgcgctggccaaggtggtctactgcgacataagcaccaccaagcacctggtcaaccacgcggccttcgcggcgcgcgcgcggccggcgcggcgcggaggcagcctcgcgcagttcttcgacgtgcacgtgggcgaggacgcggagagccgccgcaccgcggagatcttcgaccgcgagcgctcctcgctggtctcgtacgtgaagaccacggccaagcgctgcaagatcgactacggcgagatcaagcgcaccatccacggcgggcggcagacctacttctcggggcggcgctcggacgacttcctgagcaccaccgtgcgcgcggacccgagcaagccctggatcaagtccatctccaagcagctgcgcgtggacatcctgcaccacgcgatctgcacgcgcggcaagagctccatcctgcagaccatcgagatcgtgctcacgaaccgcacctgcgtgaagatattcaaggactcgaccatgcacataatcctctccaaggacgaccgcgagcgcgggctcgcggacctcgcggacaagctcttcgggacctacgcgaccaccttccgcgtcatcgcggccatcaccggcaacgcctgcttcgcggcggtggcggacgcggccgcgcgcgtggtcgcgctcccggacgcggacgcgaagctggcggcggtgcgcgggctcgcggagtgctacggcgtgcgcaacttcaaaatcggcatgttcaacctcaccttcacgggcgccatcgagcacacggtcttcccctcgctgatccccgcggagagcaagatcaagttcttcaagggcaagaagcttaacatcgtcgcggtgcgctccaccgaggagggccgcgagtgcgtggagcaggcgcaggcgctgctcgcggccatgcgcgagcgctccgcgcggctcgcggccgcggacgtggccaccgcgagcgtggacttcctcaaggagctgctggggccatagtgaaataatactgatttcttaaatatggagcaggcgctcggatacaagtttttgttgcccgaccccaaggacgacgtctactaccgcccgctccacttccagtatgagtcatacgccaacttcatcaagcaccggcttaaggacatcctcacggtgcggcgcacgctgctcaccttcaagaacggcaccgagtccatcgtgctcgagatcgacgacgtgaagatctcggcgccggagttctcgcccatcgtggccagcatcaagggccacagctacgaggcgctggtcaccttcacggtgaacatctaccggcacgtgatgaccaaggacggcctcaccgttaccaagatcaacagctacgagggcaccgactcgcacctcgtcaagctcccgctgctcatcggctacgggaacaagaacgcgctggacccctccaagttcgtggtcccgaacgccatcggcggcgtcttcatcaacaagcagtccatcgagaagctcggcatcaacatgatcgagaagatcaccacctggcccaagttccgcgccgtgaaggccaactccttcacgctctccttctcctcgatctcgcccgtgcacgtgatgcccgcgcggtaccgacactacaagatcctgctcgacgtgaaccagcccgacaacttcgtgatctcctccgcgaagaccttcatcaccgtgaacgtgatcgtgatggtgcagttcctcgcggacgtcacgctcgagttcgtggcgcgcaacctctgcttcgacatgccgcccgaggccgcgcacctggccaccgcgctcgtggagagcgcgaagaccgtgcccgcgggcgcggacgtggccgagtacgtgaacgcgctcatcgcggccgagcacgcgaagcagaagtcgacgctgtccaaggaggagttccgctacgagatgctcagcaacttcctcccgcacatgcaggacagcgccaaccagctcaagggcctgtacctgctctcgctggtgcgcaagatggtcttctgcgtgttcttcccgaaccggtacccggaccgcgactcgctggtctgccaccgcgtgtacacctacgggcgctacttcgaggcgctggccatggacgagctcgagacctacatcgggaacatccgcaacgacatcctcgcgaaccacaagaaccgcggcacctgcaccgtgaacatccacgtgctgaccacgcccggcttcaaccacgccttcgcggcgctgctcagcggcaagttccgcaagtccgacggcagcttccgcacgcacccgcactactcctggatgcagagcatctccatcccgcgcagcgtgggcttctaccccgagcaggtcaagatctcgaagatgttcaaggtgcgcatgtaccaccccagccagtacggcttcttctgcgcctcggacgtgcccgagcgcgggccgcaggtcgggctcatctcgcagctctccgtgctcgcctccatctcgaacatccgcaccgcggacttcgtcgagctcaccaagcgcgtctgcgactacgtgcgctcctaccccgcgcgcgacatcagctacttcgagaccgggttcgcggtcaccgtcgagaacgcgctcgtggcctcgctgaaccccgcgatcgtggacgcgttcgtgctcgacctgcgccggcgcaagcggctcggcttcttcgggaaccgcgagatcggcgtcgcgctcgtgcgcgaccgcatgaacgaggtgcgcatcaacttcggcgcgggccggctcatccgcccgctgctcgtggtcgagaacggcgtgctcgtcatggacgcggaggcggagcggctcgagcgcgacctcgccgcgatgaccttctcggacgtgctgcgcgagttcccgcacgtgatcgagatcgtggacgtggagcagttcagcttcagcaacgtctgcgactccgtgcagcgcttccgcacgctgccgcccgaggagcgcgcgctcttcgacttctgcgacttcccggccgagttccgcgacgggtacgtggcctcctcgctcgtgggcatcaaccacaactccgcgccgcgcgccatcctcggctgcgcgcaggccaagcaggccatctcctgcctgagcgcggacctgcgcaacaaggtcgacaacggcatccacctcatgttcgcggagcggcccatcgtggtcagcaaggcgctggagacctccaagatcgcggacaactgcttcgggcaccacgtcaccatcgcgctcatgtccttccgcggcatgaaccaggaggacggcatcatcctgaagcggcagttcgcggagcgcggcgggctcgacatcctcacctgcaagaagtaccaggtcgagatcccgctcgagaacttcaacaaccgcgagcgcgtgcgctccgcggcgtactccaagatcgacgtcaacggcgtggtgcgcctgaacgccttcctcgagcagggcgacgccatcgcgcggaacgtgtcctcgcgcacgctcgacgacgacttcgtcgccgacaaccagatcagcttcgacatcgcggagcggtactcggacatctacgccgcgcgcgtggagcgcgtgcaggccgacctcaccgacaaggtcaaggtgcgcgcgctgaccgtgcgcgagcgccgcgccatcctcggggacaagttcaccacgcgcaccagccagaagggcacggtcgcgtacgtggccgacgagactgagctgccctacgacgagaacgggatcgcgccggacgtgatcatcaactcgacctccatcttctcgcggaagacgctctccatgctcatggaggtcatcctcaccacggcctacgggcacaagcccttcgccgaggacggctccaaccgcccgatctgcttccccagcaccaacgagaccgacttcgagacctacatcgagttcgcgcggcgctgctacgcgctctcgcaccccgaggccgccgcggacgaccccgagttcgagcaccgcgtcttctgcgagcgcgtgctcttcgaccccgagaccgacgagcccttcgcggcgcgcgtcttcttcgggccgctgtactacctgcgtctgcggcacctcacgctggacaaggccacggtgcgctgccgcgggcgcaagaccaagctcatccggcaggccaacgagggccgccgccgcggcggcggcatcaagatcggcgagatggagcgcgactgcatgatctcgcacggcgcggccttcaccgtcgccgagatcctgcgcgactccgaggaggacgcgcaggaggtgctcgtctgcgagaactgcggcgacatcgcggcgcggctcaacggcacgcacgtctgcatccgctgctccaagatgagcctctcgccggtgctcacgcgcatggactccacgcacgtgagcaaggtcttcaccacgcagatgaacgcgcgcggcataaagatccgcgtggagttcgagaagcaggacccctgcttctacgggactccgaaacggttcagcctcgcgcccgacgagtcgctgttctcgccggaggactgaacccgccgtcgcgaccgcgtcgcgacgactagcttatcgttcgactgatgcgaaacgcgcggcggcgccgcgacttagcttatctcgactgatgcgaacgcgcgacctctcgcgactttctagcttctcagactgatgctaccatatcgcggcgtgctggccccaccaccagggcttctcgccgtggctgacgcggggctggctgcgacgcgcgctgcagtagctgcgcgcgccccagtcgccgcgcacgtgcgccgggggcaggctcccgtccagcgcgtgccgcgtcacctcggcgccgggccggcggcacgtgtgcacgtccgtcttgttggagacgagcaccgcgtactgccgcatggtctctatgtgatgctccaagtgcttgcccgccatccggttggactcgcagcacgtttttgcttcggctaaggttttttctagaggggatagtagcttatccacgcgctcgggcaggacgcacgcggagccgtcgaaccctactttgaacggggtcaccttgatgttcccgtcgtagcggtcccacagcatcctgaggtaggttgtaccgtcggggtctgggtctgtccacactctaagcttttcgctacagcggccgtcgtacgtaagacggtctctacgctcgtagtagtttctgcttatgttgttggggtctccatgctcgtagtagtataaatcgtacgcgcctggcttttttaagtcgttttcgtcgttgctgacgtgtatcacgtcgggataataggatatcctaactgcactacaatctatagtatttggtctagtaagctgttcgagatcaccttgttcatcatgatctactgatttgtacacggcaccgtcgtgttccgacggacgtatgaatatgtccatggtaaacgatgtacccactttggaaaacgtatcccatgcagtaaagcatagtccgtccattataaactcaggaacactcataacaaatcgaaatctgtgaagtttttcgaacaccacttttacatggtctttgtcacgaacatcattgccgtttacttcagacatgaattgaaggaacgctaaagagtttcttgtttcttcatgaatctttccattatacgtccatccagtttctagaattctatatatgctttttgcatcgaccccgtaccaccagtacatgggaactccgaaatatatagctgggtttgagtaccaatgggcaagagtgcccattgcgtttaaaaagtcttgacaaaaaaatgcagtttttctgtcgataacttgacttggactacgttcgtggacatcgtacatgtccataattggttcattggtaacggttacatgacccgtcattatctttttaacaatcataagatacagtttgcctaaagtcgaaatatgtataacgttaatttttacatgttctcctaacgtaattgcgtttttacttagccattcgtcgtctacaaaaatcttacgatacataggatttctctctacgtatcttctaaagtatagatttaccggtctaccggcgacattagcgccatctatagcaggagcaagctgtatgtatcgtcgtataatgtctcgtataagctttctgtcttctctgggaatacacgacacggaacttagagactggtgccagtgtctttcaaccaaagacttgaacctagcaaccaacgcgttgtcactctccatttataattaaataattatcccaacttcgtatgttaatccttattaccagatagcaccgctccttcctctccaccacgtactatctaaaggatacctgtaagggtaatgtctggataacgggcgtgtgagccaagacgtgttatggtgtcttccccaccaacggtccacttctctaactaccggagtgctagacgttgtcgatcccactactgttgtttctccattacctgtaatctttgaagcgcaacaagtgttagtctttgccaaatcttctaacggcttaattaggtcgttaagtctgtcaatatccatgcactgaggtgtatcgttaccagtttccccaactttgggaggaacttcctgtttagtgtccaaataacccataaacctgtctctaagcattcttctgtacgtgcttttgtcattgtctatgtctcccaaaaacctgtgatttacgcatccgttgtacgtaagtctgtcccttcgctctcgctcgttatttcctacttcttctattgtactgtaatgttgatagtccaagtaatagccactgttttcatgatttcttgtaaatataatcggtgttttattattgacatcgtgttgcctactgtacgtatcttccatggatctaggaacttgtctagaaaattgaggactagaaatacgccttccaaatcctggatgataaaccaaacgcaatgcactacagtcgacatcgtcactgtctctagttatcccatcaacaactccttctttgtgatgctccactgttttaaagttaactcctctgtatatgttgagtgttaaaaaaatatccacattgaacgcagttccatattttgttattttatcccacgacgtataacacaaaccatccataatgaattccggaaccgatactacaaaattagtatcgaacttatcaaacgaaatgtttactcgcggttgtgcttcgtattttttgtcatacacatctgtcatgtattgtacaaaatctatagctcctctagcatcgggcatgctaacgtctggaaaatcattttttaactgttctagtacgaactgttgattttggtcatctctccaccagtacattggcaatcctatcacaagagacttatttttataataatttgctacagaagctatatgccacatgaaattatagcaaaaataatccatctgtatgtttaaaaccggtttactagtctgctgagagtagctatccatgatagtgtttccctcgccaatacttcctgacattattctgtaaacaaccattaacaaaaatcttcctaccgttgttatgttgtcaaaagttcttatgttttgagcactttcgagtaaccatatgttattttcaaatatacttttgtaaactggatttctgtccacataactctttaaaaatagatttactggaaggccgcttgggttatctccctgtataggcggcgcttgctttatgtattcgcgtaacaaatctcttacaacttttctagttcttctaggtatacatgacctattattcaaaggctttgtccagttagcgtttataaacgttgtaaagtcactgactagcttctccatttataattaaataattacagacggcaacacagcggttatctaatatctgctgtatcctgtctgtacatctatttttctgttgagatcaagaagagctttacgtagactctccaagtgtctttctagtctgtctaaccggttacctgtttctctgcagcaatcagttatagttttgtaactgtctaacaagcttacgaggcgctcttccacactttctttagttggagctccagccgcgtacactccgttggttgaattgcctgtatcatcaggctgagtcaataggttttctccgtcattttcatccatattgagtccaacgaacacaaacgagtaagtgttcctctatttaaagtattgattttagaaaaaggcaagcctcgctgccctgattcggcggcaaacacgggttgaacacgcggaagtcgctcgcggccgtgaagatctcgtccgcgcacgcctccacgctcgcgaagcgcccaggcgagacgccgtcgtgcgagcggaacccgaactccgaggccgccaccgccgcgcccttgaagagcacgcagcgccacttcttgcgcacgtcgaaggcctcgtcgttggggtcgaacacgcgccggtccacgcgcgggccgcccgccgtgcgcgcgaactccagcgccgcgttcgccgcgttgaactcgcggatgttgtcgtagttctcgtagacggcccagagctgcagcgccacgaacatggcggccgccgccgcgagggccacgcagagcgcggacaccgcgtccatcttttatgtgcagaattattcgttggcgcggagctcgcgcagctccgcggcgcgcagccgcgcgaaggctgctttgagcgcgcgcagcagctcctcggtgtccgcgcgcagcatgtcgaagcggtggtagctgtccaggcgcgcgcggcagccgaagaagcgtgcgacgcacgcggtgacgatgtcgttcacgtagagcacgcccgaggccgtgcagtacacggagcgcggctcgcgcgggtccggcggcacgtccacggcgaccgcgtgcgcggccacgtcctcgagcaccttgcgctcgagcacggcgaggaagtcgcgcagctggcggcggttgtccagccaggcgtaggtggtcgcgaagagcgtgagccgcccgcgcggcgcgatcgcggtgtagggcgcgtacccgcggaactcccgggggtgcacgaccttgacgttctcgtgctcgcgacggaaggcctcggtgtcgagcagcgccgcgagcgcgtccacgagcttgtcggagacctccacgcccgcgccgaaggcgatgagctcgatcttctgctcgctcttggggcggaagtcgtggaaggtgtgcagcagcatctcgcggagctgcggcggcttctcgacggcctcgagcgcgtcgccgcggacgaggaagtagtcgaggtcgtgcagcgagacgtgctgcccggcggcgctctgcacaaacttgaggaagacgcagaggcccgcgcggcgctcgagcacgtcctcgacgtgcgcgtggaatacgtgccgcgagggcatggcctcgatcgcggagagccactcctcgttgacgcaggtggtggtgttctccagcaccacgccctgcgtgagcgcgggccactgcaggtggaaggcgaactcgtgcttgatgagcgaggccacggccgggtccaggtccacggccagcgcggcctcgccgacgaggggagcgtccgccatcacgcggaggacgcctggcccatctcctttttcgcctttttattcaggatcattattctttcgttgacgaggtccatgagcatcttgatggcggcggccgcggccgccgcgtcgccgccgcacatctgcgcgatgcgcgtgagcatgtgcagcagcgcggcctcgtttaggtcctcctccatttagaggccgtgagggcgcgcgtcgtcgcgacgaggggacgcctcccgcggcagcgtggtgcgcacggcgaaggcgagcagcgcgccggcgcactgcgtgagcacacactccgcgagcgcgacgaggagctcggagggcgcgagcaccatttagaggcgcgcgcgggtttaattgccgccgtcagagtcggcatcatcgcccttgtcgccgccgtccttgcagtcgcccttggtctcggcgtcgacgatgtcggcgagccgcgtcttcatgtgcgagaactgcgcgagcaggatgccggggtcgagacagcgcttgacgacgctctcgtcggcgaagtcgtagcagatgcgctcctggttctggcagaacaccgagtcttcgatgatcaacaccctcctggtcccggccgaccgcatgatggccatggcccggatgagcctcttcttcgatccgcgtatggacatggaccggagcacgttctccacgtcggagtcggagacgttgcagcagcagaggtgcgtgatgctggcgcgcccgttgacggggatgtgcttgtaggtctggcagagcagcaccagcgacacgttgatgtgccgcccgtagttcatgaggcccaagagtgtgggcgaccgcgtctgcgtgtcgcccatatcgtcgagaatgatgaggaacttctgcttcttcgtctgcgcgtgccgctcgatcttgcgcttggcgaccgagaggttgtactcgagctcctcgtgcgtggtgaccttgtggatgtggtccggccacacgaagccgtcgtaggcggcgttgtagacgggcgtgaagagcaggatgtgcttgaagcggcgcacgagcgtgcggaagagcgagagcaggtaggcggtcttgccggagccggagccgccgacgagcgccatcctgaagggcgcctcgatgagactctcccgcttgaagcgcacctcctgcacgacatccatcgtatatttactgtcactaaattaccggctccgagaaatatagaaattagagcctcctagagcacaccgaggcgcatcggcaagatggcacataacacgttcgaaaacgatagcgagtcggcagctaacaaccagtacgtggcgtcagtcaagcgccagaaaatgattcggcgatacattaagatgttcttccggttcgttacggcgatagctatcattgtcctggctattctagttgtgattctgtcgctatctctagacgaatgtctgcacagagaacaccctcatgactattcacatgtacaaaattcaacatgcgacggcattactttaggtggtgaaaagtgtctcagacttaatttgccagcaacgtgggaagatgctaatagacaatgtggtaatcttgggttttacctaccatctactggccttgaaaagaaatttccttggcttgtgacctatctcgacggaacttggggaaacactcagaactccgtatttggaccaaccggtgacttgcagaatgtcataggaccgaaagaatacaaatatttttgtgtgtccgattagatgattataatctaataaatgggttgctgtaaggtccctaaccgccagtctataaggactttgaaaaaggcgtcctgtccggtcgccagtctcgtcaccattctctccctagtcaccagcctcggtgcgatagtcagatacaccaatttttttctaaaagaagcatgtgacgaaggatggatgcccataaaaaacatatgcattttaaacacgcactttgaagccaccaatgacgatgcccacaggatatgtgaaaacctagacggaaagccgccggccatccctaaccctactctgttaaagggtgtcacagttctcaccggcgaaaagaaattttggatgacccatcacgaagactatactactgtgtttgagcatatagacgataggacgactcctaaaaacacagactatgacagtaaaaaacacacttgtttgatgagcgaggacggattgatacaccataactgcatgatgaacgtgactgtggtatgcatgaaggagatgcacggataactgaaaatatactgtttgaacgcaaagacgccatgtcgcgacttcaaatactgacctcatttggacaaatcttcgcacccgacgaagctcggctgcgcgagatcgcgcgtgatttgggaatatgcaccataaaacgcgcattcggcgacatgctgtacggctttatagacttcgacccggtgcccctgacccaagtaaacatgctcatgtccaactgctacttcgcggtcaacggcaacctgcttccgtgcacggaggacttccggctcagactcccggcaacggagatctctgcggcctacctgacgagaacgggacggacgatcctgtgcggcaaagacttcaacatagtagcgccgtcggggttcaagccgtccatgcggctgcgcgacctcagtcacgtgtctgcgcttgtagagatcctggaagtctacgacgagtccggggagtaccaattcgtgctcggccccagcgcgcagttcatgctgcggctgatggagaaggagaacgtctgtctgttcggcagcgggtggtgcatagtggacctgcgcaagctggacgtacccatataatcagcatccttgtttttatcctgtctttttatcagttttttagctagttaaaacataaatagtaaagctaaaaagaggagttctggagtcttgcaacaaccaggatgaaggcggtgttgttgctggcgttactgggagcgttcaccaacgcagcgcctttgttagaaagccagcgttctaacagtgaggaaaaagcaaatttctgctcgacgcataatgatgaagtgtacgccaggttcaggcttcagatgcgcgtgggtgtacgacacagtccgctctacactcccagcaacatgtgcatgctggacatagaagactccgttgaggacatagaagagtccacagaaaaagaatacgcgtctacggccacgggtgaggcggccggagtgaacgtgtcagtggcactagtgggagaaggcgtgagcataccgtttagttacataggccttggattcaacccgtcgcttgaagatagctacctgtacgtcaacgtctcgtcacgagctccttgggttaaacagacttcggacctatccgcgaacggcggctggggtatcaaacaggttctagaaaaagagttactggccatccaaatagggtgcgacaaccaaaaatttcccgaagaacccacaactacacccccctcacctgtcactacaacgctttcctcaacaactccagatctgaatgaagaaaacacagaaaatacgccgacgaccaccggcgccagtgtagacagaaagcgcaatccagctgacattgacttctcgctgctcgtggacccccgatgcgtgacctctgtagacctgcacgtcgagctcagggacgcgtgcatagactacaaacaagagtcgccgttgtcgctgaaggggaaatatggagacggcgaactagtaaaaaaggagattaaagacgtgggaaagaatcacaatatgtgcagtcttaacctcaaccctggcaattgagctgtttttattcggcaatataataggtgattattgaacattaaacaaaacttatcccacaacgccgcaacaatggaagtgctggtgatcatctctattatcgtcgccgtaatatgcttgaccggagcggtaatgtacctccttattgaactcggcttagccgccgagcgcgctaacaaacgcgcgcgcgtgaagaaaaatatgcgcaaattagccactcaattgggaaatggatctgtcgactccggcataggcataggcccgtgcataatgtcgcgcaccatggactctggacccagtcgctgggacagcgacagtgaggatgacggggacagcctgtccacgacgtccaccagcggaggggggactctcacccgagtgtgggttgggagcgccgggcctatgtacgaaaacttctgcgggaacggcacccgccactcccccaccaacgaccctggctaccactcgcgggagactctctgcagcggacctccccgtcaggcgccggcgctaccgcccaccccgaagcccgacgaggtaacggtggacgtggggcccggtcccgacgaccaacacggtccgtacgaggaacctgatcccattcccccgcaggaacccgagccgccggtgcagattgaggtaaccatcaacgggcccggtggagaaggcgaggcggaaggagaatttttctacgacgagtagccgccaaaactgaataactatcgggcttcgtaaacgcgcagacatgccgctgttccggaagctcatggtttcgcgctccctggtcaaggaatgtctgactctggacttccggcagggcgagcgtctccctacgcgatgcttcctcccggtgcccgcggggacgacattccacagagtctgcgacacctcgccgctgacggacgaagtctcccggcacgtgcaggagcccgtcatgggcaccggacgggtccagtactactacttcgagagcggccagggcatgatcggcgacaacgcgggcatggcgcgcatgctcgtgtgcacgcggtcggcgtacaacggcggcgacgtcgtcgtgcggtccacgcggagcagagcagacaagaccgtggtcgcgccctgccagggcatggcgctgctgctgagccccttctgcgccttcgacatcacgccggtcgagagcggctccgcgatattcgcggaggtcatcgtcaccgcgcccagcatggactacgtcgaggcggtcaccggcacgggcgaggcggccgtgcggatattcaactcgcaccacccgctctggccgcgacacggctcgaacgtctgcttcgcgctgcggttgctgcgagacgtgcgcacgggcgagcgcgtggtcgagcagatgttcatggacgggcgctggcacaccgtgctgaggacgtcctgcggcaacaaggtctgcgtgcccgccgacctcgtgggccagacgaacctcgaggaggtgcccttctgcgacgtgacgcccgagatcatgcgccgcgcgctggcgatcgacccgccgtacgaggccgtggcgcacccgcaccgctgcgtgtacggcgccatggacgtccggtgcgcgaacgagtacctcgtgtactgcaccttcaagacggagccgacacggcgcagcacgtcctcgccgggcccggacagcccgctgtcgcccgcgactccgtcgacctcgcgggccgcggccgcgcgcgtccccacgacgccgcaggaagtggcctcgccgaccacgaggctcctggagacctgtctgcgcgacgccctcgacggactctgacccgaaggacccaccgtccactcacattccactgccagacaactcaagctttttctgcatctacctcgctaataattgaattgttataggacaaacaggcgcactcgagcacaatggcgtgttttatcgaattgttagactccatcttcaaccgacgccaccgtaatttcgggccggaggacatgtacaggccctctgacgccccgccccccaaatcgcacacgcctcgcactccccgcaccccgcggacccagtgtcccggacacccgcggcgacaaagctcctctcccatctacggcgcttatgtggactccctgccgaggaacagaaagcggttccagaatcaacacagttgtcccggagattacgagcggtgtcaactctcggacaccatcagcctggatgcgacgctactcacggttacagtgacgtccatctccagcatatccagctctagtagctcagactctatctctctggggcagtgcagactgtccatggtgtccgcaacatcaacctccacctccacaaccttctcctcctcggaatgagcgccacacttatttttgtataatagtttgtattgaaccttagagacatccacaaatagttaggaagcatgagtagttcaagtagcgagaccacccctaagcccaagcccatccctgctcctcccatgactcaggaggagtttaacaaagaagtgaagaaacgaaaagaacagaaaaaggaaaaatctagaaccgttgaacgtgagtcagaaaccgtaactgtatcttccgacggatcagagataaaaaagacttacgagcgcgagtctgagagaacaaccgaaacagaaaagaacaacacgtcaaccgatgataataagcagaacacccctgtagagaaaccagaggaaaccaagcctgcttctactcctgaaggtgtgaagccagccgagactcctgccccgactactgacccccaacctactacacaaccacccgcagaatcaaaccctggaagtcaacccgcacctgcttcagaaccaacccccgcacctgagcctgcaccggaacccactcagcctgcatcagtaactcaacccgctccaacaccagagccaagtccagcccctaagcctactccggcttctgaaccaaccccagcatctgagcctacttctgctccagaacctacaccatccgcagaaccaactcctcaaccaactgtagaaacaccaccatctgctccagcaccaactcccgaggcccaaccacccgccaacagcaatcccactactgaaactaccactggtaccagcacctcctaagtgagtacgtaagcatttcggagtaacgtcgtagcaagcgctagtccgccgcgagcggttctcgcaagttttttcgggtaaaaagcgtacaccgtcgccttgtcgcggcggtgtacgcttttttcacgccctttttgcaaaatttaaattgtacccgcgccggctctaggaaagatggcgtgcctcagagtgttcctggcggtgctcgcgctgtgcgggagcgtgcactcggcgcaatggatcggcgagcgcgacttctgcacggcccacgcgcaggacgtcttcgcgcggctgcaggtgtggatgcgcattgaccggaacgtgaccgccgcggacaacagctcggcctgcgcgctggcgatagagacgccgccgagcaacttcgacgcggacgtctacgtcgccgcggccggcataaacgtcagcgtgtccgcgatcaactgcggcttcttcaacatgcgccaggtagagacgacgtacaacacggcacgccggcagatgtacgtgtacatggactcctgggacccttgggtgatcgacgacccccagccgctcttcagccaggagtacgaaaacgaaacgcttccgtacctgctggaggttctggagctagcgaggctgtacattcgcgtgggctgcacggtgcccggagagcagcccttcgaggtgatcccggggatcgactacccgcacaccggcatggagtttctccagcacgttctacggccgaaccgccggttcgctccggcgaagctgcacatggacctcgaggtggaccaccggtgcgtgagcgccgtccacgtgaaggcgttcctgcaggacgcctgtagcgcccgcaaggcgcggacgccactctacttcgcggggcatggctgcaaccatccagatcgccggccaaaaaacccagtaccgcgccctcagcacgtatcgtcgccgatctccaggaagtgcagcatgcagacggcgcgctaagggcgctcaccgcgctgacggcggccgtggtgtgcgcgatcgccatcgcgctcgagcgcggggcggaggccgacgccgtggaccttatccttataaaattttcaatgatatgctagtttttatgcgaccttccttagaaaattcggaattcaaaaatgaaataaaacggcgtttagcacgcatattattaataccgaccaccatggcaggcgtccgcagctgccagaagaaagtcccttctactgcgggctccatgtcatttcaacggggcaaccggagcatccagccggcgatgtccgaggcgttgcagaatgatttcagctacaacccgcgaccgcctccgccgagcgcagaagagattgacttcttctgcgtggacatgcgcaaagtactgatggaaattgaggccaagcccagcagctccaagtaccccgatttcatccacccggttgacagcagcccgccgtgcacgccggcgcgcaagcgcaacggcttcggccgcaaggcactgaacaaaacccaggtgccgcagcaggccaagcgtgacggctactcccgctaatgcagtccacacacttcacacactacatcagcactcaagcttataatcactacacaatgaatcagcccaccacgtgcgaagcacacacataaaatcacccacctgtcctgatcgttcccaatactcccaatcaccgtgctttacacgcacgttaatcaccctttccttccttcatgcgttcctgatcgttcctcctccttaatcacacacacaccccgtaattttgtacttttgtactttaatttgtacactttacacactgactttgtactgcctttgtactttatttttgtactgaaattggacgatacttatctttgtattcacatccaagttttgcaaattccacagccggtagcgaaaagtgaaatcgtaccgttttaggcttcgatccccctcccgcgcgaagactcgccagcatggactctcgtaggctcgctcttgccgtcgccttcggaggcgtcctcgccagcatgacacagcgccgccgcctggcttctctcatcgccagcatcggccaacggctgatgggcggcgacggcatgcgtcgcgtcgccgttcggttgatcgaccagctcatggccggacccccggacatcaacgacgaggccttccagcgcgagatccgcgtgggcgagctcttccaggcgctccaccgcgtggtcgagcaggcacgccgagagaagtacttcgaggtctgcggcgccggcaacgacgccgacgcgcccgtcgtcgagatggacaccgcggccgcacccccgcagccccagcccgcgcccctcgtggtcacgccgcagaacgcgttcatgttcgtgccgcaaggcagccacgtgcacgtggacgagagcgtggacccgttcttcggcatgagcccctccatcttcgggcgcgacctcccccctcagccgcccgaggagctgctgagcgactacgacccgctcatgagccaggccggcgagccgccgagcccgcggtcgccctgcgaggccgacctctggtgcttcgagacgctcggcgacagcgacagcgattgagaccgcaccacaccctacctcacccaccccacactccacctcacctcaccctaacacccgacacccaacacttcaaccggacaatgaaggagtcccacatttcactgaaggacgcggatgaagccgcactcccccacatgaaggattggcaacggtcaaacatttcacctgcaatgaaggacgatgcgcggtcgcattggcctgcgaccgacatcgcacacatgaaggacacaattggtttgttaatccggacaatgaaggacaaattgtttttgttaatcaggacaattagaacacaatcaaatttttgtacgatcataaaatcgatatttgatgcacatatattagtaagtatattagactaaattctccggggaggcaagcagttggatacggcggggcggggcacgacgtgcacggagagttcgggcgggtcccccttccccccacccccacggcaccacgatgcgcctaatcttagcgctcgtggcctgcttgttggcggcgccgatgccgttatcgggtcgttcgacaagcactccaaacacgtccccctccgcactcggctcgacgagttcggaaccaagctcggaagacgctgtggcttcgagcacaacgacaagcacactcacaagcactacaagcacactcactatgtccacaagtgtggacaccactactacctcgggcgctacaacgtccgcaaacagcactcctgcagcgagtgtgagctcctccacacccgcaactaccgaggcatcgacggcaccaacgacgccgtcgacgccgacgacagtgaaggtaacgaagggcaaggaagacacgaaggcgtctgcctacctcgttttactaatcacgttcatggtcatgaccacgctcgtgatggtcgtggtcgtggtcgtggtcgtgtacaaacagggactctgtaactgctgctgtaagtttcccctgctgcaaagagctcaaggactacctcgacgaggaggagagcgccgggctgtacgacgccttgacgtggagccactcagactccggcctccggctcgtcgtgcgcgcggaccccagatgatgaggatcggataagatcggcgtgtttttcccgcccgtcgcgaacattatgcctctaaatgccgagaattaactgaaattcaaacacgctttgggactcaactccgtgacccacactcaaccatggctggcttcctaggcgcattcagaggcgtgtgctccgacttatggcagtcgctccgtggacacggacaccactcttccagctgcccgcgacgacgcgccaacagcatggacgaccgcgaccggcgccgacaccgccaccgcgagatccccaacagctcggcgtcgctgaacagcgacccgatgccgcaacgcagtgcgggtgcgcgccggcactacgactgccgcccctcggaaaagagcagacactcctccgacaagcaccactcggcggaccgacaccactcggcggaccgacaccaatcggcggacagggacagacaccgtcgcagtcgcaagaactacgactcgcacccgtcgcgcaggaaccgcaactacgagcgggcggactaccagagacacccctcacaaacccacccagacgcccccgcgcagacctcgacgctcaaggtgacctccctaagcaccagctgcagcaccctgtcccaacatcactacgagacccccgaccacatctacgacatcccggaagacagtcgcggggcgtcggctccccctcgcgcggacctcgcgctccccccgctcgccatgcccaaatccaagccgcgtcgcacgcgcccggcgtccatgaacgactgcctgatgaagcactgcggcgccggcagacccaacctccaagacgacatatgcacactatgtactgatatagagacacagctgagcgcactagagaagtctctggagtcagagctcaacttctatcgtcgctacatacaagacactaagacattgctcgccacgcgagcagcaaacatcggcagcaaagctctgatctacaccgacgactacaacggcagtggcgacgtcggcgaaaaggagcactgctcggaggagtgctgcaaagtggaggaagttctgtgagaaagtgcgtttttctgtaatgtgaaataagatagccttatgtgtgcacagacatggcgaacaggctcgtgtttttcgaccccgagaccctagccgaggccgacggcatccccggctatggggtgttcgagcccggcaagaagaaatgcatcttcacaaagatccgcaccagcgtcgcactcgcgtgccggtacgccgtctcggacggcggcctcatcgacgagttcgtcatggctacatacgggaccagacgcgcgtgccggctcgtccggcacctgacgataggcgcggagggcgtgatgacccggcccgccagcaactgcgcgccgcacatggtgctcatctgcctcagaggcgtggccgccgtgtccagcgaggacatgggcttcggtcgctgcatcatggagcgcggcaccatgttcatggtcaagtccgcgcacagcgccgtcgtctgcggcaaccccgcctgcgagctgctcgtcctcttctacgactacttcacccccatcccccggccgctctccggagacgaggtgctgttcacccgcgacctcgcgcacgtggactacgcccccgagtcggcggtcgtcttcaagatggattacaacctcgagaccgacgtggccacgctgtttgtcggggggtacatattccgcgccaagggcctgatgatggagacgcgcgaacaagtgggcgacgagtgcgactgctgccgccacagctcgccggtgctcgtcatggatcgcgagaagatgatgtcgtcgctgcgcatgatccccagcatcgtgcccggccagcgggagatttggcttcgcgagcgcggctgggccgtcctcgagacggacgcccgcggacactgcgagcccggcgtcctgaggctggcgctcgccggcctgcggctgttcgcaggatgcctgcgctccgtcgtggggcggcgcgagctgtcgctgttctgctacggcgtcgctcccaagttcggcggggagttcgaggacgcgccgcgccccatggagatcgacggttagttgtttttatccctgtacatacgccgcaaactgaaactttagggcaccgcgtaatagtgcacgaacgcccagtggaccgcttccgcagccatggaaaacaacgacggcaacgaacgcaacaacgaacacccgcacgttcgagaattcaaggaggcgtccctgtacgggtttctggtggcggccgcggacgtgaccgtcgaagacgtgcaccggtaccttcagttcggcgcggacgtgaactacaggggcgcgtacctgtgcacgccgctgcacgcgtacctgcagtccggctgcgaaaagcgcctagacgtcgtggacgcgctgctggacgccggcgcagacatcaacgccaaggagatctgcggtctcacgcccgtgcacctgtacgcgagctacgcggacgtggacgtagagttcatgcgcgggctcatcgagcgcggcgcgagcgtgtgcggcgagagctcggtcacgggctgcctgtactcgtacctgtacacacacagcgtggacggcggcgcgcgcctggacgtggtcgagctgctcgtgcaggcgggcgcggacgtgaacgtccgcggcgaggcgcgcaagacgccgctgcacgtgcactgcgcgggcttcgaggtggattcggacatcgtggatctgctgctgcgcgcgggcgcggaccccgaggcgctcgacgaacacgggctcacgcccgcggacgtgctcgtgaagtccgtgggcgccaacgtggagacgctgcggctcttcctcgacgcgggcgtgagcgtggccacgtcgcgcgacgcgcgcggacgcacgccgctgcaccaccacgcagactccttccgggcgagtgcgggcatcgtgcgcgaactgctcgccgccggctgcgacgcggcggccgccgacgacctcggaaacacgcccctgcacagcctcgccaccttctgctcgtgccggcgctcggtgctcgaccagctcatcgccggcggcgcggacatcaacgcccgcaaccactacggccacacctgtctgtactacgcgtccatctacaacccctccgtctgctcgcggctcatcgccgcgggtgcggacgtgaccgcgcgcacgccggacggacgcacgccgctctcgggcatgatcatgcgcaagcacacgcgcgccgtgcgcgccgccctggcgacgcggcctcccgcggacgccgtcgccgcgtcgctggacgtcgcagtacagcccgagcccacggacgccactcgcgcgtgcgtgcggtacgtggtgctctgcggcggcacgctctcggcgcgcgtgcggtcgcgacacgcggacttcgtgcgagagtgcgaaagcgaggtggtcgtgctcagaaccaccgtggtggggctgcccggcacctcgctgctggacatcgtgcgtgcggcgcagccgccgccggtactgctctccccgcgcgtgcaccacgtgctgcagaagctgtgcgtgtacgcggagttggtggacgcgcggctgcgcgagatgcggcacaagaccaacctcgtggacgcggtgtcgcggctcgtgtgtccgtgcgcgctgccgccggaggtggtgcgcggcatcctcgtgcacgtgccgatagacagcctgcggcacacgttgaccctcggcgtggcgcaggcctcgcgtttccttccctcgcataaatgaaatattattttttgtggtagaccggatctccccgatggaccccgccggacaacgactgcgcgcgccagggccgtggcgcctgaacccgccgaccgcggccgcgctggaaagcgcgctgctgcggcccgcggcgtcggcgggcgccgaccgctgcgcgaacgcgcacgtggaccgccgcaacatgggcgtcggcgagggccgcgaggtgcccgcggacgtcgaggggctcatgaccgagatccacctgcggtacggaatgacgcgcgtccaccggaacgttcacttcgtgcagttctggcacggcgagcacgtgcgccggcgccccgcgcgacacgtgttcacggtctggatctgcctcagcggcgaggtgcgcatctacgcagagtgctgccaggcggggcacggcttcgtgctctgccgacagatggcggcagggtacatgttcgtgaccgaacccacggactcggtcacggtctcggtgccgcaccgactgcgcaactcgcggtcgccggtgtggctggcggcggtcttcgccacgcggcacttcgagccgctgccgccgcccatgtacgccgtgcccgggcacgtggtgctcgcgcgcagcgcctccatgctctgcgactgctggccgtcggacccgcggcgccgcaacgtgatcttctacatgcggctgtcgggcgcgatggtgcgcgtggtcgtgccgggcgcggagctcgagatcgagtgcacctcggggttccggccggaccacttctccatcaacgacgagtgcgtgtgctgcgagcgtccgcacgtcgcgcgaaccgcggtgtggacgctggcggagatttgccgcggcgccacggtggtgctcgcgccgccactgccccgcgaccgcgccgcggggctgctcgcggagatccgcctggcctcgctgcgatgggtgcgcgtgcgtgcggtccgcagcggcagagaaagcgtgggcccgttcccctcggtggtgtgggcggcggtcttctccgccgttcggctcttcctggacggaaccgtgcctgccttcccggcgtgtgtggagaatggacgcgcggcgtacggcatggtgtacgtgcccccggaggagccgcggatggacgggctctgtgtgttcccgacgcccgccgagccggcggcgctcttcgtccgcggagaccaggtgcttgaggccggcgcggccgccgccataatcgcggccgctgagaagcgcgtccaggccgccaatgggtctcctgctgccgcggaggaggacataggtgcggcggccgatgccgccgcagagagcgtggagcaggaccagcgcgtcgagttcgaccttgggcctgggcctgaccccagccaagaagcgcccgcggacgcgcagcgtgccgattcggacgacgacaccggctccgagaccgagaccggcgacgagagtgtgggcggcgaggatgacagcgactcctcctcctcttactcggtgatgtcggacgacgaaaacgacagcggcgacgagggctggggcgactctagcgactccggcatcgaggacgacgacggcggtgtcgccaggccgccgaggaagaagaggaggaagagcgcgacgtcctcggcgcagcggcccagatgctcggagactgaccggtggtgaaaacataaaaataaactgttcaacacttgtactccgggcaccaacactactatccatacccaccctccctccacacactacaatggcaaacagagaagagattgacgcctccgccgtcatggctgcctacctcgcgagagagtacgcggcggctgtagaagaacagctgacgccgcgcgagcgcgatgcgctcgaagcccttcgcgtttccggcgaggaggtccggtcgccgctgctgcaagaactctcgaacgcgggcgagcaccgcgccaaccccgaaaactcgcacatccccgccgccctcgtctccgcgcttctcgaagcccccacctcccccggccgcatggtcactgcgattgagctctgcgcgcagatgggccgggtatggacgcgcggccgccagctcgtcgaattcatgcgggtcgtgtacgtgctcctagaccgtctgccgcccacggccgacgaggacctcagcacctggctgcaggccgtcgcgcgcgtgcacggcacgcggcgccgcctgcaccgcgttctcggcgtcggggccgtcatggcaggcgtcggtatgctgctgctcggcgtgcgcgtgttgcggcgcacataactttttatctcggctcaaactgaaatacgacattggactacgaaacctatgattttgctcacggccgcgcgagataggataataaataacctttgagcaactaacatggccgatgagagagaggccgacggcgcgctgttccggtacctggagagcgaggaccgtccggacgtggagcacatgcgccggctgctggacgagggtgcggacgtgaactacgcgggcccgcgcgggtacgcgccgctgcacatgctcatgcgcggcaacccgctagaccccgacgcggtgcgactgctgctcgccgcgggcgcggacgtgaacgcgacatcgctctgcgggttcacgccgctgcactcctacatgtgcttcgggaccgtgacgccagacacgctgcgtgcgctcatgcgccacggcgcgagcgtcagcgacctcgagcgcaacatcaacgcgctgatcgagtacttcaaccgcgacggctgcatgggcggcgcggaggcgaccgtgatcgcactgctggtggagcacggcgcgcacgtgaacgccaaagacgaccttggacgaacgccgctgcacatctacctgtccggcttcttcgtgtcggcaccggtggcgctcgcgctgatcgcgctcggcgcgaacccgaacgccacggacgcgtacgggcgcacgccactgcacgccttcctgcgctcccgcgacgtggaccccgctgtgctgaagacgctcatcgccgcgggcgcagacccgctcgcgcgcgacatcatccggcgcacggcgctgcactaccactgcgagtccttcaagacgcgcgctagtgttatagagacgctggtggccgccggctgcgaccccgcgagcacagacctgctcgacaacacggcgctgcacagcatggccatgggcagctcctgccgcgcctcgctgatccgcccgctgctggccgcgggcgtgtccgtgaacgcgcgcaacgcgcggctgcagacgccgctgcacctcgcggccgtgttcaacccgccggcctgcgcgcggctgctggccgcgggcgcggaccccgcgctcgcggacctggacgagacaacgccgctgctgagcatggtgcggcacaactgcgcacgcgcgctgcgcacggcgctgcccttggcgccggacgcgctggtggccggcgcggtgaaccgcgtgaacgcgcgcacgccgagcgcggccacgcgagagtgcgtgatggcgctggcgctgcgcggcgcgctggacctgctgagcgcggagagcgttgccacccacgcggccgcgatccgcgcctgcgaggcggaggtcgcgctgctgcggagcacgcgcctgggcgcgccgccgacgacgctcttcgcgctgctgacaggacgaccgaacacgctggtttccgcgaaggcggcgcgacgcgcgatggcggacgtgtgtgtctaccgcgcggcgctggccgcgcgcgtggagcgcgtgcgccgcaagtcctcgctggtcgagcgcctcaccgccatggtgtgtccgtgcgctctgccgccagagctagtgacgcgcatcctcgcgctcctgaccgtggaggaactcgcttgcgcaatgcgcaaataataatgaactataactaggcttattagaggcactatttgtgcagagtcgttagttatagttagtgtacttaccattggaatgtcgaagaacaaaattctggtgtgtttggtaattattcttacttatacattatacacagatgcgtattgtgttgagtatgaggaaagtgaggaagataaacaacagtgcggtagtagtagtaattttcctgcgagtttaccgcacatgcttagagaactcagggcagcgttcggaaaggtaaaaactttcttccagatgaaagaccaactgaacagtatgctactcacacagtcgctcctcgacgacttcaaaggctacctcgggtgtcaggcactttctgagatgatacagttttacttggaagaggtgatgccgcaggcggaaaatcacgggccggacatcaaagagcacgttaactcgctgggagaaaaactcaaaacgctgcgtcttcgactgcgtcgctgccaccgcttcctgccgtgtgagaacaagagtaaggccgtggagcaagtcaaacgtgtgttcaacatgctgcaggaacgaggtgtttacaaggccatgagcgagttcgacatattcatcaactacatagaatcatacatgactactaaaatgtaaaaatgtatacaacttttagttatcgttcggattctcgtatcgttctgcatactatgtatataaaatgtatattaacatagttacagttacagttacagctatatttttatgctcacaagatgctatataattgaaaggaaattgttcactctctgtcagggcgccatggactttctaggcgccgcgcttcacgactacgttgccgatgcgcccaaggtctgcgccgaggaggtgcggcggctgctggccgcaggcgcctctgtggagtacgcgggcgagttcgggaagaccgcgctgcaccagtacatgggccgttccggcgcggaccccgccgtcgtgcgcgcgctgctggacgccggcgcgcgcgtggacctcccggagacctgctgcggctgcacgcccgtgcacctctgcctcatggccgccaatatcgacgtggaggttctccgcatgctcgtccacgagggccgcgtcgaggactgcggccgcgccgagctcgcctccgtggtgctcaaggagttcgtggtgaaccgcgccttcgacgagaacgtcagcgagcgagtgatgcgcgttcttgtggccgcgggcgcggacgtgaacgccgccagcgtggtcgaccgcacgccgctgcacgtctgcctcacgggcatgtccacgcacccgggcaccatcgccgcgctgctgcgcttcggcgcggacgtgaacgccgtggacctctgcggcatgtcgccgctggcggtgctcgtgcgctcgcgcgcggcgaccgcagagctggtgcgcatgctgctcgacgcgggcgcagacgcacacgctgtcgacagtcgcctggactcgctgctgcaccagcactttcagtccgcgcgcccgcggccggaggtggtgcgcgagctcatccgccacggctgctcgccgcgggcgcggaaccgaatcggcaacacgccgctgcacgaggccgcaaaacactcctcctgcaaacactcgctggtggggccgctgctggctgccggcgcgagcgtggacgcgcgaaataacacgggcaggacgccgctccacttggcggtggcgtccaacccgcgcgcgtgccgccggctgatcgcgcttggggcggacgtggtcgcgcgcagttacgcgggcgtcacgccgctggcgcagctgatcgcggacaataactccgcgctggtgaccgcggcgctgaacacgcagcccgagccgcgggccgtggcagagtcgctgcgagcgaccacgcccgtcggcgagacagcgtgctcgcggctctgtgtggcgtacgtggtggcgcgcgcgccgagcgaggtcctcggcgagcccgagcgcgccctgcacgcggccttcgtggcggagtgcttagcggaggtagcggccatacacgccgtgcgctgcggcacacctccggtctcgctgctggagatcctggtggccgcgcgcccgccgcggagcctgctctcgcgccgcgcgtggcggctggccagccggacgacagtttaccgcgcgccgctccgtgcacgcatcgcggccatgcgccatcgctcgcgactggtggagcgcgcgctgcgcacgctgcgcggctgcgtgctcccgcgcgaggtgctggagcgcgtgctgcggtgtctgtccacacaggacctgcgggcctccggactggccgagtagctttttctgagataagtgaataaacatggtgggattcgatcggcgccgccaacgccacgccatggacgccgccgagatggaggatctcgacatcaacgcggagtcggcgctgtacgactacttcatcctgaacgcggacagagcccgcgtgggcgaggtggttatgcttctcgcacagggcgcggaaataaactacgcggacagcttcgacaagacgccgctgcacctgtacttgcacacgcgacacccgcgctcggacgtgattctggcgctgatggaggcgggcgcggtcgtggacacgccggagcgctgctgcggcgcgaccgcggcgcacctgtacatcctcaacgcggccaaggtcgacctgtcggtgctggaggccatgctgacctggggcgtgcgccagaacgaccagcactcggagcgggtgctctcgagcttgttgcgcgagtacgtggtgacccgcgcctactcggatcagaccgagccgatcatggacttgctcatcggcatgggcgccgacgtggacatgccggtcggcgtgagtcgcacggggctgcacgcctgccttacgggcctgaacgcgaacccgtgcatgattcgcgcgctgcttcggcgcggcgccagcgtgaccgcaaaagacacctacgagatgacgccgctggcggtgctgctgaagtccgcgagcgcgacgccggagctcgtgcgcatcctcgtggaggcaggctccgacgtgagcgccaccgacttccgcctcaacggcatgctgcaccagcacgcgcagtccacgcgcccgcgcgcgagcgtcatgcgcgagctcatccggctggggtgcagcccagcggccaaaaacatgtttgggaacacgccgatgcacatgctggccatggaaagctcctgccgccgctcactgatcctcccgctgctggaggcagggctttccgtgaacgaggagaacccgcactacggcaccgtgcctctgcacgtggcctcggggtacgacaacacgcagggctgcctcaagctcctccgggatggaggagaccccaccgtcgtgtcggccgccggacgcacgccgctctcgaacatgctcgtcaaacgcaaccacgtggcggtcgccggcgcgctgtcgacgcacccgagcgcggcagtagtcgtgcaggctctcgagcaggctctcgagaacgtgctgaacgccgggcccagcgaggcctcgcggctcgccgtggcctttgtggtggcgcgcgccggcgcatccgcgctaccggaggccgtgcgccgtctgcacgagggctttgtcgccgactgcgagcgcgaagtcgagctgctttcccgcaccatgctcggcacaccggccgtgagcgcgctggtcgtgctggtcagcaaggaggtctttggcactgttatctcctcgcgtgcgctgcgcgtcgtgcgggaggtccgcgtgtacgcaaggccgctccgcgaggcgctcataaatctgcgccacaaatgccgcttagtttccagccttaaaaggcaggtgggaccttgctcgctgcccggcgaactggtggagcgcgtgctcgcgaccgtgccactgaccgacttgcgccgctcgtgcggccgccgcgcgcccgagtaactgcccgtcccgttgctacgcgactcgagactgcccgctgtttttctttccccgtttcttcttattaggagttgttgcccgcctccatgatcctcgcacgcgccggcgggcaacctcgcacgcccgcggcggccgcgggcgccgccgaggacggcgagcacagtgatcgccggaagcgcaagcgcaagacgcccaactgcgaagacgccgacaactccgacgacgagctagcgcagacgccgtgcgaccgcgagtggccggactgtcgcgcgagctcgatcacgagctccgactcggtctctctcggcgacgagatctacctgcgatacgtggcctcgcaggtggacttcgcgcagacctgggccccgccagtgcggctgctgcgctccttcgggaacttctcgaaggaaacgctcaaccgcatgtcgcggcgcgggtacgtgaaccgctcctacttccagatggcgcacgcgcgcttctcgcccaccaacgacgacatgtaccacatggccacgggcgggtacggcatcgtgttccgcttcgaccgctacgtggttaagtacgtcttcgagcaccgcaacggcatgtccgagatggacgcctctacggagtacacggtgccgcggttcctgcgcaataacctcaagggcgacgagcgcgagttcgtggtctgcgcgctgcccatggggctgaactaccggctgggcttcctgcactcgctgtaccggcgcgtgctgcacacgctgctgctgctcatgcgcgtggaggaaggccagcggccctcggtggagatgtccaagaagccgctgctgcgctggttcgaggcgcgcaaggacagcgagtccttcgtgcgcctgatctcgtacttctacccctcggccgtgcagagcaacgtgaacctgatcaacaacttccaccacctggtgcacttcttcgagcacgagaagcgcgcgcggtacgtgttcgaccgcggggccgtgatcgtgttccctctggcgcgcgggtccgcggactcgatctcgccggaggcggcggcggcgctgggcttcgcgccgcactcggagttcctcaagttcgtgttcctgcagatcgcgctgctgtacctgaagatctacgagctcccgggctgcacgaacttcctgcacgtggacctgaagcccgacaacgtgctcatcttcgacagtgcgcgcgcgctcagcgtgaccgcggccggcgcgactttccgcttcgaggagcccgtgcgcgcggcgctgaacgacttcgacttcgcgcgcgtggccaccatcgagaaccgcaagatcgcgggcagcgtccgcgtgccgcagaactggtactacgacttccacttcttcgcgcacacgctgctgcgcgcgtacccgcacatcgccgcggaggacccgggcttccacgcgctgctctcggagctcacggtctcgtgctcgcgcgggacctgcgaccgcttccggctgcgcgtgtcctcgccgcaccccatcgagcacctcgcgcggctggtgcgccgcgacgtcttctcccgctggataaatgccgccgcggacgcccccgacgccgccgcactctcctgagcccacgcccgcggcgccgggctcgctgtacgacgtcttcctcgcgcgcttcctgcgccggctggccgcgcgcgcggcgccggcctcggccgcctgcgccgtgcgcgtgggtgcggtgcgcggccgcctgcggaactgcgagctagtggtgctgaaccgctgccacgcggacgcggccggcgcgctcgcgctagcctccgcggcgctcgccgagacgctggcggagctcccgcgcgcggacaagctcgccgtcgcgctcgagctgggcgtggaccccgagcacccggagctgacgccggaccccgcctgcgcaggcgagagcgcactcgcacagaacatcgacatccagacgctggacctgggcgactgcggagaccccaaaggccgccgactgcgcgtggcgctggtgaacagcggccacgcggccgcgaactgcgcgctcgcgcgcgtggcgaccgcgctgacgcgccgcgtgcccgcgagccggcacggcctcgcggagggcggcacgccgccgtggacgctgctgctggcggtggccgcggtgacggtgctcggcgtggtggcggtttcactgctgcggcgcgcgctgcgggtacgctaccgcttcgcgcggccggccgcgctgcgcgcgtagccgcgcaaaatgtaaattataacgcccaacttttaagggtgaggcgccatgaagttgctcgtcggcatactagtagccgtgtgcttgcaccagtatctgctgaacgcggacagcaacacgaaaggatggtccgaagtgctgaaaggcagcgagtgcaagcctaggccgattgttgttcctgtaagcgagacgcacccagagctgacttctcagcggttcaacccgccgtgtgtcacgttgatgcgatgcggcgggtgctgcaacgacgagagcttggaatgcgtccccacggaagaagtaaacgtgacgatggaactcctgggggcgtcgggctccggtagtaacgggatgcaacgtctgagcttcgtagagcataagaaatgcgattgtagaccacgattcacaaccacgccaccgacgaccacaaggccgcccagaagacgccgctagaactttttatggaccgcagatccaaacgatgatgcgatcaggtcatgcggaagaaggcgccacggagcaaagtgaaaaaggaccgcctagcagtcgagaccctcccgccgcagccgcggacaccccacacccgccttccacccgccagacgccaacaccgcagccaacaagcatgcacccctcgccgcgcaggctgctcggcgcgctcgcgctggtggcgctgggcttcctcctcggcgggctcttccgccccgcggcgccgccgctgccggccgccctcgtggaggcgggccccgtccgcgcgaacggctccgcctcggtgacctgcctgaccgtcggcggcgacgggcggcacatggcggtggtcgcgcacggcggcgggacgctctcgccggtgtacccgctcgccgccggcatgcacgcgaccttcgcctcgctgcgcaagggcgcgctgctgctgaacgtcgcgaccgtgcacatctacgacgtgcgcgcgctcgggccggagttcgagctgacctgcgtcgcggtggcgggcggctacaacgcggcctgggcggccgcgcggcccgcggccgagtggcgccgccagctggcgcggatgcaccgctcggagctgtgaccctctccctcccggtctcccatccgtttttgtaatcggccttagtagattagaccagcatcccgcgcccttgtccgagaacaagtaacagtaaccgttacctcactcgccactcctcggaataatagaacgagagaacgagagaacgagttaaccgttgctcactcgctcactcggtgtgagagaacaagtaacgttgctcactcgctcactcggtgtgagagaacaagagaacgagtagctgttgctaactcaatcacccctcggagtaagagaacaagagcagtcaactacccactcagtcttggatgagaggcagaggacgagttgacgagttgaacagttaatcctcactcactcagagcgagagagcgagagagtggaggacgagttaacaagtcaatcctcactcagagtgagagagtgagagagtggaggacgagttaatggttaacagttatcaccactcagagtgagagcggaggacgagtcaaccactcgctcgcccactccgagttagagagggaaccagtgcgagttaacgcgcacacgagcgagagaacgtaaactcgctcgcgcgctcgctcggctaaccgtcggcctctcccaaaactcttcgtaaaactttcccgatgacagttcaccctccaaaactttgtaaaactaaactgttcggaggtcggtctgctgcctctctaactctccgtaaaacgtttgtaaactgtcggaggtcggtgacccgctcaaccgtccgcgaaaacttttcgcaggcagtgtctgcctctctcggactctccgcaaacactttcgcggaacctcggagggtggtcgacctctctccaaactcttgcaaaactttttcgcggaaccgttggaggccagtcctccctccaaactctttgtaaaatcttttcgaggccagtcctcctctccaaaacgttccgcaaaatctttgggaggtcggcctctcctctccagaacgttccgtaaaactcttggaggccgcccgcggcacgcgaggcggaggatccgaggtgtcgacctccctcaaaaactttgtaaaaactttttataaaactttccgcggaacctcggagagtaggtcgacctctctcaaaactcttataaaacttttccgcggaaccgttggaaggtaggtcgacctctctcaaaactcttataaaacttttccgcggaaccgttggaaggtaggtcgacctctctcaaaactcttataaaacttttccgcggaaccgttggaggcaggtcggcctctcaaactctttgcgagaactcttcgcgagaactcttcgataactttaggaggtcaggtcgacctcccaaaacttttgcgagaactctctgtaaaactttaggaggtcggtacctccctcaaaactttttataaaacttttcgcggaacctccggagacgggccgccgccgcgaccgcgggagcggagaggccgacctcccgagacgttccgcgttaccgtcggggtaggcgtcctctcgagaacgccaaaagacttcgtgcaaaaacttttcggaggggcgcggagggcgggtcagctcccgcgaactcccgcagaaccttttcgcgcgaccgcgaaggccggccgcctctcccacactctcaagagcttttcggaggagaggaagggcaggtcgcccccacctccgacgctttgtaaaaacgtttacgcggaacctcggaggcaggtcgcctccctcgaaaactcctcgcgaaacctttaaaaactttttgcgaaaacttttcggaggatgtcggagggcgggcggctcttccaaacctccgcagaaccttttcgcgcaaccgttggaagacaggtcggcctctctcgaaaacttttaaaactttgtaaacgcgttggcgggaccgtcgcgggagagcggccgcccgcggcacgcgagaggaggaaccgttggaaggcagtcggcctctcccgaaaactttttataaaaacttttccgcggaaccgttggaggcaggtcggcctctctcagagtctaaaaactttttgcgggactcggacggcgcggtcacccgaccacctgactcctgtctcacccgtactacttggacttctgtttccctgactcccgactccctgacctcccgactccctgactcccgactccctgactcccgactccctgactcccgactccctgactcccgactccctgactcccgactccctgactcccgactccctgactcccgactccctgactcccgactccctgactcccgactccctgactcccgactccctgactcccgactccctgactcccgactccctgactcccgactccctgactcccgactccctgactccctgactccagagcgaggtctcgcggctgcggggtgccgcctccgcggagtcgcgttcccgcggacgcccgtcctcgaaagcattcagcagttccagcctctgccgtagctcctcccgcaggaactcctggtccgcgttcgtcgcggcaccgcggctcagccgccgcgggagccggccgccgcccgcgaagccgcggatcc